(19) United States
(12) Patent Application Publication
(10) Pub. No.: US 2019/0054112 A1
(43) Pub. Date: Feb. 21, 2019

(54) POLYNUCLEOTIDE FORMULATIONS FOR USE IN THE TREATMENT OF RENAL DISEASES

(71) Applicant: Moderna Therapeutics, Inc., Cambridge, MA (US)

(72) Inventor: Francine M. GREGOIRE, Newton, MA (US)

(21) Appl. No.: 15/760,659

(22) PCT Filed: Sep. 16, 2016
(86) PCT No.: PCT/US16/52117

§ 371 Date: Mar. 16, 2018

Related U.S. Application Data
(60) Provisional application No. 62/220,282, filed on Sep. 18, 2015.

(51) Int. Cl. A61K 31/7115; A61K 38/17; C12N 15/62; C12N 15/113; A61K 47/14; A61K 47/24; A61K 47/28; A61K 9/127; C12N 15/85; A61K 47/18; A61K 47/10; C12N 15/88; A61P 13/12; A61K 48/00

(52) U.S. Cl. CPC A61K 31/7115; A61K 48/0083; C12N 15/62; C12N 15/113; A61K 47/14; A61K 47/24; A61K 47/28; A61K 9/1271; C12N 15/85; A61K 47/186; A61K 47/183; A61K 47/10; C12N 15/88; A61P 13/12; A61K 38/17

(57) ABSTRACT

The present invention relates to compositions and methods for the preparation, manufacture and therapeutic use of renal polynucleotides.

### REFERENCE TO SEQUENCE LISTING

The present application is being filed along with a Sequence Listing in electronic format. The Sequence Listing is provided as a file entitled M146SEQLST.txt, created on Sep. 17, 2015 which is 3,624,089 bytes in size. The information in the electronic format of the sequence listing is incorporated herein by reference in its entirety.

### FIELD OF THE INVENTION

The invention relates to polynucleotides encoding targets associated with renal disease and polynucleotide formulations, methods, processes, kits and devices using the polynucleotide formulations in the treatment of renal diseases.

### BACKGROUND OF THE INVENTION

Renal diseases are very common with more than 3 million diagnosed each year in the United States alone. Kidneys filter approximately 200 liters of fluid per day in order to remove waste and drugs from blood to maintain overall health. Additionally, kidneys balance water and mineral concentrations in the blood, release a hormone to regulate blood pressure, produce an active form of vitamin D, and control the production of red blood cells. Because of these vital functions, kidney, or renal, diseases pose significant, systemic dangers to human life.

Current renal disease treatments include dialysis or transplantation. Dialysis replicates the function of the kidney through machine based filtering to adjust mineral concentration and filtering products from the blood. The dialysis process is time consuming and includes risks such as bleeding, infection, low blood pressure, and air bubbles in the blood. Transplantation replaces a person's kidney with a working kidney from a donor. Transplantation involves a long waiting time for an acceptable donor to arise carries risks of blood clots, infection, organ rejection, and organ failure. The current methods fail to provide long-term solutions with little risk.

The present invention addresses the need for a better treatment methodology by providing an alternate system for treating renal diseases. By administering therapeutic formulations of nucleic acid based compounds or polynucleotides, which have structural and/or chemical modifications that avoid one or more problems in the art. For example, optimized formulations for delivery of the therapeutic polynucleotide retain structural and functional integrity in order to overcome the threshold of expression, improve expression rates, optimize expression localization, and avoid deleterious bio-responses by the immune system. These barriers may be reduced or eliminated using the present invention.

### SUMMARY OF THE INVENTION

Described herein are polynucleotides encoding targets associated with renal disease and polynucleotide formulations, methods, processes, kits and devices using the polynucleotide formulations for the treatment of renal diseases, disorders and/or conditions.

Provided herein are renal polynucleotides (e.g., mRNA) encoding at least one renal polypeptide of interest. The renal polynucleotide may comprise at least one chemical modification described herein. As a non-limiting example, the chemical modification may be 1-methylpseudouridine, 5-methylcytosine or 1-methylpseudouridine and 5-methylcytosine.

Also provided herein are pharmaceutical compositions comprising at least one mRNA encoding a renal polypeptide of interest. The mRNA may be formulated in a lipid nanoparticle comprising at least one lipid such as, but not limited to, KL10, KL22, KL52, C12-200, DLin-KC2-DMA, DOPE, and DSPC. The lipid nanoparticle may also comprise between 1% and 7% of a PEG lipid. The N:P ratio of the lipid nanoparticle may be between 2.5 and 7, the ratio of lipid to mRNA may be 10:1 or 20:1, the particle size of the lipid nanoparticle may be between 50 nm and 150 nm and the encapsulation efficicancy may be greater than 50%.

Provided herein are method of producing a renal polypeptide in a kidney of a subject using arterial administration of the renal compositions described herein (e.g., compositions comprising at least one renal polynucleotide). A subject may be dosed with 5-45 μg per 0.5 ml per kidney and the expression of the renal polypeptide in the kidney may be increased for at least 3 hours.

Provided herein are methods of treating a renal disease, disorder or condition using arterial administration of the renal compositions described herein (e.g., compositions comprising at least one renal polynucleotide). A subject may be dosed with 5-45 μg per 0.5 ml per kidney. The renal disease, disorder or condition may be, but is not limited to, primary glomerular disease, cystic renal disease and renal tubular disease. Primary glomerular diseases include, but are not limited to, Alport's syndrome (X-linked or autosomal recessive), benign familiar hematuria, congenital nephrosis I, nail patella syndrome and familial mesangial sclerosis. Cystic renal diseases include, but are not limited to, polycystic kidney disease 1 (PKD1), polycystic kidney disease 2 (PKD2), and infantile severe polycystic kidney disease with tuberous sclerosis. Renal tubular diseases include, but are not limited to, distal renal tubular acidosis, renal tubular acidosis with neural deafness, renal tubular acidosis with osteoporosis, Dent's disease, Nephrogenic diabetes insipidus (X-linked), Nephrogenic diabetes insipidus (autosomal), familial hypocalcuric hypercalcemia, pseudovitamin D deficiency rickets, X-linked hypophosphatemia, Gitelman's syndrome, Bartter's syndrome type 1, Bartter's syndrome type 2, Bartter's syndrome type 3, Pseudoaldosteronism (Liddle syndrome), Recessive pseudohypoaldosteronism type 1, dominant pseudohypoaldosteronism type I, apparent mineralocorticoid excess, Cystinuria type I and Cystinuria non-type I.

The details of various embodiments of the invention are set forth in the description below. Other features, objects, and advantages of the invention will be apparent from the description and the drawings, and from the claims.

### BRIEF DESCRIPTION OF THE DRAWINGS

The foregoing and other objects, features and advantages will be apparent from the following description of particular embodiments of the invention, as illustrated in the accompanying drawings in which like reference characters refer to the same parts throughout the different views. The drawings are not necessarily to scale, emphasis instead being placed upon illustrating the principles of various embodiments of the invention.

FIG. 1A and FIG. 1B are schematics of an IVT polynucleotide construct. FIG. 1A is a schematic of a polynucleotide construct taught in commonly owned co-pending U.S. patent application Ser. No. 13/791,922 filed Mar. 9, 2013, the contents of which are incorporated herein by reference. FIG. 1B is a schematic of a linear polynucleotide construct.

FIG. 2 is a histogram showing the expression in the kidney, spleen and liver.

### DETAILED DESCRIPTION

It is of great interest in the fields of therapeutics, diagnostics, reagents and for biological assays to be able design, synthesize and deliver a nucleic acid, e.g., a ribonucleic acid (RNA) inside a cell, whether in vitro, in vivo, in situ or ex vivo, such as to effect physiologic outcomes which are beneficial to the cell, tissue or organ and ultimately to an organism. One beneficial outcome is to cause intracellular translation of the nucleic acid and production of at least one encoded peptide or polypeptide of interest. In like manner, non-coding RNA has become a focus of much study; and utilization of non-coding polynucleotides, alone and in conjunction with coding polynucleotides, could provide beneficial outcomes in therapeutic scenarios.

Described herein are compositions (including pharmaceutical compositions) and methods for the design, preparation, manufacture and/or formulation of renal polynucleotides which may be used to treat renal disease.

Also provided are systems, processes, devices and kits for the selection, design and/or utilization of the renal polynucleotides described herein.

According to the present invention, the renal polynucleotides are preferably modified in a manner as to avoid the deficiencies of other molecules of the art.

The use of polynucleotides such as modified polynucleotides encoding polypeptides (i.e., modified mRNA) in the fields of human disease, antibodies, viruses, veterinary applications and a variety of in vivo settings has been explored previously and these studies are disclosed in for example, those listed in Table 6 of co-pending International Publication Nos. WO2013151666, WO2013151667, WO2013151668, WO2013151663, WO2013151669, WO2013151670, WO2013151664, WO2013151665, WO2013151736, WO2013151671 and WO2013151672 and Table 178 of International Publication No. WO2013151671; the contents of each of which are herein incorporated by reference in their entireties. Any of the foregoing may be synthesized as an IVT polynucleotide, chimeric polynucleotide or a circular polynucleotide and such embodiments are contemplated by the present invention.

Provided herein, therefore, are renal polynucleotides which have been designed to improve one or more of the stability and/or clearance in tissues, receptor uptake and/or kinetics, cellular access, engagement with translational machinery, mRNA half-life, translation efficiency, immune evasion, immune induction (for vaccines), protein production capacity, secretion efficiency (when applicable), accessibility to circulation, protein half-life and/or modulation of a cell's status, function and/or activity.

### I. COMPOSITIONS OF THE INVENTION

### Renal Polynucleotides

The present invention provides renal nucleic acid molecules, specifically renal polynucleotides which, in some embodiments, encode one or more renal peptides or renal polypeptides of interest. The term “nucleic acid,” in its broadest sense, includes any compound and/or substance that comprise a polymer of nucleotides. These polymers are often referred to as polynucleotides.

Exemplary renal nucleic acids or renal polynucleotides of the invention include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization), ethylene nucleic acids (ENA), cyclohexenyl nucleic acids (CeNA) or hybrids or combinations thereof.

Provided herein are pharmaceutical compositions comprising at least one reanl polynucleotide such as, but not limited to, a renal IVT polynucleotide or a renal chimeric polynucleotide.

In one embodiment, the renal polynucleotide may take the form or function as modified mRNA molecules which encode at least one renal polypeptide of interest.

In one embodiment, the length of a region encoding at least one renal polypeptide of interest of the renal polynucleotides present invention is greater than about 30 nucleotides in length (e.g., at least or greater than about 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000, 4,000, 5,000, 6,000, 7,000, 8,000, 9,000, 10,000, 20,000, 30,000, 40,000, 50,000, 60,000, 70,000, 80,000, 90,000 or up to and including 100,000 nucleotides). As used herein, such a region may be referred to as a “coding region” or “region encoding.”

In one aspect, at least the coding region of the renal polynucleotide is codon optimized.

In one embodiment, the renal polynucleotides of the present invention may encode at least one renal peptide or renal polypeptide of interest. In another embodiment, the renal polynucleotides of the present invention may be non-coding.

In one embodiment, the renal polynucleotides of the present invention is or functions as a messenger RNA (mRNA). As used herein, the term “messenger RNA” (mRNA) refers to any renal polynucleotide which encodes at least one renal peptide or renal polypeptide of interest and which is capable of being translated to produce the encoded renal peptide or polypeptide of interest in vitro, in vivo, in situ or ex vivo.

In one embodiment, the renal polynucleotides of the present invention may be structurally modified or chemically modified. When the renal polynucleotides of the present invention are chemically and/or structurally modified the renal polynucleotides may be referred to as “modified polynucleotides” or “modified renal polynucleotides.”

As used herein, a “structural” modification is one in which two or more linked nucleosides are inserted, deleted, duplicated, inverted or randomized in a renal polynucleotide without significant chemical modification to the nucleotides themselves. Because chemical bonds will necessarily be broken and reformed to effect a structural modification, structural modifications are of a chemical nature and hence are chemical modifications. However, structural modifications will result in a different sequence of nucleotides. For example, the polynucleotide “ATCG” may be chemically modified to “AT-5meC-G”. The same polynucleotide may be structurally modified from “ATCG” to “ATCCCG”. Here, the dinucleotide “CC” has been inserted, resulting in a structural modification to the polynucleotide.

In one aspect, the renal polynucleotide may comprise at least one modification such as a modified nucleoside. The at least one modification may be located on one or more nucleosides such as, but not limited to the sugar and/or the nucleobase. As a non-limiting example, the at least one modification may be 1-methylpseudouridine.

In another aspect, the renal polynucleotide may comprise at least two modifications. The at least two modifications may be located on one or more of a nucleoside and/or a backbone linkage between nucleosides, both a nucleoside and a backbone linkage. The backbone linkage may be modified by the replacement of one or more oxygen atoms or with a phosphorothioate linkage. As a non-limiting example, the at least two modifications may be 1-methylpseudouridine and 5-methycytidine.

In one embodiment, the renal polynucleotides of the present invention, may have a uniform chemical modification of all or any of the same nucleoside type or a population of modifications produced by mere downward titration of the same starting modification in all or any of the same nucleoside type, or a measured percent of a chemical modification of all any of the same nucleoside type but with random incorporation, such as where all uridines are replaced by a uridine analog, e.g., pseudouridine. In another embodiment, the renal polynucleotides may have a uniform chemical modification of two, three, or four of the same nucleoside type throughout the entire renal polynucleotide (such as all uridines and all cytosines, etc. are modified in the same way).

In one embodiment, if the renal polynucleotides of the present invention are chemically modified they may have a uniform chemical modification of all or any of the same nucleoside type or a population of modifications produced by mere downward titration of the same starting modification in all or any of the same nucleoside type, or a measured percent of a chemical modification of all any of the same nucleoside type but with random incorporation, such as where all uridines are replaced by a uridine analog, e.g., pseudouridine. In another embodiment, the renal polynucleotides may have a uniform chemical modification of two, three, or four of the same nucleoside type throughout the entire renal polynucleotide (such as all uridines and all cytosines, etc. are modified in the same way).

In one embodiment, the renal polynucleotide may include modified nucleosides such as, but not limited to, the modified nucleosides described in US Patent Publication No. US20130115272 including pseudouridine, 1-methylpseudouridine, 5-methoxyuridine and 5-methylcytosine. As a non-limiting example, the polynucleotide may include 1-methylpseudouridine and 5-methylcytosine. As another non-limiting example, the polynucleotide may include 1-methylpseudouridine. As yet another non-limiting example, the renal polynucleotide may include 5-methoxyuridine and 5-methylcytosine. As a non-limiting example, the renal polynucleotide may include 5-methoxyuridine.

In another embodiment, the renal polynucleotides of the present invention which have portions or regions which differ in size and/or chemical modification pattern, chemical modification position, chemical modification percent or chemical modification population and combinations of the foregoing are known as “chimeric polynucleotides” or “chimeric renal polynucleotides.” A “chimera” according to the present invention is an entity having two or more incongruous or heterogeneous parts or regions. As used herein a “part” or “region” of a renal polynucleotide is defined as any portion of the renal polynucleotide which is less than the entire length of the renal polynucleotide.

In one embodiment, the chimeric renal polynucleotides may take the form or function as modified mRNA molecules which encode at least one renal polypeptide of interest. In one embodiment, such chimeric renal polynucleotides are substantially non-coding.

Methods of making chimeric polynucleotides are described in International Publication No. WO2015034928, the contents of which are herein incorporated by reference in its entirety.

In one embodiment, the renal polynucleotides of the present invention are circular and they are referred to as “circular polynucleotides,” “circular renal polynucleotides” or “circP.” As used herein, “circular polynucleotides,” “circular renal polynucleotides” or “circP” means a single stranded circular renal polynucleotide which acts substantially like, and has the properties of, an RNA. The term “circular” is also meant to encompass any secondary or tertiary configuration of the circP.

Circular polynucleotides are described in International Publication No. WO2015034925, the contents of which are herein incorporated by reference in its entirey.

### Renal Polynucleotide Architecture

Traditionally, the basic components of an mRNA molecule include at least a coding region, a 5′UTR, a 3′UTR, a 5′ cap and a poly-A tail. The renal polynucleotides of the present invention may function as mRNA but are distinguished from wild-type mRNA in their functional and/or structural design features which serve to overcome existing problems of effective renal polypeptide production using nucleic-acid based therapeutics.

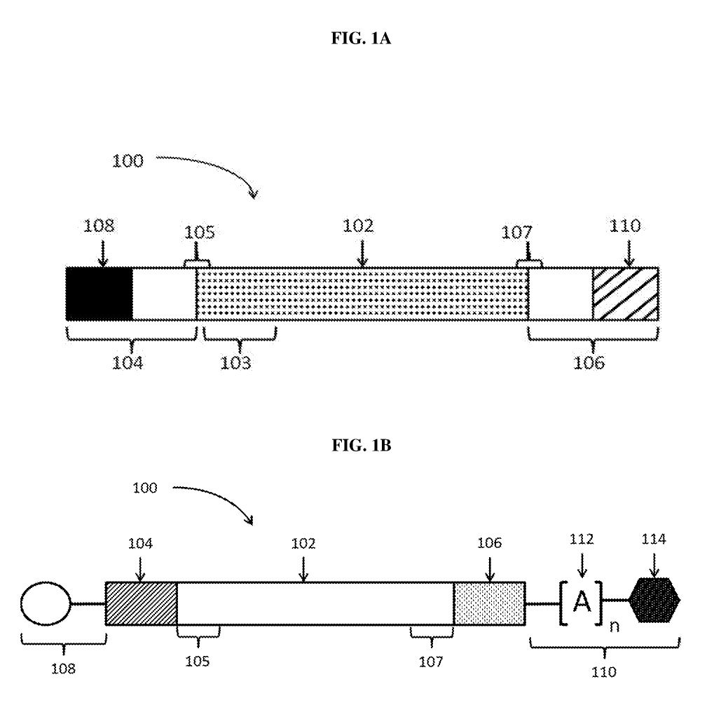

FIG. 1 shows a representative renal polynucleotide primary construct 100 of the present invention. As used herein, “primary construct” refers to a renal polynucleotide of the present invention which encodes one or more renal polypeptides of interest and which retains sufficient structural and/or chemical features to allow the renal polypeptide of interest encoded therein to be translated.

Renal polynucleotide primary construct refers to a renal polynucleotide transcript which encodes one or more renal polypeptides of interest and which retains sufficient structural and/or chemical features to allow the renal polypeptide of interest encoded therein to be translated. Non-limiting examples of renal polypeptides of interest and renal polynucleotides encoding renal polypeptide of interest are described in Table 3 herein and Table 6 of co-pending International Publication Nos. WO2013151666, WO2013151667, WO2013151668, WO2013151663, WO2013151669, WO2013151670, WO2013151664, WO2013151665, WO2013151736, WO2013151671 and WO2013151672 and Table 178 of International Publication No. WO2013151671, the contents of each of which are incorporated herein by reference in their entirety.

According to A and B of FIG. 1, the primary construct 100 of a renal polynucleotide here contains a first region of linked nucleotides 102 that is flanked by a first flanking region 104 and a second flaking region 106. As used herein, the “first region of linked nucleosides” may be referred to as a “coding region” or “region encoding” or simply the “first region.” This first region may include, but is not limited to, the encoded renal polypeptide of interest. In one aspect, the first region 102 may include, but is not limited to, the open reading frame encoding at least one renal polypeptide of interest. The open reading frame may be codon optimized in whole or in part.

The renal polypeptide of interest may comprise at its 5′ terminus one or more signal sequences encoded by a signal sequence region 103.

The first flanking region 104 may comprise a region of linked nucleosides which function as a 5′ untranslated region (UTR) such as the 5′ UTR of any of the nucleic acids encoding the native 5′UTR of the renal polypeptide or a non-native 5′UTR such as, but not limited to, a heterologous 5′UTR or a synthetic 5′UTR. The flanking region 104 may comprise a region of linked nucleotides comprising one or more complete or incomplete 5′ UTRs sequences which may be completely codon optimized or partially codon optimized. The flanking region 104 may include at least one nucleic acid sequence including, but not limited to, miR sequences, TERZAK™ sequences and translation control sequences. The flanking region 104 may also comprise a 5′ terminal cap 108. The 5′ terminal capping region 108 may include a naturally occurring cap, a synthetic cap or an optimized cap. Non-limiting examples of optimized caps include the caps taught by Rhoads in U.S. Pat. No. 7,074,596 and International Patent Publication No. WO2008157668, WO2009149253 and WO2013103659, the contents of each of which are herein incorporated by reference in its entirety. The second flanking region 106 may comprise a region of linked nucleotides comprising one or more complete or incomplete 3′ UTRs which may encode the native 3′ UTR of the renal polypeptide or a non-native 3′UTR such as, but not limited to, a heterologous 3′UTR or a synthetic 3′ UTR. The second flanking region 106 may be completely codon optimized or partially codon optimized. The flanking region 106 may include at least one nucleic acid sequence including, but not limited to, miR sequences and translation control sequences. The flanking region 106 may also comprise a 3′ tailing sequence 110. The 3′ tailing sequence 110 may be, but is not limited to, a polyA tail, a polyC tail, a polyA-G quartet and/or a stem loop sequence.

As shown in B of FIG. 1, the 3′ tailing sequence 110 may include a synthetic tailing region 112 and/or a chain terminating nucleoside 114. Non-liming examples of a synthetic tailing region include a polyA sequence, a polyC sequence, and a polyA-G quartet. Non-limiting examples of chain terminating nucleosides include 2′-O methyl, F and locked nucleic acids (LNA).

Bridging the 5′ terminus of the first region 102 and the first flanking region 104 is a first operational region 105. Traditionally this operational region comprises a Start codon. The operational region may alternatively comprise any translation initiation sequence or signal including a Start codon.

Bridging the 3′ terminus of the first region 102 and the second flanking region 106 is a second operational region 107. Traditionally this operational region comprises a Stop codon. The operational region may alternatively comprise any translation initiation sequence or signal including a Stop codon. Multiple serial stop codons may also be used in the renal polynucleotide. In one embodiment, the operation region of the present invention may comprise two stop codons. The first stop codon may be “TGA” or “UGA” and the second stop codon may be selected from the group consisting of “TAA,” “TGA,” “TAG,” “UAA,” “UGA” or “UAG.”

The shortest length of the first region of the primary construct of the renal polynucleotide of the present invention can be the length of a nucleic acid sequence that is sufficient to encode for a dipeptide, a tripeptide, a tetrapeptide, a pentapeptide, a hexapeptide, a heptapeptide, an octapeptide, a nonapeptide, or a decapeptide. In another embodiment, the length may be sufficient to encode a renal peptide of 2-30 amino acids, e.g. 5-30, 10-30, 2-25, 5-25, 10-25, or 10-20 amino acids. The length may be sufficient to encode for a renal peptide of at least 11, 12, 13, 14, 15, 17, 20, 25 or 30 amino acids, or a renal peptide that is no longer than 40 amino acids, e.g. no longer than 35, 30, 25, 20, 17, 15, 14, 13, 12, 11 or 10 amino acids. Examples of dipeptides that the renal polynucleotide sequences can encode or include, but are not limited to, carnosine and anserine.

The length of the first region of the primary construct of the renal polynucleotide encoding the renal polypeptide of interest of the present invention is greater than about 30 nucleotides in length (e.g., at least or greater than about 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000, 4,000, 5,000, 6,000, 7,000, 8,000, 9,000, 10,000, 20,000, 30,000, 40,000, 50,000, 60,000, 70,000, 80,000, 90,000 or up to and including 100,000 nucleotides).

In some embodiments, the renal polynucleotide includes from about 30 to about 100,000 nucleotides (e.g., from 30 to 50, from 30 to 100, from 30 to 250, from 30 to 500, from 30 to 1,000, from 30 to 1,500, from 30 to 3,000, from 30 to 5,000, from 30 to 7,000, from 30 to 10,000, from 30 to 25,000, from 30 to 50,000, from 30 to 70,000, from 100 to 250, from 100 to 500, from 100 to 1,000, from 100 to 1,500, from 100 to 3,000, from 100 to 5,000, from 100 to 7,000, from 100 to 10,000, from 100 to 25,000, from 100 to 50,000, from 100 to 70,000, from 100 to 100,000, from 500 to 1,000, from 500 to 1,500, from 500 to 2,000, from 500 to 3,000, from 500 to 5,000, from 500 to 7,000, from 500 to 10,000, from 500 to 25,000, from 500 to 50,000, from 500 to 70,000, from 500 to 100,000, from 1,000 to 1,500, from 1,000 to 2,000, from 1,000 to 3,000, from 1,000 to 5,000, from 1,000 to 7,000, from 1,000 to 10,000, from 1,000 to 25,000, from 1,000 to 50,000, from 1,000 to 70,000, from 1,000 to 100,000, from 1,500 to 3,000, from 1,500 to 5,000, from 1,500 to 7,000, from 1,500 to 10,000, from 1,500 to 25,000, from 1,500 to 50,000, from 1,500 to 70,000, from 1,500 to 100,000, from 2,000 to 3,000, from 2,000 to 5,000, from 2,000 to 7,000, from 2,000 to 10,000, from 2,000 to 25,000, from 2,000 to 50,000, from 2,000 to 70,000, and from 2,000 to 100,000).

According to the present invention, the first and second flanking regions of the renal polynucleotide may range independently from 15-1,000 nucleotides in length (e.g., greater than 30, 40, 45, 50, 55, 60, 70, 80, 90, 100, 120, 140, 160, 180, 200, 250, 300, 350, 400, 450, 500, 600, 700, 800, and 900 nucleotides or at least 30, 40, 45, 50, 55, 60, 70, 80, 90, 100, 120, 140, 160, 180, 200, 250, 300, 350, 400, 450, 500, 600, 700, 800, 900, and 1,000 nucleotides).

According to the present invention, the tailing sequence of the renal polynucleotide may range from absent to 500 nucleotides in length (e.g., at least 60, 70, 80, 90, 120, 140, 160, 180, 200, 250, 300, 350, 400, 450, or 500 nucleotides). Where the tailing region is a polyA tail, the length may be determined in units of or as a function of polyA Binding Protein binding. In this embodiment, the polyA tail is long enough to bind at least 4 monomers of PolyA Binding Protein. PolyA Binding Protein monomers bind to stretches of approximately 38 nucleotides. As such, it has been observed that polyA tails of about 80 nucleotides and 160 nucleotides are functional.

According to the present invention, the capping region of the renal polynucleotide may comprise a single cap or a series of nucleotides forming the cap. In this embodiment the capping region may be from 1 to 10, e.g. 2-9, 3-8, 4-7, 1-5, 5-10, or at least 2, or 10 or fewer nucleotides in length. In some embodiments, the cap is absent.

According to the present invention, the first and second operational regions of the renal polynucleotide may range from 3 to 40, e.g., 5-30, 10-20, 15, or at least 4, or 30 or fewer nucleotides in length and may comprise, in addition to a Start and/or Stop codon, one or more signal and/or restriction sequences.

In one embodiment, non-UTR sequences may be used as regions or subregions within the renal polynucleotides. For example, introns or portions of introns sequences may be incorporated into regions of the renal polynucleotides of the invention. Incorporation of intronic sequences may increase protein production as well as renal polynucleotide levels.

### Multimers of Renal Polynucleotides

According to the present invention, multiple distinct renal polynucleotides may be linked together through the 3′-end using nucleotides which are modified at the 3′-terminus. Chemical conjugation may be used to control the stoichiometry of delivery into cells. For example, the glyoxylate cycle enzymes, isocitrate lyase and malate synthase, may be supplied into cells at a 1:1 ratio to alter cellular fatty acid metabolism. This ratio may be controlled by chemically linking renal polynucleotides using a 3′-azido terminated nucleotide on one renal polynucleotide species and a C5-ethynyl or alkynyl-containing nucleotide on the opposite renal polynucleotide species. The modified nucleotide is added post-transcriptionally using terminal transferase (New England Biolabs, Ipswich, Mass.) according to the manufacturer's protocol. After the addition of the 3′-modified nucleotide, the two renal polynucleotides species may be combined in an aqueous solution, in the presence or absence of copper, to form a new covalent linkage via a click chemistry mechanism as described in the literature.

In another example, more than two renal polynucleotides may be linked together using a functionalized linker molecule. For example, a functionalized saccharide molecule may be chemically modified to contain multiple chemical reactive groups (SH—, NH₂—, N₃, etc. . . . ) to react with the cognate moiety on a 3′-functionalized mRNA molecule (i.e., a 3′-maleimide ester, 3′-NHS-ester, alkynyl). The number of reactive groups on the modified saccharide can be controlled in a stoichiometric fashion to directly control the stoichiometric ratio of conjugated renal polynucleotides.

In one embodiment, the renal polynucleotides may be linked together in a pattern. The pattern may be a simple alternating pattern such as CD[CD]ₓ where each “C” and each “D” represent a renal polynucleotide or different renal polynucleotides. The pattern may repeat x number of times, where x=1-300. Patterns may also be alternating multiples such as CCDD[CCDD]ₓ (an alternating double multiple) or CCCDDD[CCCDDD]ₓ (an alternating triple multiple) pattern. The alternating double multiple or alternating triple multiple may repeat x number of times, where x=1-300.

### Bifunctional Renal Polynucleotides

In one embodiment of the invention are bifunctional renal polynucleotides. As the name implies, bifunctional renal polynucleotides are those having or capable of at least two functions. These molecules may also by convention be referred to as multi-functional.

The multiple functionalities of bifunctional renal polynucleotides may be encoded by the RNA (the function may not manifest until the encoded product is translated) or may be a property of the renal polynucleotide itself. It may be structural or chemical. Bifunctional modified renal polynucleotides may comprise a function that is covalently or electrostatically associated with the renal polynucleotides. Further, the two functions may be provided in the context of a complex of a chimeric renal polynucleotide and another molecule.

Bifunctional renal polynucleotides may encode renal peptides which are anti-proliferative. These renal peptides may be linear, cyclic, constrained or random coil. They may function as aptamers, signaling molecules, ligands or mimics or mimetics thereof. Anti-proliferative renal peptides may, as translated, be from 3 to 50 amino acids in length. They may be 5-40, 10-30, or approximately 15 amino acids long. They may be single chain, multichain or branched and may form complexes, aggregates or any multi-unit structure once translated.

### Noncoding Renal Polynucleotides

As described herein, provided are renal polynucleotides having sequences that are partially or substantially not translatable, e.g., having a noncoding region. As one non-limiting example, the noncoding region may be the first region of the renal polynucleotide. Alternatively, the noncoding region may be a region other than the first region.

Such molecules are generally not translated, but can exert an effect on protein production by one or more of binding to and sequestering one or more translational machinery components such as a ribosomal protein or a transfer RNA (tRNA), thereby effectively reducing protein expression in the cell or modulating one or more pathways or cascades in a cell which in turn alters protein levels. The renal polynucleotide may contain or encode one or more long noncoding RNA (lncRNA, or lincRNA) or portion thereof, a small nucleolar RNA (sno-RNA), micro RNA (miRNA), small interfering RNA (siRNA) or Piwi-interacting RNA (piRNA). Examples of such lncRNA molecules and RNAi constructs designed to target such lncRNA any of which may be encoded in the renal polynucleotides are taught in International Publication, WO2012/018881 A2, the contents of which are incorporated herein by reference in their entirety.

### Cytotoxic Nucleosides

In one embodiment, the renal polynucleotides of the present invention may incorporate one or more cytotoxic nucleosides. Non-limiting examples of cytotoxic nucleosides are described in International Patent Publication No. WO2013151666, the contents of which are herein incorporated by reference in its entirety, such as in paragraphs [000201]-[000205].

### Regions of the Renal Polynucleotides

### Untranslated Regions (UTRs)

The renal polynucleotides of the present invention may comprise one or more regions or parts which act or function as an untranslated region. Where renal polynucleotides are designed to encode at least one renal polypeptide of interest, the renal polynucleotides may comprise one or more of these untranslated regions.

By definition, wild type untranslated regions (UTRs) of a gene are transcribed but not translated. In mRNA, the 5′UTR starts at the transcription start site and continues to the start codon but does not include the start codon; whereas, the 3′UTR starts immediately following the stop codon and continues until the transcriptional termination signal. There is growing body of evidence about the regulatory roles played by the UTRs in terms of stability of the nucleic acid molecule and translation. The regulatory features of a UTR can be incorporated into the renal polynucleotides of the present invention to, among other things, enhance the stability of the molecule. The specific features can also be incorporated to ensure controlled down-regulation of the transcript in case they are misdirected to undesired organs sites.

Combinations of features may be included in flanking regions and may be contained within other features. For example, the ORF may be flanked by a 5′ UTR which may contain a strong Kozak translational initiation signal and/or a 3′ UTR which may include an oligo(dT) sequence for templated addition of a poly-A tail. 5′UTR may comprise a first renal polynucleotide fragment and a second renal polynucleotide fragment from the same and/or different genes such as the 5′UTRs described in US Patent Application Publication No. 20100293625, herein incorporated by reference in its entirety.

Tables 1 and 2 provide a listing of exemplary UTRs which may be utilized in the renal polynucleotides of the present invention. Shown in Table 1 is a listing of a 5′-untranslated region of the invention. Variants of 5′ UTRs may be utilized wherein one or more nucleotides are added or removed to the termini, including A, T, C or G.

TABLE 1
5′-Untranslated Regions

| 5′ UTR | | |
|---|---|---|
| Identifier | Name/Description | SEQ ID NO. |
| 5UTR-001 | Upstream UTR | 1 |
| 5UTR-002 | Upstream UTR | 2 |
| 5UTR-003 | Upstream UTR | 3 |
| 5UTR-004 | Upstream UTR | 4 |
| 5UTR-005 | Upstream UTR | 5 |
| 5UTR-006 | Upstream UTR | 6 |
| 5UTR-007 | Upstream UTR | 7 |
| 5UTR-008 | Upstream UTR | 8 |
| 5UTR-009 | Upstream UTR | 9 |
| 5UTR-010 | Upstream UTR | 10 |
| 5UTR-011 | Upstream UTR | 11 |
| 5UTR-012 | Upstream UTR | 12 |
| 5UTR-013 | Upstream UTR | 13 |
| 5UTR-014 | Upstream UTR | 14 |
| 5UTR-015 | Upstream UTR | 15 |
| 5UTR-016 | Upstream UTR | 16 |
| 5UTR-017 | Upstream UTR | 17 |

Shown in Table 2 is a listing of 3′-untranslated regions of the invention. Variants of 3′ UTRs may be utilized wherein one or more nucleotides are added or removed to the termini, including A, T, C or G.

TABLE 2
3′-Untranslated Regions

| 3′ UTR | | |
|---|---|---|
| Identifier | Name/Description | SEQ ID NO. |
| 3UTR-001 | Creatine Kinase | 18 |
| 3UTR-002 | Myoglobin | 19 |
| 3UTR-003 | α-actin | 20 |
| 3UTR-004 | Albumin | 21 |
| 3UTR-005 | α-globin | 22 |
| 3UTR-006 | G-CSF | 23 |
| 3UTR-007 | Col1a2; collagen, type I, alpha 2 | 24 |
| 3UTR-008 | Col6a2; collagen, type VI, alpha 2 | 25 |
| 3UTR-009 | RPN1; ribophorin I | 26 |
| 3UTR-010 | LRP1; low density lipoprotein receptor- | 27 |
| | related protein 1 | |
| 3UTR-011 | Nnt1; cardiotrophin-like cytokine factor 1 | 28 |
| 3UTR-012 | Col6a1; collagen, type VI, alpha 1 | 29 |
| 3UTR-013 | Calr; calreticulin | 30 |
| 3UTR-014 | Col1a1; collagen, type I, alpha 1 | 31 |
| 3UTR-015 | Plod1; procollagen-lysine, | 32 |
| | 2-oxoglutarate 5-dioxygenase 1 | |
| 3UTR-016 | Nucb1; nucleobindin 1 | 33 |
| 3UTR-017 | α-globin | 34 |

### 5′ UTR

Co-pending, co-owned International Patent Publication No. WO2014164253 (Attorney Docket No. M042.20) provides a listing of exemplary UTRs which may be utilized in the renal polynucleotide of the present invention as flanking regions. Variants of 5′ or 3′ UTRs may be utilized wherein one or more nucleotides are added or removed to the termini, including A, T, C or G.

It should be understood that any UTR from any gene may be incorporated into the regions of the renal polynucleotide. Furthermore, multiple wild-type UTRs of any known gene may be utilized. It is also within the scope of the present invention to provide artificial UTRs which are not variants of wild type regions. These UTRs or portions thereof may be placed in the same orientation as in the transcript from which they were selected or may be altered in orientation or location. Hence a 5′ or 3′ UTR may be inverted, shortened, lengthened, made with one or more other 5′ UTRs or 3′ UTRs. As used herein, the term “altered” as it relates to a UTR sequence, means that the UTR has been changed in some way in relation to a reference sequence. For example, a 3′ or 5′ UTR may be altered relative to a wild type or native UTR by the change in orientation or location as taught above or may be altered by the inclusion of additional nucleotides, deletion of nucleotides, swapping or transposition of nucleotides. Any of these changes producing an “altered” UTR (whether 3′ or 5′) comprise a variant UTR.

In one embodiment, a double, triple or quadruple UTR such as a 5′ or 3′ UTR may be used. As used herein, a “double” UTR is one in which two copies of the same UTR are encoded either in series or substantially in series. For example, a double beta-globin 3′ UTR may be used as described in US Patent publication 20100129877, the contents of which are incorporated herein by reference in its entirety.

It is also within the scope of the present invention to have patterned UTRs. As used herein “patterned UTRs” are those UTRs which reflect a repeating or alternating pattern, such as ABABAB or AABBAABBAABB or ABCABCABC or variants thereof repeated once, twice, or more than 3 times. In these patterns, each letter, A, B, or C represent a different UTR at the nucleotide level.

In one embodiment, flanking regions are selected from a family of transcripts whose proteins share a common function, structure, feature of property. For example, renal polypeptides of interest may belong to a family of proteins which are expressed in a particular cell, tissue or at some time during development. The UTRs from any of these genes may be swapped for any other UTR of the same or different family of proteins to create a new renal polynucleotide. As used herein, a “family of proteins” is used in the broadest sense to refer to a group of two or more renal polypeptides of interest which share at least one function, structure, feature, localization, origin, or expression pattern.

The untranslated region may also include translation enhancer elements (TEE). As a non-limiting example, the TEE may include those described in US Application No. 20090226470, herein incorporated by reference in its entirety, and those known in the art.

### 5′ UTR and Translation Initiation

Natural 5′UTRs bear features which play roles in translation initiation. They harbor signatures like Kozak sequences which are commonly known to be involved in the process by which the ribosome initiates translation of many genes. Kozak sequences have the consensus CCR(A/G)CCAUGG, where R is a purine (adenine or guanine) three bases upstream of the start codon (AUG), which is followed by another ‘G’. 5′UTR also have been known to form secondary structures which are involved in elongation factor binding.

By engineering the features typically found in abundantly expressed genes of specific target organs, one can enhance the stability and protein production of the renal polynucleotides of the invention. For example, introduction of 5′ UTR of liver-expressed mRNA, such as albumin, serum amyloid A, Apolipoprotein A/B/E, transferrin, alpha fetoprotein, erythropoietin, or Factor VIII, could be used to enhance expression of a nucleic acid molecule, such as a renal polynucleotides, in hepatic cell lines or liver. Likewise, use of 5′ UTR from other tissue-specific mRNA to improve expression in that tissue is possible for muscle (MyoD, Myosin, Myoglobin, Myogenin, Herculin), for endothelial cells (Tie-1, CD36), for myeloid cells (C/EBP, AML1, G-CSF, GM-CSF, CD11b, MSR, Fr-1, i-NOS), for leukocytes (CD45, CD18), for adipose tissue (CD36, GLUT4, ACRP30, adiponectin) and for lung epithelial cells (SP-A/B/C/D). Untranslated regions useful in the design and manufacture of renal polynucleotides include, but are not limited, to those disclosed in co-pending, co-owned International Patent Publication No. WO2014164253 (Attorney Docket No. M042.20), the contents of which are incorporated herein by reference in its entirety.

### 5′UTR and Histone Stem Loops

In one embodiment, the renal polynucleotides may include a nucleic acid sequence which is derived from the 5′UTR of a 5′-terminal oligopyrimidine (TOP) gene and at least one histone stem loop. Non-limiting examples of nucleic acid sequences which are derived from the 5′UTR of a TOP gene are taught in International Patent Publication No. WO2013143699, the contents of which are herein incorporated by reference in its entirety. 3′UTR

In one embodiment, the renal polynucleotides of the present invention may include a 3′UTR which may be heterologous to the 5′UTR and/or the coding region. In another embodiment, the renal polynucleotides described herein may include a 3′ UTR derived from a gene which is a different than the gene the 5′ UTR is derived from. In yet another embodiment, the renal polynucleotides described herein may include a 3′ UTR which is derived from a different protein than the protein encoded by the coding region.

In one embodiment, 3′ UTRs of the renal polynucleotides described herein may comprise a nucleic acid sequence which is derived from the 3′ UTR of an albumin gene or from a variant of the 3′UTR of the Albumin Gene.

In another embodiment, 3′ UTRs of the renal polynucleotides described herein may comprise a nucleic acid sequence which is derived from the globin gene or from a variant of the globin gene. As a non-limiting example, the 3′UTR may be derived from the 3′UTR of a globin gene (e.g., alpha globin or beta globin).

### 3′ UTR and the AU Rich Elements

Natural or wild type 3′ UTRs are known to have stretches of Adenosines and Uridines embedded in them. These AU rich signatures are particularly prevalent in genes with high rates of turnover. Based on their sequence features and functional properties, the AU rich elements (AREs) can be separated into three classes (Chen et al, 1995): Class I AREs contain several dispersed copies of an AUUUA motif within U-rich regions. C-Myc and MyoD contain class I AREs. Class II AREs possess two or more overlapping UUAUUUA(U/A)(U/A) nonamers. Molecules containing this type of AREs include GM-CSF and TNF-α. Class III ARES are less well defined. These U rich regions do not contain an AUUUA motif. c-Jun and Myogenin are two well-studied examples of this class. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Introduction, removal or modification of 3′ UTR AU rich elements (AREs) can be used to modulate the stability of renal polynucleotides of the invention. When engineering specific renal polynucleotides, one or more copies of an ARE can be introduced to make renal polynucleotides of the invention less stable and thereby curtail translation and decrease production of the resultant protein. Likewise, AREs can be identified and removed or mutated to increase the intracellular stability and thus increase translation and production of the resultant protein. Transfection experiments can be conducted in relevant cell lines, using renal polynucleotides of the invention and protein production can be assayed at various time points post-transfection. For example, cells can be transfected with different ARE-engineering molecules and by using an ELISA kit to the relevant protein and assaying protein produced at 6 hour, 12 hour, 24 hour, 48 hour, and 7 days post-transfection.

Untranslated Regions and microRNA Binding Sites

microRNAs (or miRNA) are 19-25 nucleotide long noncoding RNAs that bind to the 3′UTR of nucleic acid molecules and down-regulate gene expression either by reducing nucleic acid molecule stability or by inhibiting translation. The renal polynucleotides of the invention may comprise one or more microRNA target sequences, microRNA sequences, or microRNA seeds. Such sequences may correspond to any known microRNA such as those taught in US Publication US2005/0261218 and US Publication US2005/0059005, the contents of which are incorporated herein by reference in their entirety.

A microRNA sequence comprises a “seed” region, i.e., a sequence in the region of positions 2-8 of the mature microRNA, which sequence has perfect Watson-Crick complementarity to the miRNA target sequence. A microRNA seed may comprise positions 2-8 or 2-7 of the mature microRNA. In some embodiments, a microRNA seed may comprise 7 nucleotides (e.g., nucleotides 2-8 of the mature microRNA), wherein the seed-complementary site in the corresponding miRNA target is flanked by an adenine (A) opposed to microRNA position 1. In some embodiments, a microRNA seed may comprise 6 nucleotides (e.g., nucleotides 2-7 of the mature microRNA), wherein the seed-complementary site in the corresponding miRNA target is flanked by an adenine (A) opposed to microRNA position 1. See for example, Grimson A, Farh K K, Johnston W K, Garrett-Engele P, Lim L P, Bartel D P; Mol Cell. 2007 Jul. 6; 27(1):91-105; each of which is herein incorporated by reference in their entirety. The bases of the microRNA seed have complete complementarity with the target sequence. By engineering microRNA target sequences into the renal polynucleotides (e.g., in a 3′UTR like region or other region) of the invention one can target the molecule for degradation or reduced translation, provided the microRNA in question is available. This process will reduce the hazard of off target effects upon nucleic acid molecule delivery. Identification of microRNA, microRNA target regions, and their expression patterns and role in biology have been reported (Bonauer et al., Curr Drug Targets 2010 11:943-949; Anand and Cheresh Curr Opin Hematol 2011 18:171-176; Contreras and Rao Leukemia 2012 26:404-413 (2011 Dec. 20. doi: 10.1038/Ieu.2011.356); Bartel Cell 2009 136:215-233; Landgraf et al, Cell, 2007 129:1401-1414; each of which is herein incorporated by reference in its entirety).

For example, if the nucleic acid molecule is an mRNA and is not intended to be delivered to the liver but ends up there, then miR-122, a microRNA abundant in liver, can inhibit the expression of the gene of interest if one or multiple target sites of miR-122 are engineered into the 3′ UTR region of the renal polynucleotides. Introduction of one or multiple binding sites for different microRNA can be engineered to further decrease the longevity, stability, and protein translation of renal polynucleotides.

As used herein, the term “microRNA site” refers to a microRNA target site or a microRNA recognition site, or any nucleotide sequence to which a microRNA binds or associates. It should be understood that “binding” may follow traditional Watson-Crick hybridization rules or may reflect any stable association of the microRNA with the target sequence at or adjacent to the microRNA site.

Conversely, for the purposes of the renal polynucleotides of the present invention, microRNA binding sites can be engineered out of (i.e. removed from) sequences in which they occur, e.g., in order to increase protein expression in specific tissues. For example, miR-192, miR-194 or miR-204 binding sites may be removed to improve protein expression in the kidney. Regulation of expression in multiple tissues can be accomplished through introduction or removal or one or several microRNA binding sites.

Expression profiles, microRNA and cell lines useful in the present invention include those taught in for example, International Patent Publication No. WO2014081507 (Attorney Docket Number M39.20) and WO2014113089 (Attorney Docket Number M37.20), the contents of which are incorporated by reference in their entirety.

In the renal polynucleotides of the present invention, binding sites for microRNAs that are involved in such processes may be removed or introduced, in order to tailor the expression of the renal polynucleotides expression to biologically relevant cell types or to the context of relevant biological processes. A listing of microRNA, miR sequences and miR binding sites is listed in Table 9 of U.S. Provisional Application No. 61/753,661 filed Jan. 17, 2013, in Table 9 of U.S. Provisional Application No. 61/754,159 filed Jan. 18, 2013, and in Table 7 of U.S. Provisional Application No. 61/758,921 filed Jan. 31, 2013, each of which are herein incorporated by reference in their entireties.

Lastly, through an understanding of the expression patterns of microRNA in different cell types, renal polynucleotides can be engineered for more targeted expression in specific cell types or only under specific biological conditions. Through introduction of tissue-specific microRNA binding sites, renal polynucleotides could be designed that would be optimal for protein expression in a tissue or in the context of a biological condition.

Transfection experiments can be conducted in relevant cell lines, using engineered renal polynucleotides and protein production can be assayed at various time points post-transfection. For example, cells can be transfected with different microRNA binding site-engineering renal polynucleotides and by using an ELISA kit to the relevant protein and assaying protein produced at 6 hour, 12 hour, 24 hour, 48 hour, 72 hour and 7 days post-transfection. In vivo experiments can also be conducted using microRNA-binding site-engineered molecules to examine changes in tissue-specific expression of formulated renal polynucleotides.

### Insertions and Substitution of Untranslated Regions

In one embodiment, the UTRs of the renal polynucleotide may be, independently, replaced by the insertion of at least one region and/or string of nucleosides of the same base. The region and/or string of nucleotides may include, but is not limited to, at least 3, at least 4, at least 5, at least 6, at least 7 or at least 8 nucleotides and the nucleotides may be natural and/or unnatural. As a non-limiting example, the group of nucleotides may include 5-8 adenine, cytosine, thymine, a string of any of the other nucleotides disclosed herein and/or combinations thereof.

In one embodiment, the UTRs of the renal polynucleotide may be replaced by the insertion of at least two regions and/or strings of nucleotides of two different bases such as, but not limited to, adenine, cytosine, thymine, any of the other nucleotides disclosed herein and/or combinations thereof. For example, the 5′UTR may be replaced by inserting 5-8 adenine bases followed by the insertion of 5-8 cytosine bases. In another example, the 5′UTR may be replaced by inserting 5-8 cytosine bases followed by the insertion of 5-8 adenine bases.

In one embodiment, the renal polynucleotide may include at least one substitution and/or insertion downstream of the transcription start site which may be recognized by an RNA polymerase. As a non-limiting example, at least one substitution and/or insertion may occur downstream the transcription start site by substituting at least one nucleic acid in the region just downstream of the transcription start site (such as, but not limited to, +1 to +6). Changes to region of nucleotides just downstream of the transcription start site may affect initiation rates, increase apparent nucleotide triphosphate (NTP) reaction constant values, and increase the dissociation of short transcripts from the transcription complex curing initial transcription (Brieba et al, Biochemistry (2002) 41: 5144-5149; herein incorporated by reference in its entirety). The modification, substitution and/or insertion of at least one nucleoside may cause a silent mutation of the sequence or may cause a mutation in the amino acid sequence.

In one embodiment, the renal polynucleotide may include the substitution of at least 1, at least 2, at least 3, at least 4, at least 5, at least 6, at least 7, at least 8, at least 9, at least 10, at least 11, at least 12 or at least 13 guanine bases downstream of the transcription start site.

In one embodiment, the renal polynucleotide may include the substitution of at least 1, at least 2, at least 3, at least 4, at least 5 or at least 6 guanine bases in the region just downstream of the transcription start site. As a non-limiting example, if the nucleotides in the region are GGGAGA the guanine bases may be substituted by at least 1, at least 2, at least 3 or at least 4 adenine nucleotides. In another non-limiting example, if the nucleotides in the region are GGGAGA the guanine bases may be substituted by at least 1, at least 2, at least 3 or at least 4 cytosine bases. In another non-limiting example, if the nucleotides in the region are GGGAGA the guanine bases may be substituted by at least 1, at least 2, at least 3 or at least 4 thymine, and/or any of the nucleotides described herein.

In one embodiment, the renal polynucleotide may include at least one substitution and/or insertion upstream of the start codon. For the purpose of clarity, one of skill in the art would appreciate that the start codon is the first codon of the protein coding region whereas the transcription start site is the site where transcription begins. The renal polynucleotide may include, but is not limited to, at least 1, at least 2, at least 3, at least 4, at least 5, at least 6, at least 7 or at least 8 substitutions and/or insertions of nucleotide bases. The nucleotide bases may be inserted or substituted at 1, at least 1, at least 2, at least 3, at least 4 or at least 5 locations upstream of the start codon. The nucleotides inserted and/or substituted may be the same base (e.g., all A or all C or all T or all G), two different bases (e.g., A and C, A and T, or C and T), three different bases (e.g., A, C and T or A, C and T) or at least four different bases. As a non-limiting example, the guanine base upstream of the coding region in the renal polynucleotide may be substituted with adenine, cytosine, thymine, or any of the nucleotides described herein. In another non-limiting example the substitution of guanine bases in the renal polynucleotide may be designed so as to leave one guanine base in the region downstream of the transcription start site and before the start codon (see Esvelt et al. Nature (2011) 472(7344):499-503; the contents of which is herein incorporated by reference in its entirety). As a non-limiting example, at least 5 nucleotides may be inserted at 1 location downstream of the transcription start site but upstream of the start codon and the at least 5 nucleotides may be the same base type.

### Incorporating Post Transcriptional Control Modulators in the Untranslated Region

In one embodiment, the renal polynucleotides of the present invention may include at least one post transcriptional control modulator. These post transcriptional control modulators may be, but are not limited to, small molecules, compounds and regulatory sequences. As a non-limiting example, post transcriptional control may be achieved using small molecules identified by PTC Therapeutics Inc. (South Plainfield, N.J.) using their GEMS™ (Gene Expression Modulation by Small-Molecules) screening technology.

The post transcriptional control modulator may be a gene expression modulator which is screened by the method detailed in or a gene expression modulator described in International Publication No. WO2006022712, herein incorporated by reference in its entirety. Methods identifying RNA regulatory sequences involved in translational control are described in International Publication No. WO2004067728, herein incorporated by reference in its entirety; methods identifying compounds that modulate untranslated region dependent expression of a gene are described in International Publication No. WO2004065561, herein incorporated by reference in its entirety.

In one embodiment, the renal polynucleotides of the present invention may include at least one post transcriptional control modulator is located in the 5′ and/or the 3′ untranslated region of the renal polynucleotides of the present invention.

In another embodiment, the renal polynucleotides of the present invention may include at least one post transcription control modulator to modulate premature translation termination. The post transcription control modulators may be compounds described in or a compound found by methods outlined in International Publication Nos. WO2004010106, WO2006044456, WO2006044682, WO2006044503 and WO2006044505, each of which is herein incorporated by reference in its entirety. As a non-limiting example, the compound may bind to a region of the 28S ribosomal RNA in order to modulate premature translation termination (See e.g., WO2004010106, herein incorporated by reference in its entirety).

In one embodiment, renal polynucleotides of the present invention may include at least one post transcription control modulator to alter protein expression. As a non-limiting example, the expression of VEGF may be regulated using the compounds described in or a compound found by the methods described in International Publication Nos. WO2005118857, WO2006065480, WO2006065479 and WO2006058088, each of which is herein incorporated by reference in its entirety.

The renal polynucleotides of the present invention may include at least one post transcription control modulator to control translation. In one embodiment, the post transcription control modulator may be a RNA regulatory sequence. As a non-limiting example, the RNA regulatory sequence may be identified by the methods described in International Publication No. WO2006071903, herein incorporated by reference in its entirety.

### Regions Having a 5′ Cap

The 5′ cap structure of a natural mRNA is involved in nuclear export, increasing mRNA stability and binds the mRNA Cap Binding Protein (CBP), which is responsible for mRNA stability in the cell and translation competency through the association of CBP with poly(A) binding protein to form the mature cyclic mRNA species. The cap further assists the removal of 5′ proximal introns removal during mRNA splicing.

Endogenous mRNA molecules may be 5′-end capped generating a 5′-ppp-5′-triphosphate linkage between a terminal guanosine cap residue and the 5′-terminal transcribed sense nucleotide of the mRNA molecule. This 5′-guanylate cap may then be methylated to generate an N7-methyl-guanylate residue. The ribose sugars of the terminal and/or anteterminal transcribed nucleotides of the 5′ end of the mRNA may optionally also be 2′-O-methylated. 5′-decapping through hydrolysis and cleavage of the guanylate cap structure may target a nucleic acid molecule, such as an mRNA molecule, for degradation.

In some embodiments, renal polynucleotides may be designed to incorporate a cap moiety. Modifications to the renal polynucleotides of the present invention may generate a non-hydrolyzable cap structure preventing decapping and thus increasing mRNA half-life. Because cap structure hydrolysis requires cleavage of 5′-ppp-5′ phosphorodiester linkages, modified nucleotides may be used during the capping reaction. For example, a Vaccinia Capping Enzyme from New England Biolabs (Ipswich, Mass.) may be used with α-thio-guanosine nucleotides according to the manufacturer's instructions to create a phosphorothioate linkage in the 5′-ppp-5′ cap. Additional modified guanosine nucleotides may be used such as α-methyl-phosphonate and seleno-phosphate nucleotides.

Additional modifications include, but are not limited to, 2′-O-methylation of the ribose sugars of 5′-terminal and/or 5′-anteterminal nucleotides of the renal polynucleotide (as mentioned above) on the 2′-hydroxyl group of the sugar ring. Multiple distinct 5′-cap structures can be used to generate the 5′-cap of a nucleic acid molecule, such as a renal polynucleotide which functions as an mRNA molecule.

Cap analogs, which herein are also referred to as synthetic cap analogs, chemical caps, chemical cap analogs, or structural or functional cap analogs, differ from natural (i.e. endogenous, wild-type or physiological) 5′-caps in their chemical structure, while retaining cap function. Cap analogs may be chemically (i.e. non-enzymatically) or enzymatically synthesized and/or linked to the renal polynucleotides of the invention.

For example, the Anti-Reverse Cap Analog (ARCA) cap contains two guanines linked by a 5′-5′-triphosphate group, wherein one guanine contains an N7 methyl group as well as a 3′-O-methyl group (i.e., N7,3′-O-dimethyl-guanosine-5′-triphosphate-5′-guanosine (m⁷G-3′mppp-G; which may equivalently be designated 3′ O-Me-m7G(5′)ppp(5′)G). The 3′-O atom of the other, unmodified, guanine becomes linked to the 5′-terminal nucleotide of the capped renal polynucleotide. The N7- and 3′-O-methlyated guanine provides the terminal moiety of the capped renal polynucleotide.

Another exemplary cap is mCAP, which is similar to ARCA but has a 2′-O-methyl group on guanosine (i.e., N7,2′-O-dimethyl-guanosine-5′-triphosphate-5′-guanosine, m⁷Gm-ppp-G).

According to the present invention, 5′ terminal caps may include endogenous caps or cap analogs. According to the present invention, a 5′ terminal cap may comprise a guanine analog. Useful guanine analogs include, but are not limited to, inosine, N1-methyl-guanosine, 2′fluoro-guanosine, 7-deaza-guanosine, 8-oxo-guanosine, 2-amino-guanosine, LNA-guanosine, and 2-azido-guanosine.

In one embodiment, the cap is a dinucleotide cap analog. As a non-limiting example, the dinucleotide cap analog may be modified at different phosphate positions with a boranophosphate group or a phophoroselenoate group such as the dinucleotide cap analogs described in U.S. Pat. No. 8,519,110, the contents of which are herein incorporated by reference in its entirety.

In another embodiment, the cap is a cap analog is a N7-(4-chlorophenoxyethyl) substituted dinucleotide form of a cap analog known in the art and/or described herein. Non-limiting examples of a N7-(4-chlorophenoxyethyl) substituted dinucleotide form of a cap analog include a N7-(4-chlorophenoxyethyl)-G(5′)ppp(5′)G and a N7-(4-chlorophenoxyethyl)-m^{3′-O}G(5′)ppp(5′)G cap analog (See e.g., the various cap analogs and the methods of synthesizing cap analogs described in Kore et al. Bioorganic & Medicinal Chemistry 2013 21:4570-4574; the contents of which are herein incorporated by reference in its entirety). In another embodiment, a cap analog of the present invention is a 4-chloro/bromophenoxyethyl analog.

While cap analogs allow for the concomitant capping of a renal polynucleotide or a region thereof, in an in vitro transcription reaction, up to 20% of transcripts can remain uncapped. This, as well as the structural differences of a cap analog from an endogenous 5′-cap structures of nucleic acids produced by the endogenous, cellular transcription machinery, may lead to reduced translational competency and reduced cellular stability.

Renal polynucleotides of the invention may also be capped post-manufacture, using enzymes, in order to generate more authentic 5′-cap structures. As used herein, the phrase “more authentic” refers to a feature that closely mirrors or mimics, either structurally or functionally, an endogenous or wild type feature. That is, a “more authentic” feature is better representative of an endogenous, wild-type, natural or physiological cellular function and/or structure as compared to synthetic features or analogs, etc., of the prior art, or which outperforms the corresponding endogenous, wild-type, natural or physiological feature in one or more respects. Non-limiting examples of more authentic 5′cap structures of the present invention are those which, among other things, have enhanced binding of cap binding proteins, increased half-life, reduced susceptibility to 5′ endonucleases and/or reduced 5′decapping, as compared to synthetic 5′cap structures known in the art (or to a wild-type, natural or physiological 5′cap structure). For example, recombinant Vaccinia Virus Capping Enzyme and recombinant 2′-O-methyltransferase enzyme can create a canonical 5′-5′-triphosphate linkage between the 5′-terminal nucleotide of a renal polynucleotide and a guanine cap nucleotide wherein the cap guanine contains an N7 methylation and the 5′-terminal nucleotide of the mRNA contains a 2′-O-methyl. Such a structure is termed the Cap1 structure. This cap results in a higher translational-competency and cellular stability and a reduced activation of cellular pro-inflammatory cytokines, as compared, e.g., to other 5′cap analog structures known in the art. Cap structures include, but are not limited to, 7mG(5′)ppp(5′)N,pN2p (cap 0), 7mG(5′)ppp(5′)NImpNp (cap 1), and 7mG(5′)-ppp(5′)NImpN2mp (cap 2).

### Viral and Viral Derived Sequence Regions

### Viral Sequences

Additional viral sequences such as, but not limited to, the translation enhancer sequence of the barley yellow dwarf virus (BYDV-PAV), the Jaagsiekte sheep retrovirus (JSRV) and/or the Enzootic nasal tumor virus (See e.g., International Pub. No. WO2012129648; herein incorporated by reference in its entirety) can be engineered and inserted in the renal polynucleotides of the invention and can stimulate the translation of the construct in vitro and in vivo. Transfection experiments can be conducted in relevant cell lines at and protein production can be assayed by ELISA at 12 hr, 24 hr, 48 hr, 72 hr and day 7 post-transfection.

### IRES Sequences

Further, provided are renal polynucleotides which may contain an internal ribosome entry site (IRES). First identified as a feature Picorna virus RNA, IRES plays an important role in initiating protein synthesis in absence of the 5′ cap structure. An IRES may act as the sole ribosome binding site, or may serve as one of multiple ribosome binding sites of an mRNA. Renal polynucleotides containing more than one functional ribosome binding site may encode several renal peptides or renal polypeptides that are translated independently by the ribosomes (“multicistronic nucleic acid molecules”). When renal polynucleotides are provided with an IRES, further optionally provided is a second translatable region. Examples of IRES sequences that can be used according to the invention include without limitation, those from picornaviruses (e.g. FMDV), pest viruses (CFFV), polio viruses (PV), encephalomyocarditis viruses (ECMV), foot-and-mouth disease viruses (FMDV), hepatitis C viruses (HCV), classical swine fever viruses (CSFV), murine leukemia virus (MLV), simian immune deficiency viruses (SIV) or cricket paralysis viruses (CrPV).

### Tailing Regions

### Poly-A Tails

During RNA processing, a long chain of adenine nucleotides (poly-A tail) may be added to a renal polynucleotide such as an mRNA molecule in order to increase stability. Immediately after transcription, the 3′ end of the transcript may be cleaved to free a 3′ hydroxyl. Then poly-A polymerase adds a chain of adenine nucleotides to the RNA. The process, called polyadenylation, adds a poly-A tail that can be between, for example, approximately 80 to approximately 250 residues long, including approximately 80, 90, 100, 110, 120, 130, 140, 150, 160, 170, 180, 190, 200, 210, 220, 230, 240 or 250 residues long.

PolyA tails may also be added after the construct is exported from the nucleus.

According to the present invention, terminal groups on the poly A tail may be incorporated for stabilization. Renal polynucleotides of the present invention may include des-3′ hydroxyl tails. They may also include structural moieties or 2′-Omethyl modifications as taught by Junjie Li, et al. (Current Biology, Vol. 15, 1501-1507, Aug. 23, 2005, the contents of which are incorporated herein by reference in its entirety).

The renal polynucleotides of the present invention may be designed to encode transcripts with alternative polyA tail structures including histone mRNA. According to Norbury, “Terminal uridylation has also been detected on human replication-dependent histone mRNAs. The turnover of these mRNAs is thought to be important for the prevention of potentially toxic histone accumulation following the completion or inhibition of chromosomal DNA replication. These mRNAs are distinguished by their lack of a 3′ poly(A) tail, the function of which is instead assumed by a stable stem-loop structure and its cognate stem-loop binding protein (SLBP); the latter carries out the same functions as those of PABP on polyadenylated mRNAs” (Norbury, “Cytoplasmic RNA: a case of the tail wagging the dog,” Nature Reviews Molecular Cell Biology; AOP, published online 29 Aug. 2013; doi:10.1038/nrm3645) the contents of which are incorporated herein by reference in its entirety.

Unique poly-A tail lengths provide certain advantages to the renal polynucleotides of the present invention.

Generally, the length of a poly-A tail, when present, is greater than 30 nucleotides in length. In another embodiment, the poly-A tail is greater than 35 nucleotides in length (e.g., at least or greater than about 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000 nucleotides). In some embodiments, the renal polynucleotide or region thereof includes from about 30 to about 3,000 nucleotides (e.g., from 30 to 50, from 30 to 100, from 30 to 250, from 30 to 500, from 30 to 750, from 30 to 1,000, from 30 to 1,500, from 30 to 2,000, from 30 to 2,500, from 50 to 100, from 50 to 250, from 50 to 500, from 50 to 750, from 50 to 1,000, from 50 to 1,500, from 50 to 2,000, from 50 to 2,500, from 50 to 3,000, from 100 to 500, from 100 to 750, from 100 to 1,000, from 100 to 1,500, from 100 to 2,000, from 100 to 2,500, from 100 to 3,000, from 500 to 750, from 500 to 1,000, from 500 to 1,500, from 500 to 2,000, from 500 to 2,500, from 500 to 3,000, from 1,000 to 1,500, from 1,000 to 2,000, from 1,000 to 2,500, from 1,000 to 3,000, from 1,500 to 2,000, from 1,500 to 2,500, from 1,500 to 3,000, from 2,000 to 3,000, from 2,000 to 2,500, and from 2,500 to 3,000).

In one embodiment, the poly-A tail is designed relative to the length of the overall renal polynucleotide or the length of a particular region of the renal polynucleotide. This design may be based on the length of a coding region, the length of a particular feature or region or based on the length of the ultimate product expressed from the renal polynucleotides.

In this context the poly-A tail may be 10, 20, 30, 40, 50, 60, 70, 80, 90, or 100% greater in length than the renal polynucleotide or feature thereof. The poly-A tail may also be designed as a fraction of the renal polynucleotides to which it belongs. In this context, the poly-A tail may be 10, 20, 30, 40, 50, 60, 70, 80, or 90% or more of the total length of the construct, a construct region or the total length of the construct minus the poly-A tail. Further, engineered binding sites and conjugation of renal polynucleotides for Poly-A binding protein may enhance expression.

Additionally, multiple distinct renal polynucleotides may be linked together via the PABP (Poly-A binding protein) through the 3′-end using modified nucleotides at the 3′-terminus of the poly-A tail. Transfection experiments can be conducted in relevant cell lines at and protein production can be assayed by ELISA at 12 hr, 24 hr, 48 hr, 72 hr and day 7 post-transfection.

In one embodiment, the renal polynucleotides of the present invention are designed to include a polyA-G Quartet region. The G-quartet is a cyclic hydrogen bonded array of four guanine nucleotides that can be formed by G-rich sequences in both DNA and RNA. In this embodiment, the G-quartet is incorporated at the end of the poly-A tail. The resultant renal polynucleotide is assayed for stability, protein production and other parameters including half-life at various time points. It has been discovered that the polyA-G quartet results in protein production from an mRNA equivalent to at least 75% of that seen using a poly-A tail of 120 nucleotides alone.

### Start Codon Region

In some embodiments, the renal polynucleotides of the present invention may have regions that are analogous to or function like a start codon region.

In one embodiment, the translation of a renal polynucleotide may initiate on a codon which is not the start codon AUG/ATG. Translation of the renal polynucleotide may initiate on an alternative start codon such as, but not limited to, ACG, AGG, AAG, CTG/CUG, GTG/GUG, ATA/AUA, ATT/AUU, TTG/UUG (see Touriol et al. Biology of the Cell 95 (2003) 169-178 and Matsuda and Mauro PLoS ONE, 2010 5:11; the contents of each of which are herein incorporated by reference in its entirety). As a non-limiting example, the translation of a renal polynucleotide begins on the alternative start codon ACG. As another non-limiting example, renal polynucleotide translation begins on the alternative start codon CTG or CUG. As yet another non-limiting example, the translation of a renal polynucleotide begins on the alternative start codon GTG or GUG.

Nucleotides flanking a codon that initiates translation such as, but not limited to, a start codon or an alternative start codon, are known to affect the translation efficiency, the length and/or the structure of the renal polynucleotide. (See e.g., Matsuda and Mauro PLoS ONE, 2010 5:11; the contents of which are herein incorporated by reference in its entirety). Masking any of the nucleotides flanking a codon that initiates translation may be used to alter the position of translation initiation, translation efficiency, length and/or structure of a renal polynucleotide.

In one embodiment, a masking agent may be used near the start codon or alternative start codon in order to mask or hide the codon to reduce the probability of translation initiation at the masked start codon or alternative start codon. Non-limiting examples of masking agents include antisense locked nucleic acids (LNA) renal polynucleotides and exon-junction complexes (EJCs) (See e.g., Matsuda and Mauro describing masking agents LNA renal polynucleotides and EJCs (PLoS ONE, 2010 5:11); the contents of which are herein incorporated by reference in its entirety).

In another embodiment, a masking agent may be used to mask a start codon of a renal polynucleotide in order to increase the likelihood that translation will initiate on an alternative start codon.

In one embodiment, a masking agent may be used to mask a first start codon or alternative start codon in order to increase the chance that translation will initiate on a start codon or alternative start codon downstream to the masked start codon or alternative start codon.

In one embodiment, a start codon or alternative start codon may be located within a perfect complement for a miR binding site. The perfect complement of a miR binding site may help control the translation, length and/or structure of the renal polynucleotide similar to a masking agent. As a non-limiting example, the start codon or alternative start codon may be located in the middle of a perfect complement for a miR-122 binding site. The start codon or alternative start codon may be located after the first nucleotide, second nucleotide, third nucleotide, fourth nucleotide, fifth nucleotide, sixth nucleotide, seventh nucleotide, eighth nucleotide, ninth nucleotide, tenth nucleotide, eleventh nucleotide, twelfth nucleotide, thirteenth nucleotide, fourteenth nucleotide, fifteenth nucleotide, sixteenth nucleotide, seventeenth nucleotide, eighteenth nucleotide, nineteenth nucleotide, twentieth nucleotide or twenty-first nucleotide.

In another embodiment, the start codon of a renal polynucleotide may be removed from the renal polynucleotide sequence in order to have the translation of the renal polynucleotide begin on a codon which is not the start codon. Translation of the renal polynucleotide may begin on the codon following the removed start codon or on a downstream start codon or an alternative start codon. In a non-limiting example, the start codon ATG or AUG is removed as the first 3 nucleotides of the renal polynucleotide sequence in order to have translation initiate on a downstream start codon or alternative start codon. The renal polynucleotide sequence where the start codon was removed may further comprise at least one masking agent for the downstream start codon and/or alternative start codons in order to control or attempt to control the initiation of translation, the length of the renal polynucleotide and/or the structure of the renal polynucleotide.

### Stop Codon Region

In one embodiment, the renal polynucleotides of the present invention may include at least two stop codons before the 3′ untranslated region (UTR). The stop codon may be selected from TGA, TAA and TAG. In one embodiment, the renal polynucleotides of the present invention include the stop codon TGA and one additional stop codon. In a further embodiment the addition stop codon may be TAA. In another embodiment, the renal polynucleotides of the present invention include three stop codons.

### Signal Sequence Region

### Signal Sequences

The renal polynucleotides may also encode additional features which facilitate trafficking of the renal polypeptides to therapeutically relevant sites. One such feature which aids in protein trafficking is the signal sequence. As used herein, a “signal sequence” or “signal renal peptide” is a renal polynucleotide or renal polypeptide, respectively, which is from about 9 to 200 nucleotides (3-60 amino acids) in length which is incorporated at the 5′ (or N-terminus) of the coding region or renal polypeptide encoded, respectively. Addition of these sequences result in trafficking of the encoded renal polypeptide to the endoplasmic reticulum through one or more secretory pathways. Some signal renal peptides are cleaved from the protein by signal peptidase after the proteins are transported.

Additional signal sequences which may be utilized in the present invention include those taught in, for example, databases such as those found at http://www.signalpeptide.de/ or http://proline.bic.nus.edu.sg/spdb/. Those described in U.S. Pat. Nos. 8,124,379; 7,413,875 and 7,385,034 are also within the scope of the invention and the contents of each are incorporated herein by reference in their entirety.

### Cleavage Regions: Protein Cleavage Signals and Sites

In one embodiment, the renal polypeptides of the present invention may include at least one protein cleavage signal containing at least one protein cleavage site. The protein cleavage site may be located at the N-terminus, the C-terminus, at any space between the N- and the C-termini such as, but not limited to, half-way between the N- and C-termini, between the N-terminus and the half way point, between the half way point and the C-terminus, and combinations thereof.

The renal polypeptides of the present invention may include, but is not limited to, a proprotein convertase (or prohormone convertase), thrombin or Factor Xa protein cleavage signal. Proprotein convertases are a family of nine proteinases, comprising seven basic amino acid-specific subtilisin-like serine proteinases related to yeast kexin, known as prohormone convertase 1/3 (PC1/3), PC2, furin, PC4, PC5/6, paired basic amino-acid cleaving enzyme 4 (PACE4) and PC7, and two other subtilases that cleave at non-basic residues, called subtilisin kexin isozyme 1 (SKI-1) and proprotein convertase subtilisin kexin 9 (PCSK9).

In one embodiment, the renal polynucleotides of the present invention may be engineered such that the renal polynucleotide contains at least one encoded protein cleavage signal. The encoded protein cleavage signal may be located in any region including but not limited to before the start codon, after the start codon, before the coding region, within the coding region such as, but not limited to, half way in the coding region, between the start codon and the half way point, between the half way point and the stop codon, after the coding region, before the stop codon, between two stop codons, after the stop codon and combinations thereof.

In one embodiment, the renal polynucleotides of the present invention may include at least one encoded protein cleavage signal containing at least one protein cleavage site. The encoded protein cleavage signal may include, but is not limited to, a proprotein convertase (or prohormone convertase), thrombin and/or Factor Xa protein cleavage signal.

As a non-limiting example, U.S. Pat. No. 7,374,930 and U.S. Pub. No. 20090227660, herein incorporated by reference in their entireties, use a furin cleavage site to cleave the N-terminal methionine of GLP-1 in the expression product from the Golgi apparatus of the cells. In one embodiment, the renal polypeptides of the present invention include at least one protein cleavage signal and/or site with the proviso that the renal polypeptide is not GLP-1.

In one embodiment, the renal polynucleotides of the present invention may include a sequence encoding a self-cleaving renal peptide. The self-cleaving renal peptide may be, but is not limited to, a 2A peptide. As a non-limiting example, the 2A peptide may have the protein sequence: GSGATNFSLLKQAGDVEENPGP (SEQ ID NO: 1), fragments or variants thereof. In one embodiment, the 2A renal peptide cleaves between the last glycine and last proline. As another non-limiting example, the renal polynucleotides of the present invention may include a renal polynucleotide sequence encoding the 2A renal peptide having the protein sequence GSGATNFSLLKQAGDVEENPGP (SEQ ID NO: 1) fragments or variants thereof.

One such renal polynucleotide sequence encoding the 2A renal peptide is GGAAGCGGAGCTACTAACTTCAGCCTGCTGAAGCAGGCTGGAGACGTGGAGGAGAACCCTGGACC T (SEQ ID NO: 2). The renal polynucleotide sequence of the 2A renal peptide may be modified or codon optimized by the methods described herein and/or are known in the art.

In one embodiment, this sequence may be used to separate the coding region of two or more renal polypeptides of interest. As a non-limiting example, the sequence encoding the 2A renal peptide may be between a first coding region A and a second coding region B (A-2Apep-B). The presence of the 2A renal peptide would result in the cleavage of one long protein into protein A, protein B and the 2A renal peptide. Protein A and protein B may be the same or different renal peptides or renal polypeptides of interest. In another embodiment, the 2A renal peptide may be used in the renal polynucleotides of the present invention to produce two, three, four, five, six, seven, eight, nine, ten or more proteins.

In one embodiment, linear renal polynucleotides of the present invention which are made using only in vitro transcription (IVT) enzymatic synthesis methods are referred to as “IVT renal polynucleotides.” Formulations and compositions comprising IVT renal polynucleotides and methods of making, using and administering IVT renal polynucleotides are known in the art and are described in co-pending International Publication Nos. WO2013151666, WO2013151667, WO2013151668, WO2013151663, WO2013151669, WO2013151670, WO2013151664, WO2013151665, WO2013151736, WO2013151671 and WO2013151672; the contents of each of which are herein incorporated by reference in their entireties.

### Renal Polypeptides of Interest

Renal polynucleotides of the present invention may encode one or more renal peptides or renal polypeptides of interest. They may also affect the levels, signaling or function of one or more renal peptides or renal polypeptides. Renal polypeptides of interest, according to the present invention include any of the renal polypeptides described herein in Table 3 or any of the renal polypeptides taught in, for example, those listed in Table 6 of co-pending International Publication Nos. WO2013151666, WO2013151667, WO2013151668, WO2013151663, WO2013151669, WO2013151670, WO2013151664, WO2013151665, WO2013151736, WO2013151671 and WO2013151672 and Table 178 of International Publication No. WO2013151671; the contents of each of which are herein incorporated by reference in their entireties.

According to the present invention, the renal polynucleotide may be designed to encode one or more renal polypeptides of interest or fragments thereof. Such renal polypeptide of interest may include, but is not limited to, whole renal polypeptides, a plurality of renal polypeptides or fragments of renal polypeptides, which independently may be encoded by one or more regions or parts or the whole of a renal polynucleotide. As used herein, the term “renal polypeptides of interest” refer to any renal polypeptide which is selected to be encoded within, or whose function is affected by, the renal polynucleotides of the present invention.

As used herein, “renal polypeptide” means a polymer of amino acid residues (natural or unnatural) linked together most often by renal peptide bonds. The term, as used herein, refers to proteins, renal polypeptides, and renal peptides of any size, structure, or function. In some instances the renal polypeptide encoded is smaller than about 50 amino acids and the renal polypeptide is then termed a renal peptide. If the renal polypeptide is a renal peptide, it will be at least about 2, 3, 4, or at least 5 amino acid residues long. Thus, renal polypeptides include gene products, naturally occurring renal polypeptides, synthetic renal polypeptides, homologs, orthologs, paralogs, fragments and other equivalents, variants, and analogs of the foregoing. A renal polypeptide may be a single molecule or may be a multi-molecular complex such as a dimer, trimer or tetramer. They may also comprise single chain or multichain renal polypeptides such as antibodies or insulin and may be associated or linked. Most commonly disulfide linkages are found in multichain renal polypeptides. The term renal polypeptide may also apply to amino acid polymers in which one or more amino acid residues are an artificial chemical analogue of a corresponding naturally occurring amino acid.

The term “renal polypeptide variant” refers to molecules which differ in their amino acid sequence from a native or reference sequence. The amino acid sequence variants may possess substitutions, deletions, and/or insertions at certain positions within the amino acid sequence, as compared to a native or reference sequence. Ordinarily, variants will possess at least about 50% identity (homology) to a native or reference sequence, and preferably, they will be at least about 80%, more preferably at least about 90% identical (homologous) to a native or reference sequence.

In some embodiments “variant mimics” are provided. As used herein, the term “variant mimic” is one which contains one or more amino acids which would mimic an activated sequence. For example, glutamate may serve as a mimic for phosphoro-threonine and/or phosphoro-serine. Alternatively, variant mimics may result in deactivation or in an inactivated product containing the mimic, e.g., phenylalanine may act as an inactivating substitution for tyrosine; or alanine may act as an inactivating substitution for serine.

“Homology” as it applies to amino acid sequences is defined as the percentage of residues in the candidate amino acid sequence that are identical with the residues in the amino acid sequence of a second sequence after aligning the sequences and introducing gaps, if necessary, to achieve the maximum percent homology. Methods and computer programs for the alignment are well known in the art. It is understood that homology depends on a calculation of percent identity but may differ in value due to gaps and penalties introduced in the calculation.

By “homologs” as it applies to renal polypeptide sequences means the corresponding sequence of other species having substantial identity to a second sequence of a second species.

“Analogs” is meant to include renal polypeptide variants which differ by one or more amino acid alterations, e.g., substitutions, additions or deletions of amino acid residues that still maintain one or more of the properties of the parent or starting renal polypeptide.

The present invention contemplates several types of compositions which are renal polypeptide based including variants and derivatives. These include substitutional, insertional, deletion and covalent variants and derivatives. The term “derivative” is used synonymously with the term “variant” but generally refers to a molecule that has been modified and/or changed in any way relative to a reference molecule or starting molecule.

As such, renal polynucleotides encoding renal peptides or renal polypeptides containing substitutions, insertions and/or additions, deletions and covalent modifications with respect to reference sequences, in particular the renal polypeptide sequences disclosed herein, are included within the scope of this invention. For example, sequence tags or amino acids, such as one or more lysines, can be added to the renal peptide sequences of the invention (e.g., at the N-terminal or C-terminal ends). Sequence tags can be used for renal peptide purification or localization. Lysines can be used to increase renal peptide solubility or to allow for biotinylation. Alternatively, amino acid residues located at the carboxy and amino terminal regions of the amino acid sequence of a renal peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble, or linked to a solid support.

“Substitutional variants” when referring to renal polypeptides are those that have at least one amino acid residue in a native or starting sequence removed and a different amino acid inserted in its place at the same position. The substitutions may be single, where only one amino acid in the molecule has been substituted, or they may be multiple, where two or more amino acids have been substituted in the same molecule.

As used herein the term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine and leucine for another non-polar residue. Likewise, examples of conservative substitutions include the substitution of one polar (hydrophilic) residue for another such as between arginine and lysine, between glutamine and asparagine, and between glycine and serine. Additionally, the substitution of a basic residue such as lysine, arginine or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

“Insertional variants” when referring to renal polypeptides are those with one or more amino acids inserted immediately adjacent to an amino acid at a particular position in a native or starting sequence. “Immediately adjacent” to an amino acid means connected to either the alpha-carboxy or alpha-amino functional group of the amino acid.

“Deletional variants” when referring to renal polypeptides are those with one or more amino acids in the native or starting amino acid sequence removed. Ordinarily, deletional variants will have one or more amino acids deleted in a particular region of the molecule.

“Covalent derivatives” when referring to renal polypeptides include modifications of a native or starting protein with an organic proteinaceous or non-proteinaceous derivatizing agent, and/or post-translational modifications. Covalent modifications are traditionally introduced by reacting targeted amino acid residues of the protein with an organic derivatizing agent that is capable of reacting with selected side-chains or terminal residues, or by harnessing mechanisms of post-translational modifications that function in selected recombinant host cells. The resultant covalent derivatives are useful in programs directed at identifying residues important for biological activity, for immunoassays, or for the preparation of anti-protein antibodies for immunoaffinity purification of the recombinant glycoprotein. Such modifications are within the ordinary skill in the art and are performed without undue experimentation.

Certain post-translational modifications are the result of the action of recombinant host cells on the expressed renal polypeptide. Glutaminyl and asparaginyl residues are frequently post-translationally deamidated to the corresponding glutamyl and aspartyl residues. Alternatively, these residues are deamidated under mildly acidic conditions. Either form of these residues may be present in the renal polypeptides produced in accordance with the present invention.

Other post-translational modifications include hydroxylation of proline and lysine, phosphorylation of hydroxyl groups of seryl or threonyl residues, methylation of the alpha-amino groups of lysine, arginine, and histidine side chains (T. E. Creighton, Proteins: Structure and Molecular Properties, W.H. Freeman & Co., San Francisco, pp. 79-86 (1983)).

“Features” when referring to renal polypeptides are defined as distinct amino acid sequence-based components of a molecule. Features of the renal polypeptides encoded by the renal polynucleotides of the present invention include surface manifestations, local conformational shape, folds, loops, half-loops, domains, half-domains, sites, termini or any combination thereof.

As used herein when referring to renal polypeptides the term “surface manifestation” refers to a renal polypeptide based component of a protein appearing on an outermost surface.

As used herein when referring to renal polypeptides the term “local conformational shape” means a renal polypeptide based structural manifestation of a protein which is located within a definable space of the protein.

As used herein when referring to renal polypeptides the term “fold” refers to the resultant conformation of an amino acid sequence upon energy minimization. A fold may occur at the secondary or tertiary level of the folding process. Examples of secondary level folds include beta sheets and alpha helices. Examples of tertiary folds include domains and regions formed due to aggregation or separation of energetic forces. Regions formed in this way include hydrophobic and hydrophilic pockets, and the like.

As used herein the term “turn” as it relates to protein conformation means a bend which alters the direction of the backbone of a renal peptide or renal polypeptide and may involve one, two, three or more amino acid residues.

As used herein when referring to renal polypeptides the term “loop” refers to a structural feature of a renal polypeptide which may serve to reverse the direction of the backbone of a renal peptide or renal polypeptide. Where the loop is found in a renal polypeptide and only alters the direction of the backbone, it may comprise four or more amino acid residues. Oliva et al. have identified at least 5 classes of protein loops (J. Mol Biol 266 (4): 814-830; 1997). Loops may be open or closed. Closed loops or “cyclic” loops may comprise 2, 3, 4, 5, 6, 7, 8, 9, 10 or more amino acids between the bridging moieties. Such bridging moieties may comprise a cysteine-cysteine bridge (Cys-Cys) typical in renal polypeptides having disulfide bridges or alternatively bridging moieties may be non-protein based such as the dibromozylyl agents used herein.

As used herein when referring to renal polypeptides the term “half-loop” refers to a portion of an identified loop having at least half the number of amino acid resides as the loop from which it is derived. It is understood that loops may not always contain an even number of amino acid residues. Therefore, in those cases where a loop contains or is identified to comprise an odd number of amino acids, a half-loop of the odd-numbered loop will comprise the whole number portion or next whole number portion of the loop (number of amino acids of the loop/2+/−0.5 amino acids). For example, a loop identified as a 7 amino acid loop could produce half-loops of 3 amino acids or 4 amino acids (7/2=3.5+7-0.5 being 3 or 4).

As used herein when referring to renal polypeptides the term “domain” refers to a motif of a renal polypeptide having one or more identifiable structural or functional characteristics or properties (e.g., binding capacity, serving as a site for protein-protein interactions).

As used herein when referring to renal polypeptides the term “half-domain” means a portion of an identified domain having at least half the number of amino acid resides as the domain from which it is derived. It is understood that domains may not always contain an even number of amino acid residues. Therefore, in those cases where a domain contains or is identified to comprise an odd number of amino acids, a half-domain of the odd-numbered domain will comprise the whole number portion or next whole number portion of the domain (number of amino acids of the domain/2+/−0.5 amino acids). For example, a domain identified as a 7 amino acid domain could produce half-domains of 3 amino acids or 4 amino acids (7/2=3.5+7-0.5 being 3 or 4). It is also understood that sub-domains may be identified within domains or half-domains, these subdomains possessing less than all of the structural or functional properties identified in the domains or half domains from which they were derived. It is also understood that the amino acids that comprise any of the domain types herein need not be contiguous along the backbone of the renal polypeptide (i.e., nonadjacent amino acids may fold structurally to produce a domain, half-domain or subdomain).

As used herein when referring to renal polypeptides the terms “site” as it pertains to amino acid based embodiments is used synonymously with “amino acid residue” and “amino acid side chain.” A site represents a position within a renal peptide or renal polypeptide that may be modified, manipulated, altered, derivatized or varied within the renal polypeptide based molecules of the present invention.

As used herein the terms “termini” or “terminus” when referring to renal polypeptides refers to an extremity of a renal peptide or renal polypeptide. Such extremity is not limited only to the first or final site of the renal peptide or renal polypeptide but may include additional amino acids in the terminal regions. The renal polypeptide based molecules of the present invention may be characterized as having both an N-terminus (terminated by an amino acid with a free amino group (NH2)) and a C-terminus (terminated by an amino acid with a free carboxyl group (COOH)). Proteins of the invention are in some cases made up of multiple renal polypeptide chains brought together by disulfide bonds or by non-covalent forces (multimers, oligomers). These sorts of proteins will have multiple N- and C-termini. Alternatively, the termini of the renal polypeptides may be modified such that they begin or end, as the case may be, with a non-renal polypeptide based moiety such as an organic conjugate.

Once any of the features have been identified or defined as a desired component of a renal polypeptide to be encoded by the renal polynucleotide of the invention, any of several manipulations and/or modifications of these features may be performed by moving, swapping, inverting, deleting, randomizing or duplicating. Furthermore, it is understood that manipulation of features may result in the same outcome as a modification to the molecules of the invention. For example, a manipulation which involved deleting a domain would result in the alteration of the length of a molecule just as modification of a nucleic acid to encode less than a full length molecule would.

Modifications and manipulations can be accomplished by methods known in the art such as, but not limited to, site directed mutagenesis or a priori incorporation during chemical synthesis. The resulting modified molecules may then be tested for activity using in vitro or in vivo assays such as those described herein or any other suitable screening assay known in the art.

According to the present invention, the renal polypeptides may comprise a consensus sequence which is discovered through rounds of experimentation. As used herein a “consensus” sequence is a single sequence which represents a collective population of sequences allowing for variability at one or more sites.

As recognized by those skilled in the art, protein fragments, functional protein domains, and homologous proteins are also considered to be within the scope of renal polypeptides of interest of this invention. For example, provided herein is any protein fragment (meaning a renal polypeptide sequence at least one amino acid residue shorter than a reference renal polypeptide sequence but otherwise identical) of a reference protein 10, 20, 30, 40, 50, 60, 70, 80, 90, 100 or greater than 100 amino acids in length. In another example, any protein that includes a stretch of about 20, about 30, about 40, about 50, or about 100 amino acids which are about 40%, about 50%, about 60%, about 70%, about 80%, about 90%, about 95%, or about 100% identical to any of the sequences described herein can be utilized in accordance with the invention. In certain embodiments, a renal polypeptide to be utilized in accordance with the invention includes 2, 3, 4, 5, 6, 7, 8, 9, 10, or more mutations as shown in any of the sequences provided or referenced herein.

### Categories of Renal Polypeptides of Interest

The renal polynucleotides of the present invention may be designed to encode renal polypeptides of interest selected from any of several target categories or types including, but not limited to, biologics, antibodies, vaccines, therapeutic proteins or renal peptides, cell penetrating renal peptides, secreted proteins, plasma membrane proteins, cytoplasmic or cytoskeletal proteins, intracellular membrane bound proteins, nuclear proteins, proteins associated with human disease, targeting moieties or those proteins encoded by the human genome for which no therapeutic indication has been identified but which nonetheless have utility in areas of research and discovery. Each of these target categories are described in co-pending International Publication Nos. WO2013151666, WO2013151667, WO2013151668, WO2013151663, WO2013151669, WO2013151670, WO2013151664, WO2013151665, WO2013151736, WO2013151671, WO2013151672 and WO2013151671, the contents of each of which are herein incorporated by reference in their entirety.

In one embodiment, renal polynucleotides may encode variant renal polypeptides which have a certain identity with a reference renal polypeptide sequence. As used herein, a “reference renal polypeptide sequence” refers to a starting renal polypeptide sequence. Reference sequences may be wild type sequences or any sequence to which reference is made in the design of another sequence. A “reference renal polypeptide sequence” may, e.g., be any one of those renal polypeptides disclosed in Table 6 of co-pending International Publication Nos. WO2013151666, WO2013151667, WO2013151668, WO2013151663, WO2013151669, WO2013151670, WO2013151664, WO2013151665, WO2013151736, WO2013151671 and WO2013151672 and Table 178 of International Publication No. WO2013151671; the contents of each of which are herein incorporated by reference in their entireties.

Reference molecules (renal polypeptides or renal polynucleotides) may share a certain identity with the designed molecules (renal polypeptides or renal polynucleotides). The term “identity” as known in the art, refers to a relationship between the sequences of two or more renal peptides, renal polypeptides or renal polynucleotides, as determined by comparing the sequences. In the art, identity also means the degree of sequence relatedness between them as determined by the number of matches between strings of two or more amino acid residues or nucleosides. Identity measures the percent of identical matches between the smaller of two or more sequences with gap alignments (if any) addressed by a particular mathematical model or computer program (i.e., “algorithms”). Identity of related renal peptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math. 48, 1073 (1988).

In some embodiments, the encoded renal polypeptide variant may have the same or a similar activity as the reference renal polypeptide. Alternatively, the variant may have an altered activity (e.g., increased or decreased) relative to a reference renal polypeptide. Generally, variants of a particular renal polynucleotide or renal polypeptide of the invention will have at least about 40%, 45%, 50%, 55%, 60%, 65%, 70%, 75%, 80%, 85%, 90%, 91%, 92%, 93%, 94%, 95%, 96%, 97%, 98%, 99% but less than 100% sequence identity to that particular reference renal polynucleotide or renal polypeptide as determined by sequence alignment programs and parameters described herein and known to those skilled in the art. Such tools for alignment include those of the BLAST suite (Stephen F. Altschul, Thomas L. Madden, Alejandro A. Schïffer, Jinghui Zhang, Zheng Zhang, Webb Miller, and David J. Lipman (1997), “Gapped BLAST and PSI-BLAST: a new generation of protein database search programs”, Nucleic Acids Res. 25:3389-3402.) Other tools are described herein, specifically in the definition of “Identity.”

Default parameters in the BLAST algorithm include, for example, an expect threshold of 10, Word size of 28, Match/Mismatch Scores 1, −2, Gap costs Linear. Any filter can be applied as well as a selection for species specific repeats, e.g., Homo sapiens.

### Targeting Moieties

In some embodiments of the invention, the renal polynucleotides are provided to express a targeting moiety. These include a protein-binding partner or a receptor on the surface of the cell, which functions to target the cell to a specific tissue space or to interact with a specific moiety, either in vivo or in vitro. Suitable protein-binding partners include, but are not limited to, antibodies and functional fragments thereof, scaffold proteins, or renal peptides. Additionally, renal polynucleotides can be employed to direct the synthesis and extracellular localization of lipids, carbohydrates, or other biological moieties or biomolecules.

### Target Selection

According to the present invention, the renal polynucleotides may comprise at least a first region of linked nucleosides encoding at least one renal polypeptide of interest. Non limiting examples of renal polypeptides of interest or “Targets” of the present invention are described herein in Table 3 and those listed in Table 6 of co-pending International Publication Nos. WO2013151666, WO2013151667, WO2013151668, WO2013151663, WO2013151669, WO2013151670, WO2013151664, WO2013151665, WO2013151736, WO2013151671 and WO2013151672 and Table 178 of International Publication No. WO2013151671; the contents of each of which are herein incorporated by reference in their entireties.

In one embodiment, the renal polypeptides of interest or “Targets” of the present invention may be a target associated with a renal disease and/or disorder. Non-limiting examples of these renal targets are shown in Table 3, in addition to the name and description of the gene encoding the renal polypeptide of interest are the ENSEMBL Transcript ID (ENST), the ENSEMBL Protein ID (ENSP) and when available the optimized transcript sequence ID (Optim Trans SEQ ID) or optimized open reading frame sequence ID (Optim ORF SEQ ID). For any particular gene there may exist one or more variants or isoforms. Where these exist, they are shown in the table as well. It will be appreciated by those of skill in the art that disclosed in the Table are potential flanking regions. These are encoded in each ENST transcript either to the 5′ (upstream) or 3′ (downstream) of the ORF or coding region. The coding region is definitively and specifically disclosed by teaching the ENSP sequence. Consequently, the sequences taught flanking that encoding the protein are considered flanking regions. It is also possible to further characterize the 5′ and 3′ flanking regions by utilizing one or more available databases or algorithms. Databases have annotated the features contained in the flanking regions of the ENST transcripts and these are available in the art.

TABLE 3
Renal Targets

| | | | Trans | | | |
|---|---|---|---|---|---|---|
| | | | SEQ | | Peptide | |
| Target | | | ID | | SEQ ID | Optimized ORF SEQ |
| No. | Target Description | ENST | NO | ENSP | NO | ID |
| 1 | collagen, type IV, alpha 5 | 328300 | 37 | 331902 | 143 | 249, 355, 461, 567, |
| | | | | | | 673 |
| 2 | collagen, type IV, alpha 5 | 361603 | 38 | 354505 | 144 | 250, 356, 462, 568, |
| | | | | | | 674 |
| 3 | collagen, type IV, alpha 5 | 508186 | 39 | 425614 | 145 | 251, 357, 463, 569, |
| | | | | | | 675 |
| 4 | collagen, type IV, alpha 3 | 315699 | 40 | 323334 | 146 | 252, 358, 464, 570, |
| | (Goodpasture antigen) | | | | | 676 |
| 5 | collagen, type IV, alpha 3 | 328380 | 41 | 327594 | 147 | 253, 359, 465, 571, |
| | (Goodpasture antigen) | | | | | 677 |
| 6 | collagen, type IV, alpha 3 | 335583 | 42 | 335120 | 148 | 254, 360, 466, 572, |
| | (Goodpasture antigen) | | | | | 678 |
| 7 | collagen, type IV, alpha 3 | 396574 | 43 | 379819 | 149 | 255, 361, 467, 573, |
| | (Goodpasture antigen) | | | | | 679 |
| 8 | collagen, type IV, alpha 3 | 396578 | 44 | 379823 | 150 | 256, 362, 468, 574, |
| | (Goodpasture antigen) | | | | | 680 |
| 9 | collagen, type IV, alpha 4 | 329662 | 45 | 328553 | 151 | 257, 363, 469, 575, |
| | | | | | | 681 |
| 10 | collagen, type IV, alpha 4 | 396625 | 46 | 379866 | 152 | 258, 364, 470, 576, |
| | | | | | | 682 |
| 11 | nephrosis 1, congenital, | 353632 | 47 | 343634 | 153 | 259, 365, 471, 577, |
| | Finnish type (nephrin) | | | | | 683 |
| 12 | nephrosis 1, congenital, | 378910 | 48 | 368190 | 154 | 260, 366, 472, 578, |
| | Finnish type (nephrin) | | | | | 684 |
| 13 | LIM homeobox | 355497 | 49 | 347684 | 155 | 261, 367, 473, 579, |
| | transcription factor 1, beta | | | | | 685 |
| 14 | LIM homeobox | 373474 | 50 | 362573 | 156 | 262, 368, 474, 580, |
| | transcription factor 1, beta | | | | | 686 |
| 15 | LIM homeobox | 425646 | 51 | 390923 | 157 | 263, 369, 475, 581, |
| | transcription factor 1, beta | | | | | 687 |
| 16 | LIM homeobox | 526117 | 52 | 436930 | 158 | 264, 370, 476, 582, |
| | transcription factor 1, beta | | | | | 688 |
| 17 | Wilms tumor 1 | 332351 | 53 | 331327 | 159 | 265, 371, 477, 583, |
| | | | | | | 689 |
| 18 | Wilms tumor 1 | 379077 | 54 | 368368 | 160 | 266, 372, 478, 584, |
| | | | | | | 690 |
| 19 | Wilms tumor 1 | 379079 | 55 | 368370 | 161 | 267, 373, 479, 585, |
| | | | | | | 691 |
| 20 | Wilms tumor 1 | 448076 | 56 | 413452 | 162 | 268, 374, 480, 586, |
| | | | | | | 692 |
| 21 | Wilms tumor 1 | 452863 | 57 | 415516 | 163 | 269, 375, 481, 587, |
| | | | | | | 693 |
| 22 | Wilms tumor 1 | 527775 | 58 | 435351 | 164 | 270, 376, 482, 588, |
| | | | | | | 694 |
| 23 | Wilms tumor 1 | 527882 | 59 | 435624 | 165 | 271, 377, 483, 589, |
| | | | | | | 695 |
| 24 | Wilms tumor 1 | 530998 | 60 | 435307 | 166 | 272, 378, 484, 590, |
| | | | | | | 696 |
| 25 | polycystic kidney disease 1 | 262304 | 61 | 262304 | 167 | 273, 379, 485, 591, |
| | (autosomal dominant) | | | | | 697 |
| 26 | polycystic kidney disease 1 | 306101 | 62 | 302503 | 168 | 274, 380, 486, 592, |
| | (autosomal dominant) | | | | | 698 |
| 27 | polycystic kidney disease 1 | 382481 | 63 | 371921 | 169 | 275, 381, 487, 593, |
| | (autosomal dominant) | | | | | 699 |
| 28 | polycystic kidney disease 1 | 423118 | 64 | 399501 | 170 | 276, 382, 488, 594, |
| | (autosomal dominant) | | | | | 700 |
| 29 | polycystic kidney disease 2 | 237596 | 65 | 237596 | 171 | 277, 383, 489, 595, |
| | (autosomal dominant) | | | | | 701 |
| 30 | polycystic kidney disease 2 | 502363 | 66 | 425289 | 172 | 278, 384, 490, 596, |
| | (autosomal dominant) | | | | | 702 |
| 31 | polycystic kidney disease 2 | 508588 | 67 | 427131 | 173 | 279, 385, 491, 597, |
| | (autosomal dominant) | | | | | 703 |
| 32 | tuberous sclerosis 2 | 219476 | 68 | 219476 | 174 | 280, 386, 492, 598, |
| | | | | | | 704 |
| 33 | tuberous sclerosis 2 | 350773 | 69 | 344383 | 175 | 281, 387, 493, 599, |
| | | | | | | 705 |
| 34 | tuberous sclerosis 2 | 353929 | 70 | 248099 | 176 | 282, 388, 494, 600, |
| | | | | | | 706 |
| 35 | tuberous sclerosis 2 | 382538 | 71 | 371978 | 177 | 283, 389, 495, 601, |
| | | | | | | 707 |
| 36 | tuberous sclerosis 2 | 401874 | 72 | 384468 | 178 | 284, 390, 496, 602, |
| | | | | | | 708 |
| 37 | tuberous sclerosis 2 | 439673 | 73 | 399232 | 179 | 285, 391, 497, 603, |
| | | | | | | 709 |
| 38 | mal, T-cell differentiation | 272462 | 74 | 272462 | 180 | 286, 392, 498, 604, |
| | protein-like | | | | | 710 |
| 39 | solute carrier family 4, | 262418 | 75 | 262418 | 181 | 287, 393, 499, 605, |
| | anion exchanger, member | | | | | 711 |
| | 1 (erythrocyte membrane | | | | | |
| | protein band 3, Diego | | | | | |
| | blood group) | | | | | |
| 40 | claudin 16 | 264734 | 76 | 264734 | 182 | 288, 394, 500, 606, |
| | | | | | | 712 |
| 41 | claudin 16 | 456423 | 77 | 414136 | 183 | 289, 395, 501, 607, |
| | | | | | | 713 |
| 42 | ATPase, H+ transporting, | 234396 | 78 | 234396 | 184 | 290, 396, 502, 608, |
| | lysosomal 56/58 kDa, V1 | | | | | 714 |
| | subunit B1 | | | | | |
| 43 | ATPase, H+ transporting, | 412314 | 79 | 388353 | 185 | 291, 397, 503, 609, |
| | lysosomal 56/58 kDa, V1 | | | | | 715 |
| | subunit B1 | | | | | |
| 44 | ATPase, H+ transporting, | 483246 | 80 | 443856 | 186 | 292, 398, 504, 610, |
| | lysosomal 56/58 kDa, V1 | | | | | 716 |
| | subunit B1 | | | | | |
| 45 | carbonic anhydrase II | 285379 | 81 | 285379 | 187 | 293, 399, 505, 611, |
| | | | | | | 717 |
| 46 | chloride channel 5 | 307367 | 82 | 304257 | 188 | 294, 400, 506, 612, |
| | | | | | | 718 |
| 47 | chloride channel 5 | 376088 | 83 | 365256 | 189 | 295, 401, 507, 613, |
| | | | | | | 719 |
| 48 | chloride channel 5 | 376091 | 84 | 365259 | 190 | 296, 402, 508, 614, |
| | | | | | | 720 |
| 49 | chloride channel 5 | 376108 | 85 | 365276 | 191 | 297, 403, 509, 615, |
| | | | | | | 721 |
| 50 | chloride channel 5 | 450422 | 86 | 400415 | 192 | 298, 404, 510, 616, |
| | | | | | | 722 |
| 51 | arginine vasopressin | 337474 | 87 | 338072 | 193 | 299, 405, 511, 617, |
| | receptor 2 | | | | | 723 |
| 52 | arginine vasopressin | 358927 | 88 | 351805 | 194 | 300, 406, 512, 618, |
| | receptor 2 | | | | | 724 |
| 53 | arginine vasopressin | 370049 | 89 | 359066 | 195 | 301, 407, 513, 619, |
| | receptor 2 | | | | | 725 |
| 54 | aquaporin 2 (collecting | 199280 | 90 | 199280 | 196 | 302, 408, 514, 620, |
| | duct) | | | | | 726 |
| 55 | calcium-sensing receptor | 296154 | 91 | 296154 | 197 | 303, 409, 515, 621, |
| | | | | | | 727 |
| 56 | calcium-sensing receptor | 490131 | 92 | 418685 | 198 | 304, 410, 516, 622, |
| | | | | | | 728 |
| 57 | calcium-sensing receptor | 498619 | 93 | 420194 | 199 | 305, 411, 517, 623, |
| | | | | | | 729 |
| 58 | phosphate regulating | 379374 | 94 | 368682 | 200 | 306, 412, 518, 624, |
| | endopeptidase homolog, | | | | | 730 |
| | X-linked | | | | | |
| 59 | phosphate regulating | 418858 | 95 | 443531 | 201 | 307, 413, 519, 625, |
| | endopeptidase homolog, | | | | | 731 |
| | X-linked | | | | | |
| 60 | phosphate regulating | 535894 | 96 | 439418 | 202 | 308, 414, 520, 626, |
| | endopeptidase homolog, | | | | | 732 |
| | X-linked | | | | | |
| 61 | phosphate regulating | 537599 | 97 | 440362 | 203 | 309, 415, 521, 627, |
| | endopeptidase homolog, | | | | | 733 |
| | X-linked | | | | | |
| 62 | cytochrome P450, family | 228606 | 98 | 228606 | 204 | 310, 416, 522, 628, |
| | 27, subfamily B, | | | | | 734 |
| | polypeptide 1 | | | | | |
| 63 | solute carrier family 12 | 330289 | 99 | 331550 | 205 | 311, 417, 523, 629, |
| | (sodium/potassium/chloride | | | | | 735 |
| | transporters), member 1 | | | | | |
| 64 | solute carrier family 12 | 380993 | 100 | 370381 | 206 | 312, 418, 524, 630, |
| | (sodium/potassium/chloride | | | | | 736 |
| | transporters), member 1 | | | | | |
| 65 | solute carrier family 12 | 396577 | 101 | 379822 | 207 | 313, 419, 525, 631, |
| | (sodium/potassium/chloride | | | | | 737, 781-867 |
| | transporters), member 1 | | | | | |
| 66 | solute carrier family 12 | 428362 | 102 | 410367 | 208 | 314, 420, 526, 632, |
| | (sodium/potassium/chloride | | | | | 738 |
| | transporters), member 1 | | | | | |
| 67 | solute carrier family 12 | 546071 | 103 | 441148 | 209 | 315, 421, 527, 633, |
| | (sodium/potassium/chloride | | | | | 739 |
| | transporters), member 1 | | | | | |
| 68 | solute carrier family 12 | 558405 | 104 | 453409 | 210 | 316, 422, 528, 634, |
| | (sodium/potassium/chloride | | | | | 740 |
| | transporters), member 1 | | | | | |
| 69 | solute carrier family 12 | 559641 | 105 | 453230 | 211 | 317, 423, 529, 635, |
| | (sodium/potassium/chloride | | | | | 741 |
| | transporters), member 1 | | | | | |
| 70 | solute carrier family 12 | 563236 | 106 | 456149 | 212 | 318, 424, 530, 636, |
| | (sodium/chloride | | | | | 742 |
| | transporters) member 3 | | | | | |
| 71 | solute carrier family 12 | 566786 | 107 | 457552 | 213 | 319, 425, 531, 637, |
| | (sodium/chloride | | | | | 743 |
| | transporters) member 3 | | | | | |
| 72 | solute carrier family 12 | 262502 | 108 | 262502 | 214 | 320, 426, 532, 638, |
| | (sodium/chloride | | | | | 744 |
| | transporters), member 3 | | | | | |
| 73 | solute carrier family 12 | 438926 | 109 | 402152 | 215 | 321, 427, 533, 639, |
| | (sodium/chloride | | | | | 745 |
| | transporters), member 3 | | | | | |
| 74 | potassium inwardly- | 392665 | 110 | 376433 | 216 | 322, 428, 534, 640, |
| | rectifying channel | | | | | 746 |
| | subfamily J member 1 | | | | | |
| 75 | potassium inwardly- | 440599 | 111 | 406320 | 217 | 323, 429, 535, 641, |
| | rectifying channel | | | | | 747 |
| | subfamily J member 1 | | | | | |
| 76 | potassium inwardly- | 324036 | 112 | 316233 | 218 | 324, 430, 536, 642, |
| | rectifying channel, | | | | | 748 |
| | subfamily J, member 1 | | | | | |
| 77 | potassium inwardly- | 392664 | 113 | 376432 | 219 | 325, 431, 537, 643, |
| | rectifying channel, | | | | | 749 |
| | subfamily J, member 1 | | | | | |
| 78 | potassium inwardly- | 392666 | 114 | 376434 | 220 | 326, 432, 538, 644, |
| | rectifying channel, | | | | | 750 |
| | subfamily J, member 1 | | | | | |
| 79 | chloride channel Kb | 331579 | 115 | 332055 | 221 | 327, 433, 539, 645, |
| | | | | | | 751 |
| 80 | chloride channel Kb | 375667 | 116 | 364819 | 222 | 328, 434, 540, 646, |
| | | | | | | 752 |
| 81 | chloride channel Kb | 375668 | 117 | 364820 | 223 | 329, 435, 541, 647, |
| | | | | | | 753 |
| 82 | chloride channel Kb | 375679 | 118 | 364831 | 224 | 330, 436, 542, 648, |
| | | | | | | 754 |
| 83 | chloride channel Kb | 431772 | 119 | 389344 | 225 | 331, 437, 543, 649, |
| | | | | | | 755 |
| 84 | sodium channel, | 300061 | 120 | 300061 | 226 | 332, 438, 544, 650, |
| | nonvoltage-gated 1, | | | | | 756 |
| | gamma | | | | | |
| 85 | sodium channel, | 307331 | 121 | 302874 | 227 | 333, 439, 545, 651, |
| | nonvoltage-gated 1, beta | | | | | 757 |
| 86 | sodium channel, | 343070 | 122 | 345751 | 228 | 334, 440, 546, 652, |
| | nonvoltage-gated 1, beta | | | | | 758 |
| 87 | sodium channel, | 338748 | 123 | 345028 | 229 | 335, 441, 547, 653, |
| | nonvoltage-gated 1 alpha | | | | | 759 |
| 88 | sodium channel, | 360168 | 124 | 353292 | 230 | 336, 442, 548, 654, |
| | nonvoltage-gated 1 alpha | | | | | 760 |
| 89 | sodium channel, | 543768 | 125 | 438739 | 231 | 337, 443, 549, 655, |
| | nonvoltage-gated 1 alpha | | | | | 761 |
| 90 | sodium channel, | 228916 | 126 | 228916 | 232 | 338, 444, 550, 656, |
| | nonvoltage-gated 1 alpha | | | | | 762 |
| 91 | sodium channel, | 358945 | 127 | 351825 | 233 | 339, 445, 551, 657, |
| | nonvoltage-gated 1 alpha | | | | | 763 |
| 92 | nuclear receptor subfamily | 342437 | 128 | 343907 | 234 | 340, 446, 552, 658, |
| | 3, group C, member 2 | | | | | 764 |
| 93 | nuclear receptor subfamily | 344721 | 129 | 341390 | 235 | 341, 447, 553, 659, |
| | 3, group C, member 2 | | | | | 765 |
| 94 | nuclear receptor subfamily | 355292 | 130 | 347441 | 236 | 342, 448, 554, 660, |
| | 3, group C, member 2 | | | | | 766 |
| 95 | nuclear receptor subfamily | 358102 | 131 | 350815 | 237 | 343, 449, 555, 661, |
| | 3, group C, member 2 | | | | | 767 |
| 96 | nuclear receptor subfamily | 511528 | 132 | 421481 | 238 | 344, 450, 556, 662, |
| | 3, group C, member 2 | | | | | 768 |
| 97 | nuclear receptor subfamily | 512865 | 133 | 423510 | 239 | 345, 451, 557, 663, |
| | 3, group C, member 2 | | | | | 769 |
| 98 | nuclear receptor subfamily | 544252 | 134 | 444458 | 240 | 346, 452, 558, 664, |
| | 3, group C, member 2 | | | | | 770 |
| 99 | hydroxysteroid (11-beta) | 261465 | 135 | 261465 | 241 | 347, 453, 559, 665, |
| | dehydrogenase 1 | | | | | 771 |
| 100 | hydroxysteroid (11-beta) | 367027 | 136 | 355994 | 242 | 348, 454, 560, 666, |
| | dehydrogenase 1 | | | | | 772 |
| 101 | hydroxysteroid (11-beta) | 367028 | 137 | 355995 | 243 | 349, 455, 561, 667, |
| | dehydrogenase 1 | | | | | 773 |
| 102 | solute carrier family 7 | 590341 | 138 | 464822 | 244 | 350, 456, 562, 668, |
| | (glycoprotein-associated | | | | | 774 |
| | amino acid transporter light | | | | | |
| | chain bo + system) | | | | | |
| | member 9 | | | | | |
| 103 | solute carrier family 7 | 23064 | 139 | 23064 | 245 | 351, 457, 563, 669, |
| | (glycoprotein-associated | | | | | 775, 779, 780 |
| | amino acid transporter light | | | | | |
| | chain, bo, + system), | | | | | |
| | member 9 | | | | | |
| 104 | solute carrier family 3 | 260649 | 140 | 260649 | 246 | 352, 458, 564, 670, |
| | (cystine, dibasic and | | | | | 776 |
| | neutral amino acid | | | | | |
| | transporters, activator of | | | | | |
| | cystine, dibasic and neutral | | | | | |
| | amino acid transport), | | | | | |
| | member 1 | | | | | |
| 105 | solute carrier family 3 | 540334 | 141 | 439253 | 247 | 353, 459, 565, 671, |
| | (cystine, dibasic and | | | | | 777 |
| | neutral amino acid | | | | | |
| | transporters, activator of | | | | | |
| | cystine, dibasic and neutral | | | | | |
| | amino acid transport), | | | | | |
| | member 1 | | | | | |
| 106 | solute carrier family 3 | 541289 | 142 | 439705 | 248 | 354, 460, 566, 672, |
| | (cystine, dibasic and | | | | | 778 |
| | neutral amino acid | | | | | |
| | transporters, activator of | | | | | |
| | cystine, dibasic and neutral | | | | | |
| | amino acid transport), | | | | | |
| | member 1 | | | | | |

### II. DESIGN, SYNTHESIS, QUANTITATION AND PURIFICATION OF RENAL RENAL POLYNUCLEOTIDES

### Codon Optimization

The renal polynucleotides, their regions or parts or subregions may be codon optimized. Codon optimization methods are known in the art and may be useful in efforts to achieve one or more of several goals. These goals include to match codon frequencies in target and host organisms to ensure proper folding, bias GC content to increase mRNA stability or reduce secondary structures, minimize tandem repeat codons or base runs that may impair gene construction or expression, customize transcriptional and translational control regions, insert or remove protein trafficking sequences, remove/add post translation modification sites in encoded protein (e.g. glycosylation sites), add, remove or shuffle protein domains, insert or delete restriction sites, modify ribosome binding sites and mRNA degradation sites, to adjust translational rates to allow the various domains of the protein to fold properly, or to reduce or eliminate problem secondary structures within the renal polynucleotide. Codon optimization tools, algorithms and services are known in the art, non-limiting examples include services from GeneArt (Life Technologies), DNA2.0 (Menlo Park Calif.) and/or proprietary methods. In one embodiment, the ORF sequence is optimized using optimization algorithms. Codon options for each amino acid are given in Table 4.

TABLE 4
Codon Options

| | Single Letter | |
|---|---|---|
| Amino Acid | Code | Codon Options |
| Isoleucine | I | ATT, ATC, ATA |
| Leucine | L | CTT, CTC, CTA, CTG, TTA, TTG |
| Valine | V | GTT, GTC, GTA, GTG |
| Phenylalanine | F | TTT, TTC |
| Methionine | M | ATG |
| Cysteine | C | TGT, TGC |
| Alanine | A | GCT, GCC, GCA, GCG |
| Glycine | G | GGT, GGC, GGA, GGG |
| Proline | P | CCT, CCC, CCA, CCG |
| Threonine | T | ACT, ACC, ACA, ACG |
| Serine | S | TCT, TCC, TCA, TCG, AGT, AGC |
| Tyrosine | Y | TAT, TAC |
| Tryptophan | W | TGG |
| Glutamine | Q | CAA, CAG |
| Asparagine | N | AAT, AAC |
| Histidine | H | CAT, CAC |
| Glutamic acid | E | GAA, GAG |
| Aspartic acid | D | GAT, GAC |
| Lysine | K | AAA, AAG |
| Arginine | R | CGT, CGC, CGA, CGG, AGA, AGG |
| Selenocysteine | Sec | UGA in mRNA in presence of |
| | | Selenocystein insertion element |
| | | (SECIS) |
| Stop codons | Stop | TAA, TAG, TGA |

Features, which may be considered beneficial in some embodiments of the present invention, may be encoded by regions of the renal polynucleotide and such regions may be upstream (5′) or downstream (3′) to a region which encodes a renal polypeptide. These regions may be incorporated into the renal polynucleotide before and/or after codon optimization of the protein encoding region or open reading frame (ORF). It is not required that a renal polynucleotide contain both a 5′ and 3′ flanking region. Examples of such features include, but are not limited to, untranslated regions (UTRs), Kozak sequences, an oligo(dT) sequence, and detectable tags and may include multiple cloning sites which may have Xbal recognition.

In some embodiments, a 5′ UTR and/or a 3′ UTR region may be provided as flanking regions. Multiple 5′ or 3′ UTRs may be included in the flanking regions and may be the same or of different sequences. Any portion of the flanking regions, including none, may be codon optimized and any may independently contain one or more different structural or chemical modifications, before and/or after codon optimization.

After optimization (if desired), the renal polynucleotides components are reconstituted and transformed into a vector such as, but not limited to, plasmids, viruses, cosmids, and artificial chromosomes. For example, the optimized renal polynucleotide may be reconstituted and transformed into chemically competent E. coli, yeast, neurospora, maize, drosophila, etc. where high copy plasmid-like or chromosome structures occur by methods described herein.

Synthetic renal polynucleotides and their nucleic acid analogs play an important role in the research and studies of biochemical processes. Various enzyme-assisted and chemical-based methods have been developed to synthesize renal polynucleotides and nucleic acids.

Methods of Synthesizing Renal polynucleotides

The renal polynucleotides of the present invention may be synthesized by any of the methods described herein and/or are known in the art such as, but not limited to, enzymatic methods, solid-phase chemical synthesis, liquid phase chemical synthesis, a combination of different synthetic methods, small region synthesis, and ligation of renal polynucleotide regions or subregions.

### Enzymatic Methods

The renal polynucleotides of the present invention may be synthesized using enzymatic methods known in the art. As a non-limiting example, enzymatic methods, in vitro transcription-enzymatic synthesis and RNA polymerases useful for synthesis are described in co-pending International Publication No. WO2015034928, the contents of which are herein incorporated by reference, such as in paragraphs [000276]-[000297].

### Solid-Phase Chemical Synthesis

The renal polynucleotides of the present invention (e.g., chimeric renal polynucleotides or circular renal polynucleotides) may be manufactured in whole or in part using solid phase techniques. As a non-limiting example, solid phase techniques useful for synthesis are described in co-pending International Publication No. WO2015034928, the contents of which are herein incorporated by reference, such as in paragraphs [000298]-[000307].

### Liquid Phase Chemical Synthesis

The synthesis of chimeric renal polynucleotides or circular renal polynucleotides of the present invention by the sequential addition of monomer building blocks may be carried out in a liquid phase. As a non-limiting example, solid phase techniques useful for synthesis are described in co-pending International Publication No. WO2015034928, the contents of which are herein incorporated by reference, such as in paragraph [000308].

### Combination of Different Synthetic Methods

The synthetic methods discussed above each has its own advantages and limitations. Attempts have been conducted to combine these methods to overcome the limitations. Such combinations of methods are within the scope of the present invention. As a non-limiting example, combinations of the different synthetic methods useful for the present invention are described in co-pending International Publication No. WO2015034928, the contents of which are herein incorporated by reference, such as in paragraphs [000309]-[000312].

### Small Region Synthesis

Regions or subregions of the renal polynucleotides of the present invention may comprise small RNA molecules such as siRNA, and therefore may be synthesized in the same manner. There are several methods for preparing siRNA, such as chemical synthesis using appropriately protected ribonucleoside phosphoramidites, in vitro transcription, siRNA expression vectors, and PCR expression cassettes. As a non-limiting example, synthesis of small regions useful in the present invention are described in co-pending International Publication No. WO2015034928, the contents of which are herein incorporated by reference, such as in paragraphs [000313]-[000314].

### Ligation of Renal Polynucleotide Regions or Subregions

Renal polynucleotides such as chimeric renal polynucleotides and/or circular renal polynucleotides may be prepared by ligation of one or more regions or subregions. As a non-limiting example, methods for the ligation of renal polynucleotide regions or subregions useful in the present invention are described in co-pending International Publication No. WO2015034928, the contents of which are herein incorporated by reference, such as in paragraphs [000315]-[000322].

### Modification and Conjugation of Renal Polynucleotides

Non-natural modified nucleotides may be introduced to renal polynucleotides or nucleic acids during synthesis or post-synthesis of the chains to achieve desired functions or properties. The modifications may be on internucleotide lineage, the purine or pyrimidine bases, or sugar. The modification may be introduced at the terminal of a chain or anywhere else in the chain; with chemical synthesis or with a polymerase enzyme. For example, hexitol nucleic acids (HNAs) are nuclease resistant and provide strong hybridization to RNA. Short messenger RNAs (mRNAs) with hexitol residues in two codons have been constructed (Lavrik et al., Biochemistry, 40, 11777-11784 (2001), the contents of which are incorporated herein by reference in their entirety). The antisense effects of a chimeric HNA gapmer oligonucleotide comprising a phosphorothioate central sequence flanked by 5′ and 3′ HNA sequences have also been studied (See e.g., Kang et al., Nucleic Acids Research, vol. 32(4), 4411-4419 (2004), the contents of which are incorporated herein by reference in their entirety). The preparation and uses of modified nucleotides comprising 6-member rings in RNA interference, antisense therapy or other applications are disclosed in US Pat. Application No. 2008/0261905, US Pat. Application No. 2010/0009865, and PCT Application No. WO97/30064 to Herdewijn et al.; the contents of each of which are herein incorporated by reference in their entireties). Modified nucleic acids and their synthesis are disclosed in copending PCT applications No. PCT/US2012/058519 (Attorney Docket Number M09), the contents of which are incorporated herein by reference for their entirety. The synthesis and strategy of modified renal polynucleotides is reviewed by Verma and Eckstein in Annual Review of Biochemistry, vol. 76, 99-134 (1998), the contents of which are incorporated herein by reference in their entirety.

Either enzymatic or chemical ligation methods can be used to conjugate renal polynucleotides or their regions with different functional blocks, such as fluorescent labels, liquids, nanoparticles, delivery agents, etc. The conjugates of renal polynucleotides and modified renal polynucleotides are reviewed by Goodchild in Bioconjugate Chemistry, vol. 1(3), 165-187 (1990), the contents of which are incorporated herein by reference in their entirety. U.S. Pat. No. 6,835,827 and U.S. Pat. No. 6,525,183 to Vinayak et al. (the contents of each of which are herein incorporated by reference in their entireties) teach synthesis of labeled oligonucleotides using a labeled solid support.

### Quantification

In one embodiment, the renal polynucleotides of the present invention may be quantified in exosomes or when derived from one or more bodily fluid. As used herein “bodily fluids” include peripheral blood, serum, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyl cavity fluid, and μmbilical cord blood. Alternatively, exosomes may be retrieved from an organ selected from the group consisting of lung, heart, pancreas, stomach, intestine, bladder, kidney, ovary, testis, skin, colon, breast, prostate, brain, esophagus, liver, and placenta.

In the exosome quantification method, a sample of not more than 2 mL is obtained from the subject and the exosomes isolated by size exclusion chromatography, density gradient centrifugation, differential centrifugation, nanomembrane ultrafiltration, immunoabsorbent capture, affinity purification, microfluidic separation, or combinations thereof. In the analysis, the level or concentration of a renal polynucleotide may be an expression level, presence, absence, truncation or alteration of the administered construct. It is advantageous to correlate the level with one or more clinical phenotypes or with an assay for a human disease biomarker. The assay may be performed using construct specific probes, cytometry, qRT-PCR, real-time PCR, PCR, flow cytometry, electrophoresis, mass spectrometry, or combinations thereof while the exosomes may be isolated using immunohistochemical methods such as enzyme linked immunosorbent assay (ELISA) methods. Exosomes may also be isolated by size exclusion chromatography, density gradient centrifugation, differential centrifugation, nanomembrane ultrafiltration, immunoabsorbent capture, affinity purification, microfluidic separation, or combinations thereof.

These methods afford the investigator the ability to monitor, in real time, the level of renal polynucleotides remaining or delivered. This is possible because the renal polynucleotides of the present invention differ from the endogenous forms due to the structural or chemical modifications.

In one embodiment, the renal polynucleotide may be quantified using methods such as, but not limited to, ultraviolet visible spectroscopy (UV/Vis). A non-limiting example of a UV/Vis spectrometer is a NANODROP® spectrometer (ThermoFisher, Waltham, Mass.). The quantified renal polynucleotide may be analyzed in order to determine if the renal polynucleotide may be of proper size, check that no degradation of the renal polynucleotide has occurred. Degradation of the renal polynucleotide may be checked by methods such as, but not limited to, agarose gel electrophoresis, HPLC based purification methods such as, but not limited to, strong anion exchange HPLC, weak anion exchange HPLC, reverse phase HPLC (RP-HPLC), and hydrophobic interaction HPLC (HIC-HPLC), liquid chromatography-mass spectrometry (LCMS), capillary electrophoresis (CE) and capillary gel electrophoresis (CGE).

### Purification

Purification of the renal polynucleotides described herein may include, but is not limited to, renal polynucleotide clean-up, quality assurance and quality control. Clean-up may be performed by methods known in the arts such as, but not limited to, AGENCOURT® beads (Beckman Coulter Genomics, Danvers, Mass.), poly-T beads, LNA™ oligo-T capture probes (EXIQON® Inc, Vedbaek, Denmark) or HPLC based purification methods such as, but not limited to, strong anion exchange HPLC, weak anion exchange HPLC, reverse phase HPLC (RP-HPLC), and hydrophobic interaction HPLC (HIC-HPLC). The term “purified” when used in relation to a renal polynucleotide such as a “purified renal polynucleotide” refers to one that is separated from at least one contaminant. As used herein, a “contaminant” is any substance which makes another unfit, impure or inferior. Thus, a purified renal polynucleotide (e.g., DNA and RNA) is present in a form or setting different from that in which it is found in nature, or a form or setting different from that which existed prior to subjecting it to a treatment or purification method.

A quality assurance and/or quality control check may be conducted using methods such as, but not limited to, gel electrophoresis, UV absorbance, or analytical HPLC.

In another embodiment, the renal polynucleotides may be sequenced by methods including, but not limited to reverse-transcriptase-PCR.

### III. MODIFICATIONS

As used herein in a renal polynucleotide (such as a chimeric renal polynucleotide, IVT renal polynucleotide or a circular renal polynucleotide), the terms “chemical modification” or, as appropriate, “chemically modified” refer to modification with respect to adenosine (A), guanosine (G), uridine (U), thymidine (T) or cytidine (C) ribo- or deoxyribnucleosides in one or more of their position, pattern, percent or population. Generally, herein, these terms are not intended to refer to the ribonucleotide modifications in naturally occurring 5′-terminal mRNA cap moieties.

In a renal polypeptide, the term “modification” refers to a modification as compared to the canonical set of 20 amino acids.

The modifications may be various distinct modifications. In some embodiments, the regions may contain one, two, or more (optionally different) nucleoside or nucleotide modifications. In some embodiments, a modified renal polynucleotide, introduced to a cell may exhibit reduced degradation in the cell, as compared to an unmodified renal polynucleotide.

Modifications which are useful in the present invention include, but are not limited to those in Table 4 of International Patent Publication No. WO2015038892, the contents of which are herein incorporated by reference in its entirety. As a non-limiting example, the modification may be 2-methylthio-N6-(cis-hydroxyisopentenyl)adenosine, 2-methylthio-N6-methyladenosine, 2-methylthio-N6-threonyl carbamoyladenosine, N6-glycinylcarbamoyladenosine, N6-isopentenyladenosine, N6-methyladenosine, N6-threonylcarbamoyladenosine, 1,2′-O-dimethyladenosine, 1-methyladenosine, 2′-O-methyladenosine, 2′-O-ribosyladenosine (phosphate), 2-methyladenosine, 2-methylthio-N6 isopentenyladenosine, 2-methylthio-N6-hydroxynorvalyl carbamoyladenosine, 2′-O-methyladenosine, 2′-O-ribosyladenosine (phosphate), isopentenyladenosine, N6-(cis-hydroxyisopentenyl)adenosine, N6,2′-O-dimethyladenosine, N6,2′-O-dimethyladenosine, N6,N6,2′-O-trimethyladenosine, N6,N6-dimethyladenosine, N6-acetyladenosine, N6-hydroxynorvalylcarbamoyladenosine, N6-methyl-N6-threonylcarbamoyladenosine, 2-methyladenosine, 2-methylthio-N6-isopentenyladenosine, 7-deaza-adenosine, N1-methyl-adenosine, N6, N6 (dimethyl)adenine, N6-cis-hydroxy-isopentenyl-adenosine, α-thio-adenosine, 2 (amino)adenine, 2 (aminopropyl)adenine, 2 (methylthio) N6 (isopentenyl)adenine, 2-(alkyl)adenine, 2-(aminoalkyl)adenine, 2-(aminopropyl)adenine, 2-(halo)adenine, 2-(halo)adenine, 2-(propyl)adenine, 2′-Amino-2′-deoxy-ATP, 2′-Azido-2′-deoxy-ATP, 2′-Deoxy-2′-α-aminoadenosine TP, 2′-Deoxy-2′-a-azidoadenosine TP, 6 (alkyl)adenine, 6 (methyl)adenine, 6-(alkyl)adenine, 6-(methyl)adenine, 7 (deaza)adenine, 8 (alkenyl)adenine, 8 (alkynyl)adenine, 8 (amino)adenine, 8 (thioalkyl)adenine, 8-(alkenyl)adenine, 8-(alkyl)adenine, 8-(alkynyl)adenine, 8-(amino)adenine, 8-(halo)adenine, 8-(hydroxyl)adenine, 8-(thioalkyl)adenine, 8-(thiol)adenine, 8-azido-adenosine, aza adenine, deaza adenine, N6 (methyl)adenine, N6-(isopentyl)adenine, 7-deaza-8-aza-adenosine, 7-methyladenine, 1-Deazaadenosine TP, 2′Fluoro-N6-Bz-deoxyadenosine TP, 2′-OMe-2-Amino-ATP, 2′O-methyl-N6-Bz-deoxyadenosine TP, 2′-a-Ethynyladenosine TP, 2-aminoadenine, 2-Aminoadenosine TP, 2-Amino-ATP, 2′-a-Trifluoromethyladenosine TP, 2-Azidoadenosine TP, 2′-b-Ethynyladenosine TP, 2-Bromoadenosine TP, 2′-b-Trifluoromethyladenosine TP, 2-Chloroadenosine TP, 2′-Deoxy-2′, 2′-difluoroadenosine TP, 2′-Deoxy-2′-a-mercaptoadenosine TP, 2′-Deoxy-2′-a-thiomethoxyadenosine TP, 2′-Deoxy-2′-b-aminoadenosine TP, 2′-Deoxy-2′-b-azidoadenosine TP, 2′-Deoxy-2′-b-bromoadenosine TP, 2′-Deoxy-2′-b-chloroadenosine TP, 2′-Deoxy-2′-b-fluoroadenosine TP, 2′-Deoxy-2′-b-iodoadenosine TP, 2′-Deoxy-2′-b-mercaptoadenosine TP, 2′-Deoxy-2′-b-thiomethoxyadenosine TP, 2-Fluoroadenosine TP, 2-Iodoadenosine TP, 2-Mercaptoadenosine TP, 2-methoxy-adenine, 2-methylthio-adenine, 2-Trifluoromethyladenosine TP, 3-Deaza-3-bromoadenosine TP, 3-Deaza-3-chloroadenosine TP, 3-Deaza-3-fluoroadenosine TP, 3-Deaza-3-iodoadenosine TP, 3-Deazaadenosine TP, 4′-Azidoadenosine TP, 4′-Carbocyclic adenosine TP, 4′-Ethynyladenosine TP, 5′-Homo-adenosine TP, 8-Aza-ATP, 8-bromoadenosine TP, 8-Trifluoromethyladenosine TP, 9-Deazaadenosine TP, 2-aminopurine, 7-deaza-2,6-diaminopurine, 7-deaza-8-aza-2,6-diaminopurine, 7-deaza-8-aza-2-aminopurine, 2,6-diaminopurine, 7-deaza-8-aza-adenine, 7-deaza-2-aminopurine, 2-thiocytidine, 3-methylcytidine, 5-formylcytidine, 5-hydroxymethylcytidine, 5-methylcytidine, N4-acetylcytidine, 2′-O-methylcytidine, 2′-O-methylcytidine, 5,2′-O-dimethylcytidine, 5-formyl-2′-O-methylcytidine, lysidine, N4,2′-O-dimethylcytidine, N4-acetyl-2′-O-methylcytidine, N4-methylcytidine, N4,N4-Dimethyl-2′-OMe-Cytidine TP, 4-methylcytidine, 5-aza-cytidine, Pseudo-iso-cytidine, pyrrolo-cytidine, α-thio-cytidine, 2-(thio)cytosine, 2′-Amino-2′-deoxy-CTP, 2′-Azido-2′-deoxy-CTP, 2′-Deoxy-2′-a-aminocytidine TP, 2′-Deoxy-2′-a-azidocytidine TP, 3 (deaza) 5 (aza)cytosine, 3 (methyl)cytosine, 3-(alkyl)cytosine, 3-(deaza) 5 (aza)cytosine, 3-(methyl)cytidine, 4,2′-O-dimethylcytidine, 5 (halo)cytosine, 5 (methyl)cytosine, 5 (propynyl)cytosine, 5 (trifluoromethyl)cytosine, 5-(alkyl)cytosine, 5-(alkynyl)cytosine, 5-(halo)cytosine, 5-(propynyl)cytosine, 5-(trifluoromethyl)cytosine, 5-bromo-cytidine, 5-iodo-cytidine, 5-propynyl cytosine, 6-(azo)cytosine, 6-aza-cytidine, aza cytosine, deaza cytosine, N4 (acetyl)cytosine, 1-methyl-1-deaza-pseudoisocytidine, 1-methyl-pseudoisocytidine, 2-methoxy-5-methyl-cytidine, 2-methoxy-cytidine, 2-thio-5-methyl-cytidine, 4-methoxy-1-methyl-pseudoisocytidine, 4-methoxy-pseudoisocytidine, 4-thio-1-methyl-1-deaza-pseudoisocytidine, 4-thio-1-methyl-pseudoisocytidine, 4-thio-pseudoisocytidine, 5-aza-zebularine, 5-methyl-zebularine, pyrrolo-pseudoisocytidine, zebularine, (E)-5-(2-Bromo-vinyl)cytidine TP, 2,2′-anhydro-cytidine TP hydrochloride, 2′Fluor-N4-Bz-cytidine TP, 2′Fluoro-N4-Acetyl-cytidine TP, 2′-O-Methyl-N4-Acetyl-cytidine TP, 2′O-methyl-N4-Bz-cytidine TP, 2′-a-Ethynylcytidine TP, 2′-a-Trifluoromethylcytidine TP, 2′-b-Ethynylcytidine TP, 2′-b-Trifluoromethylcytidine TP, 2′-Deoxy-2′,2′-difluorocytidine TP, 2′-Deoxy-2′-a-mercaptocytidine TP, 2′-Deoxy-2′-a-thiomethoxycytidine TP, 2′-Deoxy-2′-b-aminocytidine TP, 2′-Deoxy-2′-b-azidocytidine TP, 2′-Deoxy-2′-b-bromocytidine TP, 2′-Deoxy-2′-b-chlorocytidine TP, 2′-Deoxy-2′-b-fluorocytidine TP, 2′-Deoxy-2′-b-iodocytidine TP, 2′-Deoxy-2′-b-mercaptocytidine TP, 2′-Deoxy-2′-b-thiomethoxycytidine TP, 2′-O-Methyl-5-(1-propynyl)cytidine TP, 3′-Ethynylcytidine TP, 4′-Azidocytidine TP, 4′-Carbocyclic cytidine TP, 4′-Ethynylcytidine TP, 5-(1-Propynyl)ara-cytidine TP, 5-(2-Chloro-phenyl)-2-thiocytidine TP, 5-(4-Amino-phenyl)-2-thiocytidine TP, 5-Aminoallyl-CTP, 5-Cyanocytidine TP, 5-Ethynylara-cytidine TP, 5-Ethynylcytidine TP, 5′-Homo-cytidine TP, 5-Methoxycytidine TP, 5-Trifluoromethyl-Cytidine TP, N4-Amino-cytidine TP, N4-Benzoyl-cytidine TP, pseudoisocytidine, 7-methylguanosine, N2,2′-O-dimethylguanosine, N2-methylguanosine, wyosine, 1,2′-O-dimethylguanosine, 1-methylguanosine, 2′-O-methylguanosine, 2′-O-ribosylguanosine (phosphate), 2′-O-methylguanosine, 2′-O-ribosylguanosine (phosphate), 7-aminomethyl-7-deazaguanosine, 7-cyano-7-deazaguanosine, archaeosine, methylwyosine, N2,7-dimethylguanosine, N2,N2,2′-O-trimethylguanosine, N2,N2,7-trimethylguanosine, N2,N2-dimethylguanosine, N2,7,2′-O-trimethylguanosine, 6-thio-guanosine, 7-deaza-guanosine, 8-oxo-guanosine, N1-methyl-guanosine, α-thio-guanosine, 2 (propyl)guanine, 2-(alkyl)guanine, 2′-Amino-2′-deoxy-GTP, 2′-Azido-2′-deoxy-GTP, 2′-Deoxy-2′-a-aminoguanosine TP, 2′-Deoxy-2′-a-azidoguanosine TP, 6 (methyl)guanine, 6-(alkyl)guanine, 6-(methyl)guanine, 6-methyl-guanosine, 7 (alkyl)guanine, 7 (deaza)guanine, 7 (methyl)guanine, 7-(alkyl)guanine, 7-(deaza)guanine, 7-(methyl)guanine, 8 (alkyl)guanine, 8 (alkynyl)guanine, 8 (halo)guanine, 8 (thioalkyl)guanine, 8-(alkenyl)guanine, 8-(alkyl)guanine, 8-(alkynyl)guanine, 8-(amino)guanine, 8-(halo)guanine, 8-(hydroxyl)guanine, 8-(thioalkyl)guanine, 8-(thiol)guanine, aza guanine, deaza guanine, N (methyl)guanine, N-(methyl)guanine, 1-methyl-6-thio-guanosine, 6-methoxy-guanosine, 6-thio-7-deaza-8-aza-guanosine, 6-thio-7-deaza-guanosine, 6-thio-7-methyl-guanosine, 7-deaza-8-aza-guanosine, 7-methyl-8-oxo-guanosine, N2,N2-dimethyl-6-thio-guanosine, N2-methyl-6-thio-guanosine, 1-Me-GTP, 2′Fluoro-N2-isobutyl-guanosine TP, 2′O-methyl-N2-isobutyl-guanosine TP, 2′-a-Ethynylguanosine TP, 2′-a-Trifluoromethylguanosine TP, 2′-b-Ethynylguanosine TP, 2′-b-Trifluoromethylguanosine TP, 2′-Deoxy-2′,2′-difluoroguanosine TP, 2′-Deoxy-2′-a-mercaptoguanosine TP, 2′-Deoxy-2′-a-thiomethoxyguanosine TP, 2′-Deoxy-2′-b-aminoguanosine TP, 2′-Deoxy-2′-b-azidoguanosine TP, 2′-Deoxy-2′-b-bromoguanosine TP, 2′-Deoxy-2′-b-chloroguanosine TP, 2′-Deoxy-2′-b-fluoroguanosine TP, 2′-Deoxy-2′-b-iodoguanosine TP, 2′-Deoxy-2′-b-mercaptoguanosine TP, 2′-Deoxy-2′-b-thiomethoxyguanosine TP, 4′-Azidoguanosine TP, 4′-Carbocyclic guanosine TP, 4′-Ethynylguanosine TP, 5′-Homo-guanosine TP, 8-bromo-guanosine TP, 9-Deazaguanosine TP, N2-isobutyl-guanosine TP, 1-methylinosine, inosine, 1,2′-O-dimethylinosine, 2′-O-methylinosine, 7-methylinosine, 2′-O-methylinosine, epoxyqueuosine, galactosyl-queuosine, mannosylqueuosine, queuosine, allyamino-thymidine, aza thymidine, deaza thymidine, deoxy-thymidine, 2′-O-methyluridine, 2-thiouridine, 3-methyluridine, 5-carboxymethyluridine, 5-hydroxyuridine, 5-methyluridine, 5-taurinomethyl-2-thiouridine, 5-taurinomethyluridine, dihydrouridine, pseudouridine, (3-(3-amino-3-carboxypropyl)uridine, 1-methyl-3-(3-amino-5-carboxypropyl)pseudouridine, 1-methylpseduouridine, 1-methyl-pseudouridine, 2′-O-methyluridine, 2′-O-methylpseudouridine, 2′-O-methyluridine, 2-thio-2′-O-methyluridine, 3-(3-amino-3-carboxypropyl)uridine, 3,2′-O-dimethyluridine, 3-Methyl-pseudo-Uridine TP, 4-thiouridine, 5-(carboxyhydroxymethyl)uridine, 5-(carboxyhydroxymethyl)uridine methyl ester, 5,2′-O-dimethyluridine, 5,6-dihydro-uridine, 5-aminomethyl-2-thiouridine, 5-carbamoylmethyl-2′-O-methyluridine, 5-carbamoylmethyluridine, 5-carboxyhydroxymethyluridine, 5-carboxyhydroxymethyl uridine methyl ester, 5-carboxymethylam inomethyl-2′-O-methyluridine, 5-carboxymethylaminomethyl-2-thiouridine, 5-carboxymethylaminomethyl-2-thiouridine, 5-carboxymethylaminomethyluridine, 5-carboxymethylaminomethyluridine, 5-Carbamoylmethyluridine TP, 5-methoxycarbonylmethyl-2′-O-methyluridine, 5-methoxycarbonylmethyl-2-thiouridine, 5-methoxycarbonylmethyluridine, 5-methoxyuridine, 5-methyl-2-thiouridine, 5-methylaminomethyl-2-selenouridine, 5-methylaminomethyl-2-thiouridine, 5-methylaminomethyluridine, 5-Methyldihydrouridine, 5-Oxyacetic acid-Uridine TP, 5-Oxyacetic acid-methyl ester-Uridine TP, N1-methyl-pseudo-uridine, uridine 5-oxyacetic acid, uridine 5-oxyacetic acid methyl ester, 3-(3-Amino-3-carboxypropyl)-Uridine TP, 5-(iso-Pentenylaminomethyl)-2-thiouridine TP, 5-(iso-Pentenylaminomethyl)-2′-O-methyluridine TP, 5-(iso-Pentenylaminomethyl)uridine TP, 5-propynyl uracil, α-thio-uridine, 1 (aminoalkylamino-carbonylethylenyl)-2(thio)-pseudouracil, 1 (aminoalkylaminocarbonylethylenyl)-2,4-(dithio)pseudouracil, 1 (aminoalkylaminocarbonylethylenyl)-4 (thio)pseudouracil, 1 (aminoalkylaminocarbonylethylenyl)-pseudouracil, 1 (aminocarbonylethylenyl)-2(thio)-pseudouracil, 1 (aminocarbonylethylenyl)-2,4-(dithio)pseudouracil, 1 (aminocarbonylethylenyl)-4 (thio)pseudouracil, 1 (aminocarbonylethylenyl)-pseudouracil, 1 substituted 2(thio)-pseudouracil, 1 substituted 2,4-(dithio)pseudouracil, 1 substituted 4 (thio)pseudouracil, 1 substituted pseudouracil, 1-(aminoalkylamino-carbonylethylenyl)-2-(thio)-pseudouracil, 1-Methyl-3-(3-amino-3-carboxypropyl) pseudouridine TP, 1-Methyl-3-(3-amino-3-carboxypropyl)pseudo-UTP, 1-Methyl-pseudo-UTP, 2 (thio)pseudouracil, 2′ deoxy uridine, 2′ fluorouridine, 2-(thio)uracil, 2,4-(dithio)psuedouracil, 2′ methyl, 2′amino, 2′azido, 2′fluro-guanosine, 2′-Amino-2′-deoxy-UTP, 2′-Azido-2′-deoxy-UTP, 2′-Azido-deoxyuridine TP, 2′-O-methylpseudouridine, 2′ deoxy uridine, 2′ fluorouridine, 2′-Deoxy-2′-a-aminouridine TP, 2′-Deoxy-2′-a-azidouridine TP, 2-methylpseudouridine, 3 (3 amino-3 carboxypropyl)uracil, 4 (thio)pseudouracil, 4-(thio)pseudouracil, 4-(thio)uracil, 4-thiouracil, 5 (1,3-diazole-1-alkyl)uracil, 5 (2-aminopropyl)uracil, 5 (aminoalkyl)uracil, 5 (dimethylaminoalkyl)uracil, 5 (guanidiniumalkyl)uracil, 5 (methoxycarbonylmethyl)-2-(thio)uracil, 5 (methoxycarbonyl-methyl)uracil, 5 (methyl) 2 (thio)uracil, 5 (methyl) 2,4 (dithio)uracil, 5 (methyl) 4 (thio)uracil, 5 (methylaminomethyl)-2 (thio)uracil, 5 (methylaminomethyl)-2,4 (dithio)uracil, 5 (methylaminomethyl)-4 (thio)uracil, 5 (propynyl)uracil, 5 (trifluoromethyl)uracil, 5-(2-aminopropyl)uracil, 5-(alkyl)-2-(thio)pseudouracil, 5-(alkyl)-2,4 (dithio)pseudouracil, 5-(alkyl)-4 (thio)pseudouracil, 5-(alkyl)pseudouracil, 5-(alkyl)uracil, 5-(alkynyl)uracil, 5-(allylamino)uracil, 5-(cyanoalkyl)uracil, 5-(dialkylaminoalkyl)uracil, 5-(dimethylaminoalkyl)uracil, 5-(guanidiniumalkyl)uracil, 5-(halo)uracil, 5-(1,3-diazole-1-alkyl)uracil, 5-(methoxy)uracil, 5-(methoxycarbonylmethyl)-2-(thio)uracil, 5-(methoxycarbonyl-methyl)uracil, 5-(methyl) 2(thio)uracil, 5-(methyl) 2,4 (dithio)uracil, 5-(methyl) 4 (thio)uracil, 5-(methyl)-2-(thio)pseudouracil, 5-(methyl)-2,4 (dithio)pseudouracil, 5-(methyl)-4 (thio)pseudouracil, 5-(methyl)pseudouracil, 5-(methylaminomethyl)-2 (thio)uracil, 5-(methylaminomethyl)-2,4(dithio)uracil, 5-(methylaminomethyl)-4-(thio)uracil, 5-(propynyl)uracil, 5-(trifluoromethyl)uracil, 5-aminoallyl-uridine, 5-bromo-uridine, 5-iodo-uridine, 5-uracil, 6 (azo)uracil, 6-(azo)uracil, 6-aza-uridine, allyamino-uracil, aza uracil, deaza uracil, N3 (methyl)uracil, P seudo-UTP-1-2-ethanoic acid, pseudouracil, 4-Thio-pseudo-UTP, 1-carboxymethyl-pseudouridine, 1-methyl-1-deaza-pseudouridine, 1-propynyl-uridine, 1-taurinomethyl-1-methyl-uridine, 1-taurinomethyl-4-thio-uridine, 1-taurinomethyl-pseudouridine, 2-methoxy-4-thio-pseudouridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, (±)1-(2-Hydroxypropyl)pseudouridine TP, (2R)-1-(2-Hydroxypropyl)pseudouridine TP, (2S)-1-(2-Hydroxypropyl)pseudouridine TP, (E)-5-(2-Bromo-vinyl)ara-uridine TP, (E)-5-(2-Bromo-vinyl)uridine TP, (Z)-5-(2-Bromo-vinyl)ara-uridine TP, (Z)-5-(2-Bromo-vinyl)uridine TP, 1-(2,2,2-Trifluoroethyl)-pseudo-UTP, 1-(2,2,3,3,3-Pentafluoropropyl)pseudouridine TP, 1-(2,2-Diethoxyethyl)pseudouridine TP, 1-(2,4,6-Trimethylbenzyl)pseudouridine TP, 1-(2,4,6-Trimethyl-benzyl)pseudo-UTP, 1-(2,4,6-Trimethyl-phenyl)pseudo-UTP, 1-(2-Amino-2-carboxyethyl)pseudo-UTP, 1-(2-Amino-ethyl)pseudo-UTP, 1-(2-Hydroxyethyl)pseudouridine TP, 1-(2-Methoxyethyl)pseudouridine TP, 1-(3,4-Bis-trifluoromethoxybenzyl)pseudouridine TP, 1-(3,4-Dimethoxybenzyl)pseudouridine TP, 1-(3-Amino-3-carboxypropyl)pseudo-UTP, 1-(3-Amino-propyl)pseudo-UTP, 1-(3-Cyclopropyl-prop-2-ynyl)pseudouridine TP, 1-(4-Amino-4-carboxybutyl)pseudo-UTP, 1-(4-Amino-benzyl)pseudo-UTP, 1-(4-Amino-butyl)pseudo-UTP, 1-(4-Amino-phenyl)pseudo-UTP, 1-(4-Azidobenzyl)pseudouridine TP, 1-(4-Bromobenzyl)pseudouridine TP, 1-(4-Chlorobenzyl)pseudouridine TP, 1-(4-Fluorobenzyl)pseudouridine TP, 1-(4-Iodobenzyl)pseudouridine TP, 1-(4-Methanesulfonylbenzyl)pseudouridine TP, 1-(4-Methoxybenzyl)pseudouridine TP, 1-(4-Methoxy-benzyl)pseudo-UTP, 1-(4-Methoxy-phenyl)pseudo-UTP, 1-(4-Methylbenzyl)pseudouridine TP, 1-(4-Methyl-benzyl)pseudo-UTP, 1-(4-Nitrobenzyl)pseudouridine TP, 1-(4-Nitro-benzyl)pseudo-UTP, 1(4-Nitro-phenyl)pseudo-UTP, 1-(4-Thiomethoxybenzyl)pseudouridine TP, 1-(4-Trifluoromethoxybenzyl)pseudouridine TP, 1-(4-Trifluoromethylbenzyl)pseudouridine TP, 1-(5-Amino-pentyl)pseudo-UTP, 1-(6-Amino-hexyl)pseudo-UTP, 1,6-Dimethyl-pseudo-UTP, 1-[3-(2-{2-[2-(2-Aminoethoxy)-ethoxy]-ethoxy}-ethoxy)-propionyl]pseudouridine TP, 1-{3-[2-(2-Aminoethoxy)-ethoxy]-propionyl} pseudouridine TP, 1-Acetylpseudouridine TP, 1-Alkyl-6-(1-propynyl)-pseudo-UTP, 1-Alkyl-6-(2-propynyl)-pseudo-UTP, 1-Alkyl-6-allyl-pseudo-UTP, 1-Alkyl-6-ethynyl-pseudo-UTP, 1-Alkyl-6-homoallyl-pseudo-UTP, 1-Alkyl-6-vinyl-pseudo-UTP, 1-Allylpseudouridine TP, 1-Aminomethyl-pseudo-UTP, 1-Benzoylpseudouridine TP, 1-Benzyloxymethylpseudouridine TP, 1-Benzyl-pseudo-UTP, 1-Biotinyl-PEG2-pseudouridine TP, 1-Biotinylpseudouridine TP, 1-Butyl-pseudo-UTP, 1-Cyanomethylpseudouridine TP, 1-Cyclobutylmethyl-pseudo-UTP, 1-Cyclobutyl-pseudo-UTP, 1-Cycloheptylmethyl-pseudo-UTP, 1-Cycloheptyl-pseudo-UTP, 1-Cyclohexylmethyl-pseudo-UTP, 1-Cyclohexyl-pseudo-UTP, 1-Cyclooctylmethyl-pseudo-UTP, 1-Cyclooctyl-pseudo-UTP, 1-Cyclopentylmethyl-pseudo-UTP, 1-Cyclopentyl-pseudo-UTP, 1-Cyclopropylmethyl-pseudo-UTP, 1-Cyclopropyl-pseudo-UTP, 1-Ethyl-pseudo-UTP, 1-Hexyl-pseudo-UTP, 1-Homoallylpseudouridine TP, 1-Hydroxymethylpseudouridine TP, 1-iso-propyl-pseudo-UTP, 1-Me-2-thio-pseudo-UTP, 1-Me-4-thio-pseudo-UTP, 1-Me-alpha-thio-pseudo-UTP, 1-Methanesulfonylmethylpseudouridine TP, 1-Methoxymethylpseudouridine TP, 1-Methyl-6-(2,2,2-Trifluoroethyl)pseudo-UTP, 1-Methyl-6-(4-morpholino)-pseudo-UTP, 1-Methyl-6-(4-thiomorpholino)-pseudo-UTP, 1-Methyl-6-(substituted phenyl)pseudo-UTP, 1-Methyl-6-amino-pseudo-UTP, 1-Methyl-6-azido-pseudo-UTP, 1-Methyl-6-bromo-pseudo-UTP, 1-Methyl-6-butyl-pseudo-UTP, 1-Methyl-6-chloro-pseudo-UTP, 1-Methyl-6-cyano-pseudo-UTP, 1-Methyl-6-dimethylamino-pseudo-UTP, 1-Methyl-6-ethoxy-pseudo-UTP, 1-Methyl-6-ethylcarboxylate-pseudo-UTP, 1-Methyl-6-ethyl-pseudo-UTP, 1-Methyl-6-fluoro-pseudo-UTP, 1-Methyl-6-formyl-pseudo-UTP, 1-Methyl-6-hydroxyamino-pseudo-UTP, 1-Methyl-6-hydroxy-pseudo-UTP, 1-Methyl-6-iodo-pseudo-UTP, 1-Methyl-6-iso-propyl-pseudo-UTP, 1-Methyl-6-methoxy-pseudo-UTP, 1-Methyl-6-methylamino-pseudo-UTP, 1-Methyl-6-phenyl-pseudo-UTP, 1-Methyl-6-propyl-pseudo-UTP, 1-Methyl-6-tert-butyl-pseudo-UTP, 1-Methyl-6-trifluoromethoxy-pseudo-UTP, 1-Methyl-6-trifluoromethyl-pseudo-UTP, 1-Morpholinomethylpseudouridine TP, 1-Pentyl-pseudo-UTP, 1-Phenyl-pseudo-UTP, 1-Pivaloylpseudouridine TP, 1-Propargylpseudouridine TP, 1-Propyl-pseudo-UTP, 1-propynyl-pseudouridine, 1-p-tolyl-pseudo-UTP, 1-tert-Butyl-pseudo-UTP, 1-Thiomethoxymethylpseudouridine TP, 1-Thiomorpholinomethylpseudouridine TP, 1-Trifluoroacetylpseudouridine TP, 1-Trifluoromethyl-pseudo-UTP, 1-Vinylpseudouridine TP, 2,2′-anhydro-uridine TP, 2′-bromo-deoxyuridine TP, 2′-F-5-Methyl-2′-deoxy-UTP, 2′-OMe-5-Me-UTP, 2′-OMe-pseudo-UTP, 2′-a-Ethynyluridine TP, 2′-a-Trifluoromethyluridine TP, 2′-b-Ethynyluridine TP, 2′-b-Trifluoromethyluridine TP, 2′-Deoxy-2′,2′-difluorouridine TP, 2′-Deoxy-2′-a-mercaptouridine TP, 2′-Deoxy-2′-a-thiomethoxyuridine TP, 2′-Deoxy-2′-b-aminouridine TP, 2′-Deoxy-2′-b-azidouridine TP, 2′-Deoxy-2′-b-bromouridine TP, 2′-Deoxy-2′-b-chlorouridine TP, 2′-Deoxy-2′-b-fluorouridine TP, 2′-Deoxy-2′-b-iodouridine TP, 2′-Deoxy-2′-b-mercaptouridine TP, 2′-Deoxy-2′-b-thiomethoxyuridine TP, 2-methoxy-4-thio-uridine, 2-methoxyuridine, 2′-O-Methyl-5-(1-propynyOuridine TP, 3-Alkyl-pseudo-UTP, 4′-Azidouridine TP, 4′-Carbocyclic uridine TP, 4′-Ethynyluridine TP, 5-(1-Propynyl)ara-uridine TP, 5-(2-Furanyl)uridine TP, 5-Cyanouridine TP, 5-Dimethylaminouridine TP, 5′-Homo-uridine TP, 5-iodo-2′-fluoro-deoxyuridine TP, 5-Phenylethynyluridine TP, 5-Trideuteromethyl-6-deuterouridine TP, 5-Trifluoromethyl-Uridine TP, 5-Vinylarauridine TP, 6-(2,2,2-Trifluoroethyl)-pseudo-UTP, 6-(4-Morpholino)-pseudo-UTP, 6-(4-Thiomorpholino)-pseudo-UTP, 6-(Substituted-Phenyl)-pseudo-UTP, 6-Amino-pseudo-UTP, 6-Azido-pseudo-UTP, 6-Bromo-pseudo-UTP, 6-Butyl-pseudo-UTP, 6-Chloro-pseudo-UTP, 6-Cyano-pseudo-UTP, 6-Dimethylamino-pseudo-UTP, 6-Ethoxy-pseudo-UTP, 6-Ethylcarboxylate-pseudo-UTP, 6-Ethyl-pseudo-UTP, 6-Fluoro-pseudo-UTP, 6-Formyl-pseudo-UTP, 6-Hydroxyamino-pseudo-UTP, 6-Hydroxy-pseudo-UTP, 6-Iodo-pseudo-UTP, 6-iso-Propyl-pseudo-UTP, 6-Methoxy-pseudo-UTP, 6-Methylamino-pseudo-UTP, 6-Methyl-pseudo-UTP, 6-Phenyl-pseudo-UTP, 6-Phenyl-pseudo-UTP, 6-Propyl-pseudo-UTP, 6-tert-Butyl-pseudo-UTP, 6-Trifluoromethoxy-pseudo-UTP, 6-Trifluoromethyl-pseudo-UTP, Alpha-thio-pseudo-UTP, Pseudouridine 1-(4-methylbenzenesulfonic acid) TP, Pseudouridine 1-(4-methylbenzoic acid) TP, Pseudouridine TP 1-[3-(2-ethoxy)]propionic acid, Pseudouridine TP 1-[3-{2-(2-[2-(2-ethoxy)-ethoxy]-ethoxy)-ethoxy}]propionic acid, Pseudouridine TP 1-[3-{2-(2-[2-{2(2-ethoxy)-ethoxy}-ethoxy]-ethoxy)-ethoxy}]propionic acid, Pseudouridine TP 1-[3-{2-(2-[2-ethoxy]-ethoxy)-ethoxy}]propionic acid, Pseudouridine TP 1-[3-{2-(2-ethoxy)-ethoxy}]propionic acid, Pseudouridine TP 1-methylphosphonic acid, Pseudouridine TP 1-methylphosphonic acid diethyl ester, Pseudo-UTP-N1-3-propionic acid, Pseudo-UTP-N1-4-butanoic acid, Pseudo-UTP-N1-5-pentanoic acid, Pseudo-UTP-N1-6-hexanoic acid, Pseudo-UTP-N1-7-heptanoic acid, Pseudo-UTP-N1-methyl-p-benzoic acid, Pseudo-UTP-N1-p-benzoic acid, wybutosine, hydroxywybutosine, isowyosine, peroxywybutosine, or undermodified hydroxywybutosine, 4-demethylwyosine.

Other modifications which may be useful in the renal polynucleotides of the present invention are listed in Table 5 of International Patent Publication No. WO2015038892, the contents of which are herein incorporated by reference in its entirety. As a non-limiting example, the modification may be 2,6-(diamino)purine, 1-(aza)-2-(thio)-3-(aza)-phenoxazin-1-yl, 1,3-(diaza)-2-(oxo)-phenthiazin-1-yl, 1,3-(diaza)-2-(oxo)-phenoxazin-1-yl, 1,3,5-(triaza)-2,6-(dioxa)-naphthalene, 2 (amino)purine, 2,4,5-(trimethyl)phenyl, 2′ methyl, 2′amino, 2′azido, 2′fluro-cytidine, 2′ methyl, 2′amino, 2′azido, 2′fluro-adenine, 2′methyl, 2′amino, 2′azido, 2′fluro-uridine, 2′-amino-2′-deoxyribose, 2-amino-6-Chloro-purine, 2-aza-inosinyl, 2′-azido-2′-deoxyribose, 2′fluoro-2′-deoxyribose, 2′-fluoro-modified bases, 2′-O-methyl-ribose, 2-oxo-7-aminopyridopyrimidin-3-yl, 2-oxo-pyridopyrimidine-3-yl, 2-pyridinone, 3 nitropyrrole, 3-(methyl)-7-(propynyl)isocarbostyrilyl, 3-(methyl)isocarbostyrilyl, 4-(fluoro)-6-(methyl)benzimidazole, 4-(methyl)benzimidazole, 4-(methyl)indolyl, 4,6-(dimethyl)indolyl, 5 nitroindole, 5 substituted pyrimidines, 5-(methyl)isocarbostyrilyl, 5-nitroindole, 6-(aza)pyrimidine, 6-(azo)thymine, 6-(methyl)-7-(aza)indolyl, 6-chloro-purine, 6-phenyl-pyrrolo-pyrimidin-2-on-3-yl, 7-(aminoalkylhydroxy)-1-(aza)-2-(thio)-3-(aza)-phenthiazin-I-yl, 7-(aminoalkylhydroxy)-1-(aza)-2-(thio)-3-(aza)-phenoxazin-1-yl, 7-(aminoalkylhydroxy)-1,3-(diaza)-2-(oxo)-phenoxazin-1-yl, 7-(aminoalkylhydroxy)-1,3-(diaza)-2-(oxo)-phenthiazin-I-yl, 7-(aminoalkylhydroxy)-I,3-(diaza)-2-(oxo)-phenoxazin-1-yl, 7-(aza)indolyl, 7-(guanidiniumalkylhydroxy)-1-(aza)-2-(thio)-3-(aza)-phenoxazinl-yl, 7-(guanidiniumalkylhydroxy)-1-(aza)-2-(thio)-3-(aza)-phenthiazin-1-yl, 7-(guanidiniumalkylhydroxy)-1-(aza)-2-(thio)-3-(aza)-phenoxazin-1-yl, 7-(guanidiniumalkylhydroxy)-1,3-(diaza)-2-(oxo)-phenoxazin-1-yl, 7-(guanidiniumalkyl-hydroxy)-1,3-(diaza)-2-(oxo)-phenthiazin-1-yl, 7-(guanidiniumalkylhydroxy)-1,3-(diaza)-2-(oxo)-phenoxazin-1-yl, 7-(propynyl)isocarbostyrilyl, 7-(propynyl)isocarbostyrilyl, propynyl-7-(aza)indolyl, 7-deaza-inosinyl, 7-substituted 1-(aza)-2-(thio)-3-(aza)-phenoxazin-1-yl, 7-substituted 1,3-(diaza)-2-(oxo)-phenoxazin-1-yl, 9-(methyl)-imidizopyridinyl, aminoindolyl, anthracenyl, bis-ortho-(aminoalkylhydroxy)-6-phenyl-pyrrolo-pyrimidin-2-on-3-yl, bis-ortho-substituted-6-phenyl-pyrrolo-pyrimidin-2-on-3-yl, difluorotolyl, hypoxanthine, imidizopyridinyl, inosinyl, isocarbostyrilyl, isoguanisine, N2-substituted purines, N6-methyl-2-amino-purine, N6-substituted purines, N-alkylated derivative, napthalenyl, nitrobenzimidazolyl, nitroimidazolyl, nitroindazolyl, nitropyrazolyl, nubularine, O6-substituted purines, O-alkylated derivative, ortho-(aminoalkylhydroxy)-6-phenyl-pyrrolo-pyrimidin-2-on-3-yl, ortho-substituted-6-phenyl-pyrrolo-pyrimidin-2-on-3-yl, Oxoformycin TP, para-(aminoalkylhydroxy)-6-phenyl-pyrrolo-pyrimidin-2-on-3-yl, para-substituted-6-phenyl-pyrrolo-pyrimidin-2-on-3-yl, pentacenyl, phenanthracenyl, phenyl, propynyl-7-(aza)indolyl, pyrenyl, pyridopyrimidin-3-yl, pyridopyrimidin-3-yl, 2-oxo-7-amino-pyridopyrimidin-3-yl, pyrrolo-pyrimidin-2-on-3-yl, pyrrolopyrimidinyl, pyrrolopyrizinyl, stilbenzyl, substituted 1,2,4-triazoles, tetracenyl, tubercidine, xanthine, Xanthosine-5′-TP, 2-thio-zebularine, 5-aza-2-thio-zebularine, 7-deaza-2-amino-purine, pyridin-4-one ribonucleoside, 2-Amino-riboside-TP, Formycin A TP, Formycin B TP, Pyrrolosine TP, 2′-OH-ara-adenosine TP, 2′-OH-ara-cytidine TP, 2′-OH-ara-uridine TP, 2′-OH-ara-guanosine TP, 5-(2-carbomethoxyvinyl)uridine TP, and N6-(19-Amino-pentaoxanonadecyl)adenosine TP.

### Linkers and Backbone Modifications

The renal polynucleotides can include any useful linker between the nucleosides. Non-limiting examples of linkers and linker modifications include 3′-alkylene phosphonates, 3′-amino phosphoramidate, alkene containing backbones, am inoalkylphosphoramidates, aminoalkylphosphotriesters, boranophosphates, —CH2-O—N(CH3)-CH2-, —CH2-N(CH3)-N(CH3)-CH2-, —CH2-NH—CH2-, chiral phosphonates, chiral phosphorothioates, formacetyl and thioformacetyl backbones, methylene (methylimino), methylene formacetyl and thioformacetyl backbones, methyleneimino and methylenehydrazino backbones, morpholino linkages, —N(CH3)-CH2-CH2-, oligonucleosides with heteroatom internucleoside linkage, phosphinates, phosphoramidates, phosphorodithioates, phosphorothioate internucleoside linkages, phosphorothioates, phosphotriesters, PNA, siloxane backbones, sulfamate backbones, sulfide sulfoxide and sulfone backbones, sulfonate and sulfonamide backbones, thionoalkylphosphonates, thionoalkylphosphotriesters, and thionophosphoramidates.

The renal polynucleotides can include any useful modification, such as to the sugar, the nucleobase, or the internucleoside linkage (e.g. to a linking phosphate/to a phosphodiester linkage/to the phosphodiester backbone). One or more atoms of a pyrimidine nucleobase may be replaced or substituted with optionally substituted amino, optionally substituted thiol, optionally substituted alkyl (e.g., methyl or ethyl), or halo (e.g., chloro or fluoro). In certain embodiments, modifications (e.g., one or more modifications) are present in each of the sugar and the internucleoside linkage. Modifications according to the present invention may be modifications of ribonucleic acids (RNAs) to deoxyribonucleic acids (DNAs), threose nucleic acids (TNAs), glycol nucleic acids (GNAs), renal peptide nucleic acids (PNAs), locked nucleic acids (LNAs) or hybrids thereof). Additional modifications are described herein.

In some embodiments, the renal polynucleotides of the invention do not substantially induce an innate immune response of a cell into which the mRNA is introduced. Featues of an induced innate immune response include 1) increased expression of pro-inflammatory cytokines, 2) activation of intracellular PRRs (RIG-I, MDAS, etc, and/or 3) termination or reduction in protein translation.

In certain embodiments, it may desirable to intracellularly degrade a polynulcleotide introduced into the cell. For example, degradation of a polynulcleotide may be preferable if precise timing of protein production is desired. Thus, in some embodiments, the invention provides a polynulcleotide containing a degradation domain, which is capable of being acted on in a directed manner within a cell.

Traditionally, the basic components of an mRNA molecule include at least a coding region, a 5′UTR, a 3′UTR, a 5′ cap and a poly-A tail. Building on this wild type modular structure, the present invention expands the scope of functionality of traditional mRNA molecules by providing renal polynucleotides which maintain a modular organization, but which comprise one or more structural and/or chemical modifications or alterations which impart useful properties to the renal polynucleotide including, in some embodiments, the lack of a substantial induction of the innate immune response of a cell into which the renal polynucleotides are introduced. As used herein, a “structural” feature or modification is one in which two or more linked nucleotides are inserted, deleted, duplicated, inverted or randomized in a renal polynucleotide without significant chemical modification to the nucleotides themselves. Because chemical bonds will necessarily be broken and reformed to effect a structural modification, structural modifications are of a chemical nature and hence are chemical modifications. However, structural modifications will result in a different sequence of nucleotides. For example, the renal polynucleotide “ATCG” may be chemically modified to “AT-5meC-G”. The same renal polynucleotide may be structurally modified from “ATCG” to “ATCCCG”. Here, the dinucleotide “CC” has been inserted, resulting in a structural modification to the renal polynucleotide.

Any of the regions of the renal polynucleotides may be chemically modified as taught herein or as taught in International Publication Numbers WO2013052523 (Attorney Docket Number M9) and WO2014093924 (Attorney Docket Number M36), the contents of each of which are incorporated herein by reference in its entirety.

### Modified Renal Polynucleotide Molecules

The present invention also includes building blocks, e.g., modified ribonucleosides, and modified ribonucleotides, of renal polynucleotide molecules. For example, these building blocks can be useful for preparing the renal polynucleotides of the invention. Such building blocks are taught in International Publication Numbers WO2013052523 (Attorney Docket Number M9) and WO2014093924 (Attorney Docket Number M36), the contents of each of which are incorporated herein by reference in its entirety.

### Modifications on the Sugar

The modified nucleosides and nucleotides (e.g., building block molecules), which may be incorporated into a renal polynucleotide (e.g., RNA or mRNA, as described herein), can be modified on the sugar of the ribonucleic acid. For example, the 2′ hydroxyl group (OH) can be modified or replaced with a number of different substituents. Exemplary substitutions at the 2′-position include, but are not limited to, H, halo, optionally substituted C₁₋₆ alkyl; optionally substituted C₁₋₆ alkoxy; optionally substituted C₆₋₁₀ aryloxy; optionally substituted C₃₋₈ cycloalkyl; optionally substituted C₃₋₈ cycloalkoxy; optionally substituted C₆₋₁₀ aryloxy; optionally substituted C₆₋₁₀ aryl-C₁₋₆ alkoxy, optionally substituted C₁₋₁₂ (heterocyclyl)oxy; a sugar (e.g., ribose, pentose, or any described herein); a polyethyleneglycol (PEG), —O(CH₂CH₂O)ₙCH₂CH₂OR, where R is H or optionally substituted alkyl, and n is an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20); “locked” nucleic acids (LNA) in which the 2′-hydroxyl is connected by a C₁₋₆ alkylene or C₁₋₆ heteroalkylene bridge to the 4′-carbon of the same ribose sugar, where exemplary bridges included methylene, propylene, ether, or amino bridges; aminoalkyl, as defined herein; aminoalkoxy, as defined herein; amino as defined herein; and amino acid, as defined herein

Generally, RNA includes the sugar group ribose, which is a 5-membered ring having an oxygen. Exemplary, non-limiting modified nucleotides include replacement of the oxygen in ribose (e.g., with S, Se, or alkylene, such as methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for anhydrohexitol, altritol, mannitol, cyclohexanyl, cyclohexenyl, and morpholino that also has a phosphoramidate backbone); multicyclic forms (e.g., tricyclo; and “unlocked” forms, such as glycol nucleic acid (GNA) (e.g., R-GNA or S-GNA, where ribose is replaced by glycol units attached to phosphodiester bonds), threose nucleic acid (TNA, where ribose is replace with a-L-threofuranosyl-(3′→2′)), and renal peptide nucleic acid (PNA, where 2-amino-ethyl-glycine linkages replace the ribose and phosphodiester backbone). The sugar group can also contain one or more carbons that possess the opposite stereochemical configuration than that of the corresponding carbon in ribose. Thus, a renal polynucleotide molecule can include nucleotides containing, e.g., arabinose, as the sugar. Such sugar modifications are taught International Publication Numbers WO2013052523 (Attorney Docket Number M9) and WO2014093924 (Attorney Docket Number M36), the contents of each of which are incorporated herein by reference in its entirety.

### Modifications on the Nucleobase

The present disclosure provides for modified nucleosides and nucleotides. As described herein “nucleoside” is defined as a compound containing a sugar molecule (e.g., a pentose or ribose) or a derivative thereof in combination with an organic base (e.g., a purine or pyrimidine) or a derivative thereof (also referred to herein as “nucleobase”). As described herein, “nucleotide” is defined as a nucleoside including a phosphate group. The modified nucleotides may by synthesized by any useful method, as described herein (e.g., chemically, enzymatically, or recombinantly to include one or more modified or non-natural nucleosides). The renal polynucleotides may comprise a region or regions of linked nucleosides. Such regions may have variable backbone linkages. The linkages may be standard phosphoester linkages, in which case the renal polynucleotides would comprise regions of nucleotides.

The modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures. One example of such non-standard base pairing is the base pairing between the modified nucleotide inosine and adenine, cytosine or uracil.

The modified nucleosides and nucleotides can include a modified nucleobase. Examples of nucleobases found in RNA include, but are not limited to, adenine, guanine, cytosine, and uracil. Examples of nucleobase found in DNA include, but are not limited to, adenine, guanine, cytosine, and thymine. Such modified nucleobases (including the distinctions between naturally occurring and non-naturally occurring) are taught in International Publication Numbers WO2013052523 (Attorney Docket Number M9) and WO2014093924 (Attorney Docket Number M36), the contents of each of which are incorporated herein by reference in its entirety.

### Combinations of Modified Sugars, Nucleobases, and Internucleoside Linkages

The renal polynucleotides of the invention can include a combination of modifications to the sugar, the nucleobase, and/or the internucleoside linkage. These combinations can include any one or more modifications described herein.

Examples of modified nucleotides and modified nucleotide combinations include, but are not limited to, α-thio-cytidine, pseudoisocytidine, pyrrolo-cytidine, 5-methyl-cytidine, N4-acetyl-cytidine, α-thio-cytidine/5-iodo-uridine, α-thio-cytidine/N1-methyl-pseudouridine, α-thio-cytidine/α-thio-uridine, α-thio-cytidine/5-methyl-uridine, α-thio-cytidine/pseudo-uridine, about 50% of the cytosines are α-thio-cytidine, pseudoisocytidine/5-iodo-uridine, pseudoisocytidine/N1-methyl-pseudouridine, pseudoisocytidine/α-thio-uridine, pseudoisocytidine/5-methyl-uridine, pseudoisocytidine/pseudouridine, about 25% of cytosines are pseudoisocytidine, pseudoisocytidine/about 50% of uridines are N1-methyl-pseudouridine and about 50% of uridines are pseudouridine, pseudoisocytidine/about 25% of uridines are N1-methyl-pseudouridine and about 25% of uridines are pseudouridine, pyrrolo-cytidine/5-iodo-uridine, pyrrolo-cytidine/N1-methyl-pseudouridine, pyrrolo-cytidine/α-thio-uridine, pyrrolo-cytidine/5-methyl-uridine, pyrrolo-cytidine/pseudouridine, about 50% of the cytosines are pyrrolo-cytidine, 5-methyl-cytidine/5-iodo-uridine, 5-methyl-cytidine/N1-methyl-pseudouridine, 5-methyl-cytidine/α-thio-uridine, 5-methyl-cytidine/5-methyl-uridine, 5-methyl-cytidine/pseudouridine, about 25% of cytosines are 5-methyl-cytidine, about 50% of cytosines are 5-methyl-cytidine, 5-methyl-cytidine/5-methoxy-uridine, 5-methyl-cytidine/5-bromo-uridine, 5-methyl-cytidine/2-thio-uridine, 5-methyl-cytidine/about 50% of uridines are 2-thio-uridine, about 50% of uridines are 5-methyl-cytidine/about 50% of uridines are 2-thio-uridine, N4-acetyl-cytidine/5-iodo-uridine, N4-acetyl-cytidine/N1-methyl-pseudouridine, N4-acetyl-cytidine/α-thio-uridine, N4-acetyl-cytidine/5-methyl-uridine, N4-acetyl-cytidine/pseudouridine, about 50% of cytosines are N4-acetyl-cytidine, about 25% of cytosines are N4-acetyl-cytidine, N4-acetyl-cytidine/5-methoxy-uridine, N4-acetyl-cytidine/5-bromo-uridine, N4-acetyl-cytidine/2-thio-uridine, about 50% of cytosines are N4-acetyl-cytidine/about 50% of uridines are 2-thio-uridine.

Examples of modified nucleotide combinations also include, but are not limited to, 1-(2,2,2-Trifluoroethyl)pseudo-UTP, 1-Ethyl-pseudo-UTP, 1-Methyl-pseudo-U-alpha-thio-TP, 1-methyl-pseudouridine TP, ATP, GTP, CTP, 1-methyl-pseudo-UTP/5-methyl-CTP/ATP/GTP, 1-methyl-pseudo-UTP/CTP/ATP/GTP, 1-Propyl-pseudo-UTP, 25% 5-Aminoallyl-CTP+75% CTP/25% 5-Methoxy-UTP+75% UTP, 25% 5-Aminoallyl-CTP+75% CTP/75% 5-Methoxy-UTP+25% UTP, 25% 5-Bromo-CTP+75% CTP/25% 5-Methoxy-UTP+75% UTP, 25% 5-Bromo-CTP+75% CTP/75% 5-Methoxy-UTP+25% UTP, 25% 5-Bromo-CTP+75% CTP/1-Methyl-pseudo-UTP, 25% 5-Carboxy-CTP+75% CTP/25% 5-Methoxy-UTP+75% UTP, 25% 5-Carboxy-CTP+75% CTP/75% 5-Methoxy-UTP+25% UTP, 25% 5-Ethyl-CTP+75% CTP/25% 5-Methoxy-UTP+75% UTP, 25% 5-Ethyl-CTP+75% CTP/75% 5-Methoxy-UTP+25% UTP, 25% 5-Ethynyl-CTP+75% CTP/25% 5-Methoxy-UTP+75% UTP, 25% 5-Ethynyl-CTP+75% CTP/75% 5-Methoxy-UTP+25% UTP, 25% 5-Fluoro-CTP+75% CTP/25% 5-Methoxy-UTP+75% UTP, 25% 5-Fluoro-CTP+75% CTP/75% 5-Methoxy-UTP+25% UTP, 25% 5-Formyl-CTP+75% CTP/25% 5-Methoxy-UTP+75% UTP, 25% 5-Formyl-CTP+75% CTP/75% 5-Methoxy-UTP+25% UTP, 25% 5-Hydroxymethyl-CTP+75% CTP/25% 5-Methoxy-UTP+75% UTP, 25% 5-Hydroxymethyl-CTP+75% CTP/75% 5-Methoxy-UTP+25% UTP, 25% 5-Iodo-CTP+75% CTP/25% 5-Methoxy-UTP+75% UTP, 25% 5-Iodo-CTP+75% CTP/75% 5-Methoxy-UTP+25% UTP, 25% 5-Methoxy-CTP+75% CTP/25% 5-Methoxy-UTP+75% UTP, 25% 5-Methoxy-CTP+75% CTP/75% 5-Methoxy-UTP+25% UTP, 25% 5-Methyl-CTP+75% CTP/25% 5-Methoxy-UTP+75% 1-Methyl-pseudo-UTP, 25% 5-Methyl-CTP+75% CTP/25% 5-Methoxy-UTP+75% UTP, 25% 5-Methyl-CTP+75% CTP/50% 5-Methoxy-UTP+50% 1-Methyl-pseudo-UTP, 25% 5-Methyl-CTP+75% CTP/50% 5-Methoxy-UTP+50% UTP, 25% 5-Methyl-CTP+75% CTP/5-Methoxy-UTP, 25% 5-Methyl-CTP+75% CTP/75% 5-Methoxy-UTP+25% 1-Methyl-pseudo-UTP, 25% 5-Methyl-CTP+75% CTP/75% 5-Methoxy-UTP+25% UTP, 25% 5-Phenyl-CTP+75% CTP/25% 5-Methoxy-UTP+75% UTP, 25% 5-Phenyl-CTP+75% CTP/75% 5-Methoxy-UTP+25% UTP, 25% 5-Trifluoromethyl-CTP+75% CTP/25% 5-Methoxy-UTP+75% UTP, 25% 5-Trifluoromethyl-CTP+75% CTP/75% 5-Methoxy-UTP+25% UTP, 25% 5-Trifluoromethyl-CTP+75% CTP/1-Methyl-pseudo-UTP, 25% N4-Ac-CTP+75% CTP/25% 5-Methoxy-UTP+75% UTP, 25% N4-Ac-CTP+75% CTP/75% 5-Methoxy-UTP+25% UTP, 25% N4-Bz-CTP+75% CTP/25% 5-Methoxy-UTP+75% UTP, 25% N4-Bz-CTP+75% CTP/75% 5-Methoxy-UTP+25% UTP, 25% N4-Methyl-CTP+75% CTP/25% 5-Methoxy-UTP+75% UTP, 25% N4-Methyl-CTP+75% CTP/75% 5-Methoxy-UTP+25% UTP, 25% Pseudo-iso-CTP+75% CTP/25% 5-Methoxy-UTP+75% UTP, 25% Pseudo-iso-CTP+75% CTP/75% 5-Methoxy-UTP+25% UTP, 25% 5-Bromo-CTP/75% CTP/Pseudo-UTP, 25% 5-methoxy-UTP/25% 5-methyl-CTP/ATP/GTP, 25% 5-methoxy-UTP/5-methyl-CTP/ATP/GTP, 25% 5-methoxy-UTP/75% 5-methyl-CTP/ATP/GTP, 25% 5-methoxy-UTP/CTP/ATP/GTP, 25% 5-metoxy-UTP/50% 5-methyl-CTP/ATP/GTP, 2-Amino-ATP, 2-Thio-CTP, 2-thio-pseudouridine TP, ATP, GTP, CTP, 2-Thio-pseudo-UTP, 2-Thio-UTP, 3-Methyl-CTP, 3-Methyl-pseudo-UTP, 4-Thio-UTP, 50% 5-Bromo-CTP+50% CTP/1-Methyl-pseudo-UTP, 50% 5-Hydroxymethyl-CTP+50% CTP/1-Methyl-pseudo-UTP, 50% 5-methoxy-UTP/5-methyl-CTP/ATP/GTP, 50% 5-Methyl-CTP+50% CTP/25% 5-Methoxy-UTP+75% 1-Methyl-pseudo-UTP, 50% 5-Methyl-CTP+50% CTP/25% 5-Methoxy-UTP+75% UTP, 50% 5-Methyl-CTP+50% CTP/50% 5-Methoxy-UTP+50% 1-Methyl-pseudo-UTP, 50% 5-Methyl-CTP+50% CTP/50% 5-Methoxy-UTP+50% UTP, 50% 5-Methyl-CTP+50% CTP/5-Methoxy-UTP, 50% 5-Methyl-CTP+50% CTP/75% 5-Methoxy-UTP+25% 1-Methyl-pseudo-UTP, 50% 5-Methyl-CTP+50% CTP/75% 5-Methoxy-UTP+25% UTP, 50% 5-Trifluoromethyl-CTP+50% CTP/1-Methyl-pseudo-UTP, 50% 5-Bromo-CTP/50% CTP/Pseudo-UTP, 50% 5-methoxy-UTP/25% 5-methyl-CTP/ATP/GTP, 50% 5-methoxy-UTP/50% 5-methyl-CTP/ATP/GTP, 50% 5-methoxy-UTP/75% 5-methyl-CTP/ATP/GTP, 50% 5-methoxy-UTP/CTP/ATP/GTP, 5-Aminoallyl-CTP, 5-Aminoallyl-CTP/5-Methoxy-UTP, 5-Aminoallyl-UTP, 5-Bromo-CTP, 5-Bromo-CTP/5-Methoxy-UTP, 5-Bromo-CTP/1-Methyl-pseudo-UTP, 5-Bromo-CTP/Pseudo-UTP, 5-bromocytidine TP, ATP, GTP, UTP, 5-Bromo-UTP, 5-Carboxy-CTP/5-Methoxy-UTP, 5-Ethyl-CTP/5-Methoxy-UTP, 5-Ethynyl-CTP/5-Methoxy-UTP, 5-Fluoro-CTP/5-Methoxy-UTP, 5-Formyl-CTP/5-Methoxy-UTP, 5-Hydroxy-methyl-CTP/5-Methoxy-UTP, 5-Hydroxymethyl-CTP, 5-Hydroxymethyl-CTP/1-Methyl-pseudo-UTP, 5-Hydroxymethyl-CTP/5-Methoxy-UTP, 5-hydroxymethylcytidine TP, ATP, GTP, UTP, 5-Iodo-CTP/5-Methoxy-UTP, 5-Me-CTP/5-Methoxy-UTP, 5-Methoxy carbonyl methyl-UTP, 5-Methoxy-CTP/5-Methoxy-UTP, 5-methoxy-uridine TP, ATP, GTP, UTP, 5-methoxy-UTP, 5-Methoxy-UTP, 5-Methoxy-UTP/N6-Isopentenyl-ATP, 5-methoxy-UTP/25% 5-methyl-CTP/ATP/GTP, 5-methoxy-UTP/5-methyl-CTP/ATP/GTP, 5-methoxy-UTP/75% 5-methyl-CTP/ATP/GTP, 5-methoxy-UTP/CTP/ATP/GTP, 5-Methyl-2-thio-UTP, 5-Methylaminomethyl-UTP, 5-Methyl-CTP/5-Methoxy-UTP, 5-Methyl-CTP/5-Methoxy-UTP(cap 0), 5-Methyl-CTP/5-Methoxy-UTP(No cap), 5-Methyl-CTP/25% 5-Methoxy-UTP+75% 1-Methyl-pseudo-UTP, 5-Methyl-CTP/25% 5-Methoxy-UTP+75% UTP, 5-Methyl-CTP/50% 5-Methoxy-UTP+50% 1-Methyl-pseudo-UTP, 5-Methyl-CTP/50% 5-Methoxy-UTP+50% UTP, 5-Methyl-CTP/5-Methoxy-UTP/N6-Me-ATP, 5-Methyl-CTP/75% 5-Methoxy-UTP+25% 1-Methyl-pseudo-UTP, 5-Methyl-CTP/75% 5-Methoxy-UTP+25% UTP, 5-Phenyl-CTP/5-Methoxy-UTP, 5-Trifluoro-methyl-CTP/5-Methoxy-UTP, 5-Trifluoromethyl-CTP, 5-Trifluoromethyl-CTP/5-Methoxy-UTP, 5-Trifluoromethyl-CTP/1-Methyl-pseudo-UTP, 5-Trifluoromethyl-CTP/Pseudo-UTP, 5-Trifluoromethyl-UTP, 5-trifluromethylcytidine TP, ATP, GTP, UTP, 75% 5-Aminoallyl-CTP+25% CTP/25% 5-Methoxy-UTP+75% UTP, 75% 5-Aminoallyl-CTP+25% CTP/75% 5-Methoxy-UTP+25% UTP, 75% 5-Bromo-CTP+25% CTP/25% 5-Methoxy-UTP+75% UTP, 75% 5-Bromo-CTP+25% CTP/75% 5-Methoxy-UTP+25% UTP, 75% 5-Carboxy-CTP+25% CTP/25% 5-Methoxy-UTP+75% UTP, 75% 5-Carboxy-CTP+25% CTP/75% 5-Methoxy-UTP+25% UTP, 75% 5-Ethyl-CTP+25% CTP/25% 5-Methoxy-UTP+75% UTP, 75% 5-Ethyl-CTP+25% CTP/75% 5-Methoxy-UTP+25% UTP, 75% 5-Ethynyl-CTP+25% CTP/25% 5-Methoxy-UTP+75% UTP, 75% 5-Ethynyl-CTP+25% CTP/75% 5-Methoxy-UTP+25% UTP, 75% 5-Fluoro-CTP+25% CTP/25% 5-Methoxy-UTP+75% UTP, 75% 5-Fluoro-CTP+25% CTP/75% 5-Methoxy-UTP+25% UTP, 75% 5-Formyl-CTP+25% CTP/25% 5-Methoxy-UTP+75% UTP, 75% 5-Formyl-CTP+25% CTP/75% 5-Methoxy-UTP+25% UTP, 75% 5-Hydroxymethyl-CTP+25% CTP/25% 5-Methoxy-UTP+75% UTP, 75% 5-Hydroxymethyl-CTP+25% CTP/75% 5-Methoxy-UTP+25% UTP, 75% 5-Iodo-CTP+25% CTP/25% 5-Methoxy-UTP+75% UTP, 75% 5-Iodo-CTP+25% CTP/75% 5-Methoxy-UTP+25% UTP, 75% 5-Methoxy-CTP+25% CTP/25% 5-Methoxy-UTP+75% UTP, 75% 5-Methoxy-CTP+25% CTP/75% 5-Methoxy-UTP+25% UTP, 75% 5-methoxy-UTP/5-methyl-CTP/ATP/GTP, 75% 5-Methyl-CTP+25% CTP/25% 5-Methoxy-UTP+75% 1-Methyl-pseudo-UTP, 75% 5-Methyl-CTP+25% CTP/25% 5-Methoxy-UTP+75% UTP, 75% 5-Methyl-CTP+25% CTP/50% 5-Methoxy-UTP+50% 1-Methyl-pseudo-UTP, 75% 5-Methyl-CTP+25% CTP/50% 5-Methoxy-UTP+50% UTP, 75% 5-Methyl-CTP+25% CTP/5-Methoxy-UTP, 75% 5-Methyl-CTP+25% CTP/75% 5-Methoxy-UTP+25% 1-Methyl-pseudo-UTP, 75% 5-Methyl-CTP+25% CTP/75% 5-Methoxy-UTP+25% UTP, 75% 5-Phenyl-CTP+25% CTP/25% 5-Methoxy-UTP+75% UTP, 75% 5-Phenyl-CTP+25% CTP/75% 5-Methoxy-UTP+25% UTP, 75% 5-Trifluoromethyl-CTP+25% CTP/25% 5-Methoxy-UTP+75% UTP, 75% 5-Trifluoromethyl-CTP+25% CTP/75% 5-Methoxy-UTP+25% UTP, 75% 5-Trifluoromethyl-CTP+25% CTP/1-Methyl-pseudo-UTP, 75% N4-Ac-CTP+25% CTP/25% 5-Methoxy-UTP+75% UTP, 75% N4-Ac-CTP+25% CTP/75% 5-Methoxy-UTP+25% UTP, 75% N4-Bz-CTP+25% CTP/25% 5-Methoxy-UTP+75% UTP, 75% N4-Bz-CTP+25% CTP/75% 5-Methoxy-UTP+25% UTP, 75% N4-Methyl-CTP+25% CTP/25% 5-Methoxy-UTP+75% UTP, 75% N4-Methyl-CTP+25% CTP/75% 5-Methoxy-UTP+25% UTP, 75% Pseudo-iso-CTP+25% CTP/25% 5-Methoxy-UTP+75% UTP, 75% Pseudo-iso-CTP+25% CTP/75% 5-Methoxy-UTP+25% UTP, 75% 5-Bromo-CTP/25% CTP/1-Methyl-pseudo-UTP, 75% 5-Bromo-CTP/25% CTP/Pseudo-UTP, 75% 5-methoxy-UTP/25% 5-methyl-CTP/ATP/GTP, 75% 5-methoxy-UTP/50% 5-methyl-CTP/ATP/GTP, 75% 5-methoxy-UTP/75% 5-methyl-CTP/ATP/GTP, 75% 5-methoxy-UTP/CTP/ATP/GTP, 8-Aza-ATP, Alpha-thio-CTP, CTP/25% 5-Methoxy-UTP+75% 1-Methyl-pseudo-UTP, CTP/25% 5-Methoxy-UTP+75% UTP, CTP/50% 5-Methoxy-UTP+50% 1-Methyl-pseudo-UTP, CTP/50% 5-Methoxy-UTP+50% UTP, CTP/5-Methoxy-UTP, CTP/5-Methoxy-UTP (cap 0), CTP/5-Methoxy-UTP(No cap), CTP/75% 5-Methoxy-UTP+25% 1-Methyl-pseudo-UTP, CTP/75% 5-Methoxy-UTP+25% UTP, CTP/UTP(No cap), N1-Me-GTP, N4-Ac-CTP, N4Ac-CTP/1-Methyl-pseudo-UTP, N4Ac-CTP/5-Methoxy-UTP, N4-acetyl-cytidine TP, ATP, GTP, UTP, N4-Bz-CTP/5-Methoxy-UTP, N4-methyl CTP, N4-Methyl-CTP/5-Methoxy-UTP, Pseudo-iso-CTP/5-Methoxy-UTP, PseudoU-alpha-thio-TP, pseudouridine TP, ATP, GTP, CTP, pseudo-UTP/5-methyl-CTP/ATP/GTP, UTP-5-oxyacetic acid Me ester, and Xanthosine.

These combinations of modified nucleotides can be used to form the renal polynucleotides of the invention. Unless otherwise noted, the modified nucleotides may be completely substituted for the natural nucleotides of the renal polynucleotides of the invention. As a non-limiting example, the natural nucleotide uridine may be substituted with a modified nucleoside described herein. In another non-limiting example, the natural nucleotide uridine may be partially substituted (e.g., about 0.1%, 1%, 5%, 10%, 15%, 20%, 25%, 30%, 35%, 40%, 45%, 50%, 55%, 60%, 65%, 70%, 75%, 80%, 85%, 90%, 95% or 99.9%) with at least one of the modified nucleoside disclosed herein. Any combination of base/sugar or linker may be incorporated into the renal polynucleotides of the invention and such modifications are taught International Publication Numbers WO2013052523 (Attorney Docket Number M9), and WO2014093924 (Attorney Docket Number M36); the contents of each of which are incorporated herein by reference in its entirety.

Non-limiting examples of combinations are described in Tables 5 and 6.

TABLE 5
Combinations

| Modified Nucleotide | Modified Nucleotide Combination |
|---|---|
| α-thio-cytidine | α-thio-cytidine/5-iodo-uridine |
| | α-thio-cytidine/N1-methyl-pseudouridine |
| | α-thio-cytidine/α-thio-uridine |
| | α-thio-cytidine/5-methyl-uridine |
| | α-thio-cytidine/pseudo-uridine |
| | about 50% of the cytosines are α-thio-cytidine |
| pseudoisocytidine | pseudoisocytidine/5-iodo-uridine |
| | pseudoisocytidine/N1-methyl-pseudouridine |
| | pseudoisocytidine/α-thio-uridine |
| | pseudoisocytidine/5-methyl-uridine |
| | pseudoisocytidine/pseudouridine |
| | about 25% of cytosines are pseudoisocytidine |
| | pseudoisocytidine/about 50% of uridines are N1- |
| | methyl-pseudouridine and about 50% of uridines |
| | are pseudouridine |
| | pseudoisocytidine/about 25% of uridines are N1- |
| | methyl-pseudouridine and about 25% of uridines |
| | are pseudouridine |
| pyrrolo-cytidine | pyrrolo-cytidine/5-iodo-uridine |
| | pyrrolo-cytidine/N1-methyl-pseudouridine |
| | pyrrolo-cytidine/α-thio-uridine |
| | pyrrolo-cytidine/5-methyl-uridine |
| | pyrrolo-cytidine/pseudouridine |
| | about 50% of the cytosines are pyrrolo-cytidine |
| 5-methyl-cytidine | 5-methyl-cytidine/5-iodo-uridine |
| | 5-methyl-cytidine/N1-methyl-pseudouridine |
| | 5-methyl-cytidine/α-thio-uridine |
| | 5-methyl-cytidine/5-methyl-uridine |
| | 5-methyl-cytidine/pseudouridine |
| | about 25% of cytosines are 5-methyl-cytidine |
| | about 50% of cytosines are 5-methyl-cytidine |
| | 5-methyl-cytidine/5-methoxy-uridine |
| | 5-methyl-cytidine/5-bromo-uridine |
| | 5-methyl-cytidine/2-thio-uridine |
| | 5-methyl-cytidine/about 50% of uridines are 2-thio- |
| | uridine |
| | about 50% of uridines are 5-methyl-cytidine/about |
| | 50% of uridines are 2-thio-uridine |
| N4-acetyl-cytidine | N4-acetyl-cytidine/5-iodo-uridine |
| | N4-acetyl-cytidine/N1-methyl-pseudouridine |
| | N4-acetyl-cytidine/α-thio-uridine |
| | N4-acetyl-cytidine/5-methyl-uridine |
| | N4-acetyl-cytidine/pseudouridine |
| | about 50% of cytosines are N4-acetyl-cytidine |
| | about 25% of cytosines are N4-acetyl-cytidine |
| | N4-acetyl-cytidine/5-methoxy-uridine |
| | N4-acetyl-cytidine/5-bromo-uridine |
| | N4-acetyl-cytidine/2-thio-uridine |
| | about 50% of cytosines are N4-acetyl-cytidine/about |
| | 50% of uridines are 2-thio-uridine |

TABLE 6
Combinations

1-(2,2,2-Trifluoroethyl)pseudo-UTP
1-Ethyl-pseudo-UTP
1-Methyl-pseudo-U-alpha-thio-TP
1-methyl-pseudouridine TP, ATP, GTP, CTP
1-methyl-pseudo-UTP/5-methyl-CTP/ATP/GTP
1-methyl-pseudo-UTP/CTP/ATP/GTP
1-Propyl-pseudo-UTP
25% 5-Aminoallyl-CTP + 75% CTP/25% 5-Methoxy-UTP + 75% UTP
25% 5-Aminoallyl-CTP + 75% CTP/75% 5-Methoxy-UTP + 25% UTP
25% 5-Bromo-CTP + 75% CTP/25% 5-Methoxy-UTP + 75% UTP
25% 5-Bromo-CTP + 75% CTP/75% 5-Methoxy-UTP + 25% UTP
25% 5-Bromo-CTP + 75% CTP/1-Methyl-pseudo-UTP
25% 5-Carboxy-CTP + 75% CTP/25% 5-Methoxy-UTP + 75% UTP
25% 5-Carboxy-CTP + 75% CTP/75% 5-Methoxy-UTP + 25% UTP
25% 5-Ethyl-CTP + 75% CTP/25% 5-Methoxy-UTP + 75% UTP
25% 5-Ethyl-CTP + 75% CTP/75% 5-Methoxy-UTP + 25% UTP
25% 5-Ethynyl-CTP + 75% CTP/25% 5-Methoxy-UTP + 75% UTP
25% 5-Ethynyl-CTP + 75% CTP/75% 5-Methoxy-UTP + 25% UTP
25% 5-Fluoro-CTP + 75% CTP/25% 5-Methoxy-UTP + 75% UTP
25% 5-Fluoro-CTP + 75% CTP/75% 5-Methoxy-UTP + 25% UTP
25% 5-Formyl-CTP + 75% CTP/25% 5-Methoxy-UTP + 75% UTP
25% 5-Formyl-CTP + 75% CTP/75% 5-Methoxy-UTP + 25% UTP
25% 5-Hydroxymethyl-CTP + 75% CTP/25% 5-Methoxy-UTP +
75% UTP
25% 5-Hydroxymethyl-CTP + 75% CTP/75% 5-Methoxy-UTP +
25% UTP
25% 5-Iodo-CTP + 75% CTP/25% 5-Methoxy-UTP + 75% UTP
25% 5-Iodo-CTP + 75% CTP/75% 5-Methoxy-UTP + 25% UTP
25% 5-Methoxy-CTP + 75% CTP/25% 5-Methoxy-UTP + 75% UTP
25% 5-Methoxy-CTP + 75% CTP/75% 5-Methoxy-UTP + 25% UTP
25% 5-Methyl-CTP + 75% CTP/25% 5-Methoxy-UTP + 75%
1-Methyl-pseudo-UTP
25% 5-Methyl-CTP + 75% CTP/25% 5-Methoxy-UTP + 75% UTP
25% 5-Methyl-CTP + 75% CTP/50% 5-Methoxy-UTP + 50%
1-Methyl-pseudo-UTP
25% 5-Methyl-CTP + 75% CTP/50% 5-Methoxy-UTP + 50% UTP
25% 5-Methyl-CTP + 75% CTP/5-Methoxy-UTP
25% 5-Methyl-CTP + 75% CTP/75% 5-Methoxy-UTP + 25%
1-Methyl-pseudo-UTP
25% 5-Methyl-CTP + 75% CTP/75% 5-Methoxy-UTP + 25% UTP
25% 5-Phenyl-CTP + 75% CTP/25% 5-Methoxy-UTP + 75% UTP
25% 5-Phenyl-CTP + 75% CTP/75% 5-Methoxy-UTP + 25% UTP
25% 5-Trifluoromethyl-CTP + 75% CTP/25% 5-Methoxy-UTP +
75% UTP
25% 5-Trifluoromethyl-CTP + 75% CTP/75% 5-Methoxy-UTP +
25% UTP
25% 5-Trifluoromethyl-CTP + 75% CTP/1-Methyl-pseudo-UTP
25% N4—Ac-CTP + 75% CTP/25% 5-Methoxy-UTP + 75% UTP
25% N4—Ac-CTP + 75% CTP/75% 5-Methoxy-UTP + 25% UTP
25% N4-Bz-CTP + 75% CTP/25% 5-Methoxy-UTP + 75% UTP
25% N4-Bz-CTP + 75% CTP/75% 5-Methoxy-UTP + 25% UTP
25% N4-Methyl-CTP + 75% CTP/25% 5-Methoxy-UTP + 75% UTP
25% N4-Methyl-CTP + 75% CTP/75% 5-Methoxy-UTP + 25% UTP
25% Pseudo-iso-CTP + 75% CTP/25% 5-Methoxy-UTP + 75% UTP
25% Pseudo-iso-CTP + 75% CTP/75% 5-Methoxy-UTP + 25% UTP
25% 5-Bromo-CTP/75% CTP/Pseudo-UTP
25% 5-methoxy-UTP/25% 5-methyl-CTP/ATP/GTP
25% 5-methoxy-UTP/5-methyl-CTP/ATP/GTP
25% 5-methoxy-UTP/75% 5-methyl-CTP/ATP/GTP
25% 5-methoxy-UTP/CTP/ATP/GTP
25% 5-metoxy-UTP/50% 5-methyl-CTP/ATP/GTP
2-Amino-ATP
2-Thio-CTP
2-thio-pseudouridine TP, ATP, GTP, CTP
2-Thio-pseudo-UTP
2-Thio-UTP
3-Methyl-CTP
3-Methyl-pseudo-UTP
4-Thio-UTP
50% 5-Bromo-CTP + 50% CTP/1-Methyl-pseudo-UTP
50% 5-Hydroxymethyl-CTP + 50% CTP/1-Methyl-pseudo-UTP
50% 5-methoxy-UTP/5-methyl-CTP/ATP/GTP
50% 5-Methyl-CTP + 50% CTP/25% 5-Methoxy-UTP + 75%
1-Methyl-pseudo-UTP
50% 5-Methyl-CTP + 50% CTP/25% 5-Methoxy-UTP + 75% UTP
50% 5-Methyl-CTP + 50% CTP/50% 5-Methoxy-UTP + 50%
1-Methyl-pseudo-UTP
50% 5-Methyl-CTP + 50% CTP/50% 5-Methoxy-UTP + 50% UTP
50% 5-Methyl-CTP + 50% CTP/5-Methoxy-UTP
50% 5-Methyl-CTP + 50% CTP/75% 5-Methoxy-UTP + 25%
1-Methyl-pseudo-UTP
50% 5-Methyl-CTP + 50% CTP/75% 5-Methoxy-UTP + 25% UTP
50% 5-Trifluoromethyl-CTP + 50% CTP/1-Methyl-pseudo-UTP
50% 5-Bromo-CTP/50% CTP/Pseudo-UTP
50% 5-methoxy-UTP/25% 5-methyl-CTP/ATP/GTP
50% 5-methoxy-UTP/50% 5-methyl-CTP/ATP/GTP
50% 5-methoxy-UTP/75% 5-methyl-CTP/ATP/GTP
50% 5-methoxy-UTP/CTP/ATP/GTP
5-Aminoallyl-CTP
5-Aminoallyl-CTP/5-Methoxy-UTP
5-Aminoallyl-UTP
5-Bromo-CTP
5-Bromo-CTP/5-Methoxy-UTP
5-Bromo-CTP/1-Methyl-pseudo-UTP
5-Bromo-CTP/Pseudo-UTP
5-bromocytidine TP, ATP, GTP, UTP
5-Bromo-UTP
5-Carboxy-CTP/5-Methoxy-UTP
5-Ethyl-CTP/5-Methoxy-UTP
5-Ethynyl-CTP/5-Methoxy-UTP
5-Fluoro-CTP/5-Methoxy-UTP
5-Formyl-CTP/5-Methoxy-UTP
5-Hydroxy-methyl-CTP/5-Methoxy-UTP
5-Hydroxymethyl-CTP
5-Hydroxymethyl-CTP/1-Methyl-pseudo-UTP
5-Hydroxymethyl-CTP/5-Methoxy-UTP
5-hydroxymethyl-cytidine TP, ATP, GTP, UTP
5-Iodo-CTP/5-Methoxy-UTP
5-Me-CTP/5-Methoxy-UTP
5-Methoxy carbonyl methyl-UTP
5-Methoxy-CTP/5-Methoxy-UTP
5-methoxy-uridine TP, ATP, GTP, UTP
5-methoxy-UTP
5-Methoxy-UTP
5-Methoxy-UTP/N6-Isopentenyl-ATP
5-methoxy-UTP/25% 5-methyl-CTP/ATP/GTP
5-methoxy-UTP/5-methyl-CTP/ATP/GTP
5-methoxy-UTP/75% 5-methyl-CTP/ATP/GTP
5-methoxy-UTP/CTP/ATP/GTP
5-Methyl-2-thio-UTP
5-Methylaminomethyl-UTP
5-Methyl-CTP/5-Methoxy-UTP
5-Methyl-CTP/5-Methoxy-UTP(cap 0)
5-Methyl-CTP/5-Methoxy-UTP(No cap)
5-Methyl-CTP/25% 5-Methoxy-UTP + 75% 1-Methyl-pseudo-UTP
5-Methyl-CTP/25% 5-Methoxy-UTP + 75% UTP
5-Methyl-CTP/50% 5-Methoxy-UTP + 50% 1-Methyl-pseudo-UTP
5-Methyl-CTP/50% 5-Methoxy-UTP + 50% UTP
5-Methyl-CTP/5-Methoxy-UTP/N6-Me-ATP
5-Methyl-CTP/75% 5-Methoxy-UTP + 25% 1-Methyl-pseudo-UTP
5-Methyl-CTP/75% 5-Methoxy-UTP + 25% UTP
5-Phenyl-CTP/5-Methoxy-UTP
5-Trifluoro- methyl-CTP/5-Methoxy-UTP
5-Trifluoromethyl-CTP
5-Trifluoromethyl-CTP/5-Methoxy-UTP
5-Trifluoromethyl-CTP/1-Methyl-pseudo-UTP
5-Trifluoromethyl-CTP/Pseudo-UTP
5-Trifluoromethyl-UTP
5-trifluromethylcytidine TP, ATP, GTP, UTP
75% 5-Aminoallyl-CTP + 25% CTP/25% 5-Methoxy-UTP + 75% UTP
75% 5-Aminoallyl-CTP + 25% CTP/75% 5-Methoxy-UTP + 25% UTP
75% 5-Bromo-CTP + 25% CTP/25% 5-Methoxy-UTP + 75% UTP
75% 5-Bromo-CTP + 25% CTP/75% 5-Methoxy-UTP + 25% UTP
75% 5-Carboxy-CTP + 25% CTP/25% 5-Methoxy-UTP + 75% UTP
75% 5-Carboxy-CTP + 25% CTP/75% 5-Methoxy-UTP + 25% UTP
75% 5-Ethyl-CTP + 25% CTP/25% 5-Methoxy-UTP + 75% UTP
75% 5-Ethyl-CTP + 25% CTP/75% 5-Methoxy-UTP + 25% UTP
75% 5-Ethynyl-CTP + 25% CTP/25% 5-Methoxy-UTP + 75% UTP
75% 5-Ethynyl-CTP + 25% CTP/75% 5-Methoxy-UTP + 25% UTP
75% 5-Fluoro-CTP + 25% CTP/25% 5-Methoxy-UTP + 75% UTP
75% 5-Fluoro-CTP + 25% CTP/75% 5-Methoxy-UTP + 25% UTP
75% 5-Formyl-CTP + 25% CTP/25% 5-Methoxy-UTP + 75% UTP
75% 5-Formyl-CTP + 25% CTP/75% 5-Methoxy-UTP + 25% UTP
75% 5-Hydroxymethyl-CTP + 25% CTP/25% 5-Methoxy-UTP +
75% UTP
75% 5-Hydroxymethyl-CTP + 25% CTP/75% 5-Methoxy-UTP +
25% UTP
75% 5-Iodo-CTP + 25% CTP/25% 5-Methoxy-UTP + 75% UTP
75% 5-Iodo-CTP + 25% CTP/75% 5-Methoxy-UTP + 25% UTP
75% 5-Methoxy-CTP + 25% CTP/25% 5-Methoxy-UTP + 75% UTP
75% 5-Methoxy-CTP + 25% CTP/75% 5-Methoxy-UTP + 25% UTP
75% 5-methoxy-UTP/5-methyl-CTP/ATP/GTP
75% 5-Methyl-CTP + 25% CTP/25% 5-Methoxy-UTP + 75%
1-Methyl-pseudo-UTP
75% 5-Methyl-CTP + 25% CTP/25% 5-Methoxy-UTP + 75% UTP
75% 5-Methyl-CTP + 25% CTP/50% 5-Methoxy-UTP + 50%
1-Methyl-pseudo-UTP
75% 5-Methyl-CTP + 25% CTP/50% 5-Methoxy-UTP + 50% UTP
75% 5-Methyl-CTP + 25% CTP/5-Methoxy-UTP
75% 5-Methyl-CTP + 25% CTP/75% 5-Methoxy-UTP + 25%
1-Methyl-pseudo-UTP
75% 5-Methyl-CTP + 25% CTP/75% 5-Methoxy-UTP + 25% UTP
75% 5-Phenyl-CTP + 25% CTP/25% 5-Methoxy-UTP + 75% UTP
75% 5-Phenyl-CTP + 25% CTP/75% 5-Methoxy-UTP + 25% UTP
75% 5-Trifluoromethyl-CTP + 25% CTP/25% 5-Methoxy-UTP +
75% UTP
75% 5-Trifluoromethyl-CTP + 25% CTP/75% 5-Methoxy-UTP +
25% UTP
75% 5-Trifluoromethyl-CTP + 25% CTP/1-Methyl-pseudo-UTP
75% N4—Ac-CTP + 25% CTP/25% 5-Methoxy-UTP + 75% UTP
75% N4—Ac-CTP + 25% CTP/75% 5-Methoxy-UTP + 25% UTP
75% N4-Bz-CTP + 25% CTP/25% 5-Methoxy-UTP + 75% UTP
75% N4-Bz-CTP + 25% CTP/75% 5-Methoxy-UTP + 25% UTP
75% N4-Methyl-CTP + 25% CTP/25% 5-Methoxy-UTP + 75% UTP
75% N4-Methyl-CTP + 25% CTP/75% 5-Methoxy-UTP + 25% UTP
75% Pseudo-iso-CTP + 25% CTP/25% 5-Methoxy-UTP +
75% UTP
75% Pseudo-iso-CTP + 25% CTP/75% 5-Methoxy-UTP +
25% UTP
75% 5-Bromo-CTP/25% CTP/1-Methyl-pseudo-UTP
75% 5-Bromo-CTP/25% CTP/Pseudo-UTP
75% 5-methoxy-UTP/25% 5-methyl-CTP/ATP/GTP
75% 5-methoxy-UTP/50% 5-methyl-CTP/ATP/GTP
75% 5-methoxy-UTP/75% 5-methyl-CTP/ATP/GTP
75% 5-methoxy-UTP/CTP/ATP/GTP
8-Aza-ATP
Alpha-thio-CTP
CTP/25% 5-Methoxy-UTP + 75% 1-Methyl-pseudo-UTP
CTP/25% 5-Methoxy-UTP + 75% UTP
CTP/50% 5-Methoxy-UTP + 50% 1-Methyl-pseudo-UTP
CTP/50% 5-Methoxy-UTP + 50% UTP
CTP/5-Methoxy-UTP
CTP/5-Methoxy-UTP (cap 0)
CTP/5-Methoxy-UTP(No cap)
CTP/75% 5-Methoxy-UTP + 25% 1-Methyl-pseudo-UTP
CTP/75% 5-Methoxy-UTP + 25% UTP
CTP/UTP(No cap)
N1—Me-GTP
N4—Ac-CTP
N4Ac-CTP/1-Methyl-pseudo-UTP
N4Ac-CTP/5-Methoxy-UTP
N4-acetyl-cytidine TP, ATP, GTP, UTP
N4-Bz-CTP/5-Methoxy-UTP
N4-methyl CTP
N4-Methyl-CTP/5-Methoxy-UTP
Pseudo-iso-CTP/5-Methoxy-UTP
PseudoU-alpha-thio-TP
pseudouridine TP, ATP, GTP, CTP
pseudo-UTP/5-methyl-CTP/ATP/GTP
UTP-5-oxyacetic acid Me ester
Xanthosine

According to the invention, renal polynucleotides of the invention may be synthesized to comprise the combinations or single modifications of Table 6.

Where a single modification is listed, the listed nucleoside or nucleotide represents 100 percent of that A, U, G or C nucleotide or nucleoside having been modified. Where percentages are listed, these represent the percentage of that particular A, U, G or C nucleobase triphosphate of the total amount of A, U, G, or C triphosphate present. For example, the combination: 25% 5-Aminoallyl-CTP+75% CTP/25% 5-Methoxy-UTP+75% UTP refers to a renal polynucleotide where 25% of the cytosine triphosphates are 5-Aminoallyl-CTP while 75% of the cytosines are CTP; whereas 25% of the uracils are 5-methoxy UTP while 75% of the uracils are UTP. Where no modified UTP is listed then the naturally occurring ATP, UTP, GTP and/or CTP is used at 100% of the sites of those nucleotides found in the renal polynucleotide. In this example all of the GTP and ATP nucleotides are left unmodified.

### IV. PHARMACEUTICAL COMPOSITIONS

### Formulation, Administration, Delivery and Dosing

The present invention provides renal polynucleotides, compositions and complexes thereof in combination with one or more pharmaceutically acceptable excipients. Pharmaceutical compositions may optionally comprise one or more additional active substances, e.g. therapeutically and/or prophylactically active substances. Pharmaceutical compositions of the present invention may be sterile and/or pyrogen-free. General considerations in the formulation and/or manufacture of pharmaceutical agents may be found, for example, in Remington: The Science and Practice of Pharmacy 21st ed., Lippincott Williams & Wilkins, 2005 (incorporated herein by reference in its entirety).

In some embodiments, compositions comprising at least one renal polynucleotide described herein are administered to humans, human patients or subjects. For the purposes of the present disclosure, the phrase “active ingredient” generally refers to the renal polynucleotides described herein.

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the invention will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, at least 80% (w/w) active ingredient.

### Formulations

The renal polynucleotides of the invention can be formulated using one or more excipients to: (1) increase stability; (2) increase cell transfection; (3) permit the sustained or delayed release (e.g., from a depot formulation of the renal polynucleotide); (4) alter the biodistribution (e.g., target the renal polynucleotide to specific tissues or cell types); (5) increase the translation of encoded protein in vivo; and/or (6) alter the release profile of encoded protein in vivo. In addition to traditional excipients such as any and all solvents, dispersion media, diluents, or other liquid vehicles, dispersion or suspension aids, surface active agents, isotonic agents, thickening or emulsifying agents, preservatives, excipients of the present invention can include, without limitation, lipidoids, liposomes, lipid nanoparticles, polymers, lipoplexes, core-shell nanoparticles, renal peptides, proteins, carbohydrates, cells transfected with renal polynucleotides (e.g., for transplantation into a subject), hyaluronidase, nanoparticle mimics and combinations thereof. Accordingly, the formulations of the invention can include one or more excipients, each in an amount that together increases the stability of the renal polynucleotide, increases cell transfection by the renal polynucleotide, increases the expression of renal polynucleotides encoded protein, and/or alters the release profile of renal polynucleotide encoded proteins. Further, the renal polynucleotides of the present invention may be formulated using self-assembled nucleic acid nanoparticles.

Formulations of the pharmaceutical compositions described herein may be prepared by any method known or hereafter developed in the art of pharmacology. In general, such preparatory methods include the step of associating the active ingredient with an excipient and/or one or more other accessory ingredients.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1% and 99% (w/w) of the active ingredient. By way of example, the composition may comprise between 0.1% and 100%, e.g., between 0.5 and 50%, between 1-30%, between 5-80%, at least 80% (w/w) active ingredient.

In some embodiments, the formulations described herein may contain at least one renal polynucleotide. As a non-limiting example, the formulations may contain 1, 2, 3, 4, 5 or more than 5 renal polynucleotides described herein. As a non-limiting example, the formulation may comprise more than one type of renal polynucleotide described herein.

In some embodiments, the formulations described herein may contain at least one renal polynucleotide encoding a renal polypeptide of interest and at least one nucleic acid sequence such as, but not limited to, siRNA, shRNA, snoRNA and miRNA.

In one embodiment the formulation may contain renal polynucleotide encoding proteins selected from categories such as, but not limited to, human proteins, veterinary proteins, bacterial proteins, biological proteins, antibodies, immunogenic proteins, therapeutic renal peptides and proteins, secreted proteins, plasma membrane proteins, cytoplasmic and cytoskeletal proteins, intracellular membrane bound proteins, nuclear proteins, proteins associated with human disease and/or proteins associated with non-human diseases. In one embodiment, the formulation contains at least three renal polynucleotides encoding proteins. In one embodiment, the formulation contains at least five renal polynucleotide encoding proteins.

Pharmaceutical formulations may additionally comprise a pharmaceutically acceptable excipient, which, as used herein, includes, but is not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, Md., 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

In some embodiments, the particle size of the lipid nanoparticle may be increased and/or decreased. The change in particle size may be able to help counter biological reaction such as, but not limited to, inflammation or may increase the biological effect of the renal polynucleotides delivered to mammals.

Pharmaceutically acceptable excipients used in the manufacture of pharmaceutical compositions include, but are not limited to, inert diluents, surface active agents and/or emulsifiers, preservatives, buffering agents, lubricating agents, and/or oils. Such excipients may optionally be included in the pharmaceutical formulations of the invention.

Non-limiting examples of formulations and methods of delivery of renal polynucleotides such as modified nucleic acid molecules and/or modified mRNA are taught in International Patent Publication Nos. WO2013090648, WO2013151666, WO2013151667, WO2013151668, WO2013151663, WO2013151669, WO2013151670, WO2013151664, WO2013151665, WO2013151736, WO2013151671, WO2013151672, and WO2014152211, the contents of each of which are herein incorporated by reference in its entirety.

### Lipidoids

The synthesis of lipidoids has been extensively described and formulations containing these compounds are particularly suited for delivery of renal polynucleotides. Non-limiting examples of lipidoids, lipidoid formulations and components thereof are described in International Patent Publication No. WO2015038892, the contents of which are herein incorporated by reference in its entirety.

### Liposomes

In one embodiment, pharmaceutical compositions of renal polynucleotides include liposomes. Non-limiting examples of liposomes, liposome formulations and components thereof are described in International Patent Publication No. WO2015038892, the contents of which are herein incorporated by reference in its entirety.

### Lipoplexes

In one embodiment, pharmaceutical compositions of renal polynucleotides include lipoplexes. Non-limiting examples of lipoplexes, lipoplex formulations and components thereof are described in International Patent Publication No. WO2015038892, the contents of which are herein incorporated by reference in its entirety.

### Lipid Nanoparticles

In one embodiment, renal polynucleotides described herein may be formulated in lipid nanoparticles. The formulation may be influenced by, but not limited to, the selection of the cationic lipid component, the degree of cationic lipid saturation, the nature of the PEGylation, ratio of all components and biophysical parameters such as size. In one example by Semple et al. (Semple et al. Nature Biotech. 2010 28:172-176; herein incorporated by reference in its entirety), the formulation was composed of 57.1% cationic lipid, 7.1% dipalmitoylphosphatidylcholine, 34.3% cholesterol, and 1.4% PEG-c-DMA. As another example, changing the composition of the cationic lipid could more effectively deliver siRNA to various antigen presenting cells (Basha et al. Mol Ther. 2011 19:2186-2200; herein incorporated by reference in its entirety).

In one embodiment, the LNP formulation may be formulated by the methods described in International Publication Nos. WO2011127255 or WO2008103276, the contents of each of which are herein incorporated by reference in their entirety. As a non-limiting example, modified RNA described herein may be encapsulated in LNP formulations as described in WO2011127255 and/or WO2008103276; the contents of each of which are herein incorporated by reference in their entirety.

In one embodiment, LNP formulations described herein may comprise a polycationic composition. As a non-limiting example, the polycationic composition may be selected from formula 1-60 of US Patent Publication No. US20050222064; the content of which is herein incorporated by reference in its entirety. In another embodiment, the LNP formulations comprising a polycationic composition may be used for the delivery of the modified RNA described herein in vivo and/or in vitro.

In one embodiment, the nanoparticle comprising at least one renal polynucleotide may be formulated using the methods described by Podobinski et al in U.S. Pat. No. 8,404,799, the contents of which are herein incorporated by reference in its entirety.

In some embodiments, such LNPs are synthesized using methods comprising microfluidic mixers. Exemplary microfluidic mixers may include, but are not limited to a slit interdigital micromixer including, but not limited to those manufactured by Microinnova (Allerheiligen bei Wildon, Austria) and/or a staggered herringbone micromixer (SHM) (Zhigaltsev, I. V. et al., Bottom-up design and synthesis of limit size lipid nanoparticle systems with aqueous and triglyceride cores using millisecond microfluidic mixing have been published (Langmuir. 2012. 28:3633-40; Belliveau, N. M. et al., Microfluidic synthesis of highly potent limit-size lipid nanoparticles for in vivo delivery of siRNA. Molecular Therapy-Nucleic Acids. 2012. 1:e37; Chen, D. et al., Rapid discovery of potent siRNA-containing lipid nanoparticles enabled by controlled microfluidic formulation. J Am Chem Soc. 2012. 134(16):6948-51; each of which is herein incorporated by reference in its entirety). In some embodiments, methods of LNP generation comprising SHM, further comprise the mixing of at least two input streams wherein mixing occurs by microstructure-induced chaotic advection (MICA). According to this method, fluid streams flow through channels present in a herringbone pattern causing rotational flow and folding the fluids around each other. This method may also comprise a surface for fluid mixing wherein the surface changes orientations during fluid cycling. Methods of generating LNPs using SHM include those disclosed in U.S. Application Publication Nos. 2004/0262223 and 2012/0276209, each of which is expressly incorporated herein by reference in their entirety.

In one embodiment, the renal polynucleotides of the present invention may be formulated in lipid nanoparticles created using a micromixer such as, but not limited to, a Slit Interdigital Microstructured Mixer (SIMM-V2) or a Standard Slit Interdigital Micro Mixer (SSIMM) or Caterpillar (CPMM) or Impinging-jet (IJMM) from the Institut für Mikrotechnik Mainz GmbH, Mainz Germany).

In one embodiment, the renal polynucleotides of the present invention may be formulated in lipid nanoparticles created using microfluidic technology (see Whitesides, George M. The Origins and the Future of Microfluidics. Nature, 2006 442: 368-373; Abraham et al. Chaotic Mixer for Microchannels. Science, 2002 295: 647-651; and Valencia et al. Microfluidic Platform for Combinatorial Synthesis and Optimization of Targeted Nanoparticles for Cancer Therapy. ACS Nano 2013 (DOI/10.1021/nn403370e); the contents of each of which is herein incorporated by reference in their entirety). As a non-limiting example, controlled microfluidic formulation includes a passive method for mixing streams of steady pressure-driven flows in micro channels at a low Reynolds number (See e.g., Abraham et al. Chaotic Mixer for Microchannels. Science, 2002 295: 647-651; which is herein incorporated by reference in its entirety).

In one embodiment, the renal polynucleotides of the present invention may be formulated in lipid nanoparticles created using a micromixer chip such as, but not limited to, those from Harvard Apparatus (Holliston, Mass.) or Dolomite Microfluidics (Royston, UK). A micromixer chip can be used for rapid mixing of two or more fluid streams with a split and recombine mechanism.

In one embodiment, the renal polynucleotides of the present invention may be formulated in lipid nanoparticles created using NanoAssemblr Y-mixer chip technology.

In one embodiment, the renal polynucleotides may be formulated in nanoparticles created using a microfluidic device such as the methods for making nanoparticles described in International Patent Publication No. WO2014016439, the contents of which are herein incorporated by reference in its entirety. As a non-limiting example, the nanoparticles may be created by adding a nanoparticle precursor to the microfluidic device through one or more flow channels, generating microplasma in the microfluidic device, causing the microplasma to interact with the nanoparticle precursor to generate nanoparticles, adding a conjugate material into the microfluidic device through one or more flow channels and causing the nanoparticles to mix with the conjugate material in a continuous flow to form conjugated nanoparticles (see e.g., International Patent Publication No. WO2014016439, the contents of which are herein incorporated by reference in its entirety).

In one embodiment, the nanoparticles may be prepared by the methods and processes outlined in US Patent Publication No. US20130302433, the contents of which are herein incorporated by reference in its entirety. The nanoparticles may comprise an active agent or therapeutic agent and one, two or three biocompatible polymers.

In one embodiment, the LNP formulations described herein may additionally comprise a permeability enhancer molecule. Non-limiting permeability enhancer molecules are described in US Patent Publication No. US20050222064; the content of which is herein incorporated by reference in its entirety.

In one embodiment, the lipid nanoparticle may further comprise a buffer such as, but not limited to, citrate or phosphate at a pH of 7, salt and/or sugar. Salt and/or sugar may be included in the formulations described herein for isotonicity.

In one embodiment, the lipid nanoparticles of the present invention may be hydrophilic polymer particles. Non-limiting examples of hydrophilic polymer particles and methods of making hydrophilic polymer particles are described in US Patent Publication No. US20130210991 and in US Patent Publication No. 20140073738 and 20140073715, the contents of each of which are herein incorporated by reference in their entirety. In another non-limiting example, the hydrophilic polymeric particles are described in and/or made according to the methods of US Patent Publication No. 20140142254, the contents of which is herein incorporated by reference in its entirety.

In another embodiment, the lipid nanoparticles of the present invention may be hydrophobic polymer particles.

The renal polynucleotides of the present invention may be formulated in inorganic nanoparticles (U.S. Pat. No. 8,257,745, herein incorporated by reference in its entirety).

In one embodiment, the lipid nanoparticle formulation may comprise from about 35 to about 45% cationic lipid or an ionizable amino lipid, from about 40% to about 50% cationic lipid or an ionizable amino lipid, from about 50% to about 60% cationic lipid or an ionizable amino lipid and/or from about 55% to about 65% cationic lipid or an ionizable amino lipid.

In one embodiment, the pharmaceutical compositions of the invention may comprise a nucleic acid lipid particle comprising a lipid formulation comprising 45-65 mol % of a lipid (e.g., either cationic lipid or an ionizable lipid), 5 mol % to about 10 mol %, of a non-cationic lipid of overall neutral charge, 25-40 mol % of a sterol, and 0.5-5 mol % of a PEG or PEG-modified lipid. Non-limiting examples of nucleic acid particles are disclosed in US Patent Publication No 20140121263, the contents of which are herein incorporated by reference in its entirety.

In one embodiment, the lipid nanoparticles described herein comprise 40-60% lipid (e.g., DODMA, DLin-KC2-DMA or DLin-MC3-DMA), 8-15% non-cationic lipid of neutral overall charge (e.g., DSPC or DOPE), 30-45% cholesterol and 1-5% PEG lipid (e.g., PEG 2000-DMG or anionic mPEG-DSPC). In another embodiment, the lipid nanoparticle comprises 50% lipid (e.g., DODMA, DLin-KC2-DMA or DLin-MC3-DMA), 10% non-cationic lipid of neutral overall charge (e.g., DSPC or DOPE), 38.5% cholesterol and 1.5% PEG lipid (e.g., PEG 2000-DMG).

In one embodiment, formulations comprising the renal polynucleotides and lipid nanoparticles described herein may comprise 0.15 mg/ml to 2 mg/ml of the renal polynucleotide described herein (e.g., mRNA), 50% lipid (e.g., DLin-MC3-DMA), 38.5% Cholesterol, 10% non-cationic lipid of neutral overall charge (e.g., DSPC), 1.5% PEG lipid (e.g., PEG-2K-DMG), 10 mM of citrate buffer and the formulation may additionally comprise up to 10% w/w of sucrose (e.g., at least 1% w/w, at least 2% w/w/, at least 3% w/w, at least 4% w/w, at least 5% w/w, at least 6% w/w, at least 7% w/w, at least 8% w/w, at least 9% w/w or 10% w/w).

In some embodiments, the ratio of lipid to mRNA in the lipid nanoparticles may be from 5:1, 10:1, 15:1, 20:1, 25:1, 30:1, 35:1, 40:1, 45:1, 50:1, 55:1, 60:1 or 70:1, or a range of about 5:1 to about 10:1, from about 5:1 to about 15:1, from about 5:1 to about 20:1, from about 5:1 to about 25:1, from about 5:1 to about 30:1, from about 5:1 to about 35:1, from about 5:1 to about 40:1, from about 5:1 to about 45:1, from about 5:1 to about 50:1, from about 5:1 to about 55:1, from about 5:1 to about 60:1, from about 5:1 to about 70:1, from about 10:1 to about 15:1, from about 10:1 to about 20:1, from about 10:1 to about 25:1, from about 10:1 to about 30:1, from about 10:1 to about 35:1, from about 10:1 to about 40:1, from about 10:1 to about 45:1, from about 10:1 to about 50:1, from about 10:1 to about 55:1, from about 10:1 to about 60:1, from about 10:1 to about 70:1, from about 15:1 to about 20:1, from about 15:1 to about 25:1, from about 15:1 to about 30:1, from about 15:1 to about 35:1, from about 15:1 to about 40:1, from about 15:1 to about 45:1, from about 15:1 to about 50:1, from about 15:1 to about 55:1, from about 15:1 to about 60:1 or from about 15:1 to about 70:1. As a non-limiting example, the ratio of lipid to mRNA is 10:1. As another non-limiting example, the ratio of lipid to mRNA is 20:1.

In one embodiment, the polydispersity index (PDI) of the lipid nanoparticle formulations comprising the renal polynucleotides described herein is between 0.03 and 0.2 such as, but not limited to, at least 0.03, at least 0.04, at least 0.05, at least 0.06, at least 0.07, at least 0.08, at least 0.09, at least 0.1, at least 0.11, at least 0.12, at least 0.13, at least 0.14, at least 0.15, at least 0.16, at least 0.17, at least 0.18, at least 0.19 or at least 0.2.

In one embodiment, the zeta potential of the lipid nanoparticle formulations comprising the renal polynucleotides described herein is from about −20 to about +20 at a pH in the range of 6-8.

In one embodiment, the renal polynucleotide formulations of the present invention may include a polymer combination. As a non-limiting example, the polymer combination may be two polymers used at a ratio of 1:1, 1:2, 1:2.5, 1:3, 1:4, 1:5, 1:6, 1:7, 1:8, 1:9, 1:10, 1:12.5, 1:15, 1:20, 1:25, 1:30, 1:40 or at least 1:50. In order to reduce the shear stress on the lipids during the delivery of the renal polynucleotides a polymer may be used to stabilize the polymers sensitive to degradation during delivery. The polymer combination may be PEG in combination with another polymer.

The amount of renal polynucleotides loaded into the formulation may be varied. The amount of renal polynucleotides loaded into the formulation may be, but is not limited to, at least 1 uL, at least 2 uL, at least 5 uL, at least 10 uL, at least 15 uL, at least 20 uL, at least 25 uL, at least 30 uL, at least 35 uL, at least 40 uL, at least 45 ul, at least 50 uL, at least 55 uL, at least 60 uL, at least 65 uL, at least 70 uL, at least 75 uL, at least 80 uL, at least 85 uL, at least 90 uL, at least 100 uL, at least 125 uL, at least 150 uL, at least 200 uL, at least 250 uL, at least 300 uL, at least 350 uL, at least 400 uL, at least 450 uL, at least 500 uL or more than 500 uL.

In one embodiment, the lipid nanoparticles described herein may comprise the renal polynucleotides described herein in a concentration from approximately 0.1 mg/ml to 2 mg/ml such as, but not limited to, 0.1 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml, 1.0 mg/ml, 1.1 mg/ml, 1.2 mg/ml, 1.3 mg/ml, 1.4 mg/ml, 1.5 mg/ml, 1.6 mg/ml, 1.7 mg/ml, 1.8 mg/ml, 1.9 mg/ml, 2.0 mg/ml or greater than 2.0 mg/ml.

In one embodiment, the amount of the renal polynucleotides in a formulation described herein may be at least 1 μg, at least 2 μg, at least 5 μg, at least 10 μg, at least 15 μg, at least 20 μg, at least 25 μg, at least 30 μg, at least 35 μg, at least 40 μg, at least 45 μg, at least 50 μg, at least 55 μg, at least 60 μg, at least 65 μg, at least 70 μg, at least 75 μg, at least 80 μg, at least 85 μg, at least 90 μg, at least 100 μg, at least 125 μg, at least 150 μg, at least 200 μg, at least 250 μg, at least 300 μg, at least 350 μg, at least 400 μg, at least 450 μg, at least 500 μg or more than 500 μg.

In one embodiment, the amount of the renal polynucleotides in a formulation described herein may be 5-10 μg, 5-15 μg, 5-20 μg, 5-25 μg, 5-30 μg, 5-35 μg, 5-40 μg, 5-45 μg, 5-50 μg, 10-20 μg, 10-30 μg, 10-40 μg, 10-50 μg, 20-30 μg, 20-40 μg, 20-50 μg, 30-40 μg, 30-50 μg, or 40-50 μg.

In one embodiment, the concentration of the renal polynucleotides in a formulation described herein may be at least 1 μg/ml, at least 2 μg/ml, at least 5 μg/ml, at least 10 μg/ml, at least 15 μg/ml, at least 20 μg/ml, at least 25 μg/ml, at least 30 μg/ml, at least 35 μg/ml, at least 40 μg/ml, at least 45 μg/ml, at least 50 μg/ml, at least 55 μg/ml, at least 60 μg/ml, at least 65 μg/ml, at least 70 μg/ml, at least 75 μg/ml, at least 80 μg/ml, at least 85 μg/ml, at least 90 μg/ml, at least 100 μg/ml, at least 125 μg/ml, at least 150 μg/ml, at least 200 μg/ml, at least 250 μg/ml, at least 300 μg/ml, at least 350 μg/ml, at least 400 μg/ml, at least 450 μg/ml, at least 500 μg/ml or more than 500 μg/ml.

In one embodiment, the concentration of the renal polynucleotides in a formulation described herein may be 5-10 μg/ml, 5-15 μg/ml, 5-20 μg/ml, 5-25 μg/ml, 5-30 μg/ml, 5-35 μg/ml, 5-40 μg/ml, 5-45 μg/ml, 5-50 μg/ml, 10-20 μg/ml, 10-30 μg/ml, 10-40 μg/ml, 10-50 μg/ml, 20-30 μg/ml, 20-40 μg/ml, 20-50 μg/ml, 30-40 μg/ml, 30-50 μg/ml, or 40-50 μg/ml.

In one embodiment, the concentration of the renal polynucleotides in a formulation described herein may be at least 1 μg/0.5 ml, at least 2 μg/0.5 ml, at least 5 μg/0.5 ml, at least 10 μg/0.5 ml, at least 15 μg/0.5 ml, at least 20 μg/0.5 ml, at least 25 μg/0.5 ml, at least 30 μg/0.5 ml, at least 35 μg/0.5 ml, at least 40 μg/0.5 ml, at least 45 μg/0.5 ml, at least 50 μg/0.5 ml, at least 55 μg/0.5 ml, at least 60 μg/0.5 ml, at least 65 μg/0.5 ml, at least 70 μg/0.5 ml, at least 75 μg/0.5 ml, at least 80 μg/0.5 ml, at least 85 μg/0.5 ml, at least 90 μg/0.5 ml, at least 100 μg/0.5 ml, at least 125 μg/0.5 ml, at least 150 μg/0.5 ml, at least 200 μg/0.5 ml, at least 250 μg/0.5 ml, at least 300 μg/0.5 ml, at least 350 μg/0.5 ml, at least 400 μg/0.5 ml, at least 450 μg/0.5 ml, at least 500 μg/0.5 ml or more than 500 μg/0.5 ml. As a non-limiting example, the concentration of the renal polynucleotides in a formulation may be 5 μg/0.5 ml. As another non-limiting example, the concentration of the renal polynucleotides in a formulation may be 15 μg/0.5 ml. As yet another non-limiting example, the concentration of the renal polynucleotides in a formulation may be 30 μg/0.5 ml. As another non-limiting example, the concentration of the renal polynucleotides in a formulation may be 45 μg/0.5 ml.

In one embodiment, the concentration of the renal polynucleotides in a formulation described herein may be 5-10 μg/0.5 ml, 5-15 μg/0.5 ml, 5-20 μg/0.5 ml, 5-25 μg/0.5 ml, 5-30 μg/0.5 ml, 5-35 μg/0.5 ml, 5-40 μg/0.5 ml, 5-45 μg/0.5 ml, 5-50 μg/0.5 ml, 10-20 μg/0.5 ml, 10-30 μg/0.5 ml, 10-40 μg/0.5 ml, 10-50 μg/0.5 ml, 20-30 μg/0.5 ml, 20-40 μg/0.5 ml, 20-50 μg/0.5 ml, 30-40 μg/0.5 ml, 30-50 μg/0.5 ml, or 40- 50 μg/0.5 ml.

In one embodiment, the concentration of the renal polynucleotide administered to the kidney of a subject in a formulation described herein may be at least 1 μg/0.5 ml/kidney, at least 2 μg/0.5 ml/kidney, at least 5 μg/0.5 ml/kidney, at least 10 μg/0.5 ml/kidney, at least 15 μg/0.5 ml/kidney, at least 20 μg/0.5 ml/kidney, at least 25 μg/0.5 ml/kidney, at least 30 μg/0.5 ml/kidney, at least 35 μg/0.5 ml/kidney, at least 40 μg/0.5 ml/kidney, at least 45 μg/0.5 ml/kidney, at least 50 μg/0.5 ml/kidney, at least 55 μg/0.5 ml/kidney, at least 60 μg/0.5 ml/kidney, at least 65 μg/0.5 ml/kidney, at least 70 μg/0.5 ml/kidney, at least 75 μg/0.5 ml/kidney, at least 80 μg/0.5 ml/kidney, at least 85 μg/0.5 ml/kidney, at least 90 μg/0.5 ml/kidney, at least 100 μg/0.5 ml/kidney, at least 125 μg/0.5 ml/kidney, at least 150 μg/0.5 ml/kidney, at least 200 μg/0.5 ml/kidney, at least 250 μg/0.5 ml/kidney, at least 300 μg/0.5 ml/kidney, at least 350 μg/0.5 ml/kidney, at least 400 μg/0.5 ml/kidney, at least 450 μg/0.5 ml/kidney, at least 500 μg/0.5 ml/kidney or more than 500 μg/0.5 ml/kidney. As a non-limiting example, the concentration of the renal polynucleotide administered to the kidney of a subject may be 5 μg/0.5 ml/kidney. As another non-limiting example, the concentration of the renal polynucleotide administered to the kidney of a subject may be 15 μg/0.5 ml/kidney. As yet another non-limiting example, the concentration of the renal polynucleotide administered to the kidney of a subject may be 30 μg/0.5 ml/kidney. As another non-limiting example, the concentration of the renal polynucleotide administered to the kidney of a subject may be 45 μg/0.5 ml/kidney.

In one embodiment, the concentration of the renal polynucleotide administered to the kidney of a subject in a formulation described herein may be 5-10 μg/0.5 ml, 5-15 μg/0.5 ml, 5-20 μg/0.5 ml, 5-25 μg/0.5 ml, 5-30 μg/0.5 ml, 5-35 μg/0.5 ml, 5-40 μg/0.5 ml, 5-45 μg/0.5 ml, 5-50 μg/0.5 ml, 10-20 μg/0.5 ml, 10-30 μg/0.5 ml, 10-40 μg/0.5 ml, 10-50 μg/0.5 ml, 20-30 μg/0.5 ml, 20-40 μg/0.5 ml, 20-50 μg/0.5 ml, 30-40 μg/0.5 ml, 30-50 μg/0.5 ml, or 40-50 μg/0.5 ml.

In one embodiment, renal polynucleotides may be delivered using LNPs which may have a diameter, average size or mean size from about 1 nm to about 100 nm, from about 1 nm to about 10 nm, about 1 nm to about 20 nm, from about 1 nm to about 30 nm, from about 1 nm to about 40 nm, from about 1 nm to about 50 nm, from about 1 nm to about 60 nm, from about 1 nm to about 70 nm, from about 1 nm to about 80 nm, from about 1 nm to about 90 nm, from about 5 nm to about from 100 nm, from about 5 nm to about 10 nm, about 5 nm to about 20 nm, from about 5 nm to about 30 nm, from about 5 nm to about 40 nm, from about 5 nm to about 50 nm, from about 5 nm to about 60 nm, from about 5 nm to about 70 nm, from about 5 nm to about 80 nm, from about 5 nm to about 90 nm, from about 10 nm to about from 100 nm, about 10 nm to about 20 nm, from about 10 nm to about 30 nm, from about 10 nm to about 40 nm, from about 10 nm to about 50 nm, from about 10 nm to about 60 nm, from about 10 nm to about 70 nm, from about 10 nm to about 80 nm, from about 10 nm to about 90 nm, from about 20 nm to about from 100 nm, from about 20 nm to about 30 nm, from about 20 nm to about 40 nm, from about 20 nm to about 50 nm, from about 20 nm to about 60 nm, from about 20 nm to about 70 nm, from about 20 nm to about 80 nm, from about 20 nm to about 90 nm, from about 30 nm to about from 100 nm, from about 30 nm to about 40 nm, from about 30 nm to about 50 nm, from about 30 nm to about 60 nm, from about 30 nm to about 70 nm, from about 30 nm to about 80 nm, from about 30 nm to about 90 nm, from about 40 nm to about from 100 nm, from about 40 nm to about 50 nm, from about 40 nm to about 60 nm, from about 40 nm to about 70 nm, from about 40 nm to about 80 nm, from about 40 nm to about 90 nm, from about 50 nm to about from 100 nm, from about 50 nm to about 60 nm, from about 50 nm to about 70 nm, from about 50 nm to about 80 nm, from about 50 nm to about 90 nm, from about 60 nm to about from 100 nm, from about 60 nm to about 70 nm, from about 60 nm to about 80 nm, from about 60 nm to about 90 nm, from about 70 nm to about from 100 nm, from about 70 nm to about 80 nm, from about 70 nm to about 90 nm, from about 80 nm to about from 100 nm, from about 80 nm to about 90 nm or from about 90 nm to about from 100 nm.

In some embodiments, one or more renal polynucleotides may be delivered using LNPs which may have a diameter, average size or mean size from about 10-500 nm, from about 50-150 nm, from about 70-120 nm, from about 80-110 nm, from about 90-100 nm, from about 95-102 nm, or from about 98-100 nm.

In some embodiments, the LNPs may comprise a diameter selected from 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150 nm.

In one embodiment, the lipid nanoparticle may comprise a diameter, average size or mean size greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm or greater than 1000 nm.

In one embodiment, the nanoparticles may have a hydrodynamic diameter of about 70 to about 130 nm such as, but not limited to, the nanoparticles described in US Patent Publication No. US20130302432, the contents of which are herein incorporated by reference in its entirety. As a non-limiting example, the nanoparticles have about 0.2 to about 35 weight percent of a therapeutic agent and about 10 to about 99 weight percent of biocompatible polymer such as a diblock poly(lactic) acid-poly(ethylene)glycol (see e.g., US Patent Publication No. US20130302432, the contents of which are herein incorporated by reference in its entirety).

In one embodiment, the lipid nanoparticles comprising the renal polynucleotides described herein may produce the encoded renal polypeptide of interest for at least 3 hours in a cell, tissue, organ or subject.

In one embodiment, the lipid nanoparticles comprising the renal polynucleotides described herein may produce the encoded renal polypeptide of interest for at least 6 hours in a cell, tissue, organ or subject.

In one embodiment, the lipid nanoparticles comprising the renal polynucleotides described herein may produce the encoded renal polypeptide of interest for at least 20 hours in a cell, tissue, organ or subject.

In one embodiment, the lipid nanoparticles comprising the renal polynucleotides described herein may produce the encoded renal polypeptide of interest for at least 22 hours in a cell, tissue, organ or subject.

In one embodiment, the lipid nanoparticles comprising the renal polynucleotides described herein may produce the encoded renal polypeptide of interest for at least 24 hours in a cell, tissue, organ or subject.

In one embodiment, the components of the lipid nanoparticle may be tailored for optimal delivery of the renal polynucleotides based on the delivery route and the desired outcome. As a non-limiting example, the lipid nanoparticle may comprise 40-60% lipid (either cationic lipid or an ionizable lipid), 8-16% non-cationic lipid of neutral overall charge, 30-45% cholesterol and 1-5% PEG lipid. As another non limiting example, the lipid nanoparticle may comprise 50% lipid (either cationic lipid or an ionizable lipid), 10% non-cationic lipid of neutral overall charge, 38.5% cholesterol and 1.5% PEG lipid.

As yet another non-limiting example, the 40-60%, lipid (either cationic lipid or an ionizable lipid) may be DODMA, DLin-KC2-DMA or DLin-MC3-DMA, the 8-15% non-cationic lipid of neutral overall charge may be DSPC or DOPE and the 1-5% PEG lipid may be PEG 2000-DMG or anionic mPEG-DSPC and the lipid nanoparticle may comprise 30-45% cholesterol.

In one embodiment, the renal polynucleotides may be formulated in and/or delivered in a lipid nanoparticle as described in International Patent Publication No. WO2012170930, the contents of which are herein incorporated by reference in its entirety. The lipid nanoparticle may comprise one or more lipids (e.g., cationic lipids or ionizable amino lipids), one or more non-cationic lipids of neutral overall charge and one or more PEG-modified lipids. As a non-limiting example, the lipid nanoparticle comprises DLin-KC2-DMA, Cholesterol (CHOL), DOPE and DMG-PEG-2000. As another non-limiting example, the lipid nanoparticle comprises C12-200, DOPE, cholesterol (CHOL) and DMGPEG2K.

In one embodiment, the formulations of the renal polynucleotides described herein may comprise a component such as, but not limited to, cationic lipids, cholesterol, PEG-DMG, DOPE, DSPC, Methoxy PEG-DSPC, Hydrogenated soy phospatidyl glycerol, sphingomyelin, DOPC, DPPC, dierucoylphophadtidylcholine (DEPC), tricaprylin (C8:0), triolein (C18:1), soybean oil, methoxy-PEG-40-carbonyl-distearoylphosphatidylethanolamine, L-dimyristoylphosphatidylcholine, L-dimyristoylphosphatidylglycerol, egg phosphatidylglycerol, MPEG5000 DPPE, DPPA (dipalmitoyl phosphatide), phosphatidylcholine, DPPG, LECIVA-S90 (purified PC from soy), LECIVA-S70 (pure phospholipid from soy lecithin), LIPOVA-E120 (purified egg lecithin USP), Egg lecithin, propylene glycol, glycerol, polysorbate 80, glutathione (reduced), butylated hydroxytoluene (BHA), ascorbyl palmitate, alpha-tocopherol, sodium carbonate, TRIS, histidine, calcium chloride, sodium phosphate, sodium citrate, ammonium sulfate, mannitol, sucrose, lactose, trehalose, disodium succinate hexahydrate and nitrogen.

In one embodiment, the therapeutic nanoparticles may comprise at least one cationic polymer described herein and/or known in the art.

In one embodiment, the lipid nanoparticles described herein may comprise a lipid such as, but not limited to, a cationic lipid or an ionizable lipid, a non-cationic lipid of neutral overall charge (e.g., zwitterionic lipids and phospholipids including, but not limited to, DSPC and DOPE), cholesterol and a PEG lipid.

In one embodiment, the formulations of the renal polynucleotides described herein may comprise a lipid such as, but not limited to, DLin-DMA, DLin-K-DMA, 98N12-5, C12-200, ckk, E12, DLin-MC3-DMA, DLin-KC2-DMA, KL10, KL52, KL22, DODMA, DOPE, DSPC, PLGA, PEG, PEG-DMG, PEG-DSG, PEG-DSPE, PEG-DOMG, PEGylated lipids, polyethylenimine (PEI) and chitosan. As a non-limiting example, the lipid may be cationic lipid such as, but not limited to, C12-200, DLin-DMA, DLin-K-DMA and DODMA. As another non-limiting example, the lipid may be an ionizable lipid such as, but not limited to, DLin-MC3-DMA and DLin-KC2-DMA.

In one embodiment, the lipid nanoparticle comprising the renal polynucleotides of the present invention may comprise the lipids KL10, KL22, KL52, C12-200, DLin-KC2-DMA, DOPE and/or DSPC.

In one embodiment, the lipid nanoparticle comprising the renal polynucleotides of the present invention may comprise the lipids KL10 and DOPE or KL10 and DSPC. The lipid nanoparticle may also comprise at least one PEG lipid. The percentage of the PEG lipid in the lipid nanoparticle may be between 1-7%. As a non-limiting example, the percentage of PEG lipid is 1.5%. As another non-limiting example, the percentage of PEG lipid is 3%. As another non-limiting example, the percentage of PEG lipid is 5%.

In one embodiment, the lipid nanoparticle comprising the renal polynucleotides of the present invention may comprise the lipids C12-200 and DOPE or C12-200 and DSPC. The lipid nanoparticle may also comprise at least one PEG lipid. The percentage of the PEG lipid in the lipid nanoparticle may be between 1-7%. As a non-limiting example, the percentage of PEG lipid is 1.5%. As another non-limiting example, the percentage of PEG lipid is 3%. As another non-limiting example, the percentage of PEG lipid is 5%.

In one embodiment, the lipid nanoparticle comprising the renal polynucleotides of the present invention may comprise the lipids KL22 and DOPE or KL22 and DSPC. The lipid nanoparticle may also comprise at least one PEG lipid. The percentage of the PEG lipid in the lipid nanoparticle may be between 1-7%. As a non-limiting example, the percentage of PEG lipid is 1.5%. As another non-limiting example, the percentage of PEG lipid is 3%. As another non-limiting example, the percentage of PEG lipid is 5%.

In one embodiment, the lipid nanoparticle comprising the renal polynucleotides of the present invention may comprise the lipids KL52 and DOPE or KL52 and DSPC. The lipid nanoparticle may also comprise at least one PEG lipid. The percentage of the PEG lipid in the lipid nanoparticle may be between 1-7%. As a non-limiting example, the percentage of PEG lipid is 1.5%. As another non-limiting example, the percentage of PEG lipid is 3%. As another non-limiting example, the percentage of PEG lipid is 5%.

In one embodiment, the lipid nanoparticle comprising the renal polynucleotides of the present invention may comprise the lipids DLin-MC3-DMA and DOPE or DLin-MC3-DMA and DSPC. The lipid nanoparticle may also comprise at least one PEG lipid. The percentage of the PEG lipid in the lipid nanoparticle may be between 1-7%. As a non-limiting example, the percentage of PEG lipid is 1.5%. As another non-limiting example, the percentage of PEG lipid is 3%. As another non-limiting example, the percentage of PEG lipid is 5%.

In one embodiment, the lipid nanoparticle comprising the renal polynucleotides of the present invention may comprise the lipids DLin-KC2-DMA and DOPE or DLin-KC2-DMA and DSPC. The lipid nanoparticle may also comprise at least one PEG lipid. The percentage of the PEG lipid in the lipid nanoparticle may be between 1-7%. As a non-limiting example, the percentage of PEG lipid is 1.5%. As another non-limiting example, the percentage of PEG lipid is 3%. As another non-limiting example, the percentage of PEG lipid is 5%.

Lipid nanoparticle formulations may be improved by replacing the lipid which is either cationic or an ionizable amino lipid with a biodegradable lipid which is known as a rapidly eliminated lipid nanoparticle (reLNP). Lipids, which may be replaced with a biodegradable lipid include, but are not limited to, DLinDMA, DLin-KC2-DMA, and DLin-MC3-DMA, have been shown to accumulate in plasma and tissues over time and may be a potential source of toxicity. The rapid metabolism of the rapidly eliminated lipids can improve the tolerability and therapeutic index of the lipid nanoparticles by an order of magnitude from a 1 mg/kg dose to a 10 mg/kg dose in rat. Inclusion of an enzymatically degraded ester linkage can improve the degradation and metabolism profile of the cationic component, while still maintaining the activity of the reLNP formulation. The ester linkage can be internally located within the lipid chain or it may be terminally located at the terminal end of the lipid chain. The internal ester linkage may replace any carbon in the lipid chain.

In one embodiment, the internal ester linkage may be located on either side of the saturated carbon.

In one embodiment, the lipid nanoparticle may comprise a polymer or co-polymer. Non-limiting examples of specific polymers include poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacralate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone, polysiloxanes, polystyrene (PS), polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, poly(ortho)esters, poly(butyric acid), poly(valeric acid), poly(lactide-co-caprolactone), PEG-PLGA-PEG and trimethylene carbonate, polyvinylpyrrolidone.

In one embodiment, the nanoparticles of the present invention may comprise a polymeric matrix. As a non-limiting example, the nanoparticle may comprise two or more polymers such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), poly(4-hydroxy-L-proline ester), polyesters, polyanhydrides, polyethers, polyurethanes, polymethacrylates, polyacrylates, polycyanoacrylates or combinations thereof.

In one embodiment, the nanoparticle comprises a diblock copolymer. In one embodiment, the diblock copolymer may include PEG in combination with a polymer such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), poly(4-hydroxy-L-proline ester) or combinations thereof. In another embodiment, the diblock copolymer may comprise the diblock copolymers described in European Patent Publication No. the contents of which are herein incorporated by reference in its entirety. In yet another embodiment, the diblock copolymer may be a high-X diblock copolymer such as those described in International Patent Publication No. WO2013120052, the contents of which are herein incorporated by reference in its entirety. In another embodiment, the diblock copolymer may be, but it not limited to, a poly(lactic) acid-poly(ethylene)glycol copolymer (see e.g., International Patent Publication No. WO2013044219; herein incorporated by reference in its entirety). As a non-limiting example, the therapeutic nanoparticle may be used to treat cancer (see International publication No. WO2013044219; herein incorporated by reference in its entirety).

As a non-limiting example the nanoparticle comprises a PLGA-PEG block copolymer (see US Pub. No. US20120004293 and U.S. Pat. No. 8,236,330, each of which is herein incorporated by reference in their entirety). In another non-limiting example, the therapeutic nanoparticle is a stealth nanoparticle comprising a diblock copolymer of PEG and PLA or PEG and PLGA (see U.S. Pat. No. 8,246,968 and International Publication No. WO2012166923, the contents of each of which are herein incorporated by reference in its entirety). In yet another non-limiting example, the therapeutic nanoparticle is a stealth nanoparticle or a target-specific stealth nanoparticle as described in US Patent Publication No. US20130172406, the contents of which are herein incorporated by reference in its entirety.

In yet another non-limiting example, the lipid nanoparticle comprises the block copolymer PEG-PLGA-PEG (see e.g., the thermosensitive hydrogel (PEG-PLGA-PEG) was used as a TGF-beta1 gene delivery vehicle in Lee et al. Thermosensitive Hydrogel as a Tgfβ1 Gene Delivery Vehicle Enhances Diabetic Wound Healing. Pharmaceutical Research, 2003 20(12): 1995-2000; as a controlled gene delivery system in Li et al. Controlled Gene Delivery System Based on Thermosensitive Biodegradable Hydrogel. Pharmaceutical Research 2003 20(6):884-888; and Chang et al., Non-ionic amphiphilic biodegradable PEG-PLGA-PEG copolymer enhances gene delivery efficiency in rat skeletal muscle. J Controlled Release. 2007 118:245-253; each of which is herein incorporated by reference in its entirety). The renal polynucleotides of the present invention may be formulated in lipid nanoparticles comprising the PEG-PLGA-PEG block copolymer.

In one embodiment, the nanoparticle may comprise a multiblock copolymer (See e.g., U.S. Pat. Nos. 8,263,665 and 8,287,910 and US Patent Pub. No. US20130195987; the contents of each of which are herein incorporated by reference in its entirety). As a non-limiting example, the multiblock copolymer which may be used in the nanoparticles described herein may be a non-linear multiblock copolymer such as those described in US Patent Publication No. 20130272994, the contents of which are herein incorporated by reference in its entirety.

In one embodiment, the nanoparticle may comprise at least one acrylic polymer. Acrylic polymers include but are not limited to, acrylic acid, methacrylic acid, acrylic acid and methacrylic acid copolymers, methyl methacrylate copolymers, ethoxyethyl methacrylates, cyanoethyl methacrylate, amino alkyl methacrylate copolymer, poly(acrylic acid), poly(methacrylic acid), polycyanoacrylates and combinations thereof.

In one embodiment, the nanoparticles may comprise at least one poly(vinyl ester) polymer. The poly(vinyl ester) polymer may be a copolymer such as a random copolymer. As a non-limiting example, the random copolymer may have a structure such as those described in International Application No. WO2013032829 or US Patent Publication No US20130121954, the contents of which are herein incorporated by reference in its entirety. In one aspect, the poly(vinyl ester) polymers may be conjugated to the renal polynucleotides described herein. In another aspect, the poly(vinyl ester) polymer which may be used in the present invention may be those described in, herein incorporated by reference in its entirety.

In one embodiment, the nanoparticles may comprise at least one amine-containing polymer such as, but not limited to polylysine, polyethylene imine, poly(amidoamine) dendrimers, poly(beta-amino esters) (See e.g., U.S. Pat. Nos. 8,287,849 and 8,557,231; the contents of which are herein incorporated by reference in its entirety) and combinations thereof. As a non-limiting example, the amine-containing polymer may be any of the biodegradable poly(beta-amino esters) described in U.S. Pat. No. 8,557,231, the contents of which are herein incorporated by reference in its entirety.

In another embodiment, the nanoparticles described herein may comprise an amine cationic lipid such as those described in International Patent Application No. WO2013059496, the contents of which are herein incorporated by reference in its entirety. In one aspect the cationic lipids may have an amino-amine or an amino-amide moiety.

In some embodiments, LNPs may comprise linear amino-lipids as described in U.S. Pat. No. 8,691,750, the contents of which is herein incorporated by reference in its entirety.

In one embodiment, the nanoparticles may comprise at least one degradable polyester which may contain polycationic side chains. Degradable polyesters include, but are not limited to, poly(serine ester), poly(L-lactide-co-L-lysine), poly(4-hydroxy-L-proline ester), and combinations thereof. In another embodiment, the degradable polyesters may include a PEG conjugation to form a PEGylated polymer.

In some embodiments, LNPs comprise the lipid KL52, KL22 or KL10 (an amino-lipid disclosed in U.S. Application Publication No. 2012/0295832 expressly incorporated herein by reference in its entirety). Activity and/or safety (as measured by examining one or more of ALT/AST, white blood cell count and cytokine induction) of LNP administration may be improved by incorporation of such lipids. LNPs comprising KL52, KL22 or KL10 may be administered arterially, intravenously and/or in one or more doses. In some embodiments, administration of LNPs comprising KL52, KL10, or KL22 results in equal or improved mRNA and/or protein expression as compared to LNPs comprising DLin-MC3-DMA or DLin-KC2-DMA.

In one embodiment, the renal polynucleotide formulations of the present invention may include at least one polymer such as, but not limited to, polyethenes, polyethylene glycol (PEG), poly(I-lysine)(PLL), PEG grafted to PLL, cationic lipopolymer, biodegradable cationic lipopolymer, polyethylenimine (PEI), cross-linked branched poly(alkylene imines), a polyamine derivative, a modified poloxamer, a biodegradable polymer, elastic biodegradable polymer, biodegradable block copolymer, biodegradable random copolymer, biodegradable polyester copolymer, biodegradable polyester block copolymer, biodegradable polyester block random copolymer, multiblock copolymers, linear biodegradable copolymer, poly[α-(4-aminobutyl)-L-glycolic acid) (PAGA), biodegradable cross-linked cationic multi-block copolymers, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), poly(4-hydroxy-L-proline ester), acrylic polymers, amine-containing polymers, dextran polymers, dextran polymer derivatives or combinations thereof.

In one embodiment, the renal polynucleotide formulations of the present invention may include a polymer combination of PLGA and PEG. As a non-limiting example, PEG may be used with PLGA in the delivery and/or formulation of the renal polynucleotides to reduce the degradation of PLGA during delivery. As another non-limiting example, the PLGA and PEG lipids used in the formulation and/or delivery of the renal polynucleotides may be in a 50:50 ratio. As yet another non-limiting example, the PLGA has a size of approximately 15K and the PEG has a size of approximately 2K and used in the formulation and/or delivery of the renal polynucleotides in a 50:50 ratio.

In one embodiment, the renal polynucleotide formulations of the present invention may include at least one acrylic polymer. Acrylic polymers include but are not limited to, acrylic acid, methacrylic acid, acrylic acid and methacrylic acid copolymers, methyl methacrylate copolymers, ethoxyethyl methacrylates, cyanoethyl methacrylate, amino alkyl methacrylate copolymer, poly(acrylic acid), poly(methacrylic acid), polycyanoacrylates and combinations thereof.

In one embodiment, the formulation of the present invention may include a cationic lipopolymer such as, but is not limited to, polyethylenimine, poly(trimethylenimine), poly(tetramethylenimine), polypropylenimine, aminoglycoside-polyamine, dideoxy-diamino-b-cyclodextrin, spermine, spermidine, poly(2-dimethylamino)ethyl methacrylate, poly(lysine), poly(histidine), poly(arginine), cationized gelatin, dendrimers, chitosan, 1,2-Dioleoyl-3-Trimethylammonium-Propane(DOTAP), N-[1-(2,3-dioleoyloxy)propyl]-N,N,N-trimethylammonium chloride (DOTMA), 1-[2-(oleoyloxy)ethyl]-2-oleyl-3-(2-hydroxyethyl)imidazolinium chloride (DOTIM), 2,3-dioleyloxy-N-[2(sperminecarboxamido)ethyl]-N,N-dimethyl-1-propanaminium trifluoroacetate (DOSPA), 3B-[N—(N′,N′-Dimethylaminoethane)-carbamoyl]Cholesterol Hydrochloride (DC-Cholesterol HCl) diheptadecylamidoglycyl spermidine (DOGS), N,N-distearyl-N,N-dimethylammonium bromide (DDAB), N-(1,2-dimyristyloxyprop-3-yl)-N,N-dimethyl-N-hydroxyethyl ammonium bromide (DMRIE), N,N-dioleyl-N,N-dimethylammonium chloride DODAC) and combinations thereof. As a non-limiting example, the renal polynucleotides may be formulated with a cationic lipopolymer such as those described in U.S. Patent Application No. 20130065942, herein incorporated by reference in its entirety.

In one embodiment, the formulations described herein may comprise two or more cationic polymers. The cationic polymer may comprise a poly(ethylene imine) (PEI) such as linear PEI. In another embodiment, the polyplex comprises p(TETA/CBA) its PEGylated analog p(TETA/CBA)-g-PEG2k and mixtures thereof (see e.g., US Patent Publication No. US20130149783, the contents of which are herein incorporated by reference in its entirety.

In one embodiment, the lipid or lipids which may be used in the formulation and/or delivery of renal polynucleotides described herein may be, but is not limited to, DLin-DMA, DLin-K-DMA, 98N12-5, C12-200, DLin-MC3-DMA, DLin-KC2-DMA, DODMA, PLGA, PEG, PEG-DMG, PEGylated lipids and amino alcohol lipids. The amino alcohol cationic lipid may be the lipids described in and/or made by the methods described in US Patent Publication No. US20130150625, herein incorporated by reference in its entirety. As a non-limiting example, the cationic lipid may be 2-amino-3-[(9Z,12Z)-octadeca-9,12-dien-1-yloxy]-2-{[(9Z,2Z)-octadeca-9,12-dien-1-yloxy]methyl}propan-1-ol (Compound 1 in US20130150625); 2-amino-3-[(9Z)-octadec-9-en-1-yloxy]-2-{[(9Z)-octadec-9-en-1-yloxy]methyl}propan-1-ol (Compound 2 in US20130150625); 2-amino-3-[(9Z,12Z)-octadeca-9,12-dien-1-yloxy]-2-[(octyloxy)methyl]propan-1-ol (Compound 3 in US20130150625); and 2-(dimethylamino)-3-[(9Z,12Z)-octadeca-9,12-dien-1-yloxy]-2-{[(9Z,12Z)-octadeca-9,12-dien-1-yloxy]methyl}propan-1-ol (Compound 4 in US20130150625); or any pharmaceutically acceptable salt or stereoisomer thereof 1,2-Dioleoyl-sn-glycero-3-phosphatidylcholine (DOPC), 1,2-Dioleoyl-sn-glycero-3-phosphatidylethanolamine (DOPE), cholesterol, N-[1-(2,3-Dioleyloxy)propyl]N,N,N-trimethylammonium chloride (DOTMA), 1,2-Dioleoyloxy-3-trimethylammonium-propane (DOTAP), Dioctadecylamidoglycylspermine (DOGS), N-(3-Aminopropyl)-N,N-dimethyl-2,3-bis(dodecyloxy)-1-propanaminium bromide (GAP-DLRIE), cetyltrimethylammonium bromide (CTAB), 6-lauroxyhexyl ornithinate (LHON), 1-)2,3-Dioleoloxypropyl)2,4,6-trimethylpyridinium (20c), 2,3-Dioleyloxy-N-[2(sperminecarboxamido)-ehtyl]-N,N-dimethyl-1-propanaminium trifluoroacetate (DOSPA), 1,2-Dioleyl-3-trimethylammonium-propane (DOPA), N-(2-Hydroxyethyl)-N,N-dimethyl-2,3-bis(tetradecyloxy)-1-propanaminium bromide (MDRIE), Dimyristooxypropyl dimethyl hydroxyethyl ammonium bromide (DMRI), 3β-[N—(N′,N′-Dimethylaminoethane)-carbamoyl]cholesterol (DC-Chol), Bis-guanidium-tren-cholesterol (BGTC), 1,3-Dioleoxy-2-(6-carboxy-spermyl)-propylamide (DOSPER), Dimethyloctadecylammonium bromide (DDAB), Dioctadecylamidoglicylspermidin (DSL), rac-[(2,3-Dioctadecyloxypropyl)(2-hydroxyethyl)]-dimethylammonium chloride (CLIP-1), rac-[2(2,3-Dihexadecyloxypropyl-oxymethyloxy)ehtyl]trimethylammonium chloride (CLIP-6), Ethyldimyrisotylphosphatidylcholine (EDMPC), 1,2-Distearyloxy-N,N-dimethyl-3-aminopropane (DSDMA), 1,2-Dimyristoyl-trimethylammoniumpropane (DMTAP), O,O′-Dimyristyl-N-lysyl asparate (DMKE), 1,2-Distearoyl-sn-glycero-3-ethylphosphocholine (DSEPC), N-Palmitoyl-D-erythro-spingosyl carbamoyl-spermine (CCS), N-t-Butyl-No-tetradecyl-3-tetradecylam inopropionamidine (diC14-amidine), Octadecenolyoxy[ethyl-2-heptadecenyl-3 hydroxyethyl] imidazolinium chloride (DOTIM), N1-Cholesteryloxycarbonyl-3,7-diazanonane-1,9-diamine (CDAN) and 2-(3-[Bis-(3-amino-propyl)-amino]propylamino)-N-ditetradecylcarbamoylme-ethyl-acetamide (RPR2091290).

In one embodiment, the polymers which may be used in the formulation and/or delivery of renal polynucleotides described herein may be, but is not limited to, poly(ethylene)glycol (PEG), polyethylenimine (PEI), dithiobis(succinimidylpropionate) (DSP), Dimethyl-3,3′-dithiobispropionimidate (DTBP), poly(ethylene imine) biscarbamate (PEIC), poly(L-lysine) (PLL), histidine modified PLL, poly(N-vinylpyrrolidone) (PVP), poly(propylenimine (PPI), poly(amidoamine) (PAMAM), poly(amido ethylenimine) (SS-PAEI), triehtylenetetramine (TETA), poly(β-aminoester), poly(4-hydroxy-L-proine ester) (PHP), poly(allylamine), poly(α-[4-aminobutyl]-L-glycolic acid (PAGA), Poly(D,L-lactic-co-glycolid acid (PLGA), Poly(N-ethyl-4-vinylpyridinium bromide), poly(phosphazene)s (PPZ), poly(phosphoester)s (PPE), poly(phosphoramidate)s (PPA), poly(N-2-hydroxypropylmethacrylamide) (pHPMA), poly(2-(dimethylamino)ethyl methacrylate) (pDMAEMA), poly(2-aminoethyl propylene phosphate) PPE_EA), Chitsoan, galactosylated chitosan, N-dodecylated chitosan, histone, collagen and dextran-spermine. In one embodiment, the polymer may be an inert polymer such as, but not limited to, PEG. In one embodiment, the polymer may be a cationic polymer such as, but not limited to, PEI, PLL, TETA, poly(allylamine), Poly(N-ethyl-4-vinylpyridinium bromide), pHPMA and pDMAEMA. In one embodiment, the polymer may be a biodegradable PEI such as, but not limited to, DSP, DTBP and PEIC. In one embodiment, the polymer may be biodegradable such as, but not limited to, histine modified PLL, SS-PAEI, poly(β-aminoester), PHP, PAGA, PLGA, PPZ, PPE, PPA and PPE-EA.

In one embodiment, the lipid nanoparticles described herein may comprise a PEG lipid. The lipid nanoparticle may comprise from about 0.5% to about 3.0%, from about 1.0% to about 7%, from about 1.0% to about 5.0%, from about 1.0% to about 3.5%, from about 1.5% to about 4.0%, from about 2.0% to about 4.5%, from about 2.5% to about 5.0% and/or from about 3.0% to about 6.0% of PEG lipid. In one aspect, the lipid nanoparticles comprise about 1.5% of PEG lipid. In another aspect, the lipid nanoparticles comprise about 3.0% PEG lipid. In yet another aspect, the lipid nanoparticles comprise about 5.0% PEG lipid.

In one embodiment, the lipid nanoparticle may comprise 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%, 5.1%, 5.2%, 5.3%, 5.4%, 5.5%, 5.6%, 5.7%, 5.8%, 5.9%, 6%, 6.1%, 6.2%, 6.3%, 6.4%, 6.5%, 6.6%, 6.7%, 6.8%, 6.9%, 7% or more than 7% PEG lipid. As a non-limiting example, the lipid nanoparticle comprises 1.5% PEG lipid. As another non-limiting example, the lipid nanoparticle comprises 3% PEG lipid. As yet another example, the lipid nanoparticle comprises 5% PEG lipid.

In some embodiments, the ratio of PEG in the lipid nanoparticle (LNP) formulations may be increased or decreased and/or the carbon chain length of the PEG lipid may be modified from C14 to C18 to alter the pharmacokinetics and/or biodistribution of the LNP formulations. As a non-limiting example, LNP formulations may contain from about 0.5% to about 3.0%, from about 1.0% to about 3.5%, from about 1.5% to about 4.0%, from about 2.0% to about 4.5%, from about 2.5% to about 5.0% and/or from about 3.0% to about 6.0% of the lipid molar ratio of PEG-c-DOMG as compared to the lipid, DSPC and cholesterol. In another embodiment the PEG-c-DOMG may be replaced with a PEG lipid such as, but not limited to, PEG-DSG (1,2-Distearoyl-sn-glycerol, methoxypolyethylene glycol), PEG-DMG (1,2-Dimyristoyl-sn-glycerol) and/or PEG-DPG (1,2-Dipalmitoyl-sn-glycerol, methoxypolyethylene glycol). The lipid may be a cationic lipid or an ionizable amino lipid selected from any lipid known in the art such as, but not limited to, DLin-MC3-DMA, DLin-DMA, C12-200 and DLin-KC2-DMA, DLin-K-DMA, 98N12-5, ckk, E12, DODMA, DOPE, DSPC, PLGA, PEG-DMG, PEG-DSG, PEG-DSPE, PEG-DOMG, PEGylated lipids, polyethylenimine (PEI) and chitosan.

In one embodiment, the lipid nanoparticles described herein may comprise a PEG lipid which is a non-diffusible PEG. Non-limiting examples of non-diffusible PEGs include PEG-DSG and PEG-DSPE. As a non-limiting example, the lipid nanoparticle comprising the PEG lipid comprises 40-60% lipid (e.g., DODMA, DLin-KC2-DMA or DLin-MC3-DMA), 8-15% non-cationic lipid of neutral overall charge (e.g., DSPC or DOPE), 30-45% cholesterol and 0.5-10% PEG lipid (e.g., PEG-DSG or PEG-DSPE). As another non-limiting example, the lipid nanoparticle comprising the PEG lipid comprises 50% lipid (e.g., DODMA, DLin-KC2-DMA or DLin-MC3-DMA), 10% non-cationic lipid of neutral overall charge (e.g., DSPC or DOPE), 39.5%, 38.5%, 35% or 30% cholesterol and 0.5%, 1.5%, 5% or 10% PEG lipid (e.g., PEG-DSG, PEG-DMG, PEG-DOMG, or PEG-DSPE).

In one embodiment, the pharmaceutical compositions of the renal polynucleotides may include at least one of the PEGylated lipids described in International Publication No. WO2012099755 or PEGylated polymer described in International Publication No. WO2012099755, the contents of each of which are herein incorporated by reference.

In one embodiment, the LNP formulations of the renal polynucleotides may contain PEG-c-DOMG at 3% lipid molar ratio. In another embodiment, the LNP formulations renal polynucleotides may contain PEG-c-DOMG at 1.5% lipid molar ratio.

In one embodiment, the LNP formulation may contain PEG-DMG 2000 (1,2-dimyristoyl-sn-glycero-3-phophoethanolamine-N-[methoxy(polyethylene glycol)-2000). In one embodiment, the LNP formulation may contain PEG-DMG 2000, a cationic lipid known in the art and at least one other component. In another embodiment, the LNP formulation may contain PEG-DMG 2000, a cationic lipid known in the art, DSPC and cholesterol. As a non-limiting example, the LNP formulation may contain PEG-DMG 2000, DLin-DMA, DSPC and cholesterol. As another non-limiting example the LNP formulation may contain PEG-DMG 2000, DLin-DMA, DSPC and cholesterol in a molar ratio of 2:40:10:48 (see e.g., Geall et al., Nonviral delivery of self-amplifying RNA vaccines, PNAS 2012; PMID: 22908294; herein incorporated by reference in its entirety).

In another aspect the limit size lipid nanoparticle may comprise a polyethylene glycol-lipid such as, but not limited to, DLPE-PEG, DMPE-PEG, DPPC-PEG and DSPE-PEG.

As a non-limiting example, the nanoparticles may comprise a poly(lactic) acid-block-poly(ethylene)glycol copolymer or poly(lactic)-co-poly(glycolic) acid-block-poly(ethylene)glycol copolymer, and a therapeutic agent (e.g., renal polynucleotides).

In one embodiment, the nanoparticle may be a polyethylene glycolated (PEGylated) nanoparticle such as, but not limited to, the PEGylated nanoparticles described in US Patent Publication No. US20140044791, the contents of which are herein incorporated by reference in its entirety. The PEGylated nanoparticle may comprise at least one targeting moiety coupled to the polyethylene glycol of the nanoparticle in order to target the composition to a specific cell. Non-limiting examples, of PEGylated nanoparticles and targeting moieties are described in US Patent Publication No. US20140044791, the contents of which are herein incorporated by reference in its entirety.

In one embodiment, the renal polynucleotides of the invention may be formulated in or with at least PEGylated albumin polymer. PEGylated albumin polymer and methods of making PEGylated albumin polymer include those known in the art and described in US Patent Publication No. US20130231287, the contents of each of which are herein incorporated by reference in its entirety.

In one embodiment, the formulations described herein may comprise a lipid-terminating PEG. As a non-limiting example, PLGA may be conjugated to a lipid-terminating PEG forming PLGA-DSPE-PEG. As another non-limiting example, PEG conjugates for use with the present invention are described in International Publication No. WO2008103276, herein incorporated by reference in its entirety. The polymers may be conjugated using a ligand conjugate such as, but not limited to, the conjugates described in U.S. Pat. No. 8,273,363, herein incorporated by reference in its entirety.

In one embodiment, the formulations described herein may comprise a block copolymer is PEG-PLGA-PEG (see e.g., the thermosensitive hydrogel (PEG-PLGA-PEG) was used as a TGF-beta1 gene delivery vehicle in Lee et al. Thermosensitive Hydrogel as a Tgfβ1 Gene Delivery Vehicle Enhances Diabetic Wound Healing. Pharmaceutical Research, 2003 20(12): 1995-2000; as a controlled gene delivery system in Li et al. Controlled Gene Delivery System Based on Thermosensitive Biodegradable Hydrogel. Pharmaceutical Research 2003 20(6):884-888; and Chang et al., Non-ionic amphiphilic biodegradable PEG-PLGA-PEG copolymer enhances gene delivery efficiency in rat skeletal muscle. J Controlled Release. 2007 118:245-253; each of which is herein incorporated by reference in its entirety) may be used in the present invention. The present invention may be formulated with PEG-PLGA-PEG for administration such as, but not limited to, intramuscular and subcutaneous administration.

In another embodiment, the formulations described herein may comprise PEG-PLGA-PEG block copolymer is used in the present invention to develop a biodegradable sustained release system. In one aspect, the renal polynucleotides of the present invention are mixed with the block copolymer prior to administration. In another aspect, the renal polynucleotides acids of the present invention are co-administered with the block copolymer.

The amount of buffer and/or acid used in combination with the PEG lipids of the may also be varied. In one non-limiting example, the ratio of buffer and/or acid with PEG lipids is 1:1. As a non-limiting example, the amount of buffer and/or acid used with the PEG lipids may be increased to alter the ratio of buffer/acid to PEG in order to optimize the formulation.

In one embodiment, the formulations described herein may include at least one, at least two, at least three, at least four, at least five, at least six or more than six PEG lipids. The PEG lipids may be selected from, but are not limited to, pentaerythritol PEG ester tetra-succinimidyl and pentaerythritol PEG ether tetra-thiol, PEG-c-DOMG, PEG-DMG (1,2-Dimyristoyl-sn-glycerol, methoxypolyethylene Glycol), PEG-DSG (1,2-Distearoyl-sn-glycerol, methoxypolyethylene Glycol), PEG-DPG (1,2-Dipalmitoyl-sn-glycerol, methoxypolyethylene glycol), PEG-DSA (PEG coupled to 1,2-distearyloxypropyl-3-amine), PEG-DMA (PEG coupled to 1,2-dimyristyloxypropyl-3-amine, PEG-c-DNA, PEG-c-DMA, PEG-S-DSG, PEG-c-DMA, PEG-DPG, PEG-DMG 2000 and those described herein and/or known in the art. The concentration and/or ratio of the PEG lipids in the formulation may be varied in order to optimize the formulation for delivery and/or administration.

In one embodiment, the renal polynucleotide formulations of the present invention may include at least one polymeric compound of PEG grafted with PLL as described in U.S. Pat. No. 6,177,274; herein incorporated by reference in its entirety.

In one embodiment, the renal polynucleotide formulations of the present invention may include at least one PLGA-PEG block copolymer (see US Pub. No. US20120004293 and U.S. Pat. No. 8,236,330, herein incorporated by reference in their entireties) or PLGA-PEG-PLGA block copolymers (See U.S. Pat. No. 6,004,573, herein incorporated by reference in its entirety). As a non-limiting example, the renal polynucleotides of the invention may be formulated with a diblock copolymer of PEG and PLA or PEG and PLGA (see U.S. Pat. No. 8,246,968, herein incorporated by reference in its entirety).

In one embodiment, the lipid nanoparticles described herein may comprise 50% DLin-KC2-DMA, 10% DSPC, 39.5% cholesterol and 0.5% PEG-DSG. In one embodiment, the lipid nanoparticles described herein may comprise 50% DLin-KC2-DMA, 10% DSPC, 39.5% cholesterol and 0.5% PEG-DSPE.

In one embodiment, the lipid nanoparticles described herein may comprise 50% DLin-KC2-DMA, 10% DSPC, 38.5% cholesterol and 1.5% PEG-DSG. In one embodiment, the lipid nanoparticles described herein may comprise 50% DLin-KC2-DMA, 10% DSPC, 38.5% cholesterol and 1.5% PEG-DSPE.

In one embodiment, the lipid nanoparticles described herein may comprise 50% DLin-KC2-DMA, 10% DSPC, 35% cholesterol and 5% PEG-DSG. In one embodiment, the lipid nanoparticles described herein may comprise 50% DLin-KC2-DMA, 10% DSPC, 35% cholesterol and 5% PEG-DSPE.

In one embodiment, the lipid nanoparticles described herein may comprise 50% DLin-KC2-DMA, 10% DSPC, 39.5% cholesterol and 0.5% PEG-DSG. In one embodiment, the lipid nanoparticles described herein may comprise 50% DLin-KC2-DMA, 10% DSPC, 30% cholesterol and 10% PEG-DSPE.

In one embodiment, the LNP formulation may be formulated in a nanoparticle such as a nucleic acid-lipid particle described in U.S. Pat. No. 8,492,359, the contents of which are herein incorporated by reference in its entirety. As a non-limiting example, the lipid particle may comprise one or more active agents or therapeutic agents; one or more cationic lipids comprising from about 50 mol % to about 85 mol % of the total lipid present in the particle; one or more non-cationic lipids of neutral overall charge comprising from about 13 mol % to about 49.5 mol % of the total lipid present in the particle; and one or more conjugated lipids that inhibit aggregation of particles comprising from about 0.5 mol % to about 2 mol % of the total lipid present in the particle. The nucleic acid in the nanoparticle may be the renal polynucleotides described herein and/or are known in the art.

The nanoparticle formulations may comprise a phosphate conjugate. The phosphate conjugate may increase in vivo circulation times and/or increase the targeted delivery of the nanoparticle. Phosphate conjugates for use with the present invention may be made by the methods described in International Application No. WO2013033438 or US Patent Publication No. US20130196948, the contents of each of which are herein incorporated by reference in its entirety. As a non-limiting example, the phosphate conjugates may include a compound of any one of the formulas described in International Application No. WO2013033438, herein incorporated by reference in its entirety.

The nanoparticle formulation may comprise a polymer conjugate. The polymer conjugate may be a water soluble conjugate. The polymer conjugate may have a structure as described in U.S. Patent Application No. 20130059360, the contents of which are herein incorporated by reference in its entirety. In one aspect, polymer conjugates with the renal polynucleotides of the present invention may be made using the methods and/or segmented polymeric reagents described in U.S. Patent Application No. 20130072709, herein incorporated by reference in its entirety. In another aspect, the polymer conjugate may have pendant side groups comprising ring moieties such as, but not limited to, the polymer conjugates described in US Patent Publication No. US20130196948, the contents of which is herein incorporated by reference in its entirety.

In one embodiment, the renal polynucleotides of the invention may be part of a nucleic acid conjugate comprising a hydrophobic polymer covalently bound to the renal polynucleotide through a first linker wherein said conjugate forms nanoparticulate micelles having a hydrophobic core and a hydrophilic shell, for example, to render nucleic acids resistant to nuclease digestion, as described in International Patent Publication No. WO2014047649, the contents of which is herein incorporated by reference in its entirety.

In one embodiment, a non-linear multi-block copolymer-drug conjugate may be used to deliver active agents such as the polymer-drug conjugates and the formulas described in International Publication No. WO2013138346, incorporated by reference in its entirety. As a non-limiting example, a non-linear multi-block copolymer may be conjugated to a nucleic acid such as the renal polynucleotides described herein. As another non-limiting example, a non-linear multi-block copolymer may be conjugated to a nucleic acid such as the renal polynucleotides described herein to treat intraocular neovascular diseases.

In another embodiment, pharmaceutical compositions comprising the renal polynucleotides of the present invention and a conjugate which may have a degradable linkage. Non-limiting examples of conjugates include an aromatic moiety comprising an ionizable hydrogen atom, a spacer moiety, and a water-soluble polymer. As a non-limiting example, pharmaceutical compositions comprising a conjugate with a degradable linkage and methods for delivering such pharmaceutical compositions are described in US Patent Publication No. US20130184443, the contents of which are herein incorporated by reference in its entirety.

The lipid nanoparticle may include surface altering agents such as, but not limited to, renal polynucleotides, anionic proteins (e.g., bovine serum albumin), surfactants (e.g., cationic surfactants such as for example dimethyldioctadecyl-ammonium bromide), sugars or sugar derivatives (e.g., cyclodextrin), nucleic acids, polymers (e.g., heparin, polyethylene glycol and poloxamer), mucolytic agents (e.g., N-acetylcysteine, mugwort, bromelain, papain, clerodendrum, acetylcysteine, bromhexine, carbocisteine, eprazinone, mesna, ambroxol, sobrerol, domiodol, letosteine, stepronin, tiopronin, gelsolin, thymosin β4 dornase alfa, neltenexine, erdosteine) and various DNases including rhDNase. The surface altering agent may be embedded or enmeshed in the particle's surface or disposed (e.g., by coating, adsorption, covalent linkage, or other process) on the surface of the lipid nanoparticle. (see e.g., US Publication 20100215580 and US Publication 20080166414 and US20130164343; each of which is herein incorporated by reference in their entirety).

In one embodiment, the therapeutic nanoparticles may be formulated to be target specific.

In one embodiment such formulations may also be constructed or compositions altered such that they passively or actively are directed to different cell types in vivo, including but not limited to hepatocytes, immune cells, tumor cells, endothelial cells, antigen presenting cells, and leukocytes (Akinc et al. Mol Ther. 2010 18:1357-1364; Song et al., Nat Biotechnol. 2005 23:709-717; Judge et al., J Clin Invest. 2009 119:661-673; Kaufmann et al., Microvasc Res 2010 80:286-293; Santel et al., Gene Ther 2006 13:1222-1234; Santel et al., Gene Ther 2006 13:1360-1370; Gutbier et al., Pulm Pharmacol. Ther. 2010 23:334-344; Basha et al., Mol. Ther. 2011 19:2186-2200; Fenske and Cullis, Expert Opin Drug Deliv. 2008 5:25-44; Peer et al., Science. 2008 319:627-630; Peer and Lieberman, Gene Ther. 2011 18:1127-1133; all of which are incorporated herein by reference in its entirety). One example of passive targeting of formulations to liver cells includes the DLin-DMA, DLin-KC2-DMA and DLin-MC3-DMA-based lipid nanoparticle formulations which have been shown to bind to apolipoprotein E and promote binding and uptake of these formulations into hepatocytes in vivo (Akinc et al. Mol Ther. 2010 18:1357-1364; herein incorporated by reference in its entirety). Formulations can also be selectively targeted through expression of different ligands on their surface as exemplified by, but not limited by, folate, transferrin, N-acetylgalactosamine (GaINAc), and antibody targeted approaches (Kolhatkar et al., Curr Drug Discov Technol. 2011 8:197-206; Musacchio and Torchilin, Front Biosci. 2011 16:1388-1412; Yu et al., Mol Membr Biol. 2010 27:286-298; Patil et al., Crit Rev Ther Drug Carrier Syst. 2008 25:1-61; Benoit et al., Biomacromolecules. 2011 12:2708-2714; Zhao et al., Expert Opin Drug Deliv. 2008 5:309-319; Akinc et al., Mol Ther. 2010 18:1357-1364; Srinivasan et al., Methods Mol Biol. 2012 820:105-116; Ben-Arie et al., Methods Mol Biol. 2012 757:497-507; Peer 2010 J Control Release. 20:63-68; Peer et al., Proc Natl Acad Sci USA. 2007 104:4095-4100; Kim et al., Methods Mol Biol. 2011 721:339-353; Subramanya et al., Mol Ther. 2010 18:2028-2037; Song et al., Nat Biotechnol. 2005 23:709-717; Peer et al., Science. 2008 319:627-630; Peer and Lieberman, Gene Ther. 2011 18:1127-1133; all of which are incorporated herein by reference in its entirety).

In another embodiment, the therapeutic nanoparticle may include a conjugation of at least one targeting ligand. The targeting ligand may be any ligand known in the art such as, but not limited to, a monoclonal antibody. (Kirpotin et al, Cancer Res. 2006 66:6732-6740; herein incorporated by reference in its entirety).

In one embodiment, the nanoparticles may contain reactive groups to release the renal polynucleotides described herein (see International Pub. No. WO20120952552 and US Pub No. US20120171229, each of which is herein incorporated by reference in their entirety).

In one embodiment, the renal polynucleotides of the present invention can be formulated for controlled release and/or targeted delivery. As used herein, “controlled release” refers to a pharmaceutical composition or compound release profile that conforms to a particular pattern of release to effect a therapeutic outcome. In one embodiment, the renal polynucleotides may be encapsulated into a delivery agent described herein and/or known in the art for controlled release and/or targeted delivery. As used herein, the term “encapsulate” means to enclose, surround or encase. As it relates to the formulation of the compounds of the invention, encapsulation may be substantial, complete or partial. The term “substantially encapsulated” means that at least greater than 50, 60, 70, 80, 85, 90, 95, 96, 97, 98, 99, 99.9, 99.9 or greater than 99.999% of the pharmaceutical composition or compound of the invention may be enclosed, surrounded or encased within the delivery agent. “Partially encapsulation” means that less than 10, 10, 20, 30, 40 50 or less of the pharmaceutical composition or compound of the invention may be enclosed, surrounded or encased within the delivery agent. Advantageously, encapsulation may be determined by measuring the escape or the activity of the pharmaceutical composition or compound of the invention using fluorescence and/or electron micrograph. For example, at least 1, 5, 10, 20, 30, 40, 50, 60, 70, 80, 85, 90, 95, 96, 97, 98, 99, 99.9, 99.99 or greater than 99.99% of the pharmaceutical composition or compound of the invention are encapsulated in the delivery agent.

In one embodiment, the encapusulation efficiency of the renal polynucleotide in a lipid nanoparticle may be 50-100%, 50-99% 50-90%, 50-80%, 50-70%, 50-60%, 60-100%, 60-99%, 60-90%, 60-80%, 60-70%, 70-100%, 70-99%, 70-90%, 70-80%, 80-100%, 80-99%, 80-90%, 90-100% or 90-99%.

In one embodiment, the encapusulation efficiency of the renal polynucleotide in a lipid nanoparticle may be at least 50%, 55%, 60%, 65%, 70%, 75%, 80%, 85%, 90%, 95%, 99% or 100%. As a non-limiting example, the encapsulation efficiency may be at least 50%.

In one embodiment, the encapusulation efficiency of the renal polynucleotide in a lipid nanoparticle may be 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%.

In one embodiment, the controlled release formulation may include, but is not limited to, tri-block co-polymers. As a non-limiting example, the formulation may include two different types of tri-block co-polymers (International Pub. No. WO2012131104 and WO2012131106; each of which is herein incorporated by reference in its entirety).

In one embodiment, the renal polynucleotide formulation for controlled release and/or targeted delivery may also include at least one controlled release coating. Controlled release coatings include, but are not limited to, OPADRY®, polyvinylpyrrolidone/vinyl acetate copolymer, polyvinylpyrrolidone, hydroxypropyl methylcellulose, hydroxypropyl cellulose, hydroxyethyl cellulose, EUDRAGIT RL®, EUDRAGIT RS® and cellulose derivatives such as ethylcellulose aqueous dispersions (AQUACOAT® and SURELEASE®).

In one embodiment, the controlled release and/or targeted delivery formulation may comprise at least one degradable polyester which may contain polycationic side chains. Degradable polyesters include, but are not limited to, poly(serine ester), poly(L-lactide-co-L-lysine), poly(4-hydroxy-L-proline ester), and combinations thereof. In another embodiment, the degradable polyesters may include a PEG conjugation to form a PEGylated polymer.

In one embodiment, the controlled release and/or targeted delivery formulation comprising at least one renal polynucleotide may comprise at least one PEG and/or PEG related polymer derivatives as described in U.S. Pat. No. 8,404,222, herein incorporated by reference in its entirety.

In another embodiment, the controlled release delivery formulation comprising at least one renal polynucleotide may be the controlled release polymer system described in US20130130348 or US20140079776, the contents of each of which are herein incorporated by reference in its entirety.

In one embodiment, the nanoparticle is formulated to release the renal polynucleotides at a specified pH and/or after a desired time interval. As a non-limiting example, the nanoparticle may be formulated to release the renal polynucleotides after 24 hours and/or at a pH of 4.5 (see International Pub. Nos. WO2010138193 and WO2010138194 and US Pub Nos. US20110020388 and US20110027217, each of which is herein incorporated by reference in their entireties).

In one embodiment, the nanoparticles may be formulated for controlled and/or sustained release of the renal polynucleotides such as the methods known in the art, described herein and/or as described in International Pub No. WO2010138192 and US Pub Nos. US20100303850, US20130243848 and US20130243827, each of which is herein incorporated by reference in their entirety.

In one embodiment, the renal polynucleotides of the present invention may be encapsulated in a nanoparticle (e.g., a therapeutic nanoparticle from BIND Therapeutics). Therapeutic nanoparticles may be formulated by methods described herein and known in the art such as, but not limited to, International Pub Nos. WO2010005740, WO2010030763, WO2010005721, WO2010005723, WO2012054923 and WO2014043625, US Pub. Nos. US20110262491, US20100104645, US20100087337, US20100068285, US20110274759, US20100068286, US20120288541, US20130123351, US20130230567, US20130236500, US20130302433, US20130302432, U520140142165, US20130280339 and US20130251757 and U.S. Pat. Nos. 8,206,747, 8,293,276, 8,318,208, 8,318,211, 8,623,417, 8,617,608, 8,613,954, 8,613,951, 8,609,142, 8,603,534, 8,652,528, 8,563,041, 8,663,700, and 8,563,041; the contents of each of which are herein incorporated by reference in their entirety. In another embodiment, therapeutic polymer nanoparticles may be identified by the methods described in US Pub No. US20120140790, herein incorporated by reference in its entirety. As a non-limiting example, the therapeutic nanoparticle may comprise about 4 to about 25 weight percent of a therapeutic agent (e.g., the renal polynucleotides described herein) and about 10 to about 99 weight percent of a diblock poly(lactic) acid-poly(ethylene)glycol copolymer comprising poly(lactic) acid as described in US Patent Publication No. US20130236500 (Bind), the contents of which are herein incorporated by reference in its entirety.

In one embodiment, the renal polynucleotides of the invention may be delivered in therapeutic nanoparticles made by a process including combining a therapeutic agent with an organic acid, which may improve drug loading and/or drug release properties, as described in International Patent Publication No. WO2014043618 (BIND Therapeutics, Inc, Cambridge, Mass., US), the contents of which is herein incorporated by reference in its entirety.

In one embodiment, the nanoparticle may be formulated for sustained release. As used herein, “sustained release” refers to a pharmaceutical composition or compound that conforms to a release rate over a specific period of time. The period of time may include, but is not limited to, hours, days, weeks, months and years. As a non-limiting example, the sustained release nanoparticle may comprise a polymer and a therapeutic agent such as, but not limited to, the renal polynucleotides of the present invention (see International Pub No. WO2010075072 and US Pub No. US20100216804, US20110217377, US20120201859, US20130243848 and US20130243827, each of which is herein incorporated by reference in their entirety). In another non-limiting example, the sustained release formulation may comprise agents which permit persistent bioavailability such as, but not limited to, crystals, macromolecular gels and/or particulate suspensions (see US Patent Publication No US20130150295, the contents of which is herein incorporated by reference in its entirety). In another non-limiting example, the renal polynucleotides may be formulated in a sustained release formulation as described in International Patent Publication No. WO2014081849, the contents of which is herein incorporated by reference in its entirety. In yet another non-limiting example, the renal polynucleotides may be delivered in a sustained release formulation according to the methods of International Patent Publication No. WO2014081849, the contents of which is herein incorporated by reference in its entirety.

In one embodiment, the renal polynucleotides may be encapsulated in, linked to and/or associated with therapeutically targeted nanoparticles. Non-limiting examples of therapeutically targeted nanoparticles include synthetic nanocarriers such as, but not limited to, those described in International Pub. Nos. WO2010005740, WO2010030763, WO201213501, WO2012149252, WO2012149255, WO2012149259, WO2012149265, WO2012149268, WO2012149282, WO2012149301, WO2012149393, WO2012149405, WO2012149411, WO2012149454 and WO2013019669, and US Pub. Nos. US20110262491, US20100104645, US20100087337, US20120244222 and US20130236533, and U.S. Pat. No. 8,652,487, the content of each of which is herein incorporated by reference in their entirety. As a non-limiting example, the synthetic nanocarriers may be formulated by the methods described in International Pub Nos. WO2010005740, WO2010030763 and WO201213501 and US Pub. Nos. US20110262491, US20100104645, US20100087337 and US2012024422, each of which is herein incorporated by reference in their entirety.

In one embodiment the nanoparticles of the present invention may be developed by the methods described in US Patent Publication No. US20130172406 (Bind), US20130251817 (Bind), US2013251816 (Bind) and US20130251766 (Bind), the contents of each of which are herein incorporated by reference in its entirety. In another non-limiting example, the renal polynucleotides of the invention may be formulated in a nanoparticle with targeting agent functionalized diblock copolymers, as described in or made by the methods described in U.S. Pat. No. 8,734,846 (Bind), the contents of which is herein incorporated by reference in its entirety.

In one embodiment, the renal polynucleotides may be formulated in and/or delivered in neutral nanoparticles. As a non-limiting example, the neutral nanoparticles may be those described in or made by the methods described in International Patent Publication No. WO2013149141, the contents of which are herein incorporated by reference in its entirety.

In one embodiment, the nanoparticles may be neutralized by the methods described in International Patent Publication No. WO2013149141, the contents of which are herein incorporated by reference in its entirety.

In one embodiment, the renal polynucleotide formulations of the present invention may include at least one degradable polyester which may contain polycationic side chains. Degradable polyesters include, but are not limited to, poly(serine ester), poly(L-lactide-co-L-lysine), poly(4-hydroxy-L-proline ester), and combinations thereof. In another embodiment, the degradable polyesters may include a PEG conjugation to form a PEGylated polymer.

In one embodiment, the renal polynucleotide formulations of the present invention may include at least one crosslinkable polyester. Crosslinkable polyesters include those known in the art and described in US Pub. No. 20120269761, the contents of which is herein incorporated by reference in its entirety.

### Polymers, Biodegradable Nanoparticles, and Core-Shell Nanoparticles

In one embodiment, pharmaceutical compositions of renal polynucleotides include polymers, biodegradable nanoparticles and/or core-shell nanoparticles. Non-limiting examples of polymers, biodegradable nanoparticles and/or core-shell nanoparticles and formulations thereof are described in International Patent Publication No. WO2015038892, the contents of which are herein incorporated by reference in its entirety.

### Peptides and Proteins

The renal polynucleotides of the invention can be formulated with renal peptides and/or proteins in order to increase transfection of cells by the renal polynucleotide. In one embodiment, renal peptides such as, but not limited to, cell penetrating renal peptides and proteins and renal peptides that enable intracellular delivery may be used to deliver pharmaceutical formulations. Non-limiting examples of renal peptides, proteins and formulations thereof are described in International Patent Publication No. WO2015038892, the contents of which are herein incorporated by reference in its entirety.

### Hyaluronidase

The intramuscular or subcutaneous localized injection of renal polynucleotides of the invention can include hyaluronidase, which catalyzes the hydrolysis of hyaluronan. By catalyzing the hydrolysis of hyaluronan, a constituent of the interstitial barrier, hyaluronidase lowers the viscosity of hyaluronan, thereby increasing tissue permeability (Frost, Expert Opin. Drug Deliv. (2007) 4:427-440; herein incorporated by reference in its entirety). It is useful to speed their dispersion and systemic distribution of encoded proteins produced by transfected cells. Alternatively, the hyaluronidase can be used to increase the number of cells exposed to a renal polynucleotide of the invention administered intramuscularly or subcutaneously.

### Suspension Formulations

In some embodiments, suspension formulations are provided comprising renal polynucleotides, water immiscible oil depots, surfactants and/or co-surfactants and/or co-solvents. Combinations of oils and surfactants may enable suspension formulation with renal polynucleotides. Delivery of renal polynucleotides in a water immiscible depot may be used to improve bioavailability through sustained release of mRNA from the depot to the surrounding physiologic environment and prevent renal polynucleotides degradation by nucleases.

Suspension formulations are described in co-pending International Patent Publication No. WO2015038892, the contents of which is incorporated by reference in its entirety, such as, but not limited to, in paragraphs [000775]-[000781].

Introduction into Cells

A variety of methods are known in the art and suitable for introduction of nucleic acid into a cell, including viral and non-viral mediated techniques. Examples of typical non-viral mediated techniques include, but are not limited to, electroporation, calcium phosphate mediated transfer, nucleofection, sonoporation, heat shock, magnetofection, liposome mediated transfer, microinjection, microprojectile mediated transfer (nanoparticles), cationic polymer mediated transfer (DEAE-dextran, polyethylenimine, polyethylene glycol (PEG) and the like) or cell fusion.

The technique of sonoporation, or cellular sonication, is the use of sound (e.g., ultrasonic frequencies) for modifying the permeability of the cell plasma membrane. Sonoporation methods are known to those in the art and are used to deliver nucleic acids in vivo (Yoon and Park, Expert Opin Drug Deliv. 2010 7:321-330; Postema and Gilja, Curr Pharm Biotechnol. 2007 8:355-361; Newman and Bettinger, Gene Ther. 2007 14:465-475; all herein incorporated by reference in their entirety). Sonoporation methods are known in the art and are also taught for example as it relates to bacteria in US Patent Publication 20100196983 and as it relates to other cell types in, for example, US Patent Publication 20100009424, each of which are incorporated herein by reference in their entirety. Sonorporation may be combined with microbubbles (air-filled vesicles stabilized by surface active molecules such as albumin, polymers or phospholipids) to increase transdermal penetration of drugs. While not wishing to be bound by theory, upon absorption of the ultrasound waves, the microbubbles cavitate, oscillate, break up and release localized shock waves that can disrupt the nearby cell membranes and promote penetration of drugs. The size of the microspheres may be optimized to ensure efficient transfection of the drug. As a non-limiting example, the microbubbles may be about 1 to about 6 μm in diameter, e.g., about 1 μm, about 2 μm, about 3 μm, about 4 μm, about 5 μm or about 6 μm in diameter.

Electroporation techniques are also well known in the art and are used to deliver nucleic acids in vivo and clinically (Andre et al., Curr Gene Ther. 2010 10:267-280; Chiarella et al., Curr Gene Ther. 2010 10:281-286; Hojman, Curr Gene Ther. 2010 10:128-138; all herein incorporated by reference in their entirety). Electroporation parameters, when optimized, may produce a transfection efficiency which may be equal to the efficiency achieved by viral vectors. Electroporation devices are sold by many companies worldwide including, but not limited to BTX® Instruments (Holliston, Mass.) (e.g., the AgilePulse In Vivo System) and Inovio (Blue Bell, Pa.) (e.g., Inovio SP-5P intramuscular delivery device or the CELLECTRA® 3000 intradermal delivery device). Electroporation may be used after, before and/or during administration of the renal polynucleotides described herein. As a non-limiting example, electroporation may be used after local injection. As another non-limiting example, electroporation may be used after systemic injection. In one embodiment, renal polynucleotides may be delivered by electroporation as described in Example 9.

In one embodiment, the renal polynucleotides described herein may be administered using electroporation where the device is an integrated device where the injection and electrical pulse are coordinated. The integrated device ensures that the electrode position is consistent and the electrical field is consistent around the needle for each administration. As a non-limiting example, the renal polynucleotides described herein may be administered using TRIGRID™ technology such as the TRIGRID™ integrated device. The needle of the integrated device may be co-localized within the perimeter of the four electrodes.

In one embodiment, electroporation may be used to improve the generation of T and B cell responses from administration of a therapeutic agent (e.g., renal polynucleotides (see e.g., Cu et al. Enhanced Delivery and Potency of Self-Amplifying mRNA Vaccines by Electroporation in Situ. Vaccines 2013, 1, 367-383; the contents of which are herein incorporated by reference in its entirety)).

### Conjugates

The renal polynucleotides of the invention include conjugates, such as a renal polynucleotide covalently linked to a carrier or targeting group, or including two encoding regions that together produce a fusion protein (e.g., bearing a targeting group and therapeutic protein or renal peptide).

The conjugates of the invention include a naturally occurring substance, such as a protein (e.g., human serum albumin (HSA), low-density lipoprotein (LDL), high-density lipoprotein (HDL), or globulin); an carbohydrate (e.g., a dextran, pullulan, chitin, chitosan, inulin, cyclodextrin or hyaluronic acid); or a lipid. The ligand may also be a recombinant or synthetic molecule, such as a synthetic polymer, e.g., a synthetic polyamino acid, an oligonucleotide (e.g. an aptamer). Examples of polyamino acids include polyamino acid is a polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical renal peptide.

Representative U.S. patents that teach the preparation of renal polynucleotide conjugates, particularly to RNA, include, but are not limited to, U.S. Pat. Nos. 4,828,979; 4,948,882; 5,218,105; 5,525,465; 5,541,313; 5,545,730; 5,552,538; 5,578,717, 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941; 6,294,664; 6,320,017; 6,576,752; 6,783,931; 6,900,297; 7,037,646; each of which is herein incorporated by reference in their entireties.

In one embodiment, the conjugate of the present invention may function as a carrier for the renal polynucleotides of the present invention. The conjugate may comprise a cationic polymer such as, but not limited to, polyamine, polylysine, polyalkylenimine, and polyethylenimine which may be grafted to with poly(ethylene glycol). As a non-limiting example, the conjugate may be similar to the polymeric conjugate and the method of synthesizing the polymeric conjugate described in U.S. Pat. No. 6,586,524 herein incorporated by reference in its entirety.

A non-limiting example of a method for conjugation to a substrate is described in US Patent Publication No. US20130211249, the contents of which are herein incorporated by reference in its entirety. The method may be used to make a conjugated polymeric particle comprising a renal polynucleotide.

The conjugates can also include targeting groups, e.g., a cell or tissue targeting agent, e.g., a lectin, glycoprotein, lipid or protein, e.g., an antibody, that binds to a specified cell type such as a kidney cell. A targeting group can be a thyrotropin, melanotropin, lectin, glycoprotein, surfactant protein A, Mucin carbohydrate, multivalent lactose, multivalent galactose, N-acetyl-galactosamine, N-acetyl-D-glucosasamine, N-acetyl-glucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, biotin, an RGD renal peptide, an RGD renal peptide mimetic or an aptamer.

Targeting groups can be proteins, e.g., glycoproteins, or renal peptides, e.g., molecules having a specific affinity for a co-ligand, or antibodies e.g., an antibody, that binds to a specified cell type such as a cancer cell, endothelial cell, or bone cell. Targeting groups may also include hormones and hormone receptors. They can also include non-peptidic species, such as lipids, lectins, carbohydrates, vitamins, cofactors, multivalent lactose, multivalent galactose, N-acetyl-galactosamine, N-acetyl-D-glucosasamine, N-acetyl-glucosamine multivalent mannose, multivalent fucose, or aptamers. The ligand can be, for example, a lipopolysaccharide, or an activator of p38 MAP kinase.

The targeting group can be any ligand that is capable of targeting a specific receptor. Examples include, without limitation, folate, GaINAc, galactose, mannose, mannose-6P, aptamers, integrin receptor ligands, chemokine receptor ligands, transferrin, biotin, serotonin receptor ligands, PSMA, endothelin, GOPII, somatostatin, LDL, and HDL ligands. In particular embodiments, the targeting group is an aptamer. The aptamer can be unmodified or have any combination of modifications disclosed herein.

In still other embodiments, the renal polynucleotide is covalently conjugated to a cell penetrating renal polypeptide. The cell-penetrating renal peptide may also include a signal sequence. The conjugates of the invention can be designed to have increased stability; increased cell transfection; and/or altered the biodistribution (e.g., targeted to specific tissues or cell types).

In one embodiment, the renal polynucleotides may be conjugated to an agent to enhance delivery. As a non-limiting example, the agent may be a monomer or polymer such as a targeting monomer or a polymer having targeting blocks as described in International Publication No. WO2011062965, herein incorporated by reference in its entirety. In another non-limiting example, the agent may be a transport agent covalently coupled to the renal polynucleotides of the present invention (See e.g., U.S. Pat. Nos. 6,835,393 and 7,374,778, each of which is herein incorporated by reference in its entirety). In yet another non-limiting example, the agent may be a membrane barrier transport enhancing agent such as those described in U.S. Pat. Nos. 7,737,108 and 8,003,129, each of which is herein incorporated by reference in its entirety.

In one embodiment, the pharmaceutical compositions of the invention comprise polymeric reagents that provide a conjugate, allowing a degradable linkage between a polymer and another moiety, as described in or synthesized and conjugated to active agents and other moieties by the methods of US Patent Publication 20140107349, the contents of which is incorporated herein by reference in its entirety.

In another embodiment, renal polynucleotides may be conjugated to SMARTT POLYMER TECHNOLOGY® (PHASERX®, Inc. Seattle, Wash.).

In one embodiment, the conjugate may be an aptamer-mRNA conjugate which may be used for targeted expression. As a non-limiting example, the aptamer-mRNA conjugate may include any of the aptamers and/or conjugates described in US Patent Publication No. US20130022538, the contents of which is herein incorporated by reference in its entirety. The aptamer-mRNA conjugate may include an aptamer component that can bind to a membrane associated protein on a target cell.

In one embodiment, the conjugate may be a water-soluble polymer conjugate such as the conjugates described in U.S. Pat. No. 8,636,994, the contents of which are herein incorporated by reference in its entirety. As a non-limiting example, the water-soluble polymer conjugate may comprise at least one residue of an antimicrobial agent (see e.g., the conjugates described in U.S. Pat. No. 8,636,994, the contents of which are herein incorporated by reference in its entirety).

In one embodiment, the renal polynucleotides may be formulated in a particle comprising a conjugate for delivering nucleic acid agents such as the particles described in US Patent Publication No. US20140037573, the contents of which are herein incorporated by reference in its entirety. As a non-limiting example, the particle comprising a plurality of hydrophobic moieties, a plurality of hydrophilic-hydrophobic polymers and nucleic acid agents.

### Cations and Anions

Formulations of renal polynucleotides disclosed herein may include cations or anions. In one embodiment, the formulations include metal cations such as, but not limited to, Zn2+, Ca2+, Cu2+, Mg+ and combinations thereof. As a non-limiting example, formulations may include polymers and a renal polynucleotides complexed with a metal cation (See e.g., U.S. Pat. Nos. 6,265,389 and 6,555,525, each of which is herein incorporated by reference in its entirety).

In some embodiments, cationic nanoparticles comprising combinations of divalent and monovalent cations may be formulated with renal polynucleotides. Such nanoparticles may form spontaneously in solution over a given period (e.g. hours, days, etc.). Such nanoparticles do not form in the presence of divalent cations alone or in the presence of monovalent cations alone. The delivery of renal polynucleotides in cationic nanoparticles or in one or more depot comprising cationic nanoparticles may improve renal polynucleotide bioavailability by acting as a long-acting depot and/or reducing the rate of degradation by nucleases.

### Polymer Implant

In one embodiment, the renal polynucleotides may be formulated in or delivered using polymer implants. As a non-limiting example, the polymer implant is inserted into or onto damaged human tissue and the renal polynucleotides are released from the polymer implant. (See e.g., MariGen Omega3 from Kerecis for the treatment of damaged tissue).

In one embodiment, the renal polynucleotides may be formulated in or delivered using delivery devices comprising polymer implants.

### Excipients

Pharmaceutical formulations may additionally comprise a pharmaceutically acceptable excipient, which, as used herein, includes, but are not limited to, any and all solvents, dispersion media, diluents, or other liquid vehicles, dispersion or suspension aids, surface active agents, isotonic agents, thickening or emulsifying agents, preservatives, solid binders, lubricants, flavoring agents, stabilizers, antioxidants, osmolality adjusting agents, pH adjusting agents and the like, as suited to the particular dosage form desired. Various excipients for formulating pharmaceutical compositions and techniques for preparing the composition are known in the art (see Remington: The Science and Practice of Pharmacy, 21st Edition, A. R. Gennaro (Lippincott, Williams & Wilkins, Baltimore, Md., 2006; incorporated herein by reference in its entirety). The use of a conventional excipient medium may be contemplated within the scope of the present disclosure, except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this invention.

In some embodiments, a pharmaceutically acceptable excipient may be at least 95%, at least 96%, at least 97%, at least 98%, at least 99%, or 100% pure. In some embodiments, an excipient is approved for use for humans and for veterinary use. In some embodiments, an excipient may be approved by United States Food and Drug Administration. In some embodiments, an excipient may be of pharmaceutical grade. In some embodiments, an excipient may meet the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical compositions. The composition may also include excipients such as cocoa butter and suppository waxes, coloring agents, coating agents, sweetening, flavoring, and/or perfuming agents.

Exemplary diluents, granulating and/or dispersing agents, surface active agents and/or emulsifiers, binding agents, preservatives, buffers, lubricating agents, oils, additives, cocoa butter and suppository waxes, coloring agents, coating agents, sweetening, flavoring, and/or perfuming agents are described in co-pending International Patent Publication No. WO2015038892, the contents of which is incorporated by reference in its entirety, such as, but not limited to, in paragraphs [000828]-[000838].

Cryoprotectants for mRNA

In some embodiments, renal polynucleotide formulations may comprise cyroprotectants. As used herein, there term “cryoprotectant” refers to one or more agent that when combined with a given substance, helps to reduce or eliminate damage to that substance that occurs upon freezing. In some embodiments, cryoprotectants are combined with renal polynucleotides in order to stabilize them during freezing. Frozen storage of mRNA between −20° C. and −80° C. may be advantageous for long term (e.g. 36 months) stability of renal polynucleotide. In some embodiments, cryoprotectants are included in renal polynucleotide formulations to stabilize renal polynucleotide through freeze/thaw cycles and under frozen storage conditions. Cryoprotectants of the present invention may include, but are not limited to sucrose, trehalose, lactose, glycerol, dextrose, raffinose and/or mannitol. Trehalose is listed by the Food and Drug Administration as being generally regarded as safe (GRAS) and is commonly used in commercial pharmaceutical formulations.

### Bulking Agents

In some embodiments, renal polynucleotide formulations may comprise bulking agents. As used herein, the term “bulking agent” refers to one or more agents included in formulations to impart a desired consistency to the formulation and/or stabilization of formulation components. In some embodiments, bulking agents are included in lyophilized renal polynucleotide formulations to yield a “pharmaceutically elegant” cake, stabilizing the lyophilized renal polynucleotides during long term (e.g. 36 month) storage. Bulking agents of the present invention may include, but are not limited to sucrose, trehalose, mannitol, glycine, lactose and/or raffinose. In some embodiments, combinations of cryoprotectants and bulking agents (for example, sucrose/glycine or trehalose/mannitol) may be included to both stabilize renal polynucleotides during freezing and provide a bulking agent for lyophilization.

Non-limiting examples of formulations and methods for formulating the renal polynucleotides of the present invention are also provided in International Publication No WO2013090648 filed Dec. 14, 2012, the contents of which are incorporated herein by reference in their entirety.

### Inactive Ingredients

In some embodiments, renal polynucleotide formulations may comprise at least one excipient which is an inactive ingredient. As used herein, the term “inactive ingredient” refers to one or more inactive agents included in formulations. In some embodiments, all, none or some of the inactive ingredients which may be used in the formulations of the present invention may be approved by the US Food and Drug Administration (FDA). A non-exhaustive list of inactive ingredients and the routes of administration the inactive ingredients may be formulated in are described in Table 4 of International Patent Publication No. WO2014152211, the contents of which are herein incorporated by reference in its entirety.

### Delivery

The present disclosure encompasses the delivery of renal polynucleotides for any of therapeutic, pharmaceutical, diagnostic or imaging by any appropriate route taking into consideration likely advances in the sciences of drug delivery. Delivery may be naked or formulated.

### Naked Delivery

The renal polynucleotides of the present invention may be delivered to a cell naked. As used herein in, “naked” refers to delivering renal polynucleotides free from complexing agents, for example, lipid agents and polymer agents, etc. For example, the renal polynucleotides delivered to the cell may contain no modifications. The naked renal polynucleotides may be delivered to the cell using routes of administration known in the art and described herein.

### Formulated Delivery

The renal polynucleotides of the present invention may be formulated, using the methods described herein. The formulations may contain renal polynucleotides which may be modified and/or unmodified. The formulations may further include, but are not limited to, cell penetration agents, a pharmaceutically acceptable carrier, a delivery agent, a bioerodible or biocompatible polymer, a solvent, and a sustained-release delivery depot. The formulated renal polynucleotides may be delivered to the cell using routes of administration known in the art and described herein.

The compositions may also be formulated for direct delivery to an organ or tissue in any of several ways in the art including, but not limited to, direct soaking or bathing, via a catheter, by gels, powder, ointments, creams, gels, lotions, and/or drops, by using substrates such as fabric or biodegradable materials coated or impregnated with the compositions, and the like.

### Administration

The renal polynucleotides of the present invention may be administered by any route which results in a therapeutically effective outcome. These include, but are not limited to enteral (into the intestine), gastroenteral, epidural (into the dura matter), oral (by way of the mouth), transdermal, peridural, intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intravenous bolus, intravenous drip, intraarterial (into an artery; also called arterial administration), intramuscular (into a muscle), intracardiac (into the heart), intraosseous infusion (into the bone marrow), intrathecal (into the spinal canal), intraperitoneal, (infusion or injection into the peritoneum), intravesical infusion, intravitreal, (through the eye), intracavernous injection (into a pathologic cavity) intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), in ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracornal, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intramyocardial (within the myocardium), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration which is then covered by a dressing which occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), intramyocardial (entering the myocardium), soft tissue, subarachnoid, subconjunctival, submucosal, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis or spinal. In specific embodiments, compositions may be administered in a way which allows them cross the blood-brain barrier, vascular barrier, or other epithelial barrier. As a non-limiting example, formulations of the renal polynucleotides described herein may be delivered by intramyocardial injection. As another non-limiting example, formulations of the renal polynucleotides described herein may be delivered by intramyocardial injection into the ischemic region prior to, during or after coronary artery ligation.

In one embodiment, a formulation for a route of administration may include at least one inactive ingredient. Non-limiting examples of routes of administration and inactive ingredients which may be included in formulations for the specific route of administration is shown in Table 5 of International Patent Publication No. WO2014152211, the contents of which are herein incorporated by reference in its entirety.

In one embodiment, the renal polynucleotides may be delivered, localized and/or concentrated in a specific location using the delivery methods described in International Patent Publication No. WO2013063530, the contents of which are herein incorporated by reference in its entirety. As a non-limiting example, a subject may be administered an empty polymeric particle prior to, simultaneously with or after delivering the renal polynucleotides to the subject. The empty polymeric particle undergoes a change in volume once in contact with the subject and becomes lodged, embedded, immobilized or entrapped at a specific location in the subject.

Non-limiting routes of administration for the renal polynucleotides of the present invention are described below.

### Parenteral and Injectable Administration

Liquid dosage forms for parenteral administration include, but are not limited to, pharmaceutically acceptable emulsions, microemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents. In certain embodiments for parenteral administration, compositions are mixed with solubilizing agents such as CREMOPHOR®, alcohols, oils, modified oils, glycols, polysorbates, cyclodextrins, polymers, and/or combinations thereof.

A pharmaceutical composition for parenteral administration may comprise at least one inactive ingredient. Any or none of the inactive ingredients used may have been approved by the US Food and Drug Administration (FDA). A non-exhaustive list of inactive ingredients for use in pharmaceutical compositions for parenteral administration includes hydrochloric acid, mannitol, nitrogen, sodium acetate, sodium chloride and sodium hydroxide.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables. The sterile formulation may also comprise adjuvants such as local anesthetics, preservatives and buffering agents.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

Injectable formulations may be for direct injection into a region of a tissue, organ and/or subject. As a non-limiting example, a tissue, organ and/or subject may be directly injected a formulation by intramyocardial injection into the ischemic region. (See e.g., Zangi et al. Nature Biotechnology 2013; the contents of which are herein incorporated by reference in its entirety).

In order to prolong the effect of an active ingredient, it is often desirable to slow the absorption of the active ingredient from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are prepared by entrapping the drug in liposomes or microemulsions which are compatible with body tissues.

In one embodiment, injectable formulations may comprise an excipient in addition to the renal polynucleotides described herein. As a non-limiting example the excipient may be N-acetyl-D-glucosasamine.

In one embodiment, formulations comprising the renal polynucleotides described herein may be formulated for intramuscular delivery may comprise an excipient. As a non-limiting example the excipient may be N-acetyl-D-glucosasamine.

In one embodiment, formulations comprising the renal polynucleotides described herein may be delivered with a microneedle device with an autodisable feature for intradermal delivery, as described in International Patent Publication No. WO2014064543, the contents of which is incorporated herein by reference in its entirety.

In another embodiment, the formulations of the invention may be delivered to the blood vessel lumen and wall. In some embodiments the formulations may include for example, antirestenotic, antithrombotic, antiplatelet, antiproliferative, antineoplastic, immunosuppressive, angiogenic, antHinflammatory, or antiangiogenic agents and/or vasodilators for delivery to a blood vessel. In a non-limiting example, the formulations may be delivered with a drug delivery device having an exterior surface and an interior surface; a plurality of openings in the device body; and a first therapeutic agent and a second therapeutic agent disposed in the openings and arranged to deliver the first therapeutic agent primarily to the exterior surface and to deliver the second therapeutic agent primarily to the interior surface, as described in European Patent No. EP1635893, the contents of which is herein incorporated by reference in its entirety.

### Rectal and Vaginal Administration

Rectal and vaginal administration and corresponding dosage forms are described in co-pending International Patent Publication No. WO2015038892, the contents of which is incorporated by reference in its entirety, such as, but not limited to, in paragraphs [000856]-[000859].

### Oral Administration

Oral administration and corresponding dosage forms (e.g., liquid dosage forms) are described in co-pending International Patent Publication No. WO2015038892, the contents of which is incorporated by reference in its entirety, such as, but not limited to, in paragraphs [000860]-[000869].

### Topical, Transdermal or Transcutaneous Administration

As described herein, compositions containing the renal polynucleotides of the invention may be formulated for administration topically, transdermally and/or transcutaneously. Topical, transdermal and transcutaneous administration and corresponding dosage forms are described in co-pending International Patent Publication No. WO2015038892, the contents of which is incorporated by reference in its entirety, such as, but not limited to, in paragraphs [000870]-[000888].

### Depot Administration

As described herein, in some embodiments, the composition is formulated in depots for extended release. Generally, a specific organ or tissue (a “target tissue”) is targeted for administration.

In some aspects of the invention, the renal polynucleotides are spatially retained within or proximal to a target tissue. Provided are method of providing a composition to a target tissue of a mammalian subject by contacting the target tissue (which contains one or more target cells) with the composition under conditions such that the composition, in particular the nucleic acid component(s) of the composition, is substantially retained in the target tissue, meaning that at least 10, 20, 30, 40, 50, 60, 70, 80, 85, 90, 95, 96, 97, 98, 99, 99.9, 99.99 or greater than 99.99% of the composition is retained in the target tissue. Advantageously, retention is determined by measuring the amount of the nucleic acid present in the composition that enters one or more target cells. For example, at least 1, 5, 10, 20, 30, 40, 50, 60, 70, 80, 85, 90, 95, 96, 97, 98, 99, 99.9, 99.99 or greater than 99.99% of the nucleic acids administered to the subject are present intracellularly at a period of time following administration. For example, intramuscular injection to a mammalian subject is performed using an aqueous composition containing a ribonucleic acid and a transfection reagent, and retention of the composition is determined by measuring the amount of the ribonucleic acid present in the muscle cells.

Aspects of the invention are directed to methods of providing a composition to a target tissue of a mammalian subject, by contacting the target tissue (containing one or more target cells) with the composition under conditions such that the composition is substantially retained in the target tissue. The composition contains an effective amount of a renal polynucleotides such that the renal polypeptide of interest is produced in at least one target cell. The compositions generally contain a cell penetration agent, although “naked” nucleic acid (such as nucleic acids without a cell penetration agent or other agent) is also contemplated, and a pharmaceutically acceptable carrier.

In some circumstances, the amount of a protein produced by cells in a tissue is desirably increased. Preferably, this increase in protein production is spatially restricted to cells within the target tissue. Thus, provided are methods of increasing production of a protein of interest in a tissue of a mammalian subject. A composition is provided that contains renal polynucleotides characterized in that a unit quantity of composition has been determined to produce the renal polypeptide of interest in a substantial percentage of cells contained within a predetermined volume of the target tissue.

In some embodiments, the composition includes a plurality of different renal polynucleotides, where one or more than one of the renal polynucleotides encodes a renal polypeptide of interest. Optionally, the composition also contains a cell penetration agent to assist in the intracellular delivery of the composition. A determination is made of the dose of the composition required to produce the renal polypeptide of interest in a substantial percentage of cells contained within the predetermined volume of the target tissue (generally, without inducing significant production of the renal polypeptide of interest in tissue adjacent to the predetermined volume, or distally to the target tissue). Subsequent to this determination, the determined dose is introduced directly into the tissue of the mammalian subject.

In one embodiment, the invention provides for the renal polynucleotides to be delivered in more than one injection or by split dose injections.

In one embodiment, the invention may be retained near target tissue using a small disposable drug reservoir, patch pump or osmotic pump. Non-limiting examples of patch pumps include those manufactured and/or sold by BD® (Franklin Lakes, N.J.), Insulet Corporation (Bedford, Mass.), SteadyMed Therapeutics (San Francisco, Calif.), Medtronic (Minneapolis, Minn.) (e.g., MiniMed), UniLife (York, Pa.), Valeritas (Bridgewater, N.J.), and SpringLeaf Therapeutics (Boston, Mass.). A non-limiting example of an osmotic pump include those manufactured by DURECT® (Cupertino, Calif.) (e.g., DUROS® and ALZET®).

### Pulmonary Administration

A pharmaceutical composition may be prepared, packaged, and/or sold in a formulation suitable for pulmonary administration via the buccal cavity. Pulmonary administration and corresponding dosage forms are described in co-pending International Patent Publication No. WO2015038892, the contents of which is incorporated by reference in its entirety, such as, but not limited to, in paragraphs [000896]-[000901].

### Intranasal, Nasal and Buccal Administration

Formulations described herein as being useful for pulmonary delivery are useful for intranasal delivery of a pharmaceutical composition. Another formulation suitable for intranasal administration is a coarse powder comprising the active ingredient and having an average particle from about 0.2 μm to 500 μm. Such a formulation is administered in the manner in which snuff is taken, i.e. by rapid inhalation through the nasal passage from a container of the powder held close to the nose. Intranasal, nasal and buccal administration and corresponding dosage forms are described in co-pending International Patent Publication No. WO2015038892, the contents of which is incorporated by reference in its entirety, such as, but not limited to, in paragraphs [000902]-[000905].

### Ophthalmic and Auricular (Otic) Administration

A pharmaceutical composition may be prepared, packaged, and/or sold in a formulation suitable for delivery to and/or around the eye and/or delivery to the ear (e.g., auricular (otic) administration). Non-limiting examples of route of administration for delivery to and/or around the eye include retrobulbar, conjunctival, intracorneal, intraocular, intravitreal, ophthalmic and subconjuctiva. Ophthalmic and auricular administration and corresponding dosage forms are described in co-pending International Patent Publication No. WO2015038892, the contents of which is incorporated by reference in its entirety, such as, but not limited to, in paragraphs [000906]-[000912].

### Payload Administration: Detectable Agents and Therapeutic Agents

The renal polynucleotides described herein can be used in a number of different scenarios in which delivery of a substance (the “payload”) to a biological target is desired, for example delivery of detectable substances for detection of the target, or delivery of a therapeutic agent. Detection methods can include, but are not limited to, both imaging in vitro and in vivo imaging methods, e.g., immunohistochemistry, bioluminescence imaging (BLI), Magnetic Resonance Imaging (MRI), positron emission tomography (PET), electron microscopy, X-ray computed tomography, Raman imaging, optical coherence tomography, absorption imaging, thermal imaging, fluorescence reflectance imaging, fluorescence microscopy, fluorescence molecular tomographic imaging, nuclear magnetic resonance imaging, X-ray imaging, ultrasound imaging, photoacoustic imaging, lab assays, or in any situation where tagging/staining/imaging is required.

The renal polynucleotides can be designed to include both a linker and a payload in any useful orientation. For example, a linker having two ends is used to attach one end to the payload and the other end to the nucleobase, such as at the C-7 or C-8 positions of the deaza-adenosine or deaza-guanosine or to the N-3 or C-5 positions of cytosine or uracil. The renal polynucleotide of the invention can include more than one payload (e.g., a label and a transcription inhibitor), as well as a cleavable linker. In one embodiment, the modified nucleotide is a modified 7-deaza-adenosine triphosphate, where one end of a cleavable linker is attached to the C7 position of 7-deaza-adenine, the other end of the linker is attached to an inhibitor (e.g., to the C5 position of the nucleobase on a cytidine), and a label (e.g., Cy5) is attached to the center of the linker (see, e.g., compound 1 of A*pCp C5 Parg Capless in FIG. 5 and columns 9 and 10 of U.S. Pat. No. 7,994,304, incorporated herein by reference). Upon incorporation of the modified 7-deaza-adenosine triphosphate to an encoding region, the resulting renal polynucleotide having a cleavable linker attached to a label and an inhibitor (e.g., a polymerase inhibitor). Upon cleavage of the linker (e.g., with reductive conditions to reduce a linker having a cleavable disulfide moiety), the label and inhibitor are released. Additional linkers and payloads (e.g., therapeutic agents, detectable labels, and cell penetrating payloads) are described herein and in International Application PCT/US2013/30062 filed Mar. 9, 2013 (Attorney Docket Number M300), the contents of which are incorporated herein by reference in their entirety.

For example, the renal polynucleotides described herein can be used in reprogramming induced pluripotent stem cells (iPS cells), which can directly track cells that are transfected compared to total cells in the cluster. In another example, a drug that may be attached to the renal polynucleotides via a linker and may be fluorescently labeled can be used to track the drug in vivo, e.g. intracellularly. Other examples include, but are not limited to, the use of a renal polynucleotides in reversible drug delivery into cells.

The renal polynucleotides described herein can be used in intracellular targeting of a payload, e.g., detectable or therapeutic agent, to specific organelle. Exemplary intracellular targets can include, but are not limited to, the nuclear localization for advanced mRNA processing, or a nuclear localization sequence (NLS) linked to the mRNA containing an inhibitor.

In addition, the renal polynucleotides described herein can be used to deliver therapeutic agents to cells or tissues, e.g., in living animals. For example, the renal polynucleotides described herein can be used to deliver highly polar chemotherapeutics agents to kill cancer cells. The renal polynucleotides attached to the therapeutic agent through a linker can facilitate member permeation allowing the therapeutic agent to travel into a cell to reach an intracellular target. As a non-limiting example, a renal peptide or renal peptide composition may be used to facilitate delivery through the stratum corneum and/or the cellular membrane of viable cells such as the skin permeating and cell entering (SPACE) renal peptides described in WO2012064429, the contents of which are herein incorporated by reference in its entirety. As another non-limiting example, nanoparticles designed to have enhanced entry into cancerous cells may be used to deliver the renal polynucleotides described herein (see e.g., the nanoparticles with a first shell comprising a first shell substance, a therapeutic agent and an endocytosis-enhancing agent (different from the therapeutic agent) described in International Patent Publication No. WO2013173693, the contents of which are herein incorporated by reference in its entirety).

In one example, the linker is attached at the 2′-position of the ribose ring and/or at the 3′ and/or 5′ position of the renal polynucleotides (See e.g., International Pub. No. WO2012030683, herein incorporated by reference in its entirety). The linker may be any linker disclosed herein, known in the art and/or disclosed in International Pub. No. WO2012030683, herein incorporated by reference in its entirety.

In another example, the renal polynucleotides can be attached to the renal polynucleotides a viral inhibitory renal peptide (VIP) through a cleavable linker. The cleavable linker can release the VIP and dye into the cell. In another example, the renal polynucleotides can be attached through the linker to an ADP-ribosylate, which is responsible for the actions of some bacterial toxins, such as cholera toxin, diphtheria toxin, and pertussis toxin. These toxin proteins are ADP-ribosyltransferases that modify target proteins in human cells. For example, cholera toxin ADP-ribosylates G proteins modifies human cells by causing massive fluid secretion from the lining of the small intestine, which results in life-threatening diarrhea.

In some embodiments, the payload may be a therapeutic agent such as a cytotoxin, radioactive ion, chemotherapeutic, or other therapeutic agent. A cytotoxin or cytotoxic agent includes any agent that may be detrimental to cells. Examples include, but are not limited to, taxol, cytochalasin B, gramicidin D, ethidium bromide, emetine, mitomycin, etoposide, teniposide, vincristine, vinblastine, colchicine, doxorubicin, daunorubicin, dihydroxyanthracinedione, mitoxantrone, mithramycin, actinomycin D, 1-dehydrotestosterone, glucocorticoids, procaine, tetracaine, lidocaine, propranolol, puromycin, maytansinoids, e.g., maytansinol (see U.S. Pat. No. 5,208,020 incorporated herein in its entirety), rachelmycin (CC-1065, see U.S. Pat. Nos. 5,475,092, 5,585,499, and 5,846,545, all of which are incorporated herein by reference), and analogs or homologs thereof. Radioactive ions include, but are not limited to iodine (e.g., iodine 125 or iodine 131), strontium 89, phosphorous, palladium, cesium, iridium, phosphate, cobalt, yttrium 90, samarium 153, and praseodymium. Other therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thiotepa chlorambucil, rachelmycin (CC-1065), melphalan, carmustine (BSNU), lomustine (CCNU), cyclophosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine, vinblastine, taxol and maytansinoids).

In some embodiments, the payload may be a detectable agent, such as various organic small molecules, inorganic compounds, nanoparticles, enzymes or enzyme substrates, fluorescent materials, luminescent materials (e.g., luminol), bioluminescent materials (e.g., luciferase, luciferin, and aequorin), chemiluminescent materials, radioactive materials (e.g., ¹⁸F, ⁶⁷Ga, ^{81m}Kr, ⁸²Rb, ¹¹¹In, ¹²³I, ¹³³Xe, ²⁰¹I, ¹²⁵I, ³⁵S, ¹⁴C, ³H, or ^{99m}Tc (e.g., as pertechnetate (technetate(VII), TcO₄⁻)), and contrast agents (e.g., gold (e.g., gold nanoparticles), gadolinium (e.g., chelated Gd), iron oxides (e.g., superparamagnetic iron oxide (SPIO), monocrystalline iron oxide nanoparticles (MIONs), and ultrasmall superparamagnetic iron oxide (USPIO)), manganese chelates (e.g., Mn-DPDP), barium sulfate, iodinated contrast media (iohexol), microbubbles, or perfluorocarbons). Such optically-detectable labels include for example, without limitation, 4-acetamido-4′-isothiocyanatostilbene-2,2′disulfonic acid; acridine and derivatives (e.g., acridine and acridine isothiocyanate); 5-(2′-aminoethyl)aminonaphthalene-1-sulfonic acid (EDANS); 4-amino-N-[3-vinylsulfonyl)phenyl]naphthalimide-3,5 disulfonate; N-(4-anilino-l-naphthyl)maleimide; anthranilamide; BODIPY; Brilliant Yellow; coumarin and derivatives (e.g., coumarin, 7-amino-4-methylcoumarin (AMC, Coumarin 120), and 7-amino-4-trifluoromethylcoumarin (Coumarin 151)); cyanine dyes; cyanosine; 4′,6-diaminidino-2-phenylindole (DAPI); 5′ 5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]-naphthalene-1-sulfonyl chloride (DNS, dansylchloride); 4-dimethylaminophenylazophenyl-4′-isothiocyanate (DABITC); eosin and derivatives (e.g., eosin and eosin isothiocyanate); erythrosin and derivatives (e.g., erythrosin B and erythrosin isothiocyanate); ethidium; fluorescein and derivatives (e.g., 5-carboxyfluorescein (FAM), 5-(4,6-dichlorotriazin-2-yl)aminofluorescein (DTAF), 2′,7′-dimethoxy-4′5′-dichloro-6-carboxyfluorescein, fluorescein, fluorescein isothiocyanate, X-rhodamine-5-(and -6)-isothiocyanate (QFITC or XRITC), and fluorescamine); 2-[2-[3-[[1,3-dihydro-1,1-dimethyl-3-(3-sulfopropyl)-2H-benz[e]indol-2-ylidene]ethylidene]-2-[4-(ethoxycarbonyl)-1-piperazinyl]-1-cyclopenten-1-yl]ethenyl]-1,1-dimethyl-3-(3-sulforpropyl)-1H-benz[e]indolium hydroxide, inner salt, compound with n,n-diethylethanamine (1:1) (IR144); 5-chloro-2-[2-[3-[(5-chloro-3-ethyl-2(3H)-benzothiazol-ylidene)ethylidene]-2-(diphenylamino)-1-cyclopenten-1-yl]ethenyl]-3-ethyl benzothiazolium perchlorate (IR140); Malachite Green isothiocyanate; 4-methylumbelliferone orthocresolphthalein; nitrotyrosine; pararosaniline; Phenol Red; B-phycoerythrin; o-phthaldialdehyde; pyrene and derivatives (e.g., pyrene, pyrene butyrate, and succinimidyl 1-pyrene); butyrate quantum dots; Reactive Red 4 (CIBACRON™ Brilliant Red 3B-A); rhodamine and derivatives (e.g., 6-carboxy-X-rhodamine (ROX), 6-carboxyrhodamine (R6G), lissamine rhodamine B sulfonyl chloride rhodarnine (Rhod), rhodamine B, rhodamine 123, rhodamine X isothiocyanate, sulforhodamine B, sulforhodamine 101, sulfonyl chloride derivative of sulforhodamine 101 (Texas Red), N,N,N′,N letramethyl-6-carboxyrhodamine (TAM RA) tetramethyl rhodamine, and tetramethyl rhodamine isothiocyanate (TRITC)); riboflavin; rosolic acid; terbium chelate derivatives; Cyanine-3 (Cy3); Cyanine-5 (Cy5); cyanine-5.5 (Cy5.5), Cyanine-7 (Cy7); IRD 700; IRD 800; Alexa 647; La Jolta Blue; phthalo cyanine; and naphthalo cyanine.

In some embodiments, the detectable agent may be a non-detectable pre-cursor that becomes detectable upon activation (e.g., fluorogenic tetrazine-fluorophore constructs (e.g., tetrazine-BODIPY FL, tetrazine-Oregon Green 488, or tetrazine-BODIPY TMR-X) or enzyme activatable fluorogenic agents (e.g., PROSENSE® (VisEn Medical))). In vitro assays in which the enzyme labeled compositions can be used include, but are not limited to, enzyme linked immunosorbent assays (ELISAs), immunoprecipitation assays, immunofluorescence, enzyme immunoassays (EIA), radioimmunoassays (RIA), and Western blot analysis.

### Combinations

The renal polynucleotides may be used in combination with one or more other therapeutic, prophylactic, diagnostic, or imaging agents. By “in combination with,” it is not intended to imply that the agents must be administered at the same time and/or formulated for delivery together, although these methods of delivery are within the scope of the present disclosure. Compositions can be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of pharmaceutical, prophylactic, diagnostic, or imaging compositions in combination with agents that may improve their bioavailability, reduce and/or modify their metabolism, inhibit their excretion, and/or modify their distribution within the body. As a non-limiting example, the renal polynucleotides may be used in combination with a pharmaceutical agent for the treatment of cancer or to control hyperproliferative cells. In U.S. Pat. No. 7,964,571, herein incorporated by reference in its entirety, a combination therapy for the treatment of solid primary or metastasized tumor is described using a pharmaceutical composition including a DNA plasmid encoding for interleukin-12 with a lipopolymer and also administering at least one anticancer agent or chemotherapeutic. Further, the renal polynucleotides of the present invention that encodes anti-proliferative molecules may be in a pharmaceutical composition with a lipopolymer (see e.g., U.S. Pub. No. 20110218231, herein incorporated by reference in its entirety, claiming a pharmaceutical composition comprising a DNA plasmid encoding an anti-proliferative molecule and a lipopolymer) which may be administered with at least one chemotherapeutic or anticancer agent (See e.g., the “Combination” Section in U.S. Pat. No. 8,518,907 and International Patent Publication No. WO201218754; the contents of each of which are herein incorporated by reference in its entirety).

Examples of estrogen receptor modulators, androgen receptor modulators, retinoid receptor modulators, cytotoxic agents, a hypoxia activatable, proteasome inhibitors, microtubule inhibitors/microtubule-stabilising agents, topoisomerase inhibitors, inhibitors of mitotic kinesins, histone deacetylase inhibitors, inhibitors of kinases involved in mitotic progression, antiproliferative agents, monoclonal antibody targeted therapeutic agents, HMG-CoA reductase inhibitors, prenyl-protein transferase inhibitors, angiogenesis inhibitors, therapeutic agents that modulate or inhibit angiogenesis, agents that interfere with cell cycle checkpoints, agents that interfere with receptor tyrosine kinases (RTKs), inhibitors of cell proliferation and survival signaling pathway, apoptosis inducing agents, NSAIDs that are selective COX-2 inhibitors, inhibitors of COX-2, compounds that have been described as specific inhibitors of COX-2, angiogenesis inhibitors, tyrosine kinase inhibitors, compounds other than anti-cancer compounds, inhibitor of inherent multidrug resistance (MDR), anti-emetic agents to treat nausea or emesis, and neurokinin-1 receptor antagonists, are described in co-pending International Patent Publication No. WO2015038892, the contents of which is incorporated by reference in its entirety, such as, but not limited to, in pargraphs [000925]-[000957].

In one embodiment, renal polynucleotides may be co-administered with at least one small molecule additive. As used herein, “co-administered” means the administration of two or more components. These components for co-administration include, but are not limited to active ingredients, renal polynucleotides, amino acids, inactive ingredients and excipients. Co-administration refers to the administration of two or more components simultaneously or with a time lapse between administration such as 1 second, 5 seconds, 10 seconds, 15 seconds, 30 seconds, 45 seconds, 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 21 minutes, 22 minutes, 23 minutes, 24 minutes, 25 minutes, 26 minutes, 27 minutes, 28 minutes, 29 minutes, 30 minutes, 31 minutes, 32 minutes, 33 minutes, 34 minutes, 35 minutes, 36 minutes, 37 minutes, 38 minutes, 39 minutes, 40 minutes, 41 minutes, 42 minutes, 43 minutes, 44 minutes, 45 minutes, 46 minutes, 47 minutes, 48 minutes, 49 minutes, 50 minutes, 51 minutes, 52 minutes, 53 minutes, 54 minutes, 55 minutes, 56 minutes, 57 minutes, 58 minutes, 59 minutes, 1 hour, 1.5 hours, 2 hours, 2.5 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 1.5 days, 2 days, or more than 3 days.

In one embodiment, renal polynucleotides may be co-administered with at least one small molecule additive (e.g., amino acid) may be used to enhance cellular uptake, enhance intracellular release, increase translation, increase the duration of protein exposure, reduce the dosage requirement of the renal polynucleotide, reduce conformational diversity, improve chemical stability of the renal polynucleotide, increase the storage shelf life of formulations, conformational stability of the renal polynucleotide (e.g., in formulation, storage, during transport in vivo), reduced variability, form predictable physical structures, increase the dosage options for renal polynucleotides and/or increase the half-life of renal polynucleotide formulations. As a non-limiting example, a formulation with renal polynucleotides and at least one small molecule additive may be formulated in nanoparticles greater than 100 nm or in micron aggregates. These larger dosage forms may be used in various delivery options such as depots.

In one embodiment, the co-administration of the renal polynucleotide may be prior to the small molecule additive. In another embodiment, the co-administration of the renal polynucleotide may be after to the small molecule additive.

In one embodiment, the amount of renal polynucleotide co-administered in any dosage form may be from about 0.1 μg to about 50 mg, including 0.1 μg, 0.2 μg, 0.3 μg, 0.4 μg, 0.5 μg, 0.6 μg, 0.7 μg, 0.8 μg, 0.9 μg, 1.0 μg, 2 μg, 3 μg, 4 μg, 5 μg, 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 0.1 mg, 0.2 mg, 0.3 mg, 0.4 mg, 0.5 mg, 0.6 mg, 0.7 mg, 0.8 mg, 0.9 mg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg and 50 mg. The renal polynucleotide may be administered once a day, more than once a day, every other day, weekly, monthly, bimonthly or by a dosage schedule outlined herein.

In one embodiment, the amount of small molecule additive co-administered is from about 0.0001 mg/kg to about 100 mg/kg, from about 0.001 mg/kg to about 0.005 mg/kg, from about 0.001 mg/kg to about 0.05 mg/kg, from about 0.001 mg/kg to about 0.5 mg/kg, from about 0.001 mg/kg to about 1 mg/kg, from about 0.001 mg/kg to about 5 mg/kg, from about 0.001 mg/kg to about 10 mg/kg, from about 0.001 mg/kg to about 15 mg/kg, from about 0.001 mg/kg to about 20 mg/kg, from about 0.001 mg/kg to about 25 mg/kg, from about 0.001 mg/kg to about 30 mg/kg, from about 0.001 mg/kg to about 35 mg/kg, from about 0.001 mg/kg to about 40 mg/kg, from about 0.001 mg/kg to about 45 mg/kg, from about 0.001 mg/kg to about 50 mg/kg, from about 0.005 mg/kg to about 0.05 mg/kg, from about 0.005 mg/kg to about 0.5 mg/kg, from about 0.005 mg/kg to about 1 mg/kg, from about 0.005 mg/kg to about 5 mg/kg, from about 0.005 mg/kg to about 10 mg/kg, from about 0.005 mg/kg to about 15 mg/kg, from about 0.005 mg/kg to about 20 mg/kg, from about 0.005 mg/kg to about 25 mg/kg, from about 0.005 mg/kg to about 30 mg/kg, from about 0.005 mg/kg to about 35 mg/kg, from about 0.005 mg/kg to about 40 mg/kg, from about 0.005 mg/kg to about 45 mg/kg, from about 0.005 mg/kg to about 50 mg/kg, from about 0.05 mg/kg to about 0.5 mg/kg, from about 0.05 mg/kg to about 1 mg/kg, from about 0.05 mg/kg to about 5 mg/kg, from about 0.05 mg/kg to about 10 mg/kg, from about 0.05 mg/kg to about 15 mg/kg, from about 0.05 mg/kg to about 20 mg/kg, from about 0.05 mg/kg to about 25 mg/kg, from about 0.05 mg/kg to about 30 mg/kg, from about 0.05 mg/kg to about 35 mg/kg, from about 0.05 mg/kg to about 40 mg/kg, from about 0.05 mg/kg to about 45 mg/kg, from about 0.05 mg/kg to about 50 mg/kg, from about 0.01 mg/kg to about 0.5 mg/kg, from about 0.01 mg/kg to about 1 mg/kg, from about 0.01 mg/kg to about 5 mg/kg, from about 0.01 mg/kg to about 10 mg/kg, from about 0.01 mg/kg to about 15 mg/kg, from about 0.01 mg/kg to about 20 mg/kg, from about 0.01 mg/kg to about 25 mg/kg, from about 0.01 mg/kg to about 30 mg/kg, from about 0.01 mg/kg to about 35 mg/kg, from about 0.01 mg/kg to about 40 mg/kg, from about 0.01 mg/kg to about 45 mg/kg, from about 0.01 mg/kg to about 50 mg/kg, from about 0.1 mg/kg to about 0.5 mg/kg, from about 0.1 mg/kg to about 1 mg/kg, from about 0.1 mg/kg to about 5 mg/kg, from about 0.1 mg/kg to about 10 mg/kg, from about 0.1 mg/kg to about 15 mg/kg, from about 0.1 mg/kg to about 20 mg/kg, from about 0.1 mg/kg to about 25 mg/kg, from about 0.1 mg/kg to about 30 mg/kg, from about 0.1 mg/kg to about 35 mg/kg, from about 0.1 mg/kg to about 40 mg/kg, from about 0.1 mg/kg to about 45 mg/kg, from about 0.1 mg/kg to about 50 mg/kg, from about 0.5 mg/kg to about 30 mg/kg, from about 0.5 mg/kg to about 1 mg/kg, from about 0.5 mg/kg to about 5 mg/kg, from about 0.5 mg/kg to about 10 mg/kg, from about 0.5 mg/kg to about 15 mg/kg, from about 0.5 mg/kg to about 20 mg/kg, from about 0.5 mg/kg to about 25 mg/kg, from about 0.5 mg/kg to about 30 mg/kg, from about 0.5 mg/kg to about 35 mg/kg, from about 0.5 mg/kg to about 40 mg/kg, from about 0.5 mg/kg to about 45 mg/kg, or from about 0.5 mg/kg to about 50 mg/kg, of subject body weight (see e.g., the range of unit doses described in International Publication No WO2013078199, herein incorporated by reference in its entirety). The small molecule additive may be administered once a day, more than once a day, every other day, weekly, monthly, bimonthly or by a dosage schedule outlined herein. As a non-limiting example, the amount of small molecule additive co-administered per dose is a maximum of 48 mg/kg.

In one embodiment, the ratio of renal polynucleotide to total small molecule additive may be 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:41, 1:42, 1:43, 1:44, 1:45, 1:46, 1:47, 1:48, 1:49, 1:50, 1:51, 1:52, 1:53, 1:54, 1:55, 1:56, 1:57, 1:58, 1:59, 1:60, 1:61, 1:62, 1:63, 1:64, 1:65, 1:66, 1:67, 1:68, 1:69, 1:70, 1:71, 1:72, 1:73, 1:74, 1:75, 1:76, 1:77, 1:78, 1:79, 1:80, 1:81, 1:82, 1:83, 1:84, 1:85, 1:86, 1:87, 1:88, 1:89, 1:90, 1:91, 1:92, 1:93, 1:94,1:95, 1:96, 1:97, 1:98, 1:99, 1:100, 1:110, 1:120, 1:130, 1:140, 1:150, 1:160, 1:170, 1:180, 1:190, 1:200, 1:225, 1:250, 1:275, 1:300, 1:325, 1:350, 1:400, 1:450, 1:500, 1:550, 1:600 or greater than 1:600. The ratio may be molar, percent, weight, molar mass, nitrogen and phosphorus (N:P) ratio or any other ratio known or described herein.

In one embodiment, the ratio of total small molecule additive to renal polynucleotide may be 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:41, 1:42, 1:43, 1:44, 1:45, 1:46, 1:47, 1:48, 1:49, 1:50, 1:51, 1:52, 1:53, 1:54, 1:55, 1:56, 1:57, 1:58, 1:59, 1:60, 1:61, 1:62, 1:63, 1:64, 1:65, 1:66, 1:67, 1:68, 1:69, 1:70, 1:71, 1:72, 1:73, 1:74, 1:75, 1:76, 1:77, 1:78, 1:79, 1:80, 1:81, 1:82, 1:83, 1:84, 1:85, 1:86, 1:87, 1:88, 1:89, 1:90, 1:91, 1:92, 1:93, 1:94,1:95, 1:96, 1:97, 1:98, 1:99, 1:100, 1:110, 1:120, 1:130, 1:140, 1:150, 1:160, 1:170, 1:180, 1:190, 1:200, 1:225, 1:250, 1:275, 1:300, 1:325, 1:350, 1:400, 1:450, 1:500, 1:550, 1:600 or greater than 1:600. The ratio may be molar, percent, weight, molar mass, nitrogen and phosphorus (N:P) ratio, length, or any other ratio known or described herein.

In one embodiment, the nitrogen and phosphorous (N:P) ratio is between 0.1 and 7, 0.1 and 6, 0.1 and 6, 0.1 and 5, 0.1 and 4, 0.1 and 3.5, 0.1 and 2.5, 0.1 and 2, 0.1 and 1.5, 0.1 and 1, 0.1 and 0.5, 0.5 and 7, 0.5 and 6, 0.5 and 6, 0.5 and 5, 0.5 and 4, 0.5 and 3.5, 0.5 and 2.5, 0.5 and 2, 0.5 and 1.5, 0.5 and 1, 1 and 7, 1 and 6, 1 and 6, 1 and 5, 1 and 4, 1 and 3.5, 1 and 2.5, 1 and 2, 1 and 1.5, 1.5 and 7, 1.5 and 6, 1.5 and 6, 1.5 and 5, 1.5 and 4, 1.5 and 3.5, 1.5 and 2.5, 1.5 and 2, 2 and 7, 2.5 and 7, 2.5 and 6, 2.5 and 6, 2.5 and 5, 2.5 and 4, 2.5 and 3.5, 3.5 and 7, 3.5 and 6, 3.5 and 5, 3.5 and 4, 4 and 7, 4 and 6, 4 and 5, 5 and 7 or between 6 and 7. As a non-limiting example, the N:P ratio is between 2.5 and 7. As another non-limiting example, the N:P ratio is between 2.5 and 4. As another non-limiting example, the N:P ratio is between 4 and 6. As another non-limiting example, the N:P ratio is between 2.5 and 3.5.

In one embodiment the N:P ratio is 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.67, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, or 10. As a non-limiting example, the N:P ratio is 2.9. As another non-limiting example, the N:P ratio is 3.1. As another non-limiting example, the N:P ratio is 4. As another non-limiting example the N:P ratio is 5. As another non-limiting example, the N:P ratio is 5.67. As another non-limiting example, the N:P ratio is 6.

In one embodiment, the ratio of total small molecule additive to the renal polynucleotide may be greater than the renal polynucleotide. In another embodiment, the ratio of the mass of the total small molecule additive may be greater than the mass of the renal polynucleotide. In yet another embodiment, the ratio of the molar composition of the total small molecule additive may be greater than the molar composition of the renal polynucleotide. The ratio of total small molecule additive may be 2×, 3×, 4×, 5×, 6×, 7×, 8×, 9×, 10×, 11×, 12×, 13×, 14×, 15×, 16×, 17×, 18×, 19×, 20×, 21×, 22×, 23×, 24×, 25×, 26×, 27×, 28×, 29×, 30×, 31×, 32×, 33×, 34×, 35×, 36×, 37×, 38×, 39×, 40×, 41×, 42×, 43×, 44×, 45×, 46×, 47×, 48×, 49×, 50×, 51×, 52×, 53×, 54×, 55×, 56×, 57×, 58×, 59×, 60×, 61×, 62×, 63×, 64×, 65×, 66×, 67×, 68×, 69×, 70×, 71×, 72×, 73×, 74×, 75×, 76×, 77×, 78×, 79×, 80×, 81×, 82×, 83×, 84×, 85×, 86×, 87×, 88×, 89×, 90×, 91×, 92×, 93×, 94×, 95×, 96×, 97×, 98×, 99×, 100×, 110×, 120×, 130×, 140×, 150×, 160×, 170×, 180×, 190×, 200×, 225×, 250×, 275×, 300×, 325×, 350×, 400×, 450×, 500×, 550×, 600× or greater than 650× to the renal polynucleotide.

Amino acids which may be co-administered with the renal polynucleotides described herein may be natural or non-natural amino acids and analogs thereof. Natural amino acids, non-natural amino acids, compositions and formulations thereof with the renal polynucleotides described herein are described in co-pending International Patent Publication No. WO2015038892, the contents of which is incorporated by reference in its entirety, such as, but not limited to, in paragraphs [000976]-[0001030]. The combinations referred to above can conveniently be presented for use in the form of a pharmaceutical formulation and thus pharmaceutical compositions comprising a combination as defined above together with a pharmaceutically acceptable diluent, excipient or carrier represent a further aspect of the invention.

The individual compounds of such combinations can be administered either sequentially or simultaneously in separate or combined pharmaceutical formulations. In one embodiment, the individual compounds will be administered simultaneously in a combined pharmaceutical formulation.

It will further be appreciated that therapeutically, prophylactically, diagnostically, or imaging active agents utilized in combination may be administered together in a single composition or administered separately in different compositions. In general, it is expected that agents utilized in combination with be utilized at levels that do not exceed the levels at which they are utilized individually. In some embodiments, the levels utilized in combination will be lower than those utilized individually. In one embodiment, the combinations, each or together may be administered according to the split dosing regimens described herein.

### Dosing

The present invention provides methods comprising administering renal polynculeotides and their encoded proteins or complexes in accordance with the invention to a subject in need thereof. Nucleic acids, proteins or complexes, or pharmaceutical, imaging, diagnostic, or prophylactic compositions thereof, may be administered to a subject using any amount and any route of administration effective for preventing, treating, diagnosing, or imaging a disease, disorder, and/or condition (e.g., a disease, disorder, and/or condition relating to working memory deficits). The exact amount required will vary from subject to subject, depending on the species, age, and general condition of the subject, the severity of the disease, the particular composition, its mode of administration, its mode of activity, and the like. Compositions in accordance with the invention are typically formulated in dosage unit form for ease of administration and uniformity of dosage. It will be understood, however, that the total daily usage of the compositions of the present invention may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

In certain embodiments, compositions in accordance with the present invention may be administered at dosage levels sufficient to deliver from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic, prophylactic, or imaging effect (see e.g., the range of unit doses described in International Publication No WO2013078199, herein incorporated by reference in its entirety). The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used.

According to the present invention, it has been discovered that administration of renal polynucleotides in split-dose regimens produce higher levels of proteins in mammalian subjects. As used herein, a “split dose” is the division of single unit dose or total daily dose into two or more doses, e.g., two or more administrations of the single unit dose. As used herein, a “single unit dose” is a dose of any therapeutic administered in one dose/at one time/single route/single point of contact, i.e., single administration event. As used herein, a “total daily dose” is an amount given or prescribed in 24 hr period. It may be administered as a single unit dose. In one embodiment, the renal polynucleotides of the present invention are administered to a subject in split doses. The renal polynucleotides may be formulated in buffer only or in a formulation described herein.

### Dosage Forms

A pharmaceutical composition described herein can be formulated into a dosage form described herein, such as a topical, intranasal, intratracheal, or injectable (e.g., intravenous, intraocular, intravitreal, intramuscular, intracardiac, intraperitoneal, and subcutaneous).

### Liquid Dosage Forms

Liquid dosage forms for parenteral administration are described in co-pending International Patent Publication No. WO2015038892, the contents of which is incorporated by reference in its entirety, such as, but not limited to, in paragraph [0001037].

### Injectable

Injectable preparations, for example, sterile injectable aqueous or oleaginous suspensions may be formulated according to the known art and may include suitable dispersing agents, wetting agents, and/or suspending agents. Sterile injectable preparations may be sterile injectable solutions, suspensions, and/or emulsions in nontoxic parenterally acceptable diluents and/or solvents, for example, a solution in 1,3-butanediol. Among the acceptable vehicles and solvents that may be employed include, but are not limited to, water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of an active ingredient, it may be desirable to slow the absorption of the active ingredient from subcutaneous or intramuscular injection. This may be accomplished by the use of a liquid suspension of crystalline or amorphous material with poor water solubility. The rate of absorption of the renal polynucleotides then depends upon its rate of dissolution which, in turn, may depend upon crystal size and crystalline form. Alternatively, delayed absorption of a parenterally administered renal polynucleotides may be accomplished by dissolving or suspending the renal polynucleotides in an oil vehicle. Injectable depot forms are made by forming microencapsule matrices of the renal polynucleotides in biodegradable polymers such as polylactide-polyglycolide. Depending upon the ratio of renal polynucleotides to polymer and the nature of the particular polymer employed, the rate of renal polynucleotides release can be controlled. Examples of other biodegradable polymers include, but are not limited to, poly(orthoesters) and poly(anhydrides). Depot injectable formulations may be prepared by entrapping the renal polynucleotides in liposomes or microemulsions which are compatible with body tissues.

Localized injection of naked DNA was demonstrated intramuscularly in 1990 and later was injected into several other tissues including liver, skin and brain. The uptake of the DNA was mostly localized in the area of the needle track. Different agents may be used to enhance overall gene expression. In one embodiment, the renal polynucleotides may be administered with an agent to enhance expression. Non-limiting examples of agents include transferrin, water-immiscible solvents, nonionic polymers, surfactants, and nuclease inhibitors.

A needle-free delivery method known as jet injection may be used to deliver a drug to a tissue. The jet injection method uses a high-speed ultrafine stream of solution driven by a pressurized gas. The penetration power of this method may be adjusted by altering the gas pressure and the mechanical properties of the target tissue. The fluid being administered travels through the path of least resistance and may facilitate transport outside the traditional zone of delivery. As a non-limiting example, the solution may include the renal polynucleotides described herein. The solution (approximately 3-5 ul) may be loaded into the jet injection device and administered to a tissue at a pressure of approximately 1-3 bars. Commercial liquid jet injectors include, but are not limited to, Vitaject and bioject 2000 (Bioject), Advantagect (Activa systems), Injex 30 (Injex equidyne) and Mediject VISION (Antares Pharma).

Microneedles may be used to inject the renal polynucleotides and formulations thereof described herein. Microneedles are an array of microstructured projections which can be coated with a drug that can be administered to a subject to provide delivery of therapeutic agents (e.g., renal polynucleotides) within the epidermis. Microneedles can be approximately 1 μm in diameter and from about 1 μm to about 100 μm (e.g., about 1 μm, about 2 μm, about 3 μm, about 4 μm, about 5 μm, about 6 μm, about 7 μm, about 8 μm, about 9 μm, about 10 μm, about 12 μm, about 14 μm, about 15 μm, about 16 μm, about 18 μm, about 20 μm, about 25 μm, about 30 μm, about 35 μm, about 40 μm, about 45 μm, about 50 μm, about 55 μm, about 60 μm, about 65 μm, about 70 μm about 75 μm, about 80 μm, about 85 μm, about 90 μm, about 95 μm, or about 100 μm) in length. The material used to make microneedles may be, but is not limited to, metals, silicon, silicon dioxide, polymers, glass and other materials and the material selected may depend on the type of agent to be delivered and the tissue contacted. In one embodiment, the miconeedles may be solid and may either be straight, bend or filtered. In one embodiment, the miconeedles may be hollow and may either be straight, bend or filtered.

In one embodiment, the renal polynucleotides and formulations thereof may be administered using a microneedle drug delivery system. The microneedles may be hollow, solid or a combination thereof. As a non-limiting example, the microneedle drug delivery system may be the 3M Hollow Microstructured Transdermal System (hMTS). As another non-limiting example, the microneedle drug delivery system may be a microneedle patch comprising solid microneedle technology from 3M (3M Drug Delivery Systems).

In one embodiment, the formulations described herein may be administered using a multi-prong needle device. As a non-limiting example, the device may administer more than one formulation in a single delivery. The formulations may be delivered at the same time or the formulations may have a pre-determined interval between each formulation delivery.

In one embodiment, the formulations described herein may be administered to more than one location to a tissue, organ or subject at the same time using a multi-prong needle device. The formulations may be administered at the same time or the formulations may have a pre-determined interval between each administration of a formulation.

In one embodiment, the amount of formulation comprising the renal polynucleotides administered may be varied depending on the type of injection and/or the cell, tissue or organ administered the formulation. As a non-limiting example, for intramuscular injection the formulation may be more concentrated to produce a renal polypeptide of interest as compared to a formulation for intravenous delivery.

### Coatings or Shells

Solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type may be employed as fillers in soft and hard-filled gelatin capsules using such excipients as lactose or milk sugar as well as high molecular weight polyethylene glycols and the like.

### Multi-Dose and Repeat-Dose Administration

In some embodiments, compounds and/or compositions of the present invention may be administered in two or more doses (referred to herein as “multi-dose administration”). Such doses may comprise the same components or may comprise components not included in a previous dose. Such doses may comprise the same mass and/or volume of components or an altered mass and/or volume of components in comparison to a previous dose. In some embodiments, multi-dose administration may comprise repeat-dose administration. As used herein, the term “repeat-dose administration” refers to two or more doses administered consecutively or within a regimen of repeat doses comprising substantially the same components provided at substantially the same mass and/or volume. In some embodiments, subjects may display a repeat-dose response. As used herein, the term “repeat-dose response” refers to a response in a subject to a repeat-dose that differs from that of another dose administered within a repeat-dose administration regimen. In some embodiments, such a response may be the expression of a protein in response to a repeat-dose comprising mRNA. In such embodiments, protein expression may be elevated in comparison to another dose administered within a repeat-dose administration regimen or protein expression may be reduced in comparison to another dose administered within a repeat-dose administration regimen. Alteration of protein expression may be from about 1% to about 20%, from about 5% to about 50% from about 10% to about 60%, from about 25% to about 75%, from about 40% to about 100% and/or at least 100%. A reduction in expression of mRNA administered as part of a repeat-dose regimen, wherein the level of protein translated from the administered RNA is reduced by more than 40% in comparison to another dose within the repeat-dose regimen is referred to herein as “repeat-dose resistance.”

### Properties of the Pharmaceutical Compositions

The pharmaceutical compositions described herein can be characterized by one or more of the following properties:

### Bioavailability

The renal polynucleotides, when formulated into a composition with a delivery agent as described herein, can exhibit an increase in bioavailability as compared to a composition lacking a delivery agent as described herein. As used herein, the term “bioavailability” refers to the systemic availability of a given amount of renal polynucleotides administered to a mammal. Bioavailability can be assessed by measuring the area under the curve (AUC) or the maximum serum or plasma concentration (Cₘₐₓ) of the unchanged form of a compound following administration of the compound to a mammal. AUC is a determination of the area under the curve plotting the serum or plasma concentration of a compound along the ordinate (Y-axis) against time along the abscissa (X-axis). Generally, the AUC for a particular compound can be calculated using methods known to those of ordinary skill in the art and as described in G. S. Banker, Modern Pharmaceutics, Drugs and the Pharmaceutical Sciences, v. 72, Marcel Dekker, New York, Inc., 1996, herein incorporated by reference in its entirety.

The Cₘₐₓ value is the maximum concentration of the compound achieved in the serum or plasma of a mammal following administration of the compound to the mammal. The Cₘₐₓ value of a particular compound can be measured using methods known to those of ordinary skill in the art. The phrases “increasing bioavailability” or “improving the pharmacokinetics,” as used herein mean that the systemic availability of a first renal polynucleotides, measured as AUC, Cₘₐₓ, or Cₘᵢₙ in a mammal is greater, when co-administered with a delivery agent as described herein, than when such co-administration does not take place. In some embodiments, the bioavailability of the renal polynucleotides can increase by at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%.

In some embodiments, liquid formulations of renal polynucleotides may have varying in vivo half-life, requiring modulation of doses to yield a therapeutic effect. To address this, in some embodiments of the present invention, renal polynucleotides formulations may be designed to improve bioavailability and/or therapeutic effect during repeat administrations. Such formulations may enable sustained release of renal polynucleotides and/or reduce renal polynucleotide degradation rates by nucleases. In some embodiments, suspension formulations are provided comprising renal polynucleotides, water immiscible oil depots, surfactants and/or co-surfactants and/or co-solvents. Combinations of oils and surfactants may enable suspension formulation with renal polynucleotides. Delivery of renal polynucleotides in a water immiscible depot may be used to improve bioavailability through sustained release of renal polynucleotides from the depot to the surrounding physiologic environment and/or prevent renal polynucleotide degradation by nucleases.

In some embodiments, cationic nanoparticles comprising combinations of divalent and monovalent cations may be formulated with renal polynucleotides. Such nanoparticles may form spontaneously in solution over a given period (e.g. hours, days, etc.). Such nanoparticles do not form in the presence of divalent cations alone or in the presence of monovalent cations alone. The delivery of renal polynucleotides in cationic nanoparticles or in one or more depot comprising cationic nanoparticles may improve renal polynucleotide bioavailability by acting as a long-acting depot and/or reducing the rate of degradation by nucleases.

### Therapeutic Window

The renal polynucleotides, when formulated into a composition with a delivery agent as described herein, can exhibit an increase in the therapeutic window of the administered renal polynucleotides composition as compared to the therapeutic window of the administered renal polynucleotides composition lacking a delivery agent as described herein. As used herein “therapeutic window” refers to the range of plasma concentrations, or the range of levels of therapeutically active substance at the site of action, with a high probability of eliciting a therapeutic effect. In some embodiments, the therapeutic window of the renal polynucleotides when co-administered with a delivery agent as described herein can increase by at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%.

### Volume of Distribution

The renal polynucleotides, when formulated into a composition with a delivery agent as described herein, can exhibit an improved volume of distribution (V_{dist}), e.g., reduced or targeted, relative to a composition lacking a delivery agent as described herein. The volume of distribution (V_{dist}) relates the amount of the drug in the body to the concentration of the drug in the blood or plasma. As used herein, the term “volume of distribution” refers to the fluid volume that would be required to contain the total amount of the drug in the body at the same concentration as in the blood or plasma: Vdist equals the amount of drug in the body/concentration of drug in blood or plasma. For example, for a 10 mg dose and a plasma concentration of 10 mg/L, the volume of distribution would be 1 liter. The volume of distribution reflects the extent to which the drug is present in the extravascular tissue. A large volume of distribution reflects the tendency of a compound to bind to the tissue components compared with plasma protein binding. In a clinical setting, Vdist can be used to determine a loading dose to achieve a steady state concentration. In some embodiments, the volume of distribution of the renal polynucleotides when co-administered with a delivery agent as described herein can decrease at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%.

### Biological Effect

In one embodiment, the biological effect of the renal polynucleotides delivered to the animals may be categorized by analyzing the protein expression in the animals. The protein expression may be determined from analyzing a biological sample collected from a mammal administered the renal polynucleotides of the present invention. In one embodiment, the expression protein encoded by the renal polynucleotides administered to the mammal of at least 50 pg/ml may be preferred. For example, a protein expression of 50-200 pg/ml for the protein encoded by the renal polynucleotides delivered to the mammal may be seen as a therapeutically effective amount of protein in the mammal.

### Detection of Renal Polynucleotides by Mass Spectrometry

Mass spectrometry (MS) is an analytical technique that can provide structural and molecular mass/concentration information on molecules after their conversion to ions. The molecules are first ionized to acquire positive or negative charges and then they travel through the mass analyzer to arrive at different areas of the detector according to their mass/charge (m/z) ratio. Methods of detecting renal polynucleotides are described in co-pending International Patent Publication No. WO2015038892, the contents of which is incorporated by reference in its entirety, such as, but not limited to, in paragraphs [0001055]-[0001067].

### V. USES OF RENAL POLYNUCLEOTIDES OF THE INVENTION

The renal polynucleotides of the present invention are designed, in preferred embodiments, to provide for avoidance or evasion of deleterious bio-responses such as the immune response and/or degradation pathways, overcoming the threshold of expression and/or improving protein production capacity, improved expression rates or translation efficiency, improved drug or protein half-life and/or protein concentrations, optimized protein localization, to improve one or more of the stability and/or clearance in tissues, receptor uptake and/or kinetics, cellular access by the compositions, engagement with translational machinery, secretion efficiency (when applicable), accessibility to circulation, and/or modulation of a cell's status, function and/or activity.

### Therapeutics

### Therapeutic Agents

The renal polynucleotides of the present invention, such as, but not limited to, IVT renal polynucleotides, chimeric renal polynucleotides, modified nucleic acids and modified RNAs, and the proteins translated from them described herein can be used as therapeutic or prophylactic agents. In one embodiment, they are provided for use in medicine. For example, a renal polynucleotide described herein can be administered to a subject, wherein the renal polynucleotides is translated in vivo to produce a therapeutic or prophylactic renal polypeptide in the subject. Provided are compositions, methods, kits, and reagents for diagnosis, treatment or prevention of a disease or condition in humans and other mammals. The active therapeutic agents of the invention include renal polynucleotides, cells containing renal polynucleotides or renal polypeptides translated from the renal polynucleotides.

In certain embodiments, provided herein are combination therapeutics containing one or more renal polynucleotides containing translatable regions that encode for a protein or proteins that boost a mammalian subject's immunity along with a protein that induces antibody-dependent cellular toxicity. For example, provided herein are therapeutics containing one or more nucleic acids that encode trastuzumab and granulocyte-colony stimulating factor (G-CSF). In particular, such combination therapeutics are useful in Her2+ breast cancer patients who develop induced resistance to trastuzumab. (See, e.g., Albrecht, Immunotherapy. 2(6):795-8 (2010)).

Provided herein are methods of inducing translation of a recombinant renal polypeptide in a cell population using the renal polynucleotides described herein. Such translation can be in vivo, ex vivo, in culture, or in vitro. The cell population is contacted with an effective amount of a composition containing a nucleic acid that has at least one nucleoside modification, and a translatable region encoding the recombinant renal polypeptide. The population is contacted under conditions such that the nucleic acid is localized into one or more cells of the cell population and the recombinant renal polypeptide is translated in the cell from the nucleic acid.

An “effective amount” of the composition is provided based, at least in part, on the target tissue, target cell type, means of administration, physical characteristics of the nucleic acid (e.g., size, and extent of modified nucleosides), and other determinants. In general, an effective amount of the composition provides efficient protein production in the cell, preferably more efficient than a composition containing a corresponding unmodified nucleic acid. Increased efficiency may be demonstrated by increased cell transfection (i.e., the percentage of cells transfected with the nucleic acid), increased protein translation from the nucleic acid, decreased nucleic acid degradation (as demonstrated, e.g., by increased duration of protein translation from a modified nucleic acid), or reduced innate immune response of the host cell.

Aspects of the invention are directed to methods of inducing in vivo translation of a recombinant renal polypeptide in a mammalian subject in need thereof. Therein, an effective amount of a composition containing a nucleic acid that has at least one structural or chemical modification and a translatable region encoding the recombinant renal polypeptide is administered to the subject using the delivery methods described herein. The nucleic acid is provided in an amount and under other conditions such that the nucleic acid is localized into a cell of the subject and the recombinant renal polypeptide is translated in the cell from the nucleic acid. The cell in which the nucleic acid is localized, or the tissue in which the cell is present, may be targeted with one or more than one rounds of nucleic acid administration.

In certain embodiments, the administered renal polynucleotides directs production of one or more recombinant renal polypeptides that provide a functional activity which is substantially absent in the cell, tissue or organism in which the recombinant renal polypeptide is translated. For example, the missing functional activity may be enzymatic, structural, or gene regulatory in nature. In related embodiments, the administered renal polynucleotides directs production of one or more recombinant renal polypeptides that increases (e.g., synergistically) a functional activity which is present but substantially deficient in the cell in which the recombinant renal polypeptide is translated.

In other embodiments, the administered renal polynucleotides directs production of one or more recombinant renal polypeptides that replace a renal polypeptide (or multiple renal polypeptides) that is substantially absent in the cell in which the recombinant renal polypeptide is translated. Such absence may be due to genetic mutation of the encoding gene or regulatory pathway thereof. In some embodiments, the recombinant renal polypeptide increases the level of an endogenous protein in the cell to a desirable level; such an increase may bring the level of the endogenous protein from a subnormal level to a normal level or from a normal level to a super-normal level.

Alternatively, the recombinant renal polypeptide functions to antagonize the activity of an endogenous protein present in, on the surface of, or secreted from the cell. Usually, the activity of the endogenous protein is deleterious to the subject; for example, due to mutation of the endogenous protein resulting in altered activity or localization. Additionally, the recombinant renal polypeptide antagonizes, directly or indirectly, the activity of a biological moiety present in, on the surface of, or secreted from the cell. Examples of antagonized biological moieties include lipids (e.g., cholesterol), a lipoprotein (e.g., low density lipoprotein), a nucleic acid, a carbohydrate, a protein toxin such as shiga and tetanus toxins, or a small molecule toxin such as botulinum, cholera, and diphtheria toxins. Additionally, the antagonized biological molecule may be an endogenous protein that exhibits an undesirable activity, such as a cytotoxic or cytostatic activity.

The recombinant proteins described herein may be engineered for localization within the cell, potentially within a specific compartment such as the nucleus, or are engineered for secretion from the cell or translocation to the plasma membrane of the cell.

In some embodiments, the renal polynucleotides described herein and their encoded renal polypeptides in accordance with the present invention may be used for treatment of any of a variety of diseases, disorders, and/or conditions, including but not limited to urological disorders (e.g. renal disease); diseases characterized by dysfunctional or aberrant protein activity (e.g., cystic fibrosis, sickle cell anemia, epidermolysis bullosa, amyotrophic lateral sclerosis, and glucose-6-phosphate dehydrogenase deficiency); diseases characterized by missing (or substantially diminished such that proper (normal or physiological protein function does not occur) protein activity (e.g., cystic fibrosis, Niemann-Pick type C, β thalassemia major, Duchenne muscular dystrophy, Hurler Syndrome, and Hunter Syndrome).

In these diseases, disorders and/or conditions proteins may not be present, or are essentially non-functional. The present invention provides a method for treating such conditions or diseases in a subject by introducing nucleic acid or cell-based therapeutics containing the renal polynucleotides provided herein, wherein the renal polynucleotides encode for a protein that replaces the protein activity which is decreased, dysfunctional, or missing from the target cells of the subject.

Provided herein, are methods to prevent infection and/or sepsis in a subject at risk of developing infection and/or sepsis, the method comprising administering to a subject in need of such prevention a composition comprising a renal polynucleotide precursor encoding an anti-microbial renal polypeptide (e.g., an anti-bacterial renal polypeptide), or a partially or fully processed form thereof in an amount sufficient to prevent infection and/or sepsis.

Further provided herein, are methods to treat infection and/or sepsis in a subject, the method comprising administering to a subject in need of such treatment a composition comprising a renal polynucleotide precursor encoding an anti-microbial renal polypeptide (e.g., an anti-bacterial renal polypeptide), e.g., an anti-microbial renal polypeptide described herein, or a partially or fully processed form thereof in an amount sufficient to treat an infection and/or sepsis.

In certain embodiments, the subject may exhibits acute or chronic microbial infections (e.g., bacterial infections). In certain embodiments, the subject may have received or may be receiving a therapy.

Other aspects of the present disclosure relate to transplantation of cells containing renal polynucleotides to a mammalian subject. Administration of cells to mammalian subjects is known to those of ordinary skill in the art, and include, but is not limited to, local implantation (e.g., topical or subcutaneous administration), organ delivery or systemic injection (e.g., intravenous injection or inhalation), and the formulation of cells in pharmaceutically acceptable carrier. Such compositions containing renal polynucleotides can be formulated for administration intramuscularly, transarterially, intraperitoneally, intravenously, intranasally, subcutaneously, endoscopically, transdermally, or intrathecally. In some embodiments, the composition may be formulated for extended release.

The subject to whom the therapeutic agent may be administered suffers from or may be at risk of developing a disease, disorder, or deleterious condition. Provided are methods of identifying, diagnosing, and classifying subjects on these bases, which may include clinical diagnosis, biomarker levels, genome-wide association studies (GWAS), and other methods known in the art.

### Primary Glomerular Disease

In one embodiment, the renal polynucleotides described herein may be used to treat and/or prevent a primary glomerular disease such as, but not limited to, Alport's syndrome (X-linked or autosomal recessive), Benign familial hematuria, congenital nephrosis I (Finnish tpe), Nail Patella syndrome and/or familial mesangials sclerosis.

### Alport's Syndrome (X-Linked or Autosomal Recessive)

Alport's Syndrome causes damage to the kidneys by the formation of scar tissue (fibrosis), which eventually leads to kidney failure. The disease can also effect the inner ear, causing hearing loss, and the eye, causing a slow progressive deterioration of vision and in some cases cataracts. Alport Syndrome is caused by genetic mutations in a type IV collagen gene. These type IV collagens are key components of basement membranes, thin structures that separate cells in all tissues, including the kidney, inner ear, and eye. In 80% of cases, the disease is passed on through a mutation on the X chromosome.

In one embodiment, the renal polynucleotides described herein may be used to treat and/or prevent Alport's syndrome.

### Benign Familial Hematuria

Thin basement membrane nephropathy (TBMN) (also known as benign familial hematuria and thin basement membrane disease) is one of the most common causes of blood in the urine as a sole symptom. TBMN is associated with a thinning of the basement membrane of the kidney filters (glomeruli). Most patients with TBMN have microscopic hematuria on urinalysis but retain normal kidney function.

In one embodiment, the renal polynucleotides described herein may be used to treat and/or prevent benign familial hematuria.

### Congenital Nephrosis I (Finnish Type)

Congenital Nephrotic Syndrome of the Finish type (CNF) is a rare and severe autosomal recessive disease caused by a mutation in the gene NPHS1, a gene that encodes an adhesion molecule that functions in the filtration barrier of the kidney. The onset of the disease is in utero or within the first three months of life and the disease progresses rapidly to end stage kidney disease. Symptoms include protein in the urine, low blood protein levels, high cholesterol levels, and swelling.

In one embodiment, the renal polynucleotides described herein may be used to treat and/or prevent Congenital Nephrotic Syndrome of the Finish type (CNF).

### Nail Patella Syndrome

Nail-patella syndrome (also known as hereditary osteo-onychodysplasia) is a dominant genetic disorder linked to an abnormalcy in chromosome 9. Severity and symptoms between individuals varies, but nail abnormalities, such as underdeveloped, split, ridged or pitted nails, and skeletal abnormalities involving knees, elbows, and hips are common among most individuals.

In one embodiment, the renal polynucleotides described herein may be used to treat and/or prevent Nail-patella syndrome.

### Familial Mesangial Sclerosis

Diffuse mesangial sclerosis is a cause of primary congenital nephrotic syndrome, a rare renal disease, described histologically in 1973 by Habib and Bois, who first noted familial occurrence. Diffuse mesangial sclerosis was later associated with an autosomal recessive inheritance pattern. The disease is a type of early onset nephrotic syndrome, often resulting in end-stage renal failure.

In one embodiment, the renal polynucleotides described herein may be used to treat and/or prevent familial mesangial sclerosis.

### Cystic Renal Diseases

In one embodiment, the renal polynucleotides described herein may be used to treat and/or prevent a Cystic renal disease such as, but not limited to, polycystic kidney disease 1, polycystic kidney disease 2, infantile sever polycystic kidney disease with tuberous sclerosis and/or familial juvenile nephronophthisis.

### Polycystic Kidney Disease 1 (PKD1) and Polycystic Kidney Disease 2 (PKD2)

Polycystic kidney disease (PKD) is a disorder characterized the growth of numerous cysts in the kidneys, leading to end-stage renal disease in 50% of cases. Polycystic kidney disease can also cause cysts in the liver, and affect the blood vessels in the brain and the heart. Two types of PKD exist, the autosomal dominant form in which symptoms begin in adulthood, and the autosomal recessive form, which is apparent at birth or infancy and is lethal early on. The autosomal dominant form can be divided into type I and type II, depending on the gene mutations involved (PKD1 and PKD2).

PKD1, a gene that encodes the transmembrane protein polycystin-1, that a role in renal tubular development, is mutated in type I. PKD2, a transmembrane protein that may function as an ion channel, is mutated in type II. Both proteins work together for normal kidney function. Mutations in the PKD2 gene often cause a less severe form of the disease than PKD1 mutations which are more frequent.

In one embodiment, the renal polynucleotides described herein may be used to treat and/or prevent Polycystic kidney disease (PKD) 1.

In one embodiment, the renal polynucleotides described herein may be used to treat and/or prevent Polycystic kidney disease (PKD) 2.

Infantile Severe Polycystic Kidney Disease with Tuberous Sclerosis

Infantile severe juvenile polycystic kidney disease with tuberous sclerosis is associated with large deletions disrupting both the TSC2 and the PKD1 genes, located adjacent to each other on chromosome 16. These genes are separately linked to tuberous sclerosis (TSC) and the autosomal dominant form of polycystic kidney disease (PKD), the symptoms of both of which include renal cysts. Patients with the deletion demonstrate markedly enlarged polycystic kidneys early during childhood, with substantially more severe symptoms than in the patients with only a single dysfunctional gene.

In one embodiment, the renal polynucleotides described herein may be used to treat and/or prevent infantile severe juvenile polycystic kidney disease with tuberous sclerosis.

### Renal Tubular Diseases

In one embodiment, the renal polynucleotides described herein may be used to treat and/or prevent a Renal tubular disease such as, but not limited to, distal renal tubular acidosis, primary hypomagnesaemia, renal tubular acidosis with neural deafness, renal tubular acidosis with osteoporosis, Dent's disease, Nephrogenic diabetes insipidus (X-linked or autosomal), familial hypocalcuric hypercalcemia, X-linked hypophosphatemia, Pseduovitamin D deficiency rickets, Bartter's syndrome type 1, Bartter's syndrome type 2, Bartter's syndrome type 3, Gitelman's syndrome, Pseudoaldosteronism (Liddle syndrome), Recessive pseudohypoaldosteronism type 1 or apparent mineralocorticoid excess.

### Distal Renal Tubular Acidosis

Distal renal tubular acidosis is a disease that occurs when the kidneys are unable to remove acid properly into the urine, leading to elevated acid levels in the blood, and lowering the pH of the blood. Symptoms include confusion and fatigue, failure to thrive in infants, short stature, increased breathing rate, kidney stones, calcium deposits in the kidney, low magnesium levels in the blood, softening of the bones (osteomalacia), rickets in children, and muscle weakness.

In one embodiment, the renal polynucleotides described herein may be used to treat and/or prevent distal renal tubular acidosis.

Renal Tubular Acidosis with Neural Deafness

Renal tubular acidosis with deafness is characterized by kidney problems and resulting hearing loss. In renal tubular acidosis, the kidneys are unable to remove acid properly into the urine, leading to elevated acid levels in the blood and lowering the blood pH. Hearing loss, with both ears affected, is caused as part of renal tubular acidosis by changes in the inner ear starting in childhood and young adulthood, and deteriorating thereafter.

In one embodiment, the renal polynucleotides described herein may be used to treat and/or prevent renal tubular acidosis with deafness.

Renal Tubular Acidosis with Osteoporosis

Renal tubular acidosis with osteoporosis is an autosomal recessive disorder due to deficiency of carbonic anhydrase II. This deficiency causes a number of disorders, including osteoporosis, a condition in which the bones harden and become denser, leading to breakage. Carbonic anhydrase II deficiency also causes renal tubular acidosis. Other symptoms include cerebral calcification, short stature, cognitive defects, and frequent bone fractures.

In one embodiment, the renal polynucleotides described herein may be used to treat and/or prevent renal tubular acidosis with osteoporosis.

### Dent's Disease

Dents disease is a rare condition that affects the structure of proximal renal tubules of the kidney, leading to kidney damage, failure and end-stage renal disease in early to mid-adulthood. Dent's disease is X-linked recessive and therefore more common in men than in women. Symptoms include large amount of proteins in the urine (tubular proteinuria), signs of excess calcium in the urine (hypercalciuria), calcium deposits in the kidneys (nephrocalcinosis), and kidney stones.

In one embodiment, the renal polynucleotides described herein may be used to treat and/or prevent Dent's disease.

### Nephrogenic Diabetes Insipidus (X-Linked) and Nephrogenic Diabetes Insipidus (Autosomal)

Nephrogenic diabetes insipidus is a type of diabetes insipidus caused by kidney pathology. Diabetes insipidus occurs when the kidneys are unable to conserve water as they are filtering blood, leading to the production of excessive urine (polyuria) and causes extreme thirst (polydipsia) and dehydration. Individuals usually have few complications, if the condition is properly managed. Nephrogenic diabetes insipidus can be either acquired or hereditary.

Most frequently the hereditary form of nephrogenic diabetes insipidus results from mutations in the AVPR2 gene, which encodes vasopressin V2 receptor, which together with its ligand, vasopressin, and controls water balance in the kidney. The condition has an X-linked recessive pattern of inheritance.

Most of the remaining cases are caused by mutations in the AQP2 gene, encoding the aquaporin 2 protein that forms a water channel across kidney cell membranes. This type can demonstrate either autosomal recessive or more rarely an autosomal dominant inheritance.

In one embodiment, the renal polynucleotides described herein may be used to treat and/or prevent nephrogenic diabetes insipidus (X-linked).

In one embodiment, the renal polynucleotides described herein may be used to treat and/or prevent nephrogenic diabetes insipidus (autosomal).

### Familial Hypocalcuric Hypercalcemia

Familial Hypocalciuric Hypercalcemia (FHH) is an autosomal dominant condition that causes abnormally high levels of calcium in the blood (hypercalcemia). Most frequently, FHH is caused by genetic mutations in the CASR gene, which produces a calcium sensing receptor. Individuals with FHH may not have any signs or symptoms, but symptoms may include weakness, fatigue, thought disturbance, and/or excessive thirst (polydipsia).

In one embodiment, the renal polynucleotides described herein may be used to treat and/or prevent Familial Hypocalciuric Hypercalcemia.

### Pseudovitamin D Deficiency Rickets

Rickets is characterized by defective mineralization of bones, causing softening and weakening of the bones. Rickets often occurs in malnourished children, and has its cause in deficiency or impaired metabolism of vitamin D, phosphorus or calcium, often causing to fractures and deformations.

Pseudo-Vitamin D Deficiency Rickets (PDDR), is a hereditary defect in a kidney enzyme involved in vitamin D synthesis, called 25-hydroxyvitamin D 1 alpha-hydroxylase. This defect leads to insufficient synthesis of calcitriol, the bioactive form of vitamin D and is the cause of early onset severe rickets.

In one embodiment, the renal polynucleotides described herein may be used to treat and/or prevent Rickets.

### X-Linked Hypophosphatemia

X-linked hypophosphatemia (XLH) (also called X-linked dominant hypophosphatemic rickets, or hypophosphatemic vitamin d-resistant rickets) is an X-linked dominant form of rickets, and has been linked to the PHEX gene. In contrast to most forms of rickets, dietary supplementation vitamin D is relatively ineffective. Symptoms include bowleggedness and short stature.

In one embodiment, the renal polynucleotides described herein may be used to treat and/or prevent X-linked hypophosphatemia (XLH).

### Gitelman's Syndrome

Gitelman's syndrome is an autosomal recessive kidney disorder that causes an imbalance of potassium, magnesium, and calcium. The disorder is usually caused by defects in the SLC12A3 and CLCNKB genes that affect the renal ability to reabsorb salt, leading to the loss of excessive salt in the urine. Symptoms typically manifest themselves in adolescence. Although there is a broad range of symptoms and severity of symptoms varies, common signs and symptoms include painful prickly skin sensations, muscle spasms, muscle weakness or cramping, dizziness, and salt craving.

In one embodiment, the renal polynucleotides described herein may be used to treat and/or prevent Gitelman's syndrome.

### Bartter's Syndrome Type 1, Type 2, and Type 3

Bartter syndrome is a group of autosomal recessively inherited kidney disorders that cause an imbalance of potassium, sodium, chloride and other molecules in the body. Two major forms of the disease exist, with one very severe, often lethal form beginning before birth, and another less severe form that begins in early childhood. Bartter syndrome is classified based on the causal mutations in at least five genes.

Type I and II along with type IV, are of the first form and disease onset is before birth. Type I and II are caused by mutations in the SLC12A1 and, KCNJ1 genes, respectively.

Mutations in the CLCNKB gene are responsible for type III and are of the second form and begin in childhood.

In one embodiment, the renal polynucleotides described herein may be used to treat and/or prevent Bartter syndrome Type 1.

In one embodiment, the renal polynucleotides described herein may be used to treat and/or prevent Bartter syndrome Type 2.

In one embodiment, the renal polynucleotides described herein may be used to treat and/or prevent Bartter syndrome Type 3.

### Pseudoaldosteronism (Liddle Syndrome)

Pseudoaldosteronism (also called Liddle syndrome) is a very rare autosomal dominant disorder characterized by early and often severe high blood pressure (hypertension). The disease is associated with low plasma renin activity, metabolic alkalosis (elevated pH in the blood), low potassium levels, and low levels of aldosterone. Symptoms include weakness, fatigue, muscle pain, constipation or palpitations. Pseudoaldosteronism is caused by mutations in either the SCNN1B or SCNN1G genes, encoding sodium channel subunits beta and gamma, two of three subunits that form the amiloride-sensitive sodium channel.

In one embodiment, the renal polynucleotides described herein may be used to treat and/or prevent Pseudoaldosteronism.

Recessive pseudohypoaldosteronism type 1

Pseudohypoaldosteronism type 1 (PHA1B) is a severe autosomal recessive disorder characterized by excessive loss of salt in the urine, high levels of sodium in the blood, and increased plasma renin and high levels of aldosterone in the blood. Excessive amounts of sodium are also seen in sweat, stool, and saliva. PHA1B is caused by mutations in the SCNN1A, SCNN1B and SCNN1G genes, encoding the subunits of the amiloride-sensitive sodium channel.

In one embodiment, the renal polynucleotides described herein may be used to treat and/or prevent Pseudohypoaldosteronism type 1 (PHA1B).

### Dominant Pseudohypoaldosteronism Type I

Pseudohypoaldosteronism type 1 (adPHA1) is a rare autosomal dominant condition that is characterized by renal resistance to aldosterone, with excessive renal excretion of salt, elevated potassium levels in the blood, and metabolic acidosis. It is considered a mild disorder and treatment is not required after childhood. adPHA1 is caused by mutations in the mineralocorticoid receptor gene.

In one embodiment, the renal polynucleotides described herein may be used to treat and/or prevent Pseudohypoaldosteronism type 1 (adPHA1).

### Apparent Mineralocorticoid Excess

Apparent mineralocorticoid excess is an autosomal recessive syndrome that is characterized by severe hypertension. The onset occurs in childhood and symptoms include hypertension, elevated potassium levels in the blood, and low levels of renin and aldosterone. Inherited apparent mineralocorticoid excess is caused by mutations in the 11β-HSD-2 gene, which encodes an enzyme that catalyzes the inactivation of cortisol. The excess in cortisol can then overstimulate of the mineralocorticoid receptor, thereby inducing hypertension. Several acquired forms of apparent mineralocorticoid excess also exist and are more common than inherited forms.

In one embodiment, the renal polynucleotides described herein may be used to treat and/or prevent apparent mineralocorticoid excess.

### Cystinuria, Type I and Non-Type I

Cystinuria is a disorder caused by dysfunctional amino acid transport, and inherited as an autosomal recessive trait. It is characterized by formation of cystine stones in the kidneys, ureter, and bladder. The disease is caused by a defect in the reabsorption of cystine and other amino acids through the renal tubule and intestinal tract. Due to the increased cysteine excretion and low solubility of cystine in urine, cystine stones form in the urine. All pathological consequences of cystinuria are related to the formation of the urinary stones and include urinary tract infection, kidney infections, obstructive uropathy, and renal insufficiency.

Cystinuria is classified into two types, based on causal mutations of the disease. Type I Cystinuria has mutation in the SLC3A1 and non-type I Cystinuria is mutated in the SLC7A9 gene. Both genes encode two parts of an amino acid transporter expressed mainly in the kidney.

In one embodiment, the renal polynucleotides described herein may be used to treat and/or prevent Cystinuria.

In one embodiment, the renal polynucleotides described herein may be used to treat and/or prevent Cystinuria Type I.

In one embodiment, the renal polynucleotides described herein may be used to treat and/or prevent Cystinuria Non-Type I.

### Wound Management

The renal polynucleotides of the present invention may be used for wound treatment, e.g. of wounds exhibiting delayed healing. Methods and compositions for using the renal polynucleotides for wound treatment are described in co-pending International Patent Publication No. WO2015038892, the contents of which is incorporated by reference in its entirety, such as, but not limited to, in paragraphs [0001089]-[0001092].

### Managing Infection

In one embodiment, provided are methods for treating or preventing a microbial infection (e.g., a bacterial infection) and/or a disease, disorder, or condition associated with a microbial or viral infection, or a symptom thereof, in a subject, by administering a renal polynucleotide encoding an anti-microbial renal polypeptide. Bacterial pathogens, antibiotic combinations, antibacterial agents, conditions associate with viral infection, viral pathogens, antiviral agents, conditions associated with fungal infections, fungal pathogens, anti-fungal agents, conditions associated with protozoal infection, protozoan pathogens, anti-protozoan agents, conditions associated with parasitic infection, parasitic pathogens, anti-parasitic agents, conditions associated with prion infection and anti-prion agents as well as compositions, delivery and methods of use of the renal polynucleotides herein are described in co-pending International Patent Publication No. WO2015038892, the contents of which is incorporated by reference in its entirety, such as, but not limited to, in paragraphs [0001096]-[0001116].

### Modulation of the Immune Response

### Avoidance of the Immune Response

As described herein, a useful feature of the renal polynucleotides of the invention is the capacity to reduce, evade or avoid the innate immune response of a cell. In one aspect, provided herein are renal polynucleotides encoding a renal polypeptide of interest which when delivered to cells, results in a reduced immune response from the host as compared to the response triggered by a reference compound, e.g. an unmodified renal polynucleotide corresponding to a renal polynucleotide of the invention, or a different renal polynucleotides of the invention. As used herein, a “reference compound” is any molecule or substance which when administered to a mammal, results in an innate immune response having a known degree, level or amount of immune stimulation. A reference compound need not be a nucleic acid molecule and it need not be any of the renal polynucleotides of the invention. Hence, the measure of a renal polynucleotides avoidance, evasion or failure to trigger an immune response can be expressed in terms relative to any compound or substance which is known to trigger such a response.

The term “innate immune response” includes a cellular response to exogenous single stranded nucleic acids, generally of viral or bacterial origin, which involves the induction of cytokine expression and release, particularly the interferons, and cell death. As used herein, the innate immune response or interferon response operates at the single cell level causing cytokine expression, cytokine release, global inhibition of protein synthesis, global destruction of cellular RNA, upregulation of major histocompatibility molecules, and/or induction of apoptotic death, induction of gene transcription of genes involved in apoptosis, anti-growth, and innate and adaptive immune cell activation. Some of the genes induced by type I IFNs include PKR, ADAR (adenosine deaminase acting on RNA), OAS (2′,5′-oligoadenylate synthetase), RNase L, and Mx proteins. PKR and ADAR lead to inhibition of translation initiation and RNA editing, respectively. OAS is a dsRNA-dependent synthetase that activates the endoribonuclease RNase L to degrade ssRNA.

In some embodiments, the innate immune response comprises expression of a Type I or Type II interferon, and the expression of the Type I or Type II interferon is not increased more than two-fold compared to a reference from a cell which has not been contacted with a renal polynucleotide of the invention.

In some embodiments, the innate immune response comprises expression of one or more IFN signature genes and where the expression of the one of more IFN signature genes is not increased more than three-fold compared to a reference from a cell which has not been contacted with the renal polynucleotides of the invention.

While in some circumstances, it might be advantageous to eliminate the innate immune response in a cell, the invention provides renal polynucleotides that upon administration result in a substantially reduced (significantly less) the immune response, including interferon signaling, without entirely eliminating such a response.

In some embodiments, the immune response is lower by 10%, 20%, 30%, 40%, 50%, 60%, 70%, 80%, 90%, 95%, 99%, 99.9%, or greater than 99.9% as compared to the immune response induced by a reference compound. The immune response itself may be measured by determining the expression or activity level of Type 1 interferons or the expression of interferon-regulated genes such as the toll-like receptors (e.g., TLR7 and TLR8). Reduction of innate immune response can also be measured by measuring the level of decreased cell death following one or more administrations to a cell population; e.g., cell death is 10%, 25%, 50%, 75%, 85%, 90%, 95%, or over 95% less than the cell death frequency observed with a reference compound. Moreover, cell death may affect fewer than 50%, 40%, 30%, 20%, 10%, 5%, 1%, 0.1%, 0.01% or fewer than 0.01% of cells contacted with the renal polynucleotides.

In another embodiment, the renal polynucleotides of the present invention is significantly less immunogenic than an unmodified in vitro-synthesized renal polynucleotide with the same sequence or a reference compound. As used herein, “significantly less immunogenic” refers to a detectable decrease in immunogenicity. In another embodiment, the term refers to a fold decrease in immunogenicity. In another embodiment, the term refers to a decrease such that an effective amount of the renal polynucleotides can be administered without triggering a detectable immune response. In another embodiment, the term refers to a decrease such that the renal polynucleotides can be repeatedly administered without eliciting an immune response sufficient to detectably reduce expression of the recombinant protein. In another embodiment, the decrease is such that the renal polynucleotides can be repeatedly administered without eliciting an immune response sufficient to eliminate detectable expression of the recombinant protein.

In another embodiment, the renal polynucleotides is 2-fold less immunogenic than its unmodified counterpart or reference compound. In another embodiment, immunogenicity is reduced by a 3-fold factor. In another embodiment, immunogenicity is reduced by a 5-fold factor. In another embodiment, immunogenicity is reduced by a 7-fold factor. In another embodiment, immunogenicity is reduced by a 10-fold factor. In another embodiment, immunogenicity is reduced by a 15-fold factor. In another embodiment, immunogenicity is reduced by a fold factor. In another embodiment, immunogenicity is reduced by a 50-fold factor. In another embodiment, immunogenicity is reduced by a 100-fold factor. In another embodiment, immunogenicity is reduced by a 200-fold factor. In another embodiment, immunogenicity is reduced by a 500-fold factor. In another embodiment, immunogenicity is reduced by a 1000-fold factor. In another embodiment, immunogenicity is reduced by a 2000-fold factor. In another embodiment, immunogenicity is reduced by another fold difference.

Methods of determining immunogenicity are well known in the art, and include, e.g. measuring secretion of cytokines (e.g. IL-12, IFNalpha, TNF-alpha, RANTES, MIP-1alpha or beta, IL-6, IFN-beta, or IL-8), measuring expression of DC activation markers (e.g. CD83, HLA-DR, CD80 and CD86), or measuring ability to act as an adjuvant for an adaptive immune response.

The renal polynucleotides of the invention, including the combination of modifications taught herein may have superior properties making them more suitable as therapeutic modalities.

It has been determined that the “all or none” model in the art is sorely insufficient to describe the biological phenomena associated with the therapeutic utility of renal pol. The present inventors have determined that to improve protein production, one may consider the nature of the modification, or combination of modifications, the percent modification and survey more than one cytokine or metric to determine the efficacy and risk profile of a particular renal polynucleotide.

In one aspect of the invention, methods of determining the effectiveness of a renal polynucleotide as compared to unmodified involves the measure and analysis of one or more cytokines whose expression is triggered by the administration of the exogenous nucleic acid of the invention. These values are compared to administration of an unmodified nucleic acid or to a standard metric such as cytokine response, PolyIC, R-848 or other standard known in the art.

One example of a standard metric developed herein is the measure of the ratio of the level or amount of encoded renal polypeptide (protein) produced in the cell, tissue or organism to the level or amount of one or more (or a panel) of cytokines whose expression is triggered in the cell, tissue or organism as a result of administration or contact with the modified nucleic acid. Such ratios are referred to herein as the Protein:Cytokine Ratio or “PC” Ratio. The higher the PC ratio, the more efficacious the modified nucleic acid (renal polynucleotide encoding the protein measured). Preferred PC Ratios, by cytokine, of the present invention may be greater than 1, greater than 10, greater than 100, greater than 1000, greater than 10,000 or more. Modified nucleic acids having higher PC Ratios than a modified nucleic acid of a different or unmodified construct are preferred.

The PC ratio may be further qualified by the percent modification present in the renal polynucleotide. For example, normalized to a 100% modified nucleic acid, the protein production as a function of cytokine (or risk) or cytokine profile can be determined.

In one embodiment, the present invention provides a method for determining, across chemistries, cytokines or percent modification, the relative efficacy of any particular modified the renal polynucleotides by comparing the PC Ratio of the modified nucleic acid (renal polynucleotides).

Renal polynucleotides containing varying levels of nucleobase substitutions could be produced that maintain increased protein production and decreased immunostimulatory potential. The relative percentage of any modified nucleotide to its naturally occurring nucleotide counterpart can be varied during the IVT reaction (for instance, 100, 50, 25, 10, 5, 2.5, 1, 0.1, 0.01% 5 methyl cytidine usage versus cytidine; 100, 50, 25, 10, 5, 2.5, 1, 0.1, 0.01% pseudouridine or N1-methyl-pseudouridine usage versus uridine). Renal polynucleotides can also be made that utilize different ratios using 2 or more different nucleotides to the same base (for instance, different ratios of pseudouridine and N1-methyl-pseudouridine). Renal polynucleotides can also be made with mixed ratios at more than 1 “base” position, such as ratios of 5 methyl cytidine/cytidine and pseudouridine/N1-methyl-pseudouridine/uridine at the same time. Use of renal polynucleotides with altered ratios of modified nucleotides can be beneficial in reducing potential exposure to chemically modified nucleotides. Lastly, positional introduction of modified nucleotides into the renal polynucleotides which modulate either protein production or immunostimulatory potential or both is also possible. The ability of such renal polynucleotides to demonstrate these improved properties can be assessed in vitro (using assays such as the PBMC assay described herein), and can also be assessed in vivo through measurement of both renal polynucleotides-encoded protein production and mediators of innate immune recognition such as cytokines.

In another embodiment, the relative immunogenicity of the renal polynucleotides and its unmodified counterpart are determined by determining the quantity of the renal polynucleotides required to elicit one of the above responses to the same degree as a given quantity of the unmodified nucleotide or reference compound. For example, if twice as much renal polynucleotides is required to elicit the same response, than the renal polynucleotides is two-fold less immunogenic than the unmodified nucleotide or the reference compound.

In another embodiment, the relative immunogenicity of the renal polynucleotides and its unmodified counterpart are determined by determining the quantity of cytokine (e.g. IL-12, IFNalpha, TNF-alpha, RANTES, MIP-1alpha or beta, IL-6, IFN-beta, or IL-8) secreted in response to administration of the renal polynucleotides, relative to the same quantity of the unmodified nucleotide or reference compound. For example, if one-half as much cytokine is secreted, than the renal polynucleotides is two-fold less immunogenic than the unmodified nucleotide. In another embodiment, background levels of stimulation are subtracted before calculating the immunogenicity in the above methods.

Provided herein are also methods for performing the titration, reduction or elimination of the immune response in a cell or a population of cells. In some embodiments, the cell is contacted with varied doses of the same renal polynucleotides and dose response is evaluated. In some embodiments, a cell is contacted with a number of different renal polynucleotides at the same or different doses to determine the optimal composition for producing the desired effect. Regarding the immune response, the desired effect may be to avoid, evade or reduce the immune response of the cell. The desired effect may also be to alter the efficiency of protein production.

The renal polynucleotides of the present invention may be used to reduce the immune response using the method described in International Publication No. WO2013003475, herein incorporated by reference in its entirety.

### Activation of the Immune Response: Vaccines

According to the present invention, the renal polynucleotides disclosed herein, may encode one or more vaccines. As used herein, a “vaccine” is a biological preparation that improves immunity to a particular disease or infectious agent. A vaccine introduces an antigen into the tissues or cells of a subject and elicits an immune response, thereby protecting the subject from a particular disease or pathogen infection. The renal polynucleotides of the present invention may encode an antigen and when the renal polynucleotides yield protein expression in cells, a desired immune response is achieved. Renal polynucleotides which may be a vaccine, compositions and methods of use are described in co-pending International Patent Publication No. WO2015038892, the contents of which is incorporated by reference in its entirety, such as, but not limited to, in paragraphs [0001137]-[0001173].

### Naturally Occurring Mutants

In another embodiment, the renal polynucleotides can be utilized to express variants of naturally occurring proteins that have an improved disease modifying activity, including increased biological activity, improved patient outcomes, or a protective function, etc., as described in co-pending International Patent Publication No. WO2015038892, the contents of which is incorporated by reference in its entirety, such as, but not limited to, in paragraphs [0001174]-[0001175].

### Renal Polypeptide Libraries

In one embodiment, the renal polynucleotides may be used to produce renal polypeptide libraries. These libraries may arise from the production of a population of renal polynucleotides, each containing various structural or chemical modification designs. In this embodiment, a population of renal polynucleotides may comprise a plurality of encoded renal polypeptides, including but not limited to, an antibody or antibody fragment, protein binding partner, scaffold protein, and other renal polypeptides taught herein or known in the art. In one embodiment, the renal polynucleotides may be suitable for direct introduction into a target cell or culture which in turn may synthesize the encoded renal polypeptides.

In certain embodiments, multiple variants of a protein, each with different amino acid modification(s), may be produced and tested to determine the best variant in terms of pharmacokinetics, stability, biocompatibility, and/or biological activity, or a biophysical property such as expression level. Such a library may contain 10, 10², 10³, 10⁴, 10⁵, 10⁶, 10⁷, 10⁸, 10⁹, or over 10⁹ possible variants (including, but not limited to, substitutions, deletions of one or more residues, and insertion of one or more residues).

### Targeting of Pathogenic Organisms or Diseased Cells

Provided herein are methods for targeting pathogenic microorganisms, such as bacteria, yeast, protozoa, helminthes and the like, or diseased cells such as cancer cells using renal polynucleotides that encode cytostatic or cytotoxic renal polypeptides. Preferably the mRNA introduced contains modified nucleosides or other nucleic acid sequence modifications that are translated exclusively, or preferentially, in the target pathogenic organism, to reduce possible off-target effects of the therapeutic. Such methods are useful for removing pathogenic organisms or killing diseased cells found in any biological material, including blood, semen, eggs, and transplant materials including embryos, tissues, and organs.

### Bioprocessing

The methods provided herein may be useful for enhancing protein product yield in a cell culture process as described in co-pending International Patent Publication No. WO2015038892, the contents of which is incorporated by reference in its entirety, such as, but not limited to, in paragraphs [0001176]-[0001187].

### Cells

In one embodiment, the cells are selected from the group consisting of mammalian cells, bacterial cells, plant, microbial, algal and fungal cells. In some embodiments, the cells are mammalian cells, such as, but not limited to, human, mouse, rat, goat, horse, rabbit, hamster or cow cells. In a further embodiment, the cells may be from an established cell line, including, but not limited to, HeLa, NSO, SP2/0, KEK 293T, Vero, Caco, Caco-2, MDCK, COS-1, COS-7, K562, Jurkat, CHO-K1, DG44, CHOK1SV, CHO-S, Huvec, CV-1, Huh-7, NIH3T3, HEK293, 293, A549, HepG2, IMR-90, MCF-7, U-20S, Per.C6, SF9, SF21 or Chinese Hamster Ovary (CHO) cells.

In certain embodiments, the cells are fungal cells, such as, but not limited to, Chrysosporium cells, Aspergillus cells, Trichoderma cells, Dictyostelium cells, Candida cells, Saccharomyces cells, Schizosaccharomyces cells, and Penicillium cells.

In certain embodiments, the cells are bacterial cells such as, but not limited to, E. coli, B. subtilis, or BL21 cells. Primary and secondary cells to be transfected by the methods of the invention can be obtained from a variety of tissues and include, but are not limited to, all cell types which can be maintained in culture. For examples, primary and secondary cells which can be transfected by the methods of the invention include, but are not limited to, fibroblasts, keratinocytes, epithelial cells (e.g., mammary epithelial cells, intestinal epithelial cells), endothelial cells, glial cells, neural cells, formed elements of the blood (e.g., lymphocytes, bone marrow cells), muscle cells and precursors of these somatic cell types. Primary cells may also be obtained from a donor of the same species or from another species (e.g., mouse, rat, rabbit, cat, dog, pig, cow, bird, sheep, goat, horse).

### Purification and Isolation

Those of ordinary skill in the art should be able to make a determination of the methods to use to purify or isolate of a protein of interest from cultured cells. Generally, this is done through a capture method using affinity binding or non-affinity purification. If the protein of interest is not secreted by the cultured cells, then a lysis of the cultured cells should be performed prior to purification or isolation. One may use unclarified cell culture fluid containing the protein of interest along with cell culture media components as well as cell culture additives, such as anti-foam compounds and other nutrients and supplements, cells, cellular debris, host cell proteins, DNA, viruses and the like in the present invention. The process may be conducted in the bioreactor itself. The fluid may either be preconditioned to a desired stimulus such as pH, temperature or other stimulus characteristic or the fluid can be conditioned upon the addition of polymer(s) or the polymer(s) can be added to a carrier liquid that is properly conditioned to the required parameter for the stimulus condition required for that polymer to be solubilized in the fluid. The polymer may be allowed to circulate thoroughly with the fluid and then the stimulus may be applied (change in pH, temperature, salt concentration, etc.) and the desired protein and polymer(s) precipitate can out of the solution. The polymer and the desired protein(s) can be separated from the rest of the fluid and optionally washed one or more times to remove any trapped or loosely bound contaminants. The desired protein may then be recovered from the polymer(s) by, for example, elution and the like. Preferably, the elution may be done under a set of conditions such that the polymer remains in its precipitated form and retains any impurities to it during the selected elution of the desired protein. The polymer and protein as well as any impurities may be solubilized in a new fluid such as water or a buffered solution and the protein may be recovered by a means such as affinity, ion exchanged, hydrophobic, or some other type of chromatography that has a preference and selectivity for the protein over that of the polymer or impurities. The eluted protein may then be recovered and may be subjected to additional processing steps, either batch like steps or continuous flow through steps if appropriate.

In another embodiment, it may be useful to optimize the expression of a specific renal polypeptide in a cell line or collection of cell lines of potential interest, particularly a renal polypeptide of interest such as a protein variant of a reference protein having a known activity. In one embodiment, provided is a method of optimizing expression of a renal polypeptide of interest in a target cell, by providing a plurality of target cell types, and independently contacting with each of the plurality of target cell types a renal polynucleotide encoding a renal polypeptide. Additionally, culture conditions may be altered to increase protein production efficiency. Subsequently, the presence and/or level of the renal polypeptide of interest in the plurality of target cell types is detected and/or quantitated, allowing for the optimization of a renal polypeptide of interest's expression by selection of an efficient target cell and cell culture conditions relating thereto. Such methods may be useful when the renal polypeptide of interest contains one or more post-translational modifications or has substantial tertiary structure, which often complicate efficient protein production.

### Protein Recovery

The protein of interest may be preferably recovered from the culture medium as a secreted renal polypeptide, or it can be recovered from host cell lysates if expressed without a secretory signal. It may be necessary to purify the protein of interest from other recombinant proteins and host cell proteins in a way that substantially homogenous preparations of the protein of interest are obtained. The cells and/or particulate cell debris may be removed from the culture medium or lysate. The product of interest may then be purified from contaminant soluble proteins, renal polypeptides and nucleic acids by, for example, fractionation on immunoaffinity or ion-exchange columns, ethanol precipitation, reverse phase HPLC (RP-HPLC), SEPHADEX® chromatography, and chromatography on silica or on a cation exchange resin such as DEAE. Methods of purifying a protein heterologous expressed by a host cell are well known in the art.

Methods and compositions described herein may be used to produce proteins which are capable of attenuating or blocking the endogenous agonist biological response and/or antagonizing a receptor or signaling molecule in a mammalian subject. For example, IL-12 and IL-23 receptor signaling may be enhanced in chronic autoimmune disorders such as multiple sclerosis and inflammatory diseases such as rheumatoid arthritis, psoriasis, lupus erythematosus, ankylosing spondylitis and Chron's disease (Kikly K, Liu L, Na S, Sedgwich J D (2006) Cur. Opin. Immunol. 18(6): 670-5). In another embodiment, a nucleic acid encodes an antagonist for chemokine receptors. Chemokine receptors CXCR-4 and CCR-5 are required for HIV entry into host cells (Arenzana-Seisdedos F et al, (1996) Nature. October 3; 383 (6599):400).

### Gene Silencing

The renal polynucleotides described herein are useful to silence (i.e., prevent or substantially reduce) expression of one or more target genes in a cell population. A renal polynucleotide encoding a renal polypeptide of interest capable of directing sequence-specific histone H3 methylation is introduced into the cells in the population under conditions such that the renal polypeptide is translated and reduces gene transcription of a target gene via histone H3 methylation and subsequent heterochromatin formation. In some embodiments, the silencing mechanism is performed on a cell population present in a mammalian subject. By way of non-limiting example, a useful target gene is a mutated Janus Kinase-2 family member, wherein the mammalian subject expresses the mutant target gene suffers from a myeloproliferative disease resulting from aberrant kinase activity.

Co-administration of renal polynucleotides and RNAi agents are also provided herein.

### Modulation of Biological Pathways

The rapid translation renal polynucleotides introduced into cells provides a desirable mechanism of modulating target biological pathways. Such modulation includes antagonism or agonism of a given pathway. In one embodiment, a method is provided for antagonizing a biological pathway in a cell by contacting the cell with an effective amount of a composition comprising a renal polynucleotide encoding a renal polypeptide of interest, under conditions such that the renal polynucleotides is localized into the cell and the renal polypeptide is capable of being translated in the cell from the renal polynucleotides, wherein the renal polypeptide inhibits the activity of a renal polypeptide functional in the biological pathway. Exemplary biological pathways are those defective in an autoimmune or inflammatory disorder such as multiple sclerosis, rheumatoid arthritis, psoriasis, lupus erythematosus, ankylosing spondylitis colitis, or Crohn's disease; in particular, antagonism of the IL-12 and IL-23 signaling pathways are of particular utility. (See Kikly K, Liu L, Na S, Sedgwick J D (2006) Curr. Opin. Immunol. 18 (6): 670-5).

Further, provided are renal polynucleotides encoding an antagonist for chemokine receptors; chemokine receptors CXCR-4 and CCR-5 are required for, e.g., HIV entry into host cells (Arenzana-Seisdedos F et al, (1996) Nature. October 3; 383(6599):400).

Alternatively, provided are methods of agonizing a biological pathway in a cell by contacting the cell with an effective amount of a renal polynucleotide encoding a recombinant renal polypeptide under conditions such that the nucleic acid is localized into the cell and the recombinant renal polypeptide is capable of being translated in the cell from the nucleic acid, and the recombinant renal polypeptide induces the activity of a renal polypeptide functional in the biological pathway. Exemplary agonized biological pathways include pathways that modulate cell fate determination. Such agonization is reversible or, alternatively, irreversible.

### Expression of Ligand or Receptor on Cell Surface

In some aspects and embodiments of the aspects described herein, the renal polynucleotides described herein can be used to express a ligand or ligand receptor on the surface of a cell (e.g., a homing moiety). A ligand or ligand receptor moiety attached to a cell surface can permit the cell to have a desired biological interaction with a tissue or an agent in vivo. A ligand can be an antibody, an antibody fragment, an aptamer, a renal peptide, a vitamin, a carbohydrate, a protein or renal polypeptide, a receptor, e.g., cell-surface receptor, an adhesion molecule, a glycoprotein, a sugar residue, a therapeutic agent, a drug, a glycosaminoglycan, or any combination thereof. For example, a ligand can be an antibody that recognizes a cancer-cell specific antigen, rendering the cell capable of preferentially interacting with tumor cells to permit tumor-specific localization of a modified cell. A ligand can confer the ability of a cell composition to accumulate in a tissue to be treated, since a preferred ligand may be capable of interacting with a target molecule on the external face of a tissue to be treated. Ligands having limited cross-reactivity to other tissues are generally preferred.

In some cases, a ligand can act as a homing moiety which permits the cell to target to a specific tissue or interact with a specific ligand. Such homing moieties can include, but are not limited to, any member of a specific binding pair, antibodies, monoclonal antibodies, or derivatives or analogs thereof, including without limitation: Fv fragments, single chain Fv (scFv) fragments, Fab′ fragments, F(ab′)2 fragments, single domain antibodies, camelized antibodies and antibody fragments, humanized antibodies and antibody fragments, and multivalent versions of the foregoing; multivalent binding reagents including without limitation: monospecific or bispecific antibodies, such as disulfide stabilized Fv fragments, scFv tandems ((SCFV)2 fragments), diabodies, tribodies or tetrabodies, which typically are covalently linked or otherwise stabilized (i.e., leucine zipper or helix stabilized) scFv fragments; and other homing moieties include for example, aptamers, receptors, and fusion proteins.

In some embodiments, the homing moiety may be a surface-bound antibody, which can permit tuning of cell targeting specificity. This is especially useful since highly specific antibodies can be raised against an epitope of interest for the desired targeting site. In one embodiment, multiple antibodies are expressed on the surface of a cell, and each antibody can have a different specificity for a desired target. Such approaches can increase the avidity and specificity of homing interactions.

A skilled artisan can select any homing moiety based on the desired localization or function of the cell, for example an estrogen receptor ligand, such as tamoxifen, can target cells to estrogen-dependent breast cancer cells that have an increased number of estrogen receptors on the cell surface. Other non-limiting examples of ligand/receptor interactions include CCRI (e.g., for treatment of inflamed joint tissues or brain in rheumatoid arthritis, and/or multiple sclerosis), CCR7, CCR8 (e.g., targeting to lymph node tissue), CCR6, CCR9, CCR10 (e.g., to target to intestinal tissue), CCR4, CCR10 (e.g., for targeting to skin), CXCR4 (e.g., for general enhanced transmigration), HCELL (e.g., for treatment of inflammation and inflammatory disorders, bone marrow), Alpha4beta7 (e.g., for intestinal mucosa targeting), VLA-4/VCAM-1 (e.g., targeting to endothelium). In general, any receptor involved in targeting (e.g., cancer metastasis) can be harnessed for use in the methods and compositions described herein.

### Modulation of Cell Lineage

Provided are methods of inducing an alteration in cell fate in a target mammalian cell. The target mammalian cell may be a precursor cell and the alteration may involve driving differentiation into a lineage, or blocking such differentiation. Alternatively, the target mammalian cell may be a differentiated cell, and the cell fate alteration includes driving de-differentiation into a pluripotent precursor cell, or blocking such de-differentiation, such as the dedifferentiation of cancer cells into cancer stem cells. In situations where a change in cell fate is desired, effective amounts of mRNAs encoding a cell fate inductive renal polypeptide is introduced into a target cell under conditions such that an alteration in cell fate is induced. In some embodiments, the renal polynucleotides are useful to reprogram a subpopulation of cells from a first phenotype to a second phenotype. Such a reprogramming may be temporary or permanent. Optionally, the reprogramming induces a target cell to adopt an intermediate phenotype.

Additionally, the methods of the present invention are particularly useful to generate induced pluripotent stem cells (iPS cells) because of the high efficiency of transfection, the ability to re-transfect cells, and the tenability of the amount of recombinant renal polypeptides produced in the target cells. Further, the use of iPS cells generated using the methods described herein is expected to have a reduced incidence of teratoma formation.

Also provided are methods of reducing cellular differentiation in a target cell population. For example, a target cell population containing one or more precursor cell types is contacted with a composition having an effective amount of a renal polynucleotides encoding a renal polypeptide, under conditions such that the renal polypeptide is translated and reduces the differentiation of the precursor cell. In non-limiting embodiments, the target cell population contains injured tissue in a mammalian subject or tissue affected by a surgical procedure. The precursor cell is, e.g., a stromal precursor cell, a neural precursor cell, or a mesenchymal precursor cell.

In a specific embodiment, provided are renal polynucleotides that encode one or more differentiation factors Gata4, Mef2c and Tbx4. These mRNA-generated factors are introduced into fibroblasts and drive the reprogramming into cardiomyocytes. Such a reprogramming can be performed in vivo, by contacting an mRNA-containing patch or other material to damaged cardiac tissue to facilitate cardiac regeneration. Such a process promotes cardiomyocyte genesis as opposed to fibrosis.

### Mediation of Cell Death

In one embodiment, renal polynucleotides compositions can be used to induce apoptosis in a cell (e.g., a cancer cell) by increasing the expression of a death receptor, a death receptor ligand or a combination thereof. This method can be used to induce cell death in any desired cell and has particular usefulness in the treatment of cancer where cells escape natural apoptotic signals.

Apoptosis can be induced by multiple independent signaling pathways that converge upon a final effector mechanism consisting of multiple interactions between several “death receptors” and their ligands, which belong to the tumor necrosis factor (TNF) receptor/ligand superfamily. The best-characterized death receptors are CD95 (“Fas”), TNFRI (p55), death receptor 3 (DR3 or Apo3/TRAMO), DR4 and DR5 (apo2-TRAIL-R2). The final effector mechanism of apoptosis may be the activation of a series of proteinases designated as caspases. The activation of these caspases results in the cleavage of a series of vital cellular proteins and cell death. The molecular mechanism of death receptors/ligands-induced apoptosis is well known in the art. For example, Fas/FasL-mediated apoptosis is induced by binding of three FasL molecules which induces trimerization of Fas receptor via C-terminus death domains (DDs), which in turn recruits an adapter protein FADD (Fas-associated protein with death domain) and Caspase-8. The oligomerization of this trimolecular complex, Fas/FAIDD/caspase-8, results in proteolytic cleavage of proenzyme caspase-8 into active caspase-8 that, in turn, initiates the apoptosis process by activating other downstream caspases through proteolysis, including caspase-3. Death ligands in general are apoptotic when formed into trimers or higher order of structures. As monomers, they may serve as antiapoptotic agents by competing with the trimers for binding to the death receptors.

In one embodiment, the renal polynucleotides composition encodes for a death receptor (e.g., Fas, TRAIL, TRAMO, TNFR, and TLR etc.). Cells made to express a death receptor by transfection of renal polynucleotides become susceptible to death induced by the ligand that activates that receptor. Similarly, cells made to express a death ligand, e.g., on their surface, will induce death of cells with the receptor when the transfected cell contacts the target cell. In another embodiment, the renal polynucleotides composition encodes for a death receptor ligand (e.g., FasL, TNF, etc.). In another embodiment, the renal polynucleotides composition encodes a caspase (e.g., caspase 3, caspase 8, caspase 9 etc.). Where cancer cells often exhibit a failure to properly differentiate to a non-proliferative or controlled proliferative form, in another embodiment, the synthetic, renal polynucleotides composition encodes for both a death receptor and its appropriate activating ligand. In another embodiment, the synthetic, renal polynucleotides composition encodes for a differentiation factor that when expressed in the cancer cell, such as a cancer stem cell, will induce the cell to differentiate to a non-pathogenic or nonself-renewing phenotype (e.g., reduced cell growth rate, reduced cell division etc.) or to induce the cell to enter a dormant cell phase (e.g., Go resting phase).

One of skill in the art will appreciate that the use of apoptosis-inducing techniques may require that the renal polynucleotides are appropriately targeted to e.g., tumor cells to prevent unwanted wide-spread cell death. Thus, one can use a delivery mechanism (e.g., attached ligand or antibody, targeted liposome etc.) that recognizes a cancer antigen such that the renal polynucleotides are expressed only in cancer cells.

### Conjugates and Combinations of Renal Polynucleotides

In order to further enhance protein production, renal polynucleotides of the present invention can be designed to be conjugated to other renal polynucleotides, dyes, intercalating agents (e.g. acridines), cross-linkers (e.g. psoralene, mitomycin C), porphyrins (TPPC4, texaphyrin, Sapphyrin), polycyclic aromatic hydrocarbons (e.g., phenazine, dihydrophenazine), artificial endonucleases (e.g. EDTA), alkylating agents, phosphate, amino, mercapto, PEG (e.g., PEG-40K), MPEG, [MPEG]2, polyamino, alkyl, substituted alkyl, radiolabeled markers, enzymes, haptens (e.g. biotin), transport/absorption facilitators (e.g., aspirin, vitamin E, folic acid), synthetic ribonucleases, proteins, e.g., glycoproteins, or renal peptides, e.g., molecules having a specific affinity for a co-ligand, or antibodies e.g., an antibody, that binds to a specified cell type such as a cancer cell, endothelial cell, or bone cell, hormones and hormone receptors, non-peptidic species, such as lipids, lectins, carbohydrates, vitamins, cofactors, or a drug.

Conjugation may result in increased stability and/or half-life and may be particularly useful in targeting the renal polynucleotides to specific sites in the cell, tissue or organism.

According to the present invention, the renal polynucleotides may be administered with, conjugated to or further encode one or more of RNAi agents, siRNAs, shRNAs, miRNAs, miRNA binding sites, antisense RNAs, ribozymes, catalytic DNA, tRNA, RNAs that induce triple helix formation, aptamers or vectors, and the like.

### VI. KITS AND DEVICES

### Kits

The invention provides a variety of kits for conveniently and/or effectively carrying out methods of the present invention. Typically kits will comprise sufficient amounts and/or numbers of components to allow a user to perform multiple treatments of a subject(s) and/or to perform multiple experiments.

In one aspect, the present invention provides kits comprising the molecules (renal polynucleotides) of the invention. In one embodiment, the kit comprises one or more functional antibodies or function fragments thereof.

The kits can be for protein production, comprising a first renal polynucleotides comprising a translatable region. The kit may further comprise packaging and instructions and/or a delivery agent to form a formulation composition. The delivery agent may comprise a saline, a buffered solution, a lipidoid or any delivery agent disclosed herein.

In one embodiment, the buffer solution may include sodium chloride, calcium chloride, phosphate and/or EDTA. In another embodiment, the buffer solution may include, but is not limited to, saline, saline with 2 mM calcium, 5% sucrose, 5% sucrose with 2 mM calcium, 5% Mannitol, 5% Mannitol with 2 mM calcium, Ringer's lactate, sodium chloride, sodium chloride with 2 mM calcium and mannose (See e.g., U.S. Pub. No. 20120258046; herein incorporated by reference in its entirety). In a further embodiment, the buffer solutions may be precipitated or it may be lyophilized. The amount of each component may be varied to enable consistent, reproducible higher concentration saline or simple buffer formulations. The components may also be varied in order to increase the stability of modified RNA in the buffer solution over a period of time and/or under a variety of conditions. In one aspect, the present invention provides kits for protein production, comprising: a renal polynucleotide comprising a translatable region, provided in an amount effective to produce a desired amount of a protein encoded by the translatable region when introduced into a target cell; a second renal polynucleotide comprising an inhibitory nucleic acid, provided in an amount effective to substantially inhibit the innate immune response of the cell; and packaging and instructions.

In one aspect, the present invention provides kits for protein production, comprising a renal polynucleotide comprising a translatable region, wherein the renal polynucleotide exhibits reduced degradation by a cellular nuclease, and packaging and instructions.

In one aspect, the present invention provides kits for protein production, comprising a renal polynucleotide comprising a translatable region, wherein the renal polynucleotide exhibits reduced degradation by a cellular nuclease, and a mammalian cell suitable for translation of the translatable region of the first nucleic acid.

### Devices

The present invention provides for devices which may incorporate renal polynucleotides that encode renal polypeptides of interest. These devices contain in a stable formulation the reagents to synthesize a renal polynucleotide in a formulation available to be immediately delivered to a subject in need thereof, such as a human patient

Devices for administration may be employed to deliver the renal polynucleotides of the present invention according to single, multi- or split-dosing regimens taught herein. Such devices are taught in, for example, International Application PCT/US2013/30062 filed Mar. 9, 2013 (Attorney Docket Number M300), the contents of which are incorporated herein by reference in their entirety.

Method and devices known in the art for multi-administration to cells, organs and tissues are contemplated for use in conjunction with the methods and compositions disclosed herein as embodiments of the present invention. These include, for example, those methods and devices having multiple needles, hybrid devices employing for example lumens or catheters as well as devices utilizing heat, electric current or radiation driven mechanisms.

According to the present invention, these multi-administration devices may be utilized to deliver the single, multi- or split doses contemplated herein. Such devices are taught for example in, International Application PCT/US2013/30062 filed Mar. 9, 2013 (Attorney Docket Number M300), the contents of which are incorporated herein by reference in their entirety.

In one embodiment, the renal polynucleotide is administered subcutaneously or intramuscularly via at least 3 needles to three different, optionally adjacent, sites simultaneously, or within a 60 minutes period (e.g., administration to 4,5, 6, 7, 8, 9, or 10 sites simultaneously or within a 60 minute period).

Methods of delivering therapeutic agents using solid biodegradable microneedles are described by O'hagan et al. in US Patent Publication No. US20130287832, the contents of which are herein incorporated by reference in its entirety. The microneedles are fabricated from the therapeutic agent (e.g., influenza vaccine) in combination with at least one solid excipient. After penetrating the skin, the microneedles dissolve in situ and release the therapeutic agent to the subject. As a non-limiting example, the therapeutic agents used in the fabrication of the microneedles are the renal polynucleotides described herein.

A microneedle assembly for transdermal drug delivery is described by Ross et al. in U.S. Pat. No. 8,636,696, the contents of which are herein incorporated by reference in its entirety. The assembly has a first surface and a second surface where the microneedles project outwardly from the second surface of the support. The assembly may include a channel and aperture to form a junction which allows fluids (e.g., therapeutic agents or drugs) to pass.

Methods and Devices Utilizing Catheters and/or Lumens

Methods and devices using catheters and lumens may be employed to administer the renal polynucleotides of the present invention on a single, multi- or split dosing schedule. Such methods and devices are described in International Application PCT/US2013/30062 filed Mar. 9, 2013 (Attorney Docket Number M300), the contents of which are incorporated herein by reference in their entirety.

### Methods and Devices Utilizing Electrical Current

Methods and devices utilizing electric current may be employed to deliver the renal polynucleotides of the present invention according to the single, multi- or split dosing regimens taught herein. Such methods and devices are described in International Application PCT/US2013/30062 filed Mar. 9, 2013 (Attorney Docket Number M300), the contents of which are incorporated herein by reference in their entirety.

### VII. DEFINITIONS

At various places in the present specification, substituents of compounds of the present disclosure are disclosed in groups or in ranges. It is specifically intended that the present disclosure include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₅ alkyl. Herein a phrase of the form “optionally substituted X” (e.g., optionally substituted alkyl) is intended to be equivalent to “X, wherein X is optionally substituted” (e.g., “alkyl, wherein said alkyl is optionally substituted”). It is not intended to mean that the feature “X” (e.g. alkyl) per se is optional.

About: As used herein, the term “about” means +/−10% of the recited value.

Administered in combination: As used herein, the term “administered in combination” or “combined administration” means that two or more agents are administered to a subject at the same time or within an interval such that there may be an overlap of an effect of each agent on the patient. In some embodiments, they are administered within about 60, 30, 15, 10, 5, or 1 minute of one another. In some embodiments, the administrations of the agents are spaced sufficiently closely together such that a combinatorial (e.g., a synergistic) effect is achieved.

Adjuvant: As used herein, the term “adjuvant” means a substance that enhances a subject's immune response to an antigen.

Animal: As used herein, the term “animal” refers to any member of the animal kingdom. In some embodiments, “animal” refers to humans at any stage of development. In some embodiments, “animal” refers to non-human animals at any stage of development. In certain embodiments, the non-human animal is a mammal (e.g., a rodent, a mouse, a rat, a rabbit, a monkey, a dog, a cat, a sheep, cattle, a primate, or a pig). In some embodiments, animals include, but are not limited to, mammals, birds, reptiles, amphibians, fish, and worms. In some embodiments, the animal is a transgenic animal, genetically-engineered animal, or a clone.

Antigen: As used herein, the term “antigen” refers to the substance that binds specifically to the respective antibody. An antigen may originate either from the body, such as cancer antigen used herein, or from the external environment, for instance, from infectious agents.

Antigens of interest or desired antigens: As used herein, the terms “antigens of interest” or “desired antigens” include those proteins and other biomolecules provided herein that are immunospecifically bound by the antibodies and fragments, mutants, variants, and alterations thereof described herein. Examples of antigens of interest include, but are not limited to, insulin, insulin-like growth factor, hGH, tPA, cytokines, such as interleukins (IL), e.g., IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, interferon (IFN) alpha, IFN beta, IFN gamma, IFN omega or IFN tau, tumor necrosis factor (TNF), such as TNF alpha and TNF beta, TNF gamma, TRAIL; G-CSF, GM-CSF, M-CSF, MCP-1 and VEGF.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: As used herein, the terms “associated with,” “conjugated,” “linked,” “attached,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. An “association” need not be strictly through direct covalent chemical bonding. It may also suggest ionic or hydrogen bonding or a hybridization based connectivity sufficiently stable such that the “associated” entities remain physically associated.

Bifunctional: As used herein, the term “bifunctional” refers to any substance, molecule or moiety which is capable of or maintains at least two functions. The functions may affect the same outcome or a different outcome. The structure that produces the function may be the same or different. For example, bifunctional modified RNAs of the present invention may encode a cytotoxic renal peptide (a first function) while those nucleosides which comprise the encoding RNA are, in and of themselves, cytotoxic (second function). In this example, delivery of the bifunctional modified RNA to a cancer cell would produce not only a renal peptide or protein molecule which may ameliorate or treat the cancer but would also deliver a cytotoxic payload of nucleosides to the cell should degradation, instead of translation of the modified RNA, occur.

Biocompatible: As used herein, the term “biocompatible” means compatible with living cells, tissues, organs or systems posing little to no risk of injury, toxicity or rejection by the immune system.

Biodegradable: As used herein, the term “biodegradable” means capable of being broken down into innocuous products by the action of living things.

Biologically active: As used herein, the phrase “biologically active” refers to a characteristic of any substance that has activity in a biological system and/or organism. For instance, a substance that, when administered to an organism, has a biological effect on that organism, is considered to be biologically active. In particular embodiments, a renal polynucleotide of the present invention may be considered biologically active if even a portion of the renal polynucleotides is biologically active or mimics an activity considered biologically relevant.

Chimera: As used herein, “chimera” is an entity having two or more incongruous or heterogeneous parts or regions.

Chimeric renal polynucleotide: As used herein, “chimeric renal polynucleotides” are those nucleic acid polymers having portions or regions which differ in size and/or chemical modification pattern, chemical modification position, chemical modification percent or chemical modification population and combinations of the foregoing.

Compound: As used herein, the term “compound,” is meant to include all stereoisomers, geometric isomers, tautomers, and isotopes of the structures depicted.

The compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

Compounds of the present disclosure also include tautomeric forms. Tautomeric forms result from the swapping of a single bond with an adjacent double bond and the concomitant migration of a proton. Tautomeric forms include prototropic tautomers which are isomeric protonation states having the same empirical formula and total charge. Examples prototropic tautomers include ketone-enol pairs, amide-imidic acid pairs, lactam-lactim pairs, amide-imidic acid pairs, enamine-imine pairs, and annular forms where a proton can occupy two or more positions of a heterocyclic system, such as, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Compounds of the present disclosure also include all of the isotopes of the atoms occurring in the intermediate or final compounds. “Isotopes” refers to atoms having the same atomic number but different mass numbers resulting from a different number of neutrons in the nuclei. For example, isotopes of hydrogen include tritium and deuterium.

The compounds and salts of the present disclosure can be prepared in combination with solvent or water molecules to form solvates and hydrates by routine methods.

Conserved: As used herein, the term “conserved” refers to nucleotides or amino acid residues of a renal polynucleotide sequence or renal polypeptide sequence, respectively, that are those that occur unaltered in the same position of two or more sequences being compared. Nucleotides or amino acids that are relatively conserved are those that are conserved amongst more related sequences than nucleotides or amino acids appearing elsewhere in the sequences.

In some embodiments, two or more sequences are said to be “completely conserved” if they are 100% identical to one another. In some embodiments, two or more sequences are said to be “highly conserved” if they are at least 70% identical, at least 80% identical, at least 90% identical, or at least 95% identical to one another. In some embodiments, two or more sequences are said to be “highly conserved” if they are about 70% identical, about 80% identical, about 90% identical, about 95%, about 98%, or about 99% identical to one another. In some embodiments, two or more sequences are said to be “conserved” if they are at least 30% identical, at least 40% identical, at least 50% identical, at least 60% identical, at least 70% identical, at least 80% identical, at least 90% identical, or at least 95% identical to one another. In some embodiments, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another. Conservation of sequence may apply to the entire length of a renal polynucleotide or renal polypeptide or may apply to a portion, region or feature thereof.

Controlled Release: As used herein, the term “controlled release” refers to a pharmaceutical composition or compound release profile that conforms to a particular pattern of release to effect a therapeutic outcome.

Cyclic or Cyclized: As used herein, the term “cyclic” refers to the presence of a continuous loop. Cyclic molecules need not be circular, only joined to form an unbroken chain of subunits. Cyclic molecules such as the engineered RNA or mRNA of the present invention may be single units or multimers or comprise one or more components of a complex or higher order structure.

Cytostatic: As used herein, “cytostatic” refers to inhibiting, reducing, suppressing the growth, division, or multiplication of a cell (e.g., a mammalian cell (e.g., a human cell)), bacterium, virus, fungus, protozoan, parasite, prion, or a combination thereof.

Cytotoxic: As used herein, “cytotoxic” refers to killing or causing injurious, toxic, or deadly effect on a cell (e.g., a mammalian cell (e.g., a human cell)), bacterium, virus, fungus, protozoan, parasite, prion, or a combination thereof.

Delivery: As used herein, “delivery” refers to the act or manner of delivering a compound, substance, entity, moiety, cargo or payload.

Delivery Agent: As used herein, “delivery agent” refers to any substance which facilitates, at least in part, the in vivo delivery of a renal polynucleotide to targeted cells.

Destabilized: As used herein, the term “destable,” “destabilize,” or “destabilizing region” means a region or molecule that is less stable than a starting, wild-type or native form of the same region or molecule.

Detectable label: As used herein, “detectable label” refers to one or more markers, signals, or moieties which are attached, incorporated or associated with another entity that is readily detected by methods known in the art including radiography, fluorescence, chemiluminescence, enzymatic activity, absorbance and the like. Detectable labels include radioisotopes, fluorophores, chromophores, enzymes, dyes, metal ions, ligands such as biotin, avidin, streptavidin and haptens, quantum dots, and the like.

Detectable labels may be located at any position in the renal peptides or proteins disclosed herein. They may be within the amino acids, the renal peptides, or proteins, or located at the N- or C-termini.

Diastereomer: As used herein, the term “diastereomer,” means stereoisomers that are not mirror images of one another and are non-superimposable on one another.

Digest: As used herein, the term “digest” means to break apart into smaller pieces or components. When referring to renal polypeptides or proteins, digestion results in the production of renal peptides.

Distal: As used herein, the term “distal” means situated away from the center or away from a point or region of interest.

Dosing regimen: As used herein, a “dosing regimen” is a schedule of administration or physician determined regimen of treatment, prophylaxis, or palliative care.

Dose splitting factor (DSF)-ratio of PUD of dose split treatment divided by PUD of total daily dose or single unit dose. The value is derived from comparison of dosing regimens groups.

Enantiomer: As used herein, the term “enantiomer” means each individual optically active form of a compound of the invention, having an optical purity or enantiomeric excess (as determined by methods standard in the art) of at least 80% (i.e., at least 90% of one enantiomer and at most 10% of the other enantiomer), preferably at least 90% and more preferably at least 98%.

Encapsulate: As used herein, the term “encapsulate” means to enclose, surround or encase.

Encoded protein cleavage signal: As used herein, “encoded protein cleavage signal” refers to the nucleotide sequence which encodes a protein cleavage signal.

Engineered: As used herein, embodiments of the invention are “engineered” when they are designed to have a feature or property, whether structural or chemical, that varies from a starting point, wild type or native molecule.

Effective Amount: As used herein, the term “effective amount” of an agent is that amount sufficient to effect beneficial or desired results, for example, clinical results, and, as such, an “effective amount” depends upon the context in which it is being applied. For example, in the context of administering an agent that treats cancer, an effective amount of an agent is, for example, an amount sufficient to achieve treatment, as defined herein, of cancer, as compared to the response obtained without administration of the agent.

Exosome: As used herein, “exosome” is a vesicle secreted by mammalian cells or a complex involved in RNA degradation.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a renal polypeptide or protein; and (4) post-translational modification of a renal polypeptide or protein.

Feature: As used herein, a “feature” refers to a characteristic, a property, or a distinctive element.

Formulation: As used herein, a “formulation” includes at least a renal polynucleotide and a delivery agent.

Fragment: A “fragment,” as used herein, refers to a portion. For example, fragments of proteins may comprise renal polypeptides obtained by digesting full-length protein isolated from cultured cells.

Functional: As used herein, a “functional” biological molecule is a biological molecule in a form in which it exhibits a property and/or activity by which it is characterized.

Homology: As used herein, the term “homology” refers to the overall relatedness between polymeric molecules, e.g. between nucleic acid molecules (e.g. DNA molecules and/or RNA molecules) and/or between renal polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (renal polynucleotide or renal polypeptide sequences). In accordance with the invention, two renal polynucleotide sequences are considered to be homologous if the renal polypeptides they encode are at least about 50%, 60%, 70%, 80%, 90%, 95%, or even 99% for at least one stretch of at least about 20 amino acids. In some embodiments, homologous renal polynucleotide sequences are characterized by the ability to encode a stretch of at least 4-5 uniquely specified amino acids. For renal polynucleotide sequences less than 60 nucleotides in length, homology is determined by the ability to encode a stretch of at least 4-5 uniquely specified amino acids. In accordance with the invention, two protein sequences are considered to be homologous if the proteins are at least about 50%, 60%, 70%, 80%, or 90% identical for at least one stretch of at least about 20 amino acids.

Identity: As used herein, the term “identity” refers to the overall relatedness between polymeric molecules, e.g., between renal polynucleotide molecules (e.g. DNA molecules and/or RNA molecules) and/or between renal polypeptide molecules. Calculation of the percent identity of two renal polynucleotide sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)).

Inhibit expression of a gene: As used herein, the phrase “inhibit expression of a gene” means to cause a reduction in the amount of an expression product of the gene. The expression product can be an RNA transcribed from the gene (e.g., an mRNA) or a renal polypeptide translated from an mRNA transcribed from the gene. Typically a reduction in the level of an mRNA results in a reduction in the level of a renal polypeptide translated therefrom. The level of expression may be determined using standard techniques for measuring mRNA or protein.

Infectious agent: As used herein, an “infectious agent” refers to any microorganism, virus, infectious substance, or biological product that may be engineered as a result of biotechnology, or any naturally occurring or bioengineered component of any such microorganism, virus, infectious substance, or biological product, can cause emerging and contagious disease, death or other biological malfunction in a human, an animal, a plant or another living organism.

Isomer: As used herein, the term “isomer” means any tautomer, stereoisomer, enantiomer, or diastereomer of any compound of the invention. It is recognized that the compounds of the invention can have one or more chiral centers and/or double bonds and, therefore, exist as stereoisomers, such as double-bond isomers (i.e., geometric E/Z isomers) or diastereomers (e.g., enantiomers (i.e., (+) or (−)) or cis/trans isomers). According to the invention, the chemical structures depicted herein, and therefore the compounds of the invention, encompass all of the corresponding stereoisomers, that is, both the stereomerically pure form (e.g., geometrically pure, enantiomerically pure, or diastereomerically pure) and enantiomeric and stereoisomeric mixtures, e.g., racemates. Enantiomeric and stereoisomeric mixtures of compounds of the invention can typically be resolved into their component enantiomers or stereoisomers by well-known methods, such as chiral-phase gas chromatography, chiral-phase high performance liquid chromatography, crystallizing the compound as a chiral salt complex, or crystallizing the compound in a chiral solvent. Enantiomers and stereoisomers can also be obtained from stereomerically or enantiomerically pure intermediates, reagents, and catalysts by well-known asymmetric synthetic methods.

In vitro: As used herein, the term “in vitro” refers to events that occur in an artificial environment, e.g., in a test tube or reaction vessel, in cell culture, in a Petri dish, etc., rather than within an organism (e.g., animal, plant, or microbe).

In vivo: As used herein, the term “in vivo” refers to events that occur within an organism (e.g., animal, plant, or microbe or cell or tissue thereof).

Isolated: As used herein, the term “isolated” refers to a substance or entity that has been separated from at least some of the components with which it was associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. Substantially isolated: By “substantially isolated” is meant that the compound is substantially separated from the environment in which it was formed or detected. Partial separation can include, for example, a composition enriched in the compound of the present disclosure. Substantial separation can include compositions containing at least about 50%, at least about 60%, at least about 70%, at least about 80%, at least about 90%, at least about 95%, at least about 97%, or at least about 99% by weight of the compound of the present disclosure, or salt thereof. Methods for isolating compounds and their salts are routine in the art.

IVT Renal polynucleotide: As used herein, an “IVT renal polynucleotide” is a linear renal polynucleotide which may be made using only in vitro transcription (IVT) enzymatic synthesis methods.

Linker: As used herein, a “linker” refers to a group of atoms, e.g., 10-1,000 atoms, and can be comprised of the atoms or groups such as, but not limited to, carbon, amino, alkylamino, oxygen, sulfur, sulfoxide, sulfonyl, carbonyl, and imine. The linker can be attached to a modified nucleoside or nucleotide on the nucleobase or sugar moiety at a first end, and to a payload, e.g., a detectable or therapeutic agent, at a second end. The linker may be of sufficient length as to not interfere with incorporation into a nucleic acid sequence. The linker can be used for any useful purpose, such as to form renal polynucleotide multimers (e.g., through linkage of two or more chimeric renal polynucleotides molecules or IVT renal polynucleotides) or renal polynucleotides conjugates, as well as to administer a payload, as described herein. Examples of chemical groups that can be incorporated into the linker include, but are not limited to, alkyl, alkenyl, alkynyl, amido, amino, ether, thioether, ester, alkylene, heteroalkylene, aryl, or heterocyclyl, each of which can be optionally substituted, as described herein. Examples of linkers include, but are not limited to, unsaturated alkanes, polyethylene glycols (e.g., ethylene or propylene glycol monomeric units, e.g., diethylene glycol, dipropylene glycol, triethylene glycol, tripropylene glycol, tetraethylene glycol, or tetraethylene glycol), and dextran polymers and derivatives thereof., Other examples include, but are not limited to, cleavable moieties within the linker, such as, for example, a disulfide bond (—S—S—) or an azo bond (—N═N—), which can be cleaved using a reducing agent or photolysis. Non-limiting examples of a selectively cleavable bond include an amido bond can be cleaved for example by the use of tris(2-carboxyethyl)phosphine (TCEP), or other reducing agents, and/or photolysis, as well as an ester bond can be cleaved for example by acidic or basic hydrolysis.

MicroRNA (miRNA) binding site: As used herein, a microRNA (miRNA) binding site represents a nucleotide location or region of a nucleic acid transcript to which at least the “seed” region of a miRNA binds.

Modified: As used herein “modified” refers to a changed state or structure of a molecule of the invention. Molecules may be modified in many ways including chemically, structurally, and functionally. In one embodiment, the mRNA molecules of the present invention are modified by the introduction of non-natural nucleosides and/or nucleotides, e.g., as it relates to the natural ribonucleotides A, U, G, and C. Noncanonical nucleotides such as the cap structures are not considered “modified” although they differ from the chemical structure of the A, C, G, U ribonucleotides.

Mucus: As used herein, “mucus” refers to the natural substance that is viscous and comprises mucin glycoproteins.

Naturally occurring: As used herein, “naturally occurring” means existing in nature without artificial aid.

Non-human vertebrate: As used herein, a “non-human vertebrate” includes all vertebrates except Homo sapiens, including wild and domesticated species. Examples of non-human vertebrates include, but are not limited to, mammals, such as alpaca, banteng, bison, camel, cat, cattle, deer, dog, donkey, gayal, goat, guinea pig, horse, llama, mule, pig, rabbit, reindeer, sheep water buffalo, and yak.

Off-target: As used herein, “off target” refers to any unintended effect on any one or more target, gene, or cellular transcript.

Open reading frame: As used herein, “open reading frame” or “ORF” refers to a sequence which does not contain a stop codon in a given reading frame.

Operably linked: As used herein, the phrase “operably linked” refers to a functional connection between two or more molecules, constructs, transcripts, entities, moieties or the like.

Optionally substituted: Herein a phrase of the form “optionally substituted X” (e.g., optionally substituted alkyl) is intended to be equivalent to “X, wherein X is optionally substituted” (e.g., “alkyl, wherein the alkyl is optionally substituted”). It is not intended to mean that the feature “X” (e.g. alkyl) per se is optional.

Part: As used herein, a “part” or “region” of a renal polynucleotide is defined as any portion of the renal polynucleotide which is less than the entire length of the renal polynucleotide.

Peptide: As used herein, “peptide” is less than or equal to 50 amino acids long, e.g., about 5, 10, 15, 20, 25, 30, 35, 40, 45, or 50 amino acids long.

Paratope: As used herein, a “paratope” refers to the antigen-binding site of an antibody.

Patient: As used herein, “patient” refers to a subject who may seek or be in need of treatment, requires treatment, is receiving treatment, will receive treatment, or a subject who is under care by a trained professional for a particular disease or condition.

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipients: The phrase “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrates, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts: The present disclosure also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

Pharmaceutically acceptable solvate: The term “pharmaceutically acceptable solvate,” as used herein, means a compound of the invention wherein molecules of a suitable solvent are incorporated in the crystal lattice. A suitable solvent is physiologically tolerable at the dosage administered. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

Pharmacokinetic: As used herein, “pharmacokinetic” refers to any one or more properties of a molecule or compound as it relates to the determination of the fate of substances administered to a living organism. Pharmacokinetics is divided into several areas including the extent and rate of absorption, distribution, metabolism and excretion. This is commonly referred to as ADME where: (A) Absorption is the process of a substance entering the blood circulation; (D) Distribution is the dispersion or dissemination of substances throughout the fluids and tissues of the body; (M) Metabolism (or Biotransformation) is the irreversible transformation of parent compounds into daughter metabolites; and (E) Excretion (or Elimination) refers to the elimination of the substances from the body. In rare cases, some drugs irreversibly accumulate in body tissue.

Physicochemical: As used herein, “physicochemical” means of or relating to a physical and/or chemical property.

Renal polypeptide per unit drug (PUD): As used herein, a PUD or product per unit drug, is defined as a subdivided portion of total daily dose, usually 1 mg, pg, kg, etc., of a product (such as a renal polypeptide) as measured in body fluid or tissue, usually defined in concentration such as pmol/mL, mmol/mL, etc. divided by the measure in the body fluid.

Preventing: As used herein, the term “preventing” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

Prodrug: The present disclosure also includes prodrugs of the compounds described herein. As used herein, “prodrugs” refer to any substance, molecule or entity which is in a form predicate for that substance, molecule or entity to act as a therapeutic upon chemical or physical alteration. Prodrugs may by covalently bonded or sequestered in some way and which release or are converted into the active drug moiety prior to, upon or after administered to a mammalian subject. Prodrugs can be prepared by modifying functional groups present in the compounds in such a way that the modifications are cleaved, either in routine manipulation or in vivo, to the parent compounds. Prodrugs include compounds wherein hydroxyl, amino, sulfhydryl, or carboxyl groups are bonded to any group that, when administered to a mammalian subject, cleaves to form a free hydroxyl, amino, sulfhydryl, or carboxyl group respectively. Preparation and use of prodrugs is discussed in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are hereby incorporated by reference in their entirety.

Proliferate: As used herein, the term “proliferate” means to grow, expand or increase or cause to grow, expand or increase rapidly. “Proliferative” means having the ability to proliferate. “Anti-proliferative” means having properties counter to or inapposite to proliferative properties.

Progenitor cell: As used herein, the term “progenitor cell” refers to cells that have greater developmental potential relative to a cell which it can give rise to by differentiation.

Prophylactic: As used herein, “prophylactic” refers to a therapeutic or course of action used to prevent the spread of disease.

Prophylaxis: As used herein, a “prophylaxis” refers to a measure taken to maintain health and prevent the spread of disease. An “immune phrophylaxis” refers to a measure to produce active or passive immunity to prevent the spread of disease.

Protein cleavage site: As used herein, “protein cleavage site” refers to a site where controlled cleavage of the amino acid chain can be accomplished by chemical, enzymatic or photochemical means.

Protein cleavage signal: As used herein “protein cleavage signal” refers to at least one amino acid that flags or marks a renal polypeptide for cleavage.

Protein of interest: As used herein, the terms “proteins of interest” or “desired proteins” include those provided herein and fragments, mutants, variants, and alterations thereof.

Proximal: As used herein, the term “proximal” means situated nearer to the center or to a point or region of interest.

Pseudouridine: As used herein, pseudouridine refers to the C-glycoside isomer of the nucleoside uridine. A “pseudouridine analog” is any modification, variant, isoform or derivative of pseudouridine. For example, pseudouridine analogs include but are not limited to 1-carboxymethyl-pseudouridine, 1-propynyl-pseudouridine, 1-taurinomethyl-pseudouridine, 1-taurinomethyl-4-thio-pseudouridine, 1-methylpseudouridine (m¹ψ) 1-methyl-4-thio-pseudouridinem (m¹s⁴ψ) 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydropseudouridine, 2-thio-dihydropseudouridine, 2-methoxyuridine, 2-methoxy-4-thio-uridine, 4-methoxy-pseudouridine, 4-methoxy-2-thio-pseudouridine, N1-methyl-pseudouridine, 1-methyl-3-(3-amino-3-carboxypropyl)pseudouridine (acp³ ψ), and 2′-O-methyl-pseudouridine (ψm).

Purified: As used herein, “purify,” “purified,” “purification” means to make substantially pure or clear from unwanted components, material defilement, admixture or imperfection.

Repeated transfection: As used herein, the term “repeated transfection” refers to transfection of the same cell culture with a renal polynucleotide a plurality of times. The cell culture can be transfected at least twice, at least 3 times, at least 4 times, at least 5 times, at least 6 times, at least 7 times, at least 8 times, at least 9 times, at least 10 times, at least 11 times, at least 12 times, at least 13 times, at least 14 times, at least 15 times, at least 16 times, at least 17 times at least 18 times, at least 19 times, at least 20 times, at least 25 times, at least 30 times, at least 35 times, at least 40 times, at least 45 times, at least 50 times or more.

Sample: As used herein, the term “sample” or “biological sample” refers to a subset of its tissues, cells or component parts (e.g. body fluids, including but not limited to blood, mucus, lymphatic fluid, synovial fluid, cerebrospinal fluid, saliva, amniotic fluid, amniotic cord blood, urine, vaginal fluid and semen). A sample further may include a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, organs. A sample further refers to a medium, such as a nutrient broth or gel, which may contain cellular components, such as proteins or nucleic acid molecule.

Signal Sequences: As used herein, the phrase “signal sequences” refers to a sequence which can direct the transport or localization of a protein.

Single unit dose: As used herein, a “single unit dose” is a dose of any therapeutic administered in one dose/at one time/single route/single point of contact, i.e., single administration event.

Similarity: As used herein, the term “similarity” refers to the overall relatedness between polymeric molecules, e.g. between renal polynucleotide molecules (e.g. DNA molecules and/or RNA molecules) and/or between renal polypeptide molecules. Calculation of percent similarity of polymeric molecules to one another can be performed in the same manner as a calculation of percent identity, except that calculation of percent similarity takes into account conservative substitutions as is understood in the art.

Split dose: As used herein, a “split dose” is the division of single unit dose or total daily dose into two or more doses.

Stable: As used herein “stable” refers to a compound that is sufficiently robust to survive isolation to a useful degree of purity from a reaction mixture, and preferably capable of formulation into an efficacious therapeutic agent.

Stabilized: As used herein, the term “stabilize”, “stabilized,” “stabilized region” means to make or become stable.

Stereoisomer: As used herein, the term “stereoisomer” refers to all possible different isomeric as well as conformational forms which a compound may possess (e.g., a compound of any formula described herein), in particular all possible stereochemically and conformationally isomeric forms, all diastereomers, enantiomers and/or conformers of the basic molecular structure. Some compounds of the present invention may exist in different tautomeric forms, all of the latter being included within the scope of the present invention.

Subject: As used herein, the term “subject” or “patient” refers to any organism to which a composition in accordance with the invention may be administered, e.g., for experimental, diagnostic, prophylactic, and/or therapeutic purposes. Typical subjects include animals (e.g., mammals such as mice, rats, rabbits, non-human primates, and humans) and/or plants.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantially equal: As used herein as it relates to time differences between doses, the term means plus/minus 2%.

Substantially simultaneously: As used herein and as it relates to plurality of doses, the term means within 2 seconds.

Suffering from: An individual who is “suffering from” a disease, disorder, and/or condition has been diagnosed with or displays one or more symptoms of a disease, disorder, and/or condition.

Susceptible to: An individual who is “susceptible to” a disease, disorder, and/or condition has not been diagnosed with and/or may not exhibit symptoms of the disease, disorder, and/or condition but harbors a propensity to develop a disease or its symptoms. In some embodiments, an individual who is susceptible to a disease, disorder, and/or condition (for example, cancer) may be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein and/or nucleic acid associated with the disease, disorder, and/or condition; (4) habits and/or lifestyles associated with development of the disease, disorder, and/or condition; (5) a family history of the disease, disorder, and/or condition; and (6) exposure to and/or infection with a microbe associated with development of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Sustained release: As used herein, the term “sustained release” refers to a pharmaceutical composition or compound release profile that conforms to a release rate over a specific period of time.

Synthetic: The term “synthetic” means produced, prepared, and/or manufactured by the hand of man. Synthesis of renal polynucleotides or renal polypeptides or other molecules of the present invention may be chemical or enzymatic.

Targeted Cells: As used herein, “targeted cells” refers to any one or more cells of interest. The cells may be found in vitro, in vivo, in situ or in the tissue or organ of an organism. The organism may be an animal, preferably a mammal, more preferably a human and most preferably a patient.

Therapeutic Agent: The term “therapeutic agent” refers to any agent that, when administered to a subject, has a therapeutic, diagnostic, and/or prophylactic effect and/or elicits a desired biological and/or pharmacological effect.

Therapeutically effective amount: As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition.

Therapeutically effective outcome: As used herein, the term “therapeutically effective outcome” means an outcome that is sufficient in a subject suffering from or susceptible to an infection, disease, disorder, and/or condition, to treat, improve symptoms of, diagnose, prevent, and/or delay the onset of the infection, disease, disorder, and/or condition.

Transcription: As used herein, the term “transcription” refers to methods to introduce exogenous nucleic acids into a cell. Methods of transfection include, but are not limited to, chemical methods, physical treatments and cationic lipids or mixtures.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Unmodified: As used herein, “unmodified” refers to any substance, compound or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification.

Unipotent: As used herein, “unipotent” when referring to a cell means to give rise to a single cell lineage.

Vaccine: As used herein, the phrase “vaccine” refers to a biological preparation that improves immunity to a particular disease.

Viral protein: As used herein, the phrase “viral protein” means any protein originating from a virus.

### VIII. EQUIVALENTS AND SCOPE

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Unless otherwise defined, all technical and scientific terms used herein have the same meaning as commonly understood by one of ordinary skill in the art to which this invention belongs. Methods and materials are described herein for use in the present disclosure; other, suitable methods and materials known in the art can also be used.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any nucleic acid or protein encoded thereby; any method of production; any method of use; etc.)

can be excluded from any one or more claims, for any reason, whether or not related to the existence of prior art.

All cited sources, for example, references, publications, databases, database entries, and art cited herein, are incorporated into this application by reference, even if not expressly stated in the citation. In case of conflicting statements of a cited source and the instant application, the statement in the instant application shall control.

Section and table headings are not intended to be limiting.

### IX. EXAMPLES

### Example 1. Manufacture of Chimeric Polynucleotides

According to the present invention, the manufacture of chimeric polynucleotides and or parts or regions thereof may be accomplished utilizing the methods taught in International Patent Publication No. WO2014152027 (Attorney Docket number M500), the contents of which is incorporated herein by reference in its entirety.

Purification methods may include those taught in International Patent Publication No. WO2014152030 (Attorney Docket number M501); International Patent Publication No. WO2014152031 (Attorney Docket number M502), each of which is incorporated herein by reference in its entirety.

Characterization of the chimeric polynucleotides of the invention may be accomplished using a procedure selected from the group consisting of polynucleotide mapping, reverse transcriptase sequencing, charge distribution analysis, and detection of RNA impurities, wherein characterizing comprises determining the RNA transcript sequence, determining the purity of the RNA transcript, or determining the charge heterogeneity of the RNA transcript. Such methods are taught in, for example, International Patent Publication No. WO2014144039 (Attorney Docket number M505); International Patent Publication No. WO2014144711 (Attorney Docket number M506) and International Patent Publication No. WO2014144767 (Attorney Docket number M507) the contents of each of which is incorporated herein by reference in its entirety.

### Example 2: PCR for cDNA Production

PCR procedures for the preparation of cDNA are performed using 2×KAPA HIFI™ HotStart ReadyMix by Kapa Biosystems (Woburn, Mass.). This system includes 2×KAPA ReadyMix12.5 μl; Forward Primer (10 μm) 0.75 μl; Reverse Primer (10 μm) 0.75 μl; Template cDNA-100 ng; and dH₂O diluted to 25.0 μl. The reaction conditions are at 95° C. for 5 min. and 25 cycles of 98° C. for 20 sec, then 58° C. for 15 sec, then 72° C. for 45 sec, then 72° C. for 5 min. then 4° C. to termination.

The reverse primer of the instant invention incorporates a poly-T₁₂₀ for a poly-A₁₂₀ in the mRNA. Other reverse primers with longer or shorter poly(T) tracts can be used to adjust the length of the poly(A) tail in the polynucleotide mRNA.

The reaction is cleaned up using Invitrogen's PURELINK™ PCR Micro Kit (Carlsbad, Calif.) per manufacturer's instructions (up to 5 μg). Larger reactions will require a cleanup using a product with a larger capacity. Following the cleanup, the cDNA is quantified using the NANODROP™ and analyzed by agarose gel electrophoresis to confirm the cDNA is the expected size. The cDNA is then submitted for sequencing analysis before proceeding to the in vitro transcription reaction.

### Example 3. In Vitro Transcription (IVT)

A. Synthesis of mRNA Constructs in Preparation for IVT

### Restriction Digest of Plasmid DNA

DNA plasmid is digested by incubation at 37° C. for 2 hr in a 50 μL reaction containing DNA plasmid (50 ng/μL), BSA (1×), 1×NEBuffer 4 (50 mM potassium acetate, 20 mM Tris-acetate, 10 mM magnesium acetate, 1 mM DTT, pH 7.9), and Xbal (400 U/mL) (New England Biolabs). The restriction digest is analyzed by 1% agarose gel and used directly for PCR.

### DNA Template Amplification

The desired DNA template is amplified by PCR in 100 uL reactions using linearized plasmid (20 ng), dNTPs (0.2 μM each), forward primer (0.2 μM), reverse primer (0.2 μM), 1× Q5 reaction buffer, and Q5 high-fidelity DNA polymerase (20 U/mL) (New England Biolabs). All components are kept on ice until added to the thermocycler. The reaction conditions are at 95° C. for 4 min. and 30 cycles of 98° C. for 15 sec, then 72° C. for 45 sec, then 72° C. for 20 sec per kb, then 72° C. for 5 min. then 4° C. to termination. The PCR product is analyzed by capillary electrophoresis (CE) (Agilent 2100 Bioanalyzer) and desalted by ultrafiltration (Amicon).

### B. IVT Reaction

In vitro transcription (IVT) reactions are performed in 50 uL containing template DNA (25 ng/μL), NTPs (7.6 mM each), 1×T7 IVT buffer, RNase Inhibitor (1 U/μL), Pyrophosphatase (1 U/μL), and T7 RNA polymerase (7 U/μL) (NEB). In general, 24 50 uL reactions per construct are used. Modified mRNA may be generated using 5-methyl-CTP and 1-methyl-pseudoUTP or any chosen modified triphosphate. IVT reactions are incubated at 37° C. for 4 hr, after which 2.5 μL of DNase I (2000 U/mL) (NEB) is added and the reaction allowed to incubated for another 45 min. The reactions are combined and purified using MEGAclear spin columns (Ambion) and eluted in 250 μL water. The IVT product is analyzed by CE (Agilent 2100 Bioanalyzer).

### Example 4. Enzymatic Capping

Capping of a polynucleotide is performed as follows where the mixture includes: IVT RNA 60 μg-180 μg and dH₂O up to 72 μl. The mixture is incubated at 65° C. for 5 minutes to denature RNA, and then is transferred immediately to ice.

The protocol then involves the mixing of 10× Capping Buffer (0.5 M Tris-HCl (pH 8.0), 60 mM KCl, 12.5 mM MgCl₂) (10.0 μl); 20 mM GTP (5.0 μl); 20 mM S-Adenosyl Methionine (2.5 μl); RNase Inhibitor (100 U); 2′-O-Methyltransferase (400 U); Vaccinia capping enzyme (Guanylyl transferase) (40 U); dH₂O (Up to 28 μl); and incubation at 37° C. for 30 minutes for 60 μg RNA or up to 2 hours for 180 μg of RNA.

The polynucleotide is then purified using Ambion's MEGACLEAR™ Kit (Austin, Tex.) following the manufacturer's instructions. Following the cleanup, the RNA is quantified using the NANODROP™ (ThermoFisher, Waltham, Mass.) and analyzed by agarose gel electrophoresis to confirm the RNA is the proper size and that no degradation of the RNA has occurred. The RNA product may also be sequenced by running a reverse-transcription-PCR to generate the cDNA for sequencing.

### Example 5. PolyA Tailing Reaction

Without a poly-T in the cDNA, a poly-A tailing reaction must be performed before cleaning the final product. This is done by mixing Capped IVT RNA (100 μl); RNase Inhibitor (20 U); 10× Tailing Buffer (0.5 M Tris-HCl (pH 8.0), 2.5 M NaCl, 100 mM MgCl₂)(12.0 μl); 20 mM ATP (6.0 μl); Poly-A Polymerase (20 U); dH₂O up to 123.5 μl and incubation at 37° C. for 30 min. If the poly-A tail is already in the transcript, then the tailing reaction may be skipped and proceed directly to cleanup with Ambion's MEGACLEAR™ kit (Austin, Tex.) (up to 500 μg). Poly-A Polymerase is preferably a recombinant enzyme expressed in yeast.

It should be understood that the processivity or integrity of the polyA tailing reaction may not always result in an exact size polyA tail. Hence polyA tails of approximately between 40-200 nucleotides, e.g., about 40, 50, 60, 70, 80, 90, 91, 92, 93, 94, 95, 96, 97, 98, 99, 100, 101, 102, 103, 104, 105, 106, 107, 108, 109, 110, 150-165, 155, 156, 157, 158, 159, 160, 161, 162, 163, 164 or 165 are within the scope of the invention.

### Example 6. Natural 5′ Caps and 5′ Cap Analogues

5′-capping of polynucleotides may be completed concomitantly during the in vitro-transcription reaction using the following chemical RNA cap analogs to generate the 5′-guanosine cap structure according to manufacturer protocols: 3′-O-Me-m7G(5′)ppp(5′) G [the ARCA cap]; G(5′)ppp(5′)A; G(5′)ppp(5′)G; m7G(5′)ppp(5′)A; m7G(5′)ppp(5′)G (New England BioLabs, Ipswich, Mass.). 5′-capping of modified RNA may be completed post-transcriptionally using a Vaccinia Virus Capping Enzyme to generate the “Cap 0” structure: m7G(5′)ppp(5′)G (New England BioLabs, Ipswich, Mass.). Cap 1 structure may be generated using both Vaccinia Virus Capping Enzyme and a 2′-O methyl-transferase to generate: m7G(5′)ppp(5′)G-2′-O-methyl. Cap 2 structure may be generated from the Cap 1 structure followed by the 2′-O-methylation of the 5′-antepenultimate nucleotide using a 2′-O methyl-transferase. Cap 3 structure may be generated from the Cap 2 structure followed by the 2′-O-methylation of the 5′-preantepenultimate nucleotide using a 2′-O methyl-transferase. Enzymes are preferably derived from a recombinant source.

When transfected into mammalian cells, the modified mRNAs have a stability of between 12-18 hours or more than 18 hours, e.g., 24, 36, 48, 60, 72 or greater than 72 hours.

### Example 7. Capping Assays

A. Protein Expression Assay

Polynucleotides (e.g., chimeric polynucleotides) encoding a polypeptide, containing any of the caps taught herein can be transfected into cells at equal concentrations. 6, 12, 24 and 36 hours post-transfection the amount of protein secreted into the culture medium can be assayed by ELISA. Synthetic polynucleotides that secrete higher levels of protein into the medium would correspond to a synthetic polynucleotide with a higher translationally-competent Cap structure.

B. Purity Analysis Synthesis

Polynucleotides (e.g., chimeric polynucleotides) encoding a polypeptide, containing any of the caps taught herein can be compared for purity using denaturing Agarose-Urea gel electrophoresis or HPLC analysis. Polynucleotides with a single, consolidated band by electrophoresis correspond to the higher purity product compared to polynucleotides with multiple bands or streaking bands. Synthetic polynucleotides with a single HPLC peak would also correspond to a higher purity product. The capping reaction with a higher efficiency would provide a more pure polynucleotide population.

C. Cytokine Analysis

Polynucleotides (e.g., chimeric polynucleotides) encoding a polypeptide, containing any of the caps taught herein can be transfected into cells at multiple concentrations. 6, 12, 24 and 36 hours post-transfection the amount of pro-inflammatory cytokines such as TNF-alpha and IFN-beta secreted into the culture medium can be assayed by ELISA. Polynucleotides resulting in the secretion of higher levels of pro-inflammatory cytokines into the medium would correspond to a polynucleotides containing an immune-activating cap structure.

D. Capping Reaction Efficiency

Polynucleotides encoding a polypeptide, containing any of the caps taught herein can be analyzed for capping reaction efficiency by LC-MS after nuclease treatment. Nuclease treatment of capped polynucleotides would yield a mixture of free nucleotides and the capped 5′-5-triphosphate cap structure detectable by LC-MS. The amount of capped product on the LC-MS spectra can be expressed as a percent of total polynucleotide from the reaction and would correspond to capping reaction efficiency. The cap structure with higher capping reaction efficiency would have a higher amount of capped product by LC-MS.

### Example 8. Agarose Gel Electrophoresis of Modified RNA or RT PCR Products

Individual polynucleotides (e.g, chimeric polynucleotides) (200-400 ng in a 20 μl volume) or reverse transcribed PCR products (200-400 ng) are loaded into a well on a non-denaturing 1.2% Agarose E-Gel (Invitrogen, Carlsbad, Calif.) and run for 12-15 minutes according to the manufacturer protocol.

### Example 9. Nanodrop Modified RNA Quantification and UV Spectral Data

Modified polynucleotides (e.g, chimeric polynucleotides) in TE buffer (1 μl) are used for Nanodrop UV absorbance readings to quantitate the yield of each chimeric polynucleotide from a chemical synthesis or in vitro transcription reaction.

### Example 10. Method of Screening for Protein Expression

A. Electrospray Ionization

A biological sample which may contain proteins encoded by a polynucleotide (e.g, chimeric polynucleotide) administered to the subject is prepared and analyzed according to the manufacturer protocol for electrospray ionization (ESI) using 1, 2, 3 or 4 mass analyzers. A biologic sample may also be analyzed using a tandem ESI mass spectrometry system.

Patterns of protein fragments, or whole proteins, are compared to known controls for a given protein and identity is determined by comparison.

B. Matrix-Assisted Laser Desorption/Ionization

A biological sample which may contain proteins encoded by one or more polynucleotides (e.g, chimeric polynucleotides) administered to the subject is prepared and analyzed according to the manufacturer protocol for matrix-assisted laser desorption/ionization (MALDI). Patterns of protein fragments, or whole proteins, are compared to known controls for a given protein and identity is determined by comparison.

C. Liquid Chromatography-Mass Spectrometry-Mass Spectrometry

A biological sample, which may contain proteins encoded by one or more polynucleotides (e.g, chimeric polynucleotides), may be treated with a trypsin enzyme to digest the proteins contained within. The resulting peptides are analyzed by liquid chromatography-mass spectrometry-mass spectrometry (LC/MS/MS). The peptides are fragmented in the mass spectrometer to yield diagnostic patterns that can be matched to protein sequence databases via computer algorithms. The digested sample may be diluted to achieve 1 ng or less starting material for a given protein. Biological samples containing a simple buffer background (e.g. water or volatile salts) are amenable to direct in-solution digest; more complex backgrounds (e.g. detergent, non-volatile salts, glycerol) require an additional clean-up step to facilitate the sample analysis.

Patterns of protein fragments, or whole proteins, are compared to known controls for a given protein and identity is determined by comparison.

### Example 11. Ionizable Versus Cationic Lipid LNP Studies—Characterization and Biodistribution Following Intra-Arterial Delivery-C12-200 LNP Kidney Study

Studies were designed to test the functionality of ionizable and cationic lipids in the LNP formulations of the invention. An exemplary cationic lipid, C12-200 was tested in these studies and compared to control DLin-KC2-DMA LNPs.

To test the biodistribution of various LNP formulations following intra-arterial delivery to kidney, rats (n=2) were prepared for surgery to facilitate intra-arterial delivery to kidney. In brief, rats were anesthetized then placed face up on a heating board/pad. The operative area was shaved and cleaned with 70% alcohol. The skin and the muscle layers were sectioned using a sterile surgical scalpel or scissor. After opening the abdominal cavity, the small intestine was pulled to one side of the abdomen to expose the proximal tract of the left kidney artery. The kidney artery was carefully isolated from the left renal vein and from the connective tissue. The aorta was clamped with an aneurysm clip, the renal artery was punctured and a catheter was advanced into the artery. The kidney was first perfused with sterile saline then with 150 μg of Luciferase mRNA formulated in a lipid nanoparticle comprising the ionizable lipid, DLin-KC2-DMA (KC2), or the cationic lipid, C12-200.

Immediately after completion of the perfusion of the luciferase mRNA, the left kidney vein and ureter were clamped at the renal hilum. The clamping was maintained for 20 minutes. The catheter was then removed and all clips were released to restart kidney blood flow. The artery was pressured until the bleeding had stopped completely. The peritoneum, muscle and skin three layers were closed with non-absorbable sutures. The animals were transferred into the observation cage and they were maintained on a heating pad and/or under heat lamp until they woke up. Approximately 23 hours post-administration, the rats were injected with luciferin (100 to 200 μl of luciferin, 10-30 mg/ml). The animals were euthanized by CO2 inhalation, the kidney, spleen and liver were rapidly dissected and then placed in the IVIS light-tight chamber to measure luciferase expression in photons per second (p/s). The average expression is shown in Table 7. In Table 7, “˜” means about.

TABLE 7
Luciferase Expression

| | Amount | | Spleen | Liver | Kidney |
|---|---|---|---|---|---|
| | mRNA | Time | Expression | Expression | Expression |
| # | (ug) | (hours) | (p/s) | (p/s) | (p/s) |
| 1 | 150 | ~23 | 1.71E+07 | 3.07E+07 | 5.66E+07 |
| 2 | 150 | ~23 | 4.97E+07 | 5.65E+07 | 1.05E+06 |
| 1 | 150 | ~7 | 1.73E+08 | 9.20E+07 | 7.29E+06 |
| 2 | 75 | ~23 | 1.03E+08 | 3.45E+07 | 3.17E+07 |
| 1 | 75 | ~19 | 9.02E+07 | 1.23E+08 | 3.19E+07 |
| 2 | 75 | ~6 | 3.34E+08 | 6.90E+07 | 5.77E+05 |
| 1 | 75 | ~7 | 4.91E+07 | 1.20E+08 | 3.70E+06 |
| 1 | ~67.5 | ~7 | 3.32E+06 | 4.51E+06 | 6.39E+06 |
| 1 | 15 | ~22 | 2.70E+06 | 1.50E+06 | 9.30E+06 |
| 1 | 15 | ~7 | 4.50E+06 | 6.50E+06 | 2.50E+06 |
| 1 | 4.5 | ~22 | 1.79E+04 | 8.97E+04 | 1.35E+06 |
| 1 | 4.5 | ~6 | 3.01E+05 | 8.11E+05 | 4.25E+05 |

These data suggest more controlled biodistribution for C12-200-containing LNPs with good kidney expression and reduced leakage of mRNA to spleen and liver. Good signal was observed, in particular, in kidney, for each dose and time point tested. Notably, at lower doses of mRNA using C12-200 LNPs, liver and spleen signals exhibited dose-dependency with the signal decreasing in a dose dependent manner as demonstrated for the −22-23 hour time point. Moreover, at various doses tested (i.e., 150 to 4.5 pg mRNA) the kidney signal is higher at longer time points (˜20-22 hrs.) Without being bound in theory, it is hypothesized that optimal dosing of mRNA can result in preferential or specific delivery to target tissue, e.g., kidney, making certain cationic lipid formulations preferred for particular localized delivery regimes.

### Example 12. Intraparenchymal Delivery of Modified mRNA to the Kidney

Studies are designed to test the delivery to kidneys of formulations of the invention. The rodent is anesthetized then placed face up on a heating board/pad. The operative area is shaved and cleaned with 70% alcohol. An incision is made in the skin to the middle left flank or to the left back side of the rodent. A small incision is made in the body wall. Incision size is just slightly longer than the long axis of the kidney. Sterile moistened gauze is used to circle the incision area. The left kidney is gently extricated using cotton swaps and is placed on a sterile moistened gauze. The modified mRNA is injected directly into the kidney parenchyma using a sterile 27G (rat only) to 32G needle (mice). A single (mouse) to maximum 2 (rat) injections of modified mRNA is performed per kidney.

After modified mRNA injection, the needle is carefully removed and the kidney is carefully inspected for bleeding. If necessary, a sterile hemostatic sponge is applied to stop bleeding. The kidney is gently eased back into the body cavity. The muscle and skin layers are closed with non-absorbable suture. The rodent is transferred into the observation cage and is maintained on a heating pad until it wakes up. Two to 72 hours post-Luciferase modified mRNA administration, the rodent is injected with luciferin (100 to 200 μl of luciferin, 10-30 mg/ml). The rodent is euthanized by CO2 inhalation, the kidney is rapidly dissected then placed in the IVIS light-tight chamber to measure luciferase expression.

### Example 13. Intraparenchymal Delivery of Modified mRNA in C12-200 LNPs to the Kidney

The rodent was anesthetized then placed face up on a heating board/pad. The operative area was shaved and cleaned with 70% alcohol. An incision was made in the skin to the middle left flank or to the left back side of the rodent. A small incision was made in the body wall. Incision size was just slightly longer than the long axis of the kidney. Sterile moistened gauze was used to circle the incision area. The left kidney was gently extricated using cotton swaps and was placed on a sterile moistened gauze. The lipid nanoparticle formulations comprising the cationic lipid C12-200 and the luciferase modified mRNA was injected directly into the kidney parenchyma using a sterile 27G (rat only) to 32G needle (mice).

After the injection of modified Luciferase mRNA, the needle was carefully removed and the kidney was carefully inspected for bleeding. If necessary, a sterile hemostatic sponge was applied to stop bleeding. The kidney was gently eased back into the body cavity. The muscle and skin layers were closed with non-absorbable suture. The rodent was transferred into the observation cage and is maintained on a heating pad until it wakes up. A few hours post-Luciferase modified mRNA administration, the rodent was injected with luciferin (100 to 200 μl of luciferin, 10-30 mg/ml). The rodent was euthanized by CO2 inhalation, the kidney was rapidly dissected then placed in the IVIS light-tight chamber to measure luciferase expression. The results are shown in Table 8. In Table 8, “˜” means about.

TABLE 8
Luciferase Expression

| | Amount | | Spleen | Liver | Kidney |
|---|---|---|---|---|---|
| | mRNA | Time | Expression | Expression | Expression |
| # | (μg); | (hours) | (p/s) | (p/s) | (p/s) |
| 1 | 3 | ~23 | 3.00E+05 | 1.60E+05 | 1.20E+06 |
| 2 | 3 | ~20 | 2.50E+05 | 3.50E+05 | 1.60E+06 |

These data demonstrate successful localized kidney delivery via intra-parenchymal administration of mRNA formulated in C12-200 LNPs.

### Example 14. DLin-KC2-DMA Bladder LNP Study

To further investigate the properties of various LNPs for local (e.g., tissue-specific) delivery of mRNAs, Luciferase mRNA formulated in a lipid nanoparticle comprising the ionizable amino lipid DLin-KC2-DMA and tested in a bladder delivery system. LNPs were administered to rats by direct intra-ureter injection.

Approximately 24 hours post-administration, the rats were injected with luciferin (100 to 200 μl of luciferin, 10-30 mg/ml). The animals were euthanized by CO2 inhalation and the bladder was rapidly dissected and then placed in the IVIS light-tight chamber to measure luciferase expression in photons per second (p/s). The average expression is shown in Table 9. These data demonstrate that ionizable lipid LNPs; e.g., KC2 LNPs, provide detectable delivery of mRNAs in the harsh environment of the bladder. Moreover, the signal is dose-dependent as higher Luciferase expression was observed with the higher mRNA dose. In Table 9, “˜” means about.

TABLE 9
Luciferase Expression

| | Amount | | Bladder |
|---|---|---|---|
| | of mRNA | Time | Expression |
| # | (ug) | (hours) | (p/s) |
| 1 | 50 | ~23 | 1.2E+06 |
| 2 | 100 | ~23 | 1.5E+07 |

### Example 15. Formulations Screen for Kidney Delivery of Modified mRNA

### A. Study Purpose and Design

To test the efficacy of different formulations for kidney delivery of modified mRNA, a single dose administration of modified mRNA encoding luciferase was given to rats and the expression patterns of luciferase in different organs after certain hours of administration were analyzed and compared for delivery efficacy in kidney. The luciferase modified mRNA was formulated in lipid nanoparticles with different compositions. Each formulation was delivered to the kidney via the left kidney artery in SD rats (Sprague Dawley rats) at a single dose. The rats were imaged at different time points as outlined below after kidney delivery of luciferase modified mRNA formulations. Luciferase expression patterns in different organs of the experimental rats, particularly in kidney, liver and spleen, were examined and analyzed.

### B. Lipid Comparison: KL10 and KL52

Luciferase mRNA fully modified 1-methylpseudouridine (1 mpU) was formulated in a lipid nanoparticle comprising KL10, DOPE, Cholesterol and PEG-DMG (40:20:38.5:1.5 mole percent; lipid to mRNA weight ratio of 32:1; particle size: 83 nm; N:P ratio of 5.67; PDI: 0.257; encapsulation efficiency 98.7%) or KL22, DOPE, Cholesterol and PEG-DMG (40:20:38.5:1.5 mole percent; lipid to mRNA weight ratio of 31:1; particle size: 100 nm; N:P ratio of 5.67; PDI: 0.72; encapsulation efficiency 92.2. The expression of luciferase in the kidney, liver and spleen was imaged about 6 hours after administration of a single dose of luciferase mRNA (15 μg/kidney/0.5 mL) via delivery to the artery of the kidney. The results of the imaging suggests that KL22 DOPE formulation showed more liver leak compared to KL10 DOPE formulation.

### C. Lipid Comparison: C12-200 and DLin-KC2-DMA

To study different lipid compositions, an experiment was designed to compare a DLin-KC2-DMA formulation and a C12-200/DSPC formulation. Luciferase mRNA fully modified with 5-methylcytosine and 1-methylpseudouridine (5mC/1 mpU) was formulated in a lipid nanoparticle comprising C12-200, DSPC, cholesterol, and PEG-DMG (40:30:25:5 mole percent; 20:1 lipid to mRNA weight ratio; particle size 103 nm; N:P ratio of 2.9; PDI: 0.21; encapsulation efficiency 72%). Luciferase mRNA modified with 5-methylcytosine and 1-methylpseudouridine (5mC/1mpU) was formulated in a lipid nanoparticle comprising the lipid DLin-KC2-DMA as a positive control. The expression of luciferase in the kidney, liver and spleen was imaged about 20 hours after administration of a single dose of luciferase mRNA (15 μg/kidney/0.5 mL) via delivery to the artery of the kidney. The results of the imaging indicated that a higher kidney expression and liver leakage was observed with the DLin-KC2-DMA formulation as compared to C12-200/DSPC formulation.

### D. Lipid Comparison: KL10, KL22, C12-200 and DLin-MC3-DMA

Luciferase mRNA fully modified 1-methylpseudouridine (1 mpU) was formulated in a lipid nanoparticle comprising the lipids KL10/DOPE (composition is KL10/DOPE/Cholesterol/PEG-DMG at 40:30:25:5 mole percent; 25:1 lipid to mRNA weight ratio; particle size 60.5 nm; N:P ratio of 4; PDI: 0.2; encapsulation efficiency 98.4%), C12-200/DOPE (composition is C12-200/DOPE/Cholesterol/PEG-DMG at 30:55:10:5 mole percent; 37:1 lipid to mRNA weight ratio; particle size 68 nm; N:P ratio of 4; PDI: 0.17; encapsulation efficiency 82.2%), DLin-MC3-DMA/DSPC (DLin-MC3-DMA/DSPC/Cholesterol/PEG-DMG at 50:10:35:5 mole percent; 23:1 lipid to mRNA weight ratio; particle size 55 nm; N:P ratio of 5.67; PDI: 0.18; encapsulation efficiency 97.2%) or KL22/DOPE (DL22/DOPE/Cholesterol/PEG-DMG at 40:20:38.5:1.5 mole percent; 32:1 lipid to mRNA weight ratio; particle size 75.2 nm; N:P ratio of 5.67; PDI: 0.19; encapsulation efficiency 94.1%). The expression of luciferase in the kidney, sliced kidney, liver and spleen was imaged about 3 hours after administration of a single dose of luciferase mRNA (10 μg/kidney/0.5 mL) via delivery to the artery of the kidney. The results indicate that there was less liver/spleen leakage for KL10/DOPE formulation as compared to the other formulations. Additionally, purified mRNA in KL22/DOPE showed a stronger expression versus the other formulations and little to no kidney expression was observed with DLin-MC3-DMA/DSPC as compared to the other formulations.

### Example 16. Comparison of C12-200 Compositions for Kidney Delivery of Modified mRNA

C12-200 compositions with different lipid components (e.g., DSPC and DOPE), PEG percentages, particle sizes and N:P ratios, were tested via delivery of modified mRNA by administration to the left kidney artery in SD rats (Sprague Dawley rats) at a single dose. The rats were imaged at different time points as outlined below after kidney delivery of luciferase modified mRNA formulations. Luciferase expression patterns in different organs of the experimental rats, particularly in kidney, liver and spleen, were examined and analyzed.

A. DSPC v. DOPE and PEG 1.5% v. PEG 5%

Luciferase mRNA fully modified 1-methylpseudouridine (1 mpU) was formulated in a lipid nanoparticle comprising C12-200/DSPC/Cholesterol/PEG-DMG (40:30:25:5 mole percent; 35:1 lipid to mRNA weight ratio; particle size 83 nm; N:P ratio of 5; PDI: 0.22; encapsulation efficiency 73%), C12-200/DOPE/Cholesterol/PEG-DMG (40:30:25:5 mole percent; 35:1 lipid to mRNA weight ratio; particle size 64 nm; N:P ratio of 5; PDI: 0.21; encapsulation efficiency 58%), C12-200/DSPC/Cholesterol/PEG-DMG (50:10:38.5:1.5 mole percent; 30:1 lipid to mRNA weight ratio; particle size 142 nm; N:P ratio of 6; PDI: 0.16; encapsulation efficiency 86%) or C12-200/DOPE/Cholesterol/PEG-DMG (50:10:38.5:1.5 mole percent; 30:1 lipid to mRNA weight ratio; particle size 173 nm; N:P ratio of 6; PDI: 0.14; encapsulation efficiency 65%). The expression of luciferase in the kidney, liver and spleen was imaged about 6 hours after administration of a single dose of luciferase mRNA (15 μg/kidney/0.5 mL) via delivery to the artery of the kidney. Based on this study it appears that the DSPC formulations tends to cause more leak to liver and spleen (for both PEG 1.5% and 5%) as compared to the DOPE formulations.

### B. Lipid Comparison: DSPC and DOPE

Luciferase mRNA fully modified 1-methylpseudouridine (1 mpU) was formulated in a lipid nanoparticle comprising C12-200/DSPC/Cholesterol/PEG (40:30:25:5 mole percent; 20:1 lipid to mRNA weight ratio; particle size 111 nm; N:P ratio of 2.9; PDI: 0.21; encapsulation efficiency 57%) or C12-200/DOPE/Cholesterol/PEG (40:30:25:5 mole percent; 20:1 lipid to mRNA weight ratio; particle size 94 nm; N:P ratio of 2.9; PDI: 0.22; encapsulation efficiency 85%). The expression of luciferase in the kidney, liver and spleen was imaged about 6 hours and 22 hours after administration of a single dose of luciferase mRNA (15 μg/kidney/0.5 mL) via delivery to the artery of the kidney. The imaging results suggest that DOPE-based formulation shows more rapid decay of luciferase expression in kidney and liver/spleen as compared to the DSPC-based formulations.

### C. Lipid Comparision: DSPC and DOPE

Luciferase mRNA fully modified 1-methylpseudouridine (1 mpU) was formulated in a lipid nanoparticle comprising C12-200/DSPC/Cholesterol/PEG-DMG (40:30:25:5 mole percent; 20:1 lipid to mRNA weight ratio; particle size 121 nm; N:P ratio of 2.9; PDI: 0.22; encapsulation efficiency 74%) or C12-200/DOPE/Cholesterol/PEG-DMG (40:30:25:5 mole percent; 20:1 lipid to mRNA weight ratio; particle size 92 nm; N:P ratio of 2.9; PDI: 0.23; encapsulation efficiency 61%). The expression of luciferase in the kidney, liver and spleen was imaged about 6 hours after administration of a single dose of luciferase mRNA (15 μg/kidney/0.5 mL) via delivery to the artery of the kidney. There was no significant difference in kidney signal or leak to liver/spleen between the C12-200 DSPC formulation and the C12-200 DOPE formulation.

### D. N:P Comparison: 2.9 and 4.0

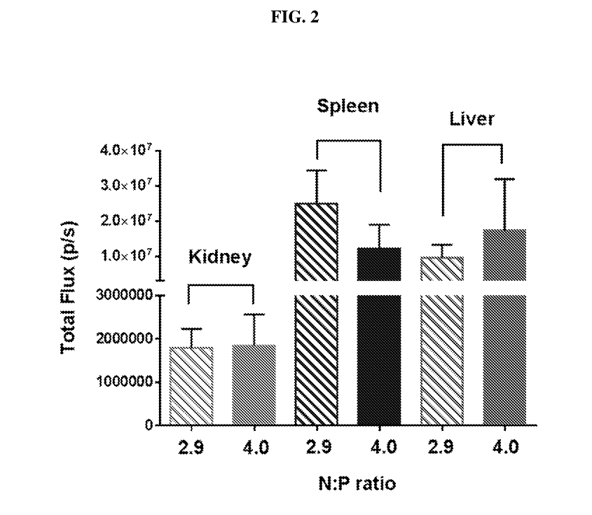

To test if different N:P ratios of C12-200 DSPC compositions affect kidney delivery, luciferase mRNA fully modified 1-methylpseudouridine (1 mpU) was formulated in a lipid nanoparticle comprising the lipids C12-200/DSPC with a N:P ratio of 2.9 or 4.0. The LNP with the N:P ratio of 2.9 comprised C12-200/DSPC/Cholesterol/PEG-DMG (30:19:49.5:1.5 mole percent; 21:1 lipid to mRNA weight ratio; particle size 122 nm; N:P ratio of 2.9; PDI: 0.17; encapsulation efficiency 92%) and the LNP with the N:P ratio of 4 comprised C12-200/DSPC/Cholesterol/PEG-DMG (30:19:49.5:1.5 mole percent; 29:1 lipid to mRNA weight ratio; particle size 125 nm; N:P ratio of 4; PDI: 0.19; encapsulation efficiency 90%). The expression of luciferase in the kidney, liver and spleen was imaged about 6 hours after administration of a single dose of luciferase mRNA (15 μg/kidney/0.5 mL) via delivery to the artery of the kidney. The luciferase expression patterns were summarized in Table 10 and a comparison between N:P 2.9 and NP4.0 is shown in FIG. 2.

TABLE 10
Luciferase expression patterns in C12-
200 DSPC NP2.9 and NP 4.0 compositions

| | Luc expression pattern | | |
|---|---|---|---|
| Formulation | kidney | Liver | Spleen |
| C12-200/DSPC | Good; diffused | High leak | High |
| N:P2.9 | expression and most | | leak |
| | in cortex; gross view | | |
| | of kidney was similar | | |
| | to normal view | | |
| C12-200/DSPC | Good; local | High leak but | High |
| N:P4.0 | expression mostly in | variable amounts | leak |
| | medullar area; gross | | |
| | view showed some | | |
| | edema and some | | |
| | bleeding in medullar | | |
| | area | | |

### Example 17. Comparison of KL10 Compositions for Kidney Delivery of Modified mRNA

KL10 compositions with different lipid components, particle sizes and N:P ratios were compared and analyzed.

### A. Lipid Comparison: DSPC and DOPE

Luciferase mRNA fully modified 1-methylpseudouridine (1 mpU) was formulated in a lipid nanoparticle comprising KL10/DOPE/Cholesterol/PEG-DMG (40:30:25:5 mole percent; 25:1 lipid to mRNA weight ratio; particle size 97 nm; N:P ratio of 4; PDI: 0.19; encapsulation efficiency 97%) or KL10/DSPC/Cholesterol/PEG-DMG (40:30:25:5 mole percent; 26:1 lipid to mRNA weight ratio; particle size 98 nm; N:P ratio of 4; PDI: 0.27; encapsulation efficiency 96%). The expression of luciferase in the kidney, liver and spleen was imaged about 6 hours after administration of a single dose of luciferase mRNA (15 μg/kidney/0.5 mL) via delivery to the artery of the kidney. The KL10/DSPC formulation showed greater expression levels in the spleen and liver as compared to the KL10/DOPE formulation suggesting a leak to the liver and spleen.

### B. Time Course

Luciferase mRNA fully modified 1-methylpseudouridine (1 mpU) was formulated in a lipid nanoparticle comprising the lipids KL10/DOPE/Cholesterol/PEG-DMG (40:30:25:5 mole percent; 25:1 lipid to mRNA weight ratio; particle size 100 nm; N:P ratio of 4; PDI: 0.23; encapsulation efficiency 99%). The expression of luciferase in the kidney, liver and spleen was imaged about 3 hours and 6 hours after administration of a single dose of luciferase mRNA (15 μg/kidney/0.5 mL) via delivery to the artery of the kidney. Higher expression levels were observed 3 hours after administration which suggests very transient expression.

### C. Particle Size

Luciferase mRNA fully modified 1-methylpseudouridine (1 mpU) was formulated in a lipid nanoparticle comprising KL10/DOPE/cholesterol/PEG-DMG at a ratio of 40:30:25:5 mole percent, a lipid to mRNA ratio of 25:1, a N:P ratio of 4 and a particle size of 62 nm (PDI: 0.31, encapsulation efficiency 98.8%), 100 nm (PDI: 0.23, encapsulation efficiency 99.3%) or 126 nm (PDI: 0.09, encapsulation efficiency 97.9%). The expression of luciferase in the kidney, liver and spleen was imaged about 3 hours after administration of a single dose of luciferase mRNA (15 μg/kidney/0.5 mL) via delivery to the artery of the kidney. The increase in particle size is correlated with an increase in liver/spleen signal.

### D. N:P Ratio

Luciferase mRNA fully modified 1-methylpseudouridine (1 mpU) was formulated in a lipid nanoparticle comprising the lipids KL10/DOPE/Cholesterol/PEG-DMG at a mole percent of 40:30:25:5, an N:P ratio of 3.1 (lipid:mRNA ratio of 20:1; size 72 nm, PDI: 0.24; encapsulation efficiency of 97.5%) or 4.0 (lipid:mRNA ratio of 25; size 58 nm, PDI: 0.18; encapsulation efficiency of 95.9%). The expression of luciferase in the kidney, liver and spleen was imaged about 3 hours after administration of a single dose of luciferase mRNA (15 μg/kidney/0.5 mL) via delivery to the artery of the kidney. The formulation with an N:P ratio of 3.1 and 4.0 showed no significant difference in expression and both formulations showed lower expression in the kidney.

### E. PEG Percentage

Luciferase expression and liver leakage was compared after kidney artery delivery of luciferase mRNA fully modified 1-methylpseudouridine (1 mpU) formulated in a lipid nanoparticle comprising KL10/DOPE/Cholesterol with PEG3% or PEG 5%. The expression of luciferase in the kidney, liver and spleen was imaged about 3 hours after administration of a single dose of luciferase mRNA (15 μg/kidney/0.5 mL) via delivery to the artery of the kidney. The data suggested that PEG content of the formulations had little to no effect on kidney expression or liver/spleen leakage.

### E. Expression and Morphology of the Kidney

Luciferase mRNA fully modified 1-methylpseudouridine (1 mpU) was formulated in a lipid nanoparticle comprising the lipids KL10/DOPE with PEG 1.5% or PEG 5.0%. The expression of luciferase in the kidney, sliced kidney, liver, spleen and kidney tissue was imaged about 3 hours after administration of a single dose of luciferase mRNA (15 μg/kidney/0.5 mL) via delivery to the artery of the kidney. The characteristics of the nanoparticle formulations and the expression and tissue results are shown in Table 11.

TABLE 11
Kidney morphology of KL10 DOPE formulation

| | Luc expression pattern | | | Kidney tissue |
|---|---|---|---|---|
| Formulation | kidney | Liver | Spleen | observation |
| KL10/DOPE (PEG 1.5%; | Similar to the PEG 5% | High leak as compared | High leak as compared | Damage in |
| cholesterol 28.5%; | formulation, but higher | to the PEG 5% | to the PEG 5% | medulla |
| particle size 87.8 nM, | than the PEG 5% | formuation | formuation | |
| PDI 0.098 and EE %98) | formulation | | | |
| KL10/DOPE (PEG 5%; | Similar to the PEG 1.5% | Low leak as compared | Low leak as compared | No clear |
| cholesterol 25%; | formulation, but higher | to the PEG 1.5% | to the PEG 1.5% | damage |
| particle size 85.0 nM, | than the PEG 1.5% | formuation | formuation | |
| PDI 0.274 and EE %99) | formulation | | | |

### Example 18. Comparison of Different Doses for KL10 and C12-200 Compositions

Three independent studies were performed to compare different doses in luciferase mRNA modified mRNA formulations for kidney delivery.

A. Dose Compare: 15 μg/Kidney/0.5 mL v. 45 μg/Kidney/0.5 mL

Luciferase mRNA fully modified 1-methylpseudouridine (1 mpU) was formulated in a lipid nanoparticle comprising KL10/DOPE/Cholesterol/PEG-DMG (40:30:25:5 mole percent; 25:1 lipid to mRNA weight ratio; particle size 64 nm; N:P ratio of 4; PDI: 0.29; encapsulation efficiency 97.4%). The expression of luciferase in the kidney, liver and spleen was imaged about 3 hours after administration of a single dose of 15 μg/kidney/0.5 mL luciferase mRNA or 45 μg/kidney/0.5 mL luciferase mRNA via delivery to the artery of the kidney. Kidney expression and liver leakage were increased in the 45 μg/kidney/0.5 mL dosing group as compared to the 15 μg/kidney/0.5 mL group.

B. Dose Compare: 15 μg/Kidney/0.5 mL v. 5 μg/Kidney/0.5 mL

Luciferase mRNA fully modified 1-methylpseudouridine (1 mpU) was formulated in a lipid nanoparticle comprising KL10/DOPE/Cholesterol/PEG-DMG (40:30:25:5 mole percent; 25:1 lipid to mRNA weight ratio; particle size 59 nm; N:P ratio of 4; PDI: 0.19; encapsulation efficiency 98%). The expression of luciferase in the kidney, sliced kidney, liver and spleen was imaged about 3 hours after administration of a single dose of 15 μg/kidney/0.5 mL luciferase mRNA or 5 μg/kidney/0.5 mL luciferase mRNA via delivery to the artery of the kidney. A dose-dependent expression the kidney was observed as lower kidney expression was seen with the 5 μg/kidney/0.5 mL dose as compared to 15 μg/kidney/0.5 mL dose.

### X. OTHER EMBODIMENTS

It is to be understood that the words which have been used are words of description rather than limitation, and that changes may be made within the purview of the appended claims without departing from the true scope and spirit of the invention in its broader aspects.

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

We claim:

1. A pharmaceutical composition comprising at least one mRNA, said at least one mRNA encoding a renal polypeptide of interest, wherein said at least one mRNA is formulated in a lipid nanoparticle.

2. The pharmaceutical composition of claim 1, wherein the at least one mRNA comprises at least one chemical modification.

3. The pharmaceutical composition of claim 2, wherein the chemical modification is 1-methylpseudouridine.

4. The pharmaceutical composition of claim 3, wherein the at least one mRNA also comprises the modification 5-methylcytosine.

5. The pharmaceutical composition of claim 1, wherein the lipid nanoparticle comprises at least one lipid selected from the group consisting of KL10, KL22, KL52, C12-200, DLin-KC2-DMA, DOPE, and DSPC.

6. The pharmaceutical composition of claim 5, wherein the lipid nanoparticle comprises the lipids KL10 and DOPE.

7. The pharmaceutical composition of claim 5, wherein the lipid nanoparticle comprises the lipids KL10 and DSPC.

8. The pharmaceutical composition of claim 5, wherein the lipid nanoparticle comprises the lipids C12-200 and DOPE.

9. The pharmaceutical composition of claim 5, wherein the lipid nanoparticle comprises the lipids C12-200 and DSPC.

10. The pharmaceutical composition of claim 5, wherein the lipid nanoparticle comprises the lipids KL22 and DOPE.

11. The pharmaceutical composition of claim 5, wherein the lipid nanoparticle comprises the lipids KL22 and DSPC.

12. The pharmaceutical composition of claim 5, wherein the lipid nanoparticle comprises the lipids DLin-MC3-DMA and DOPE.

13. The pharmaceutical composition of claim 5, wherein the lipid nanoparticle comprises the lipids DLin-MC3-DMA and DSPC.

14. The pharmaceutical composition of any of claims 5-13, wherein the lipid nanoparticle further comprises PEG.

15. The pharmaceutical composition of claim 14, wherein the lipid nanoparticle comprises between 1% and 7% of PEG.

16. The pharmaceutical composition of claim 15, wherein the amount of PEG is 1.5%.

17. The pharmaceutical composition of claim 15, wherein the amount of PEG is 3.0%.

18. The pharmaceutical composition of claim 15, wherein the amount of PEG is 5.0%.

19. The pharmaceutical composition of claim 1, wherein the lipid nanoparticle has an N:P ratio is between 2.5 and 7.

20. The pharmaceutical composition of claim 19, wherein the N:P ratio is between 2.5 and 3.5.

21. The pharmaceutical composition of claim 19, wherein the N:P ratio is between 2.5 and 4.

22. The pharmaceutical composition of claim 19, wherein the N:P ratio is between 4 and 6.

23. The pharmaceutical composition of claim 1, wherein the ratio of lipid to mRNA is 20:1.

24. The pharmaceutical composition of claim 1, wherein the ratio of lipid to mRNA is 10:1.

25. The pharmaceutical composition of claim 1, wherein the lipid nanoparticle has a particle size between 50 and 150 nm.

26. The pharmaceutical composition of claim 1, wherein the mRNA is encapsulated in the lipid nanoparticle with an encapsulation efficiency of greater than 50%.

27. A method of producing a renal polypeptide of interest in a kidney of a subject, said method comprising arterial administration to said subject of the pharmaceutical composition of claims 1-26.

28. The method of claim 27, wherein the mRNA is administered to an artery at a dose of between 5-45 μg per 0.5 mL per kidney.

29. The method of claim 28, wherein the dose is 5 μg per 0.5 mL per kidney.

30. The method of claim 28, wherein the dose is 15 μg per 0.5 mL per kidney.

31. The method of claim 28, wherein the dose is 30 μg per 0.5 mL per kidney.

32. The method of claim 28, wherein the dose is 45 μg per 0.5 mL per kidney.

33. The method of claim 1, wherein the expression of the renal polypeptide of interest is increased in the kidney for at least 3 hours.

34. The method of claim 1, wherein the expression of the renal polypeptide of interest is increased in the kidney for at least 6 hours.

35. The method of claim 1, wherein the expression of the renal polypeptide of interest is increased in the kidney for at least 20 hours.

36. A method of treating a renal disease, disorder or condition, said method comprising arterial administration to said subject of the pharmaceutical composition of claims 1-26.

37. The method of claim 36, wherein the renal disease, disorder or condition is selected from the group consisting of primary glomerular disease, cystic renal disease and renal tubular disease.

38. The method of claim 37, wherein the renal disease, disorder or condition is primary glomerular disease and wherein the primary glomerular disease is selected from the group consisting of Alport's syndrome (X-linked or autosomal recessive), benign familiar hematuria, congenital nephrosis I, nail patella syndrome and familial mesangial sclerosis.

39. The method of claim 37, wherein the renal disease, disorder or condition is cystic renal disease and wherein the cystic renal disease is selected from the group consisting of polycystic kidney disease 1 (PKD1), polycystic kidney disease 2 (PKD2), and infantile severe polycystic kidney disease with tuberous sclerosis.

40. The method of claim 37, wherein the renal disease, disorder or condition is renal tubular disease and wherein the renal tubular disease is selected from the group consisting of distal renal tubular acidosis, renal tubular acidosis with neural deafness, renal tubular acidosis with osteoporosis, Dent's disease, Nephrogenic diabetes insipidus (X-linked), Nephrogenic diabetes insipidus (autosomal), familial hypocalcuric hypercalcemia, pseudovitamin D deficiency rickets, X-linked hypophosphatemia, Gitelman's syndrome, Bartter's syndrome type 1, Bartter's syndrome type 2, Bartter's syndrome type 3, Pseudoaldosteronism (Liddle syndrome), Recessive pseudohypoaldosteronism type 1, dominant pseudohypoaldosteronism type I, apparent mineralocorticoid excess, Cystinuria type I and Cystinuria non-type I.

41. The method of claim 36, wherein the mRNA is administered to an artery at a dose of between 5-45 μg per 0.5 mL per kidney.

42. The method of claim 41, wherein the dose is 5 μg per 0.5 mL per kidney.

43. The method of claim 41, wherein the dose is 15 μg per 0.5 mL per kidney.

44. The method of claim 41, wherein the dose is 30 μg per 0.5 mL per kidney.

45. The method of claim 41, wherein the dose is 45 μg per 0.5 mL per kidney.
